(19) United States
(12) Patent Application Publication
(10) Pub. No.: US 2024/0219385 A1
(43) Pub. Date: Jul. 4, 2024

(54) MOLECULAR SIGNATURES OF LONG-TERM COVID-19 AND TREATMENT THEREOF

(71) Applicants: ALLEN INSTITUTE, Seattle, WA (US); FRED HUTCHINSON CANCER CENTER, Seattle, WA (US)

(72) Inventors: Thomas F. BUMOL, Seattle, WA (US); Xiaojun LI, Seattle, WA (US); Peter SKENE, Seattle, WA (US); Gregory Lee SZETO, Seattle, WA (US); Aarthi TALLA, Seattle, WA (US); Troy TORGERSON, Seattle, WA (US); Mark-Phillip PEBWORTH, Seattle, WA (US); Suhas VASAIKAR, Seattle, WA (US); Juliana M. MCELRATH, Seattle, WA (US)

(73) Assignees: ALLEN INSTITUTE, Seattle, WA (US); FRED HUTCHINSON CANCER CENTER, Seattle, WA (US)

(21) Appl. No.: 18/557,406

(22) PCT Filed: Apr. 28, 2022
(86) PCT No.: PCT/US22/26841

§ 371 Date: Oct. 26, 2023

Related U.S. Application Data
(60) Provisional application No. 63/181,215, filed on Apr. 28, 2021.

(51) Int. Cl. G01N 33/569

(52) U.S. Cl. CPC G01N 33/56983; G01N 2333/165; G01N 2333/521; G01N 2333/525; G01N 2333/5409; G01N 2333/5431; G01N 2333/5434; G01N 2333/5443; G01N 2333/545; G01N 2333/57; G01N 2333/70521

(57) ABSTRACT

In various embodiments, provided are immune response signatures that can be used for the diagnosis, monitoring, and treatment of long-term diseases and inflammatory disorders caused by viral infections. In some embodiments, the viral infection is SARS-CoV-2 infection. In some embodiments, the long-term disease is post-acute sequelae of SARS-CoV-2 infection (PASC).

### BACKGROUND

Severe acute respiratory syndrome-related coronavirus (SARS-CoV-2) is a novel, highly infectious betacoronavirus that was first detected in late 2019 and causes COVID-19 respiratory disease in humans. According to the COVID-19 Dashboard by the Center for Systems Science and Engineering (CSSE) at Johns Hopkins University and the World Health Organization (WHO) COVID-19 Dashboard, SARS-CoV-2 has precipitated an ongoing pandemic that has infected more than 120 million people and killed nearly three million people worldwide. Clinical presentation of COVID-19 after infection is characterized by multiple features, including ground glass opacities on lung x-rays and fever with loss of taste and smell, but wide interpatient heterogeneity exists in disease severity, with outcomes ranging from asymptomatic or mild to severe and fatal.

The scale of worldwide infection underscores the vast public health consequences of mild COVID-19 infection, defined as those receiving outpatient care. COVID-19 has an estimated 1% fatality rate and up to 20% severe disease incidence, but the vast majority (about 80%) of infections are mild (Wu and McGoogan 2020). There remains significant morbidity among mild COVID-19 patients spanning a range of symptom durations and post-infection complications, which includes post-acute sequelae of SARS-CoV-2 infection (PASC, also known as long haulers or long COVID-19). PASC is an umbrella designation for clinical symptoms persisting weeks to months post-infection with incidence estimated at 30% to more than 70% of mild infections (Davis et al. 2020; Huang et al. 2021; Logue et al. 2021; Dennis et al.; Sudre et al. 2021), but little is known about mild COVID-19 and PASC. PASC presents with heterogeneous lingering symptoms spanning nearly every bodily system, including fatigue, “brain fog”, fever, anxiety, and even symptoms mimicking new onset rheumatologic disease. There are currently no consensus diagnostic criteria for PASC, and much of its treatment relies on subjective self-reported symptomology. Thus, there remains a need for cellular and molecular phenotyping of the immune response in mild COVID-19 infection to identify the key immunological mechanisms used in early infections, the major drivers of heterogeneity in convalescent immunity, and to find signatures that predict or define clinical outcomes such as PASC.

### SUMMARY

The present technology relates to immune response signatures that can be used for the diagnosis, monitoring, and treatment of long-term diseases and inflammatory disorders caused by viral infections, including PASC.

In some aspects, provided are methods of diagnosing or classifying a subject as having a chronic or long-term infection of a virus, bacterium, fungus, and/or parasite, and/or an autoimmune disease, the methods comprising determining the level of one or more biomarkers in a biological sample obtained from a subject. In some embodiments, the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV).

In some embodiments, the one or more biomarkers comprise proteins associated with an inflammatory response, wherein the proteins associated with an inflammatory response comprise one or more of TNF, IFNLR1, BCAM, S100A16, and IL5.

In some embodiments, the one or more biomarkers comprise cytokines, chemokines, and/or immunomodulatory proteins, wherein the cytokines, chemokines, and/or immunomodulatory proteins comprise one or more of TNF, IL5, IL11, IL13, IL15, IL1B, CXCL1, CXCL8, CCL3, CCL11, IL1RL2, CD28, HLA-DRA, LAG3, and PDCD1.

In some embodiments, the one or more biomarkers comprise hormones and hormone receptors, wherein the hormones and hormone receptors comprise one or more of CRH, CRHR1, and PTH1R.

In some embodiments, the one or more biomarkers comprise transcription factors and motifs thereof, wherein the transcription factors and motifs thereof comprise one or more of AP-1, BACH, BATF, IRF, and STAT.

In some aspects, provided are methods of treating or prevent one or more symptoms in a subject diagnosed or classified as having a chronic or long-term infection of a virus, bacterium, fungus, and/or parasite, and/or an autoimmune disease, the methods comprising administering to the subject one or more therapeutic agents. In some embodiments, the subject is diagnosed or classified as having post-acute sequelae of SARS-CoV-2 infection (PASC, also known as a “long-hauler”).

In some embodiments, the one or more therapeutic agents comprise an anti-inflammatory agent.

In some embodiments, the one or more therapeutic agents are administered to the subject for a period of time of about 3 days to about 5 years.

In some embodiments, the methods further comprise monitoring the subject for the one or more symptoms of the long-term infection of the virus, bacterium, fungus, and/or parasite, and/or the autoimmune disease.

In some aspects, provided is a molecular signature for use in determining whether a subject infected with or previously infected with a virus or other pathogen is likely to suffer from a chronic inflammatory syndrome with or without a chronic or long-term infection of the virus or other pathogen, the molecular signature comprising one or more inflammatory proteins or biomarkers that are enriched in the subject relative to an uninfected or recovered control subject.

In some embodiments, the one or more inflammatory proteins or biomarkers is selected from the group consisting of: TNF, IFNLR1, BCAM, S100A16, and IL5.

In some embodiments, the one or more inflammatory proteins or biomarkers is selected from the group consisting of: TNF, IL5, IL11, IL13, IL15, IL1B, CXCL1, CXCL8, CCL3, CCL11, IL1RL2, CD28, HLA-DRA, LAG3, and PDCD1.

In some embodiments, the one or more inflammatory proteins or biomarkers is selected from the group consisting of: CRH, CRHR1, and PTH1R.

In some embodiments, the one or more inflammatory proteins or biomarkers is selected from the group consisting of: AP-1, BACH, BATF, IRF, and STAT.

In some embodiments, the one or more inflammatory proteins is selected from the group consisting of: type II interferon (IFN), NF-κB, NF-κB-activating cytokine, IL-12, p40, IFN-γ-driven chemokine, TNF-driven cytokine and chemokine, Type I IFN, cytokine, IFNA, and IL-12.

In some embodiments, the enrichment of the type II IFN is associated with the enrichment of one or more of Type II IFN-γ, IL-27, and TID.

In some embodiments, the enrichment of the Type II IFN-γ is associated with the enrichment of one or more of IL-27, IL-18, and NF-κB.

In some embodiments, the enrichment of the NF-κB is associated with the enrichment of TNF.

In some embodiments, the enrichment of the TNF is associated with the enrichment of one or more of IL-1 and IL-18.

In some embodiments, the enrichment of the NF-κB-activating cytokine is associated with the enrichment of one or more of IL-18, TNF, and IL-1.

In some embodiments, the enrichment of the TNF-driven cytokine and chemokine is associated with the enrichment of one or more of IL-6, CCL7, and MCP3.

In some embodiments, the enrichment of the Type I IFN is associated with the enrichment of one or more of SAMD9L, MNDA, DDX58, and LAMP3.

In some embodiments, the enrichment of the cytokine is associated with the enrichment of one or more of IFN-γ, IFN-β, IFN-λ1/2/3, TNF, IL-6, IL-1β, and PTX3.

In some embodiments, the molecular signature is a serum proteome signature.

In some embodiments, the enrichment is between 1.5-fold and 10-fold as compared to an uninfected or recovered control subject.

In some embodiments, the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV).

In some embodiments, the virus is SARS-CoV-2.

In some embodiments, the chronic inflammatory syndrome is post-acute sequelae of SARS-CoV-2 infection (PASC).

In some embodiments, the subject is likely to have persistent symptoms lasting a specific period after onset of the infection.

In some embodiments, the specific period is between 30 days and 2 years.

In some aspects, provided is a molecular signature for use in diagnosing a subject as having a chronic inflammatory syndrome with or without a chronic or long-term infection with a virus or other pathogen, the molecular signature comprising one or more inflammatory proteins or biomarkers that are enriched in the subject relative to an uninfected or recovered control subject.

In some embodiments, the one or more inflammatory proteins or biomarkers is selected from the group consisting of: TNF, IFNLR1, BCAM, S100A16, and IL5.

In some embodiments, the one or more inflammatory proteins or biomarkers is selected from the group consisting of: TNF, IL5, IL11, IL13, IL15, IL1B, CXCL1, CXCL8, CCL3, CCL11, IL1RL2, CD28, HLA-DRA, LAG3, and PDCD1.

In some embodiments, the one or more inflammatory proteins or biomarkers is selected from the group consisting of: CRH, CRHR1, and PTH1R.

In some embodiments, the one or more inflammatory proteins or biomarkers is selected from the group consisting of: AP-1, BACH, BATF, IRF, and STAT.

In some embodiments, the one or more inflammatory proteins is selected from the group consisting of: type II interferon (IFN), NF-κB, NF-κB-activating cytokine, IL-12, p40, IFN-γ-driven chemokine, TNF-driven cytokine and chemokine, Type I IFN, cytokine, IFNA, and IL-12.

In some embodiments, the enrichment of the type II IFN is associated with the enrichment of one or more of Type II IFN-γ, IL-27, and TID.

In some embodiments, the enrichment of the Type II IFN-γ is associated with the enrichment of one or more of IL-27, IL-18, and NF-κB.

In some embodiments, the enrichment of the NF-κB is associated with the enrichment of TNF.

In some embodiments, the enrichment of the TNF is associated with the enrichment of one or more of IL-1 and IL-18.

In some embodiments, the enrichment of the NF-κB-activating cytokine is associated with the enrichment of one or more of IL-18, TNF, and IL-1.

In some embodiments, the enrichment of the TNF-driven cytokine and chemokine is associated with the enrichment of one or more of IL-6, CCL7, and MCP3.

In some embodiments, the enrichment of the Type I IFN is associated with the enrichment of one or more of SAMD9L, MNDA, DDX58, and LAMP3.

In some embodiments, the enrichment of the cytokine is associated with the enrichment of one or more of IFN-γ, IFN-β, IFN-λ1/2/3, TNF, IL-6, IL-1β, and PTX3.

In some embodiments, the molecular signature is a serum proteome signature.

In some embodiments, the enrichment is between 1.5-fold and 10-fold as compared to an uninfected or recovered control subject.

In some embodiments, the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV).

In some embodiments, the virus is SARS-CoV-2.

In some embodiments, the chronic inflammatory syndrome is post-acute sequelae of SARS-CoV-2 infection (PASC).

In some embodiments, the subject is likely to have persistent symptoms lasting a specific period after onset of the infection.

In some embodiments, the specific period is between 30 days and 2 years.

In some aspects, provided are methods of identifying whether a subject infected with or previously infected with a virus or other pathogen is likely or not likely to suffer from a chronic inflammatory syndrome with or without a chronic or long-term infection of the virus or other pathogen, comprising: (a) determining an expression level of one or more inflammatory proteins or biomarkers of the molecular signature of any one of claims 12 to 27 or 33 to 48 in a first sample obtained from the subject; (b) comparing the first expression level to a control expression level obtained from an uninfected or recovered control subject; and (c) classifying the subject as likely to suffer from a chronic or long-term infection of the virus or other pathogen when the expression level corresponds to the molecular signature of any one of claims 12 to 27 or 33 to 48.

In some embodiments, the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV).

In some embodiments, the virus is SARS-CoV-2.

In some embodiments, the chronic inflammatory syndrome is post-acute sequelae of SARS-CoV-2 infection (PASC).

In some embodiments, the sample is obtained within the first 15 days of post-symptom onset.

In some embodiments, the subject is placed into a cohort for a clinical trial to test investigational drugs to treat PASC.

In some embodiments, the subject is administered a drug for treating PASC.

### BRIEF DESCRIPTION OF THE DRAWINGS

FIG. 1A shows cohort overview and participant demographics. Two COVID-19 participants were excluded after applying quality control criteria. FIG. 1B shows longitudinal sampling timeline. PBMCs and serum were collected for 3-5 timepoints for each participant. Younger participants (top) are light blue; older participants (bottom) are light orange. Each Gantt is annotated with documented comorbidities and presentation of PASC. FIG. 1C shows sample availability enumerated per assay type. Peripheral blood mononuclear cells (PBMCs) were analyzed by scRNAseq, scATACseq, spectral flow cytometry, and antigen-specific ICS assays. Serum were analyzed for SARS-CoV-2 antibody serology or proteomics by Olink Explore 1536. Absent samples were due to limitations on material availability or timing.

FIGS. 2A-2I show persistent immune hyperactivation and dysfunction characterized post-acute sequelae of SARS-CoV-2 infection (PASC) participants. FIG. 2A shows an overview of symptom persistence for PASC participants. None had fully recovered at time of latest follow-up for publication (233 days). FIG. 2B shows SARS-CoV-2-specific adaptive immune responses estimated at day 30 showed few differences between PASC (n=3) and recovered COVID-19 (n=15) participants. FIG. 2C shows fraction of serum protein differences over time normalized to visit 1 for each COVID-19 participant. Number of outlier proteins persisted or increased up to ˜30 days PSO in PASC while they resolved in most convalescent COVID-19 participants. Outlier analysis was performed comparing COVID-19 participants to uninfected background and selecting features >2 standard deviations from the mean. FIG. 2D shows differentially expressed serum proteins (linear mixed model, p<0.05) demonstrated enrichment of cytokine and chemokine signaling pathways and persistent expression over time. FIG. 2E shows pathway enrichment analysis of scRNAseq DEGs from CD14+ monocytes. TNF signaling and hypoxia were persistently elevated in PASC, while interferon and RIG-I responses were low and persistent throughout infection. FIG. 2F shows transcription factor motif analysis of scATACseq data in PASC in samples >30 days PSO. Innate immune cells (DCs, CD14+ monocytes, CD16+ monocytes) and effectors (CD4 TEM+CTL, CD8 TEM) showed the most enrichment suggesting persistent activation during PASC. Heatmap showed the top 50 largest motif differences (Wilcoxon FDR<0.10) calculated using ChromVar motif Z-scores. FIG. 2G shows metaclustering identified non-specific CD4+Th1 TEM cells as negatively correlated with PASC. This metacluster was significantly lower in PASC participants after acute infection, >30 days PSO. FIG. 2H shows ligand-receptor interaction analysis predicted inflammatory cytokine signaling as top signals driving hyperactivation of innate (CD14 monocyte) and effector (CD8 TEM) cells in PASC patients. FIG. 2I shows serum levels of selected upregulated proteins based on predicted ligands (TNF, CD28, IL12p70) or linear mixed effects modeling (IL-5) in PASC participants.

FIGS. 3A-3K show integrative analysis uncovering key nodes for immunomonitoring and potential therapeutic targets. FIG. 3A shows differentially expressed immune-related proteins (n=75, p<0.05) observed in early acute infection (<=15 days PSO), longitudinally and in PASC COVID-19 subjects derived from serum proteomics study. FIG. 3B shows correlation analysis between differentially expressed proteins, IgG/IgM/IgA RBD titers, and S-specific plasmablasts to identify relationships between PASC and SARS-CoV-2-specific immune responses. FIG. 3C shows Nichenet based intracellular communication analyses of single cell RNA data from early acute COVID-19 infection subjects. We retrieved top 10 inferred ligands influencing the ligand-target expression in receiver cell types. The triangle shows the ligand and circle represents the receiver cell type. The edge between nodes and ligand shows the inferred relationship in early acute COVID-19 infection from scRNA data. The size of the triangle is proportional with the number of edges outgoing. The top 10 inferred ligands (per celltype) (FIG. 3D), ligand-receptor interactions (per celltype) (FIG. 3E) and ligand-targets (per ligand) (FIG. 3F) in early acute COVID-19 infection were shown. FIG. 3G shows the overlap between the inferred ligand-receptor interactions from differential intercellular communication between early acute COVID-19, longitudinal and PASC participants with controls as healthy, COVID-19 at <=15 days PSO and non-PASC recovered COVID-19 participants, respectively. Longitudinal changes in ligand (FIG. 3H), ligand-receptor (FIG. 3I), ligand-target (FIG. 3J) usage in early acute COVID-19, longitudinal and PASC participants respectively were shown. FIG. 3K shows that overall, the data reveals possible mechanistic overview where the early acute COVID-19 infection.

FIGS. 4A-4G show serum proteomic clustering of PASC. FIG. 4A shows a heatmap of the rule-in method based unsupervised clustering of Olink serum proteome data across all patients in the cohort (PASC+recovered+uninfected). Rows represent modules, columns represent samples and the scaled ssGSEA module score across samples is depicted from low (purple) to high (yellow). The method identifies 2 clusters of subjects with higher inflammatory module signatures (4 & 5) relative to the other three clusters of subjects (1, 2, 3) that lack inflammatory signatures. Metadata including age, sex, symptoms, days post-symptom onset (PSO) are shown at the top of the heatmap. FIG. 4B shows a clinical activity score of PASC subjects in inflammatory (4 & 5) vs. non-inflammatory (2 & 3) clusters. The p-value determined by Wilcoxon rank sum test was calculated comparing, as a group, inflammatory PASC vs non-inflammatory PASC. FIG. 4C shows receptor binding domain (RBD)-specific IgG titers in PASC and recovered patients within each cluster. The p-value determined by Wilcoxon rank sum test was calculated comparing, as a group, inflammatory clusters vs non-inflammatory clusters. FIGS. 4D-4F show box and jitter plots of the ssGSEA scores (y-axis) across all clusters (x-axis) for the top ranked modules that were enriched in inflammatory clusters 4 and 5. P-values determined by wilcoxon rank sum test were calculated comparing inflammatory cluster 4 and inflammatory cluster 5 independently to clusters 1,2,3. FIG. 4G shows pair-wise Spearman's correlation coefficient heatmap between top enriched modules that define inflammatory clusters 4 and 5 demonstrating co-enrichment of modules.

FIGS. 5A-5L show key protein signals driving inflammatory PASC signatures. FIG. 5A shows top ranked differentially expressed cytokines, chemokines, and cytokine/chemokine receptors by adjusted p-value of <0.05 that are associated with inflammatory protein clusters 4 & 5. The color gradient of each node represents the −log 10 adjusted p-value. FIG. 5B shows box and jitter plots of olink Normalized Protein Expression (NPX) (y-axis) of IFN-γ and its related cytokines and chemokines across clusters (x-axis) that were significantly upregulated exclusively in cluster 4. P-values determined by wilcoxon rank sum test were calculated comparing inflammatory cluster 4 and inflammatory cluster 5 independently to clusters 1,2,3. FIG. 5C shows longitudinal loess fit plots of Olink NPX of IFN-γ and its related cytokines and chemokines on samples available from early acute infection through >60 days PSO (x-axis). PASC patients from the inflammatory clusters 4 and 5 are represented here as inflammatory PASC (red), PASC patients from clusters 2 and 3 are represented here as non-inflammatory PASC (blue) while the recovered patients are represented in black. FIG. 5D shows longitudinal loess fit plots of the ssGSEA scores (y-axis) of IFN-γ related modules over time (x-axis). FIG. 5E shows box and jitter plots of olink NPX (y-axis) expression levels of TNF, IL6 and CCL7 across clusters (x-axis) that were significantly differentially upregulated clusters 4 and 5. P-values determined by wilcoxon rank sum test were calculated comparing inflammatory cluster 4 and inflammatory cluster 5 independently to clusters 1,2,3. FIG. 5F shows longitudinal loess fit plots of Olink NPX (y-axis) of TNF, IL6 and CCL7 over time (x-axis). FIG. 5G shows longitudinal loess fit plots of the ssGSEA scores (y-axis) of TNF and NF-κB related signaling modules over time (x-axis). FIGS. 5H-5I show longitudinal loess fit plots of Olink NPX and ssGSEA scores (y-axes) of type-I IFN-driven proteins and the IFN-α module overtime (x-axis) respectively. FIG. 5J shows K-means unsupervised clustering of Olink proteomic data from Su Y et al (2022) showing 5 clusters of INCOV patients and healthy controls. Pie charts show the percentage of each cluster consisting of INCOV patients and healthy subjects. FIG. 5K shows specific cytokines/chemokines significantly upregulated in the INCOV cluster INCOV E vs. INCOV from clusters B,C,D. P-values were determined by a Wilcoxon rank sum test. (FIG. 5L) Distribution of different disease severities (as judged by WHO ordinal scale) across INCOV patients in cluster E vs INCOV patients in clusters B,C,D. Y-axis and the numbers in bar graphs represent proportion and number of patients per INCOV group in each WHO scale bin respectively.

FIGS. 6A-B show data of demographics and symptoms of a cohort of infected and uninfected subjects (FIG. 6A) and a graph of hierarchical clustering on PASC symptomatology (FIG. 6B).

FIGS. 7A-D show hierarchical clustering data for a cohort of PASC patients based on PASC symptomatology. Hierarchical clustering on PASC symptomatology alone at ≥60 days post symptom onset (PSO) did not clearly drive significant patient clustering (FIG. 7A). Subsequently, symptoms were attempted to be used to drive clustering of significantly associated serum protein signatures, but no single symptom or combination of symptoms was able to clearly distinguish patient groups (FIG. 7B, FIG. 7C, FIG. 7D).

FIGS. 8A-D show hierarchical clustering data for a cohort of PASC patients based on PASC symptomatology, with inflammatory clusters 4 and 5 including predominantly PASC subjects (91% and 80% respectively), cluster 1 consisting of only uninfected or recovered subjects, and clusters 2 and 3 consisting of a mixture of PASC (48% and 28% respectively), recovered, and uninfected subjects (FIG. 8A). PASC subjects that had an inflammatory protein signature continued to have that signature over time and most subjects remained in the same cluster throughout the study period (FIG. 8B). Non-inflammatory PASC clusters (2, 3, and 4) and Inflammatory PASC clusters (4 and 5) were mapped against % reactive CD4+ T-cells (FIG. 8C) and % reactive CD8+ T-cells (FIG. 8D).

FIG. 9 shows a depiction of those modules among the 54 PASC-symptomatology-based modules that defined the 5 clusters that were identified as significantly distinguishing each cluster by calculating the single-sample-Gene Set Enrichment Analysis (ssGSEA) score per module across samples.

FIG. 10 shows a depiction of ranking the 54 PASC-symptomatology-based modules that defined the 5 clusters by adjusted p-value, identifying those most significantly associated with clusters 4 and 5.

FIGS. 11A-B show depictions of proteins associated with type I IFN activation, characterized by increased expression, with IFN-γ at the top DEP enriched in cluster 4 among all 1463 analytes in the Olink protein panel. The Olink assay only quantified IFN-γ and IFNλ1, but increased expression of proteins associated with type I IFN activation including SAMD9L, MNDA, DDX58, LAMP3, and others was observed.

FIG. 12 shows graphs illustrating that proteins associated with type I IFN activation, including SAMD9L, MNDA, DDX58, LAMP3, and others, were characterized by increased expression.

FIG. 13 shows graphs of IFN-γ, IL-12 p40, and IFN-γ-driven chemokines that were consistently elevated within inflammatory PASC from clusters 4 and 5 compared to non-inflammatory PASC from clusters 1, 2, and 3, extending to at least 275 days after initial SARS-CoV-2 infection.

FIGS. 14A-C show graphs of IFN-γ related signaling modules showing persistent enrichment over the same time.

FIG. 15 shows graphs of patients from Cluster E showing significant enrichment of 128 of the 163 proteins that defined the inflammatory PASC described herein (78.5%).

### DETAILED DESCRIPTION

The present technology provides methods and compositions for identification of molecular and cellular features from patient samples distinguishing long-term viral infections (e.g., PASC resulting from SARS-CoV-2 infection) from recovered controls, which in turn enable confirmatory diagnosis and classification of long infection patients, disease staging, and immunomonitoring and identification of therapeutic strategies for these patients.

Coordination of immune dynamics is critical to provide early innate control of viral replication and to efficiently prime of virus-specific adaptive immune responses. Failure to coordinate dynamics or magnitude of inflammation may drive disease severity via dysregulation of both innate and adaptive immunity, with pleiotropic effects such as lymphopenia of select immune cells (Sette and Crotty 2021; Schultze and Aschenbrenner 2021; Carvalho et al. 2021). A persistent inflammatory state is a common feature among adults who experience more severe and fatal disease and multisystem inflammatory syndrome in children (MIS-C), with reports of elevated cytokine profiles bearing similarity to hemophagocytic lymphohistiocytosis (HLH) and cytokine release syndrome (CRS), immunosuppressive innate immune cells, and in some cases, autoantibodies targeting cytokines such as type I interferons (IFNs) (Bastard et al. 2020; Wang et al. 2020).

Systems immunology studies of blood and tissues from SARS-CoV-2 infected patients have focused on elucidating potential mechanisms and changes underlying moderate and severe disease for COVID-19 and MIS-C (Stephenson et al. 2021; Liu et al. 2021; Arunachalam et al. 2020; Laing et al. 2020; Combes et al. 2021; Lee et al. 2020; Chua et al. 2020; Maucourant et al. 2020; Zhang et al. 2020; Overmyer et al. 2021; Mathew et al. 2020; Zhu et al. 2020; Ren et al. 2021; Wilk et al. 2020; Filbin et al. 2020; Giroux et al. 2020; Kusnadi et al. 2021; Su et al. 2020; Pairo-Castineira et al. 2021; Zheng et al. 2020; Overholt et al. 2020; Guo et al. 2020; Huang et al. 2021; Zhou et al. 2020; Koutsakos et al. 2021; Galani et al. 2021; Lucas et al. 2020; Bolouri et al. 2021; Rodriguez et al. 2020). Many of the expected immunologic signals responding to an acute viral infection have been observed, but alterations have also been identified in every major immune cell subset during COVID-19, including effectorized and exhausted natural killer (NK) cells (Wilk et al. 2020; Maucourant et al. 2020), elevated inflammatory/non-classical monocytes and activated B cells (Stephenson et al. 2021; Ren et al. 2021), decreased proliferation and hyperexhaustion/terminal effectorization of CD4+ and CD8+ T cells (Stephenson et al. 2021; Zhou et al. 2020), reduced function and numbers of conventional and plasmacytoid dendritic cells (cDCs, pDCs) (Liu et al. 2021), and higher abundance of low-density granulocytes and immature neutrophils, megakaryocytes, and platelets. Severe and fatal infections showed delayed type I and III interferon responses, defects in T follicular helper (Tfh) and extrafollicular B cells (Kaneko et al. 2020), and poor virus-specific T cell responses. Multiple severity-associated signatures have also been reported in metabolic and serum protein profiles (Su et al. 2020). These findings suggest far-reaching disease pathogenesis driven by immune dysregulation unlike non-pandemic viral respiratory infections.

Hospitalized COVID-19 cases (moderate, severe, fatal) are immunologically distinct, but fewer differences have been noted in mild disease. Some studies report mild COVID-19 is largely indistinguishable from uninfected or healthy controls. This is surprising given data showing robust adaptive immune responses developed against SARS-CoV-2 in nearly all mild infections, and consistent estimates from studies in the US, Europe, and China that report less than 30% of COVID-19 patients of all severities report long-term persistent symptoms. Part of this discrepancy may be due to timing, where immune signaling in mild COVID-19 may largely resolve before sampling can occur. Most acute viral infections rapidly induce interferons (hours to days post-infection) which then mobilize and activate innate cells to generate adaptive immune responses via T and B cell activation. Interferons have delayed dynamics or are absent in severe COVID-19 (Galani et al. 2021; Hadjadj et al. 2020; Lucas et al. 2020), and the success of COVID-19 as a pathogen suggests insufficient innate immune control over viral replication. Interferon deficits that lead to more severe pathology have multiple causes, such as inefficient interferon response in infected cells (Blanco-Melo et al. 2020), and intrinsic type I IFN deficits such as somatic mutations or anti-interferon autoantibodies, which all result in poor priming of virus-specific adaptive immunity (Zhang et al. 2020; Wang et al. 2020; Bastard et al. 2020). This dysregulation can drive pathologic damage through a combination of untamed viral replication leading to over-exuberant and prolonged innate immune responses including persistent serum inflammatory cytokines such as interleukin (IL)-18. There are few studies that apply similar deep multi-omic analyses of immunity in outpatient mild disease for fewer than 2 longitudinal visits, partially due to the complexity of early patient recruitment and longitudinal sampling in outpatient care. Despite advances in understanding more severe COVID-19, it remains unclear what immune features characterize an average mild COVID-19 case, how these affect the priming of virus-specific adaptive immune responses, and which of these may explain convalescent heterogeneity.

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

### Definitions

The term “about,” as used herein when referring to a measurable value such as an amount or concentration and the like, is meant to encompass variations of 20%, 10%, 5%, 1%, 0.5%, or even 0.1% of the specified amount.

The term “immune cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include B cells, T cells (e.g., CD4+ T cells, CD8+ T cells, regulatory T cells), NK cells, and dendritic cells. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

The term “adaptive immune response” refers to an immunity that occurs after exposure to an antigen either from a pathogen or a vaccination. This part of the immune system usually is activated when the innate immune response is insufficient to control an infection. There are two major types of adaptive responses: the cell-mediated immune response, which is carried out by T cells; and the humoral immune response, which is controlled by activated B cells and antibodies. Activated T cells and B cells that are specific to molecular structures on the pathogen proliferate and attack the invading pathogen. Their attack can kill pathogens directly or secrete antibodies that enhance the phagocytosis of pathogens and disrupt the infection.

### Methods of Diagnosis and Classification

Long COVID or post-acute sequelae of SARS-CoV-2 (PASC) is a clinical syndrome characterized by diverse symptoms that persist for months after acute SARS-CoV-2 infection. One-third or more of surviving COVID-19 patients experience at least 1 PASC symptom during the 2-5 months after the onset of acute infection (Groff et al. 2021). PASC symptoms are numerous and varied, impacting virtually every major organ system (Nalbandian et al. 2021, Wang et al. 2022). Despite the large number of individuals affected, there are no consensus diagnostic criteria or standardized outcome measures that allow subjects to be grouped effectively for clinical comparison or for therapeutic trials (Munblit et al. 2022). There are also no clearly defined molecular markers of disease or definitive diagnostic tests. To make matters more complicated, it is recognized that similar clinical symptoms could arise after acute COVID-19 regardless of whether they were caused by persistent inflammatory disease initiated by immune response to the virus, unresolved organ or tissue damage, or delayed viral clearance. Identification of molecular features capable of mechanistically defining the heterogeneity of PASC could be transformative, allowing clinicians and researchers to better subset patients and highlighting potential targets for therapeutic intervention. The etiologies of PASC are unknown but may include persistent inflammation, unresolved tissue damage, or delayed clearance of viral protein or RNA. Attempts to classify subsets of PASC by symptoms alone have been unsuccessful.

In some embodiments, a clinically accessible tool to help define subgroups of PASC comprises analyzing serum proteome to provide insights into potential drivers of PASC symptomatology. In some embodiments, PASC is molecularly defined by evaluating the serum proteome in longitudinal samples from PASC subjects with persistent symptoms lasting a specific period after onset of a PCR-confirmed SARS-CoV-2 infection, i.e., acute infection, and comparing the results to those of symptomatically recovered SARS-CoV-2 infected and uninfected individuals. In some embodiments, the specific period is at least 30 days. In some embodiments, the specific period is at least 45 days. In some embodiments, the specific period is at least 60 days. In some embodiments, the specific period is at least 75 days. In some embodiments, the specific period is at least 90 days. In some embodiments, the specific period is between 30 days and 90 days. In some embodiments, the specific period is between 90 days and 180 days. In some embodiments, the specific period is between 180 days and 1 year. In some embodiments, the specific period is more than 1 year.

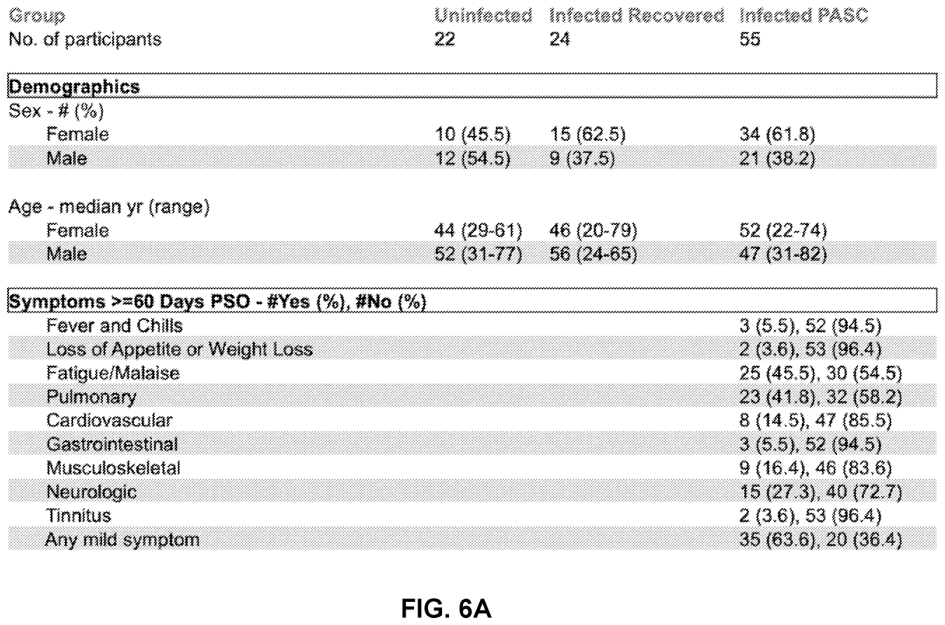

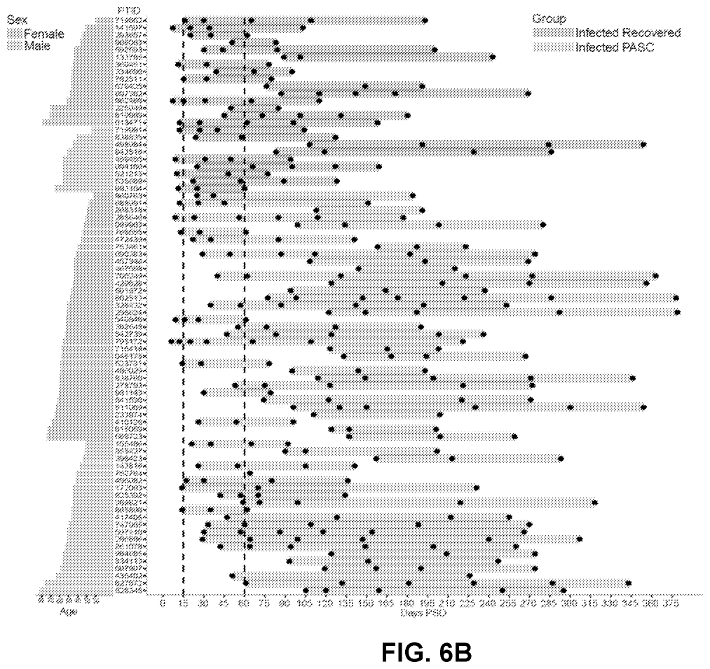

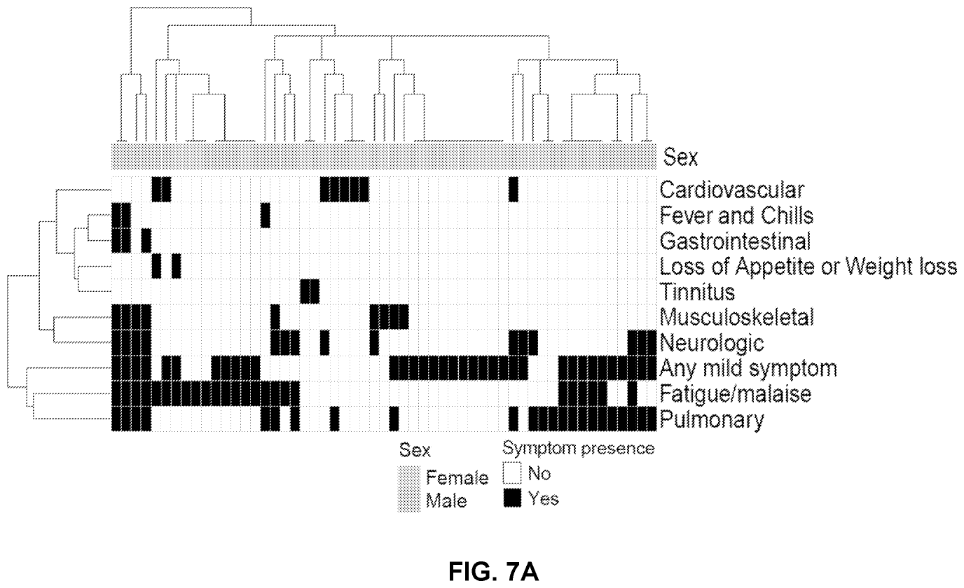

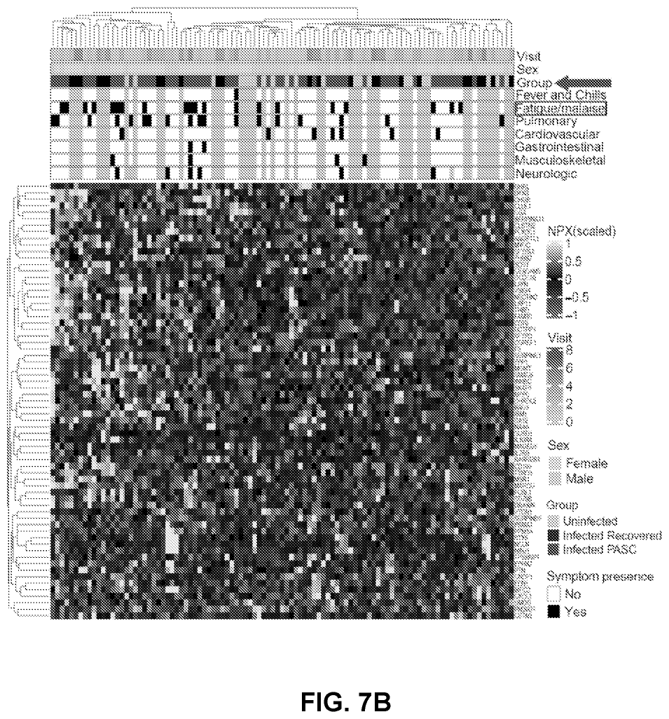

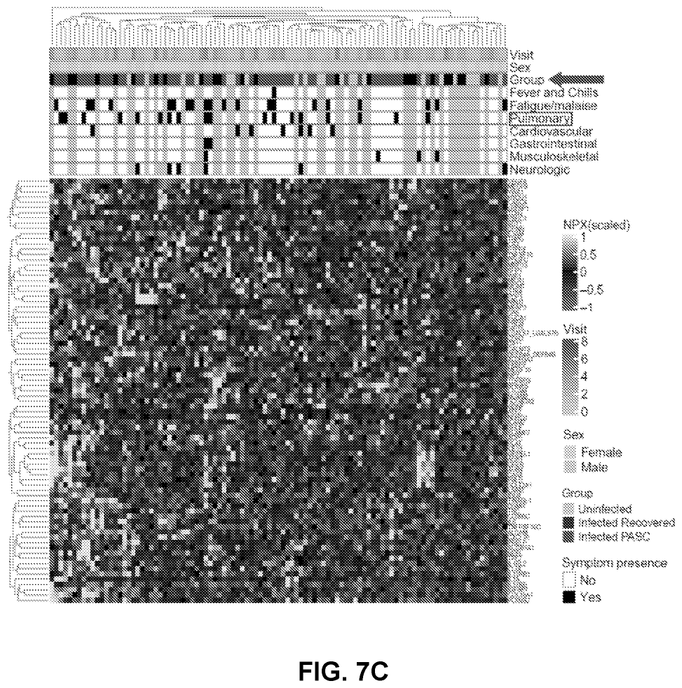

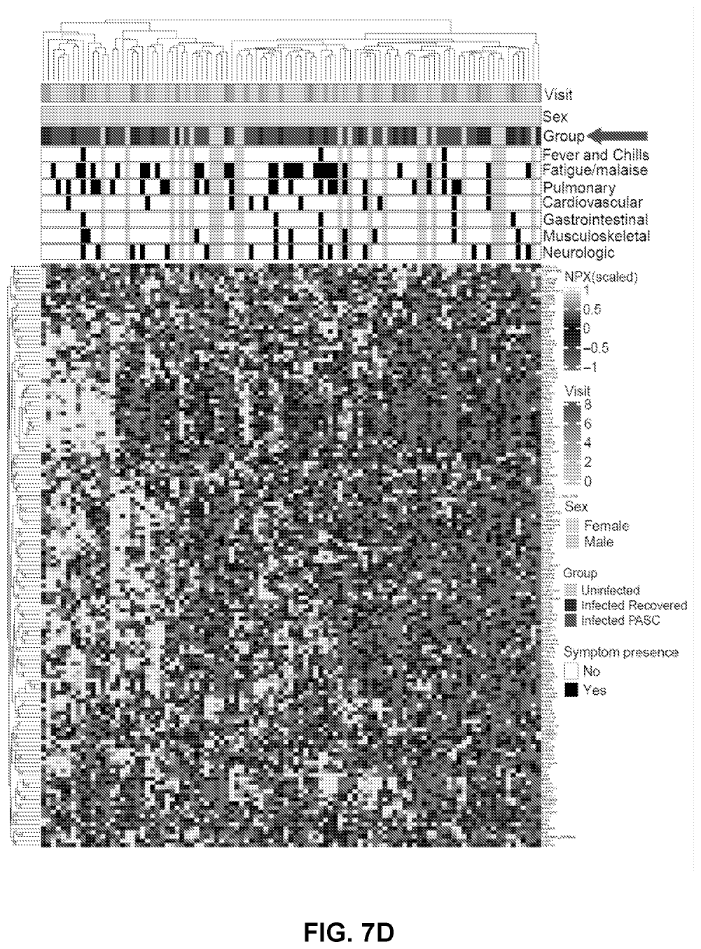

Previous studies have tried to subset PASC patients by either type, number, or severity of clinical features (Davis et al. 2021, Evans et al.). However, as disclosed herein, hierarchical clustering on PASC symptomatology alone at ≥60 days post symptom onset (PSO) does not clearly drive significant patient clustering (FIG. 6A, FIG. 7A). Additionally, using symptoms to drive clustering of significantly associated serum protein signatures result in no single symptom or combination of symptoms being able to clearly distinguish patient groups (FIG. 7B, C, D), suggesting that symptoms alone are unable to differentiate subsets of PASC.

Hence, disclosed herein are methods using unbiased clustering of the serum proteome across the entire cohort (PASC+recovered+uninfected) to find clusters of individuals that have similar serum proteome signatures regardless of their status or symptomatology.

In one aspect, disclosed herein is a method of diagnosing or classifying a subject as having a chronic inflammatory syndrome with or without a chronic or long-term infection of a virus, bacterium, fungus, and/or parasite, and/or an autoimmune disease, comprising determining a serum proteome signature of the subject. In some embodiments, the method comprises: (a) obtaining a sample from the subject with persistent symptoms lasting a specific period after onset of the infection; (b) analyzing the sample to determine the serum proteome signature; (c) diagnosing the subject based on the serum proteome signature; and (d) treating the subject by administering to the subject one or more therapeutic agents. In some embodiments, the specific period is at least 30 days. In some embodiments, the specific period is at least 45 days. In some embodiments, the specific period is at least 60 days. In some embodiments, the specific period is at least 75 days. In some embodiments, the specific period is at least 90 days. In some embodiments, the specific period is between 30 days and 90 days. In some embodiments, the specific period is between 90 days and 180 days. In some embodiments, the specific period is between 180 days and 1 year. In some embodiments, the specific period is more than 1 year. In some embodiments, the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV). In some embodiments, the subject is diagnosed or classified as having post-acute sequelae of SARS-CoV-2 infection (PASC).

In another aspect, disclosed herein is a method of diagnosing or classifying a subject as having a chronic inflammatory syndrome with or without a chronic or long-term infection of a virus, bacterium, fungus, and/or parasite, and/or an autoimmune disease, comprising: (a) obtaining a first sample from the subject with persistent symptoms lasting a specific period after onset of the infection; (b) obtaining a second sample from an uninfected or recovered individual; (c) analyzing the first sample to determine a first serum proteome signature and analyzing the second sample to determine a second serum proteome signature; (d) performing canonical pathway enrichment on the first sample and the second sample; (e) diagnosing the subject based on the first serum proteome signature; and (f) treating the subject by administering to the subject one or more therapeutic agents. In some embodiments, the method further comprises using curated canonical pathways from the Molecular Signatures Database (MSigDB) and a rule-based approach to determine a number of pathways that distinguish the first serum proteome signature from the second serum proteome signature. In some embodiments, the method further comprises merging the number of pathways into a specific number of proteomic modules to avoid gene set redundancy. In some embodiments, the method further comprises hierarchical clustering using the specific number of proteomic modules to identify discrete clusters showing distinct expression patterns of the proteomic modules. In some embodiments, the method further comprises determining a marked enrichment for inflammatory modules in a subset of the proteomic modules in the discrete clusters.

In some embodiments, the diagnosing the subject based on the first serum proteome signature comprises determining the subset of the proteomic modules in the discrete clusters comprising the marked enrichment for inflammatory modules. In some embodiments, the inflammatory modules comprise marked enrichment for one or more of Type II interferon signaling, canonical NF-κB signaling, NF-κB activating cytokine pathways, IL-12 signaling, p40 signaling, IFN-γ-driven chemokines, TNF-driven cytokines and chemokines, Type I IFN signaling, IFNA signaling, targeted cytokine signaling, and IL-12/IFN-γ axis signaling. In some embodiments, the Type II interferon signaling comprises marked enrichment for Type II IFN-γ signaling. In some embodiments, the Type II interferon signaling comprises marked enrichment for IL-27 signaling. In some embodiments, the Type II interferon signaling comprises marked enrichment for TID signaling. In some embodiments, the Type II IFN-γ signaling comprises marked enrichment for IL-27 signaling. In some embodiments, the Type II IFN-γ signaling comprises marked enrichment for IL-18 signaling. In some embodiments, the Type II IFN-γ signaling comprises marked enrichment for NF-κB signaling. In some embodiments, the canonical NF-κB signaling is particularly associated with TNF. In some embodiments, the TNF signaling comprises marked enrichment for IL-1 signaling. In some embodiments, the TNF signaling comprises marked enrichment for NF-κB signaling. In some embodiments, the TNF signaling comprises marked enrichment for IFN-α signaling. In some embodiments, the NF-κB activating cytokine pathways comprise marked enrichment for IL-18 signaling. In some embodiments, the NF-κB activating cytokine pathways comprise marked enrichment for TNF signaling. In some embodiments, the NF-κB activating cytokine pathways comprise marked enrichment for IL-1 signaling. In some embodiments, the TNF-driven cytokines and chemokines comprise marked enrichment for IL-6 signaling. In some embodiments, the TNF-driven cytokines and chemokines comprise marked enrichment for CCL7 or MCP3 signaling. In some embodiments, the Type I IFN signaling comprises marked enrichment for SAMD9L signaling. In some embodiments, the Type I IFN signaling comprises marked enrichment for MNDA signaling. In some embodiments, the Type I IFN signaling comprises marked enrichment for DDX58 signaling. In some embodiments, the Type I IFN signaling comprises marked enrichment for LAMP3 signaling. In some embodiments, the targeted cytokine signaling comprises marked enrichment for IFN-γ signaling. In some embodiments, the targeted cytokine signaling comprises marked enrichment for IFN-β signaling. In some embodiments, the targeted cytokine signaling comprises marked enrichment for IFN-λ1/2/3 signaling. In some embodiments, the targeted cytokine signaling comprises marked enrichment for TNF signaling. In some embodiments, the targeted cytokine signaling comprises marked enrichment for IL-6 signaling. In some embodiments, the targeted cytokine signaling comprises marked enrichment for IL-1B signaling. In some embodiments, the targeted cytokine signaling comprises marked enrichment for PTX3 signaling.

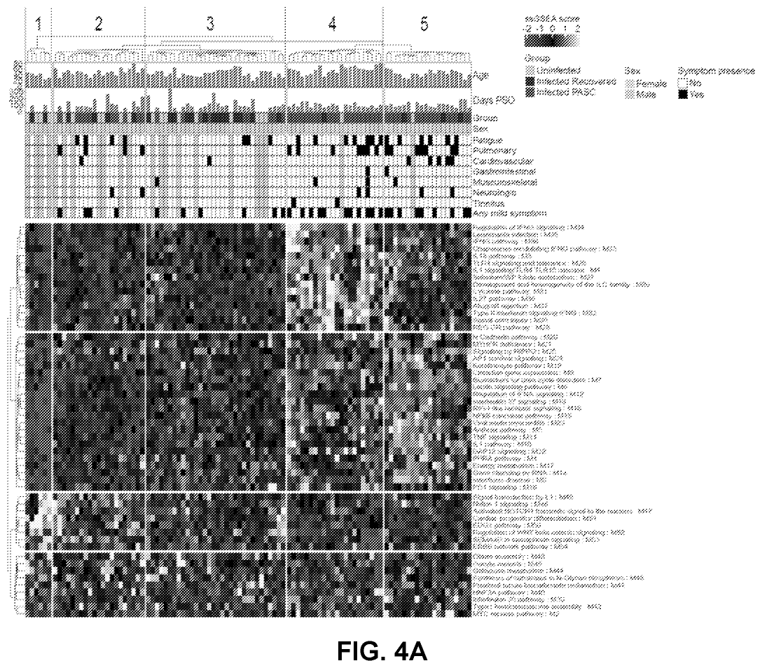

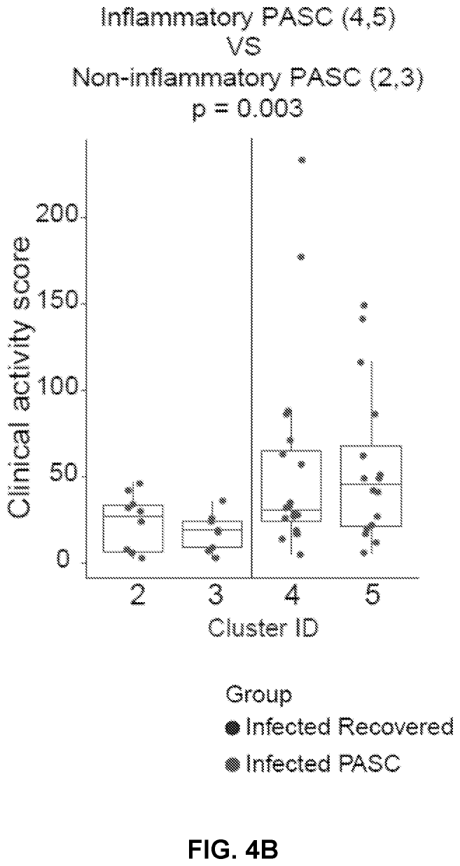

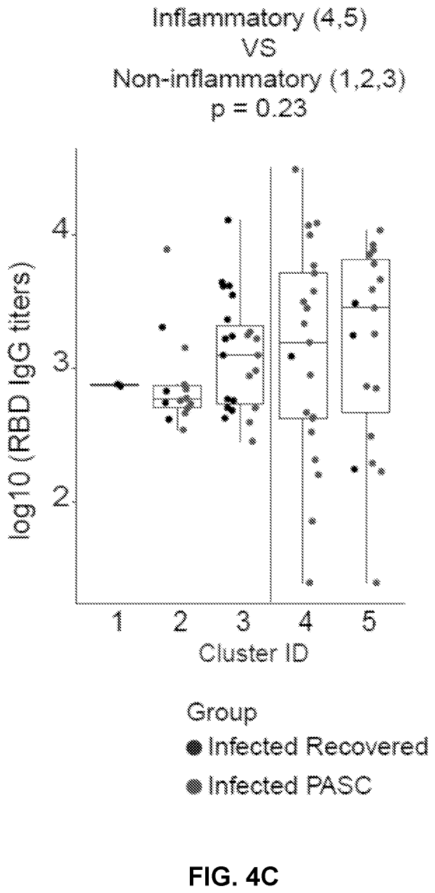

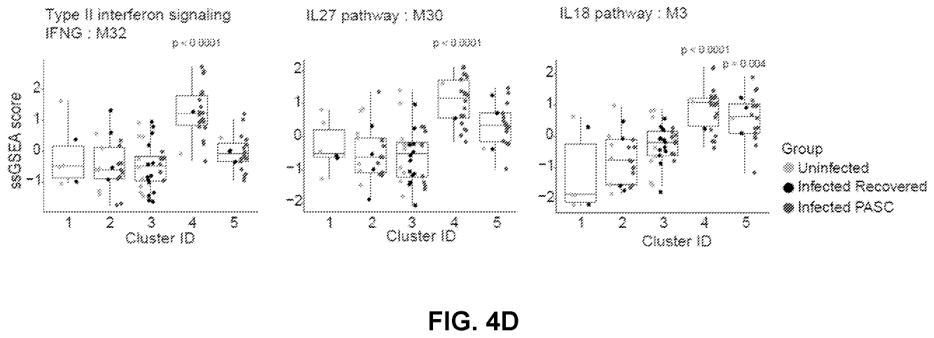

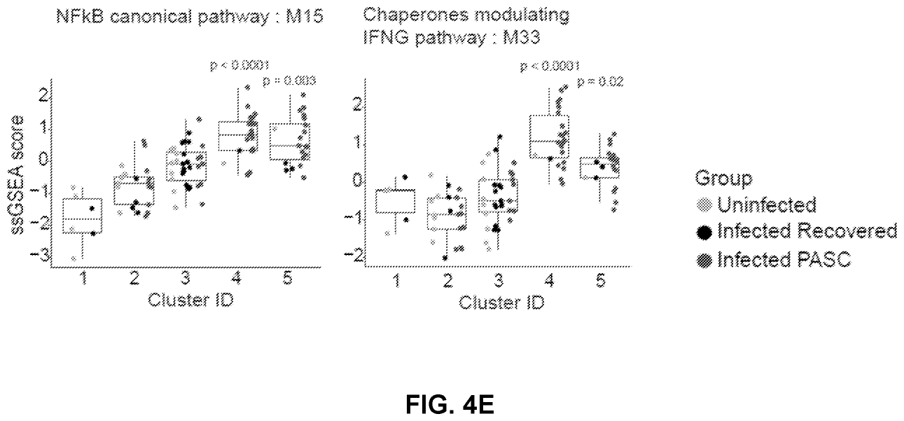

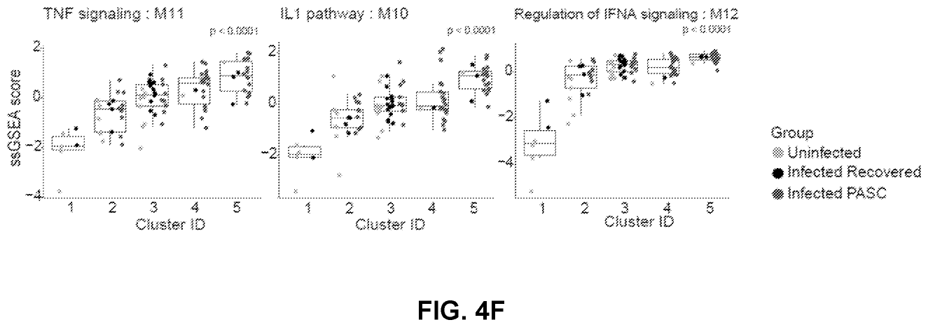

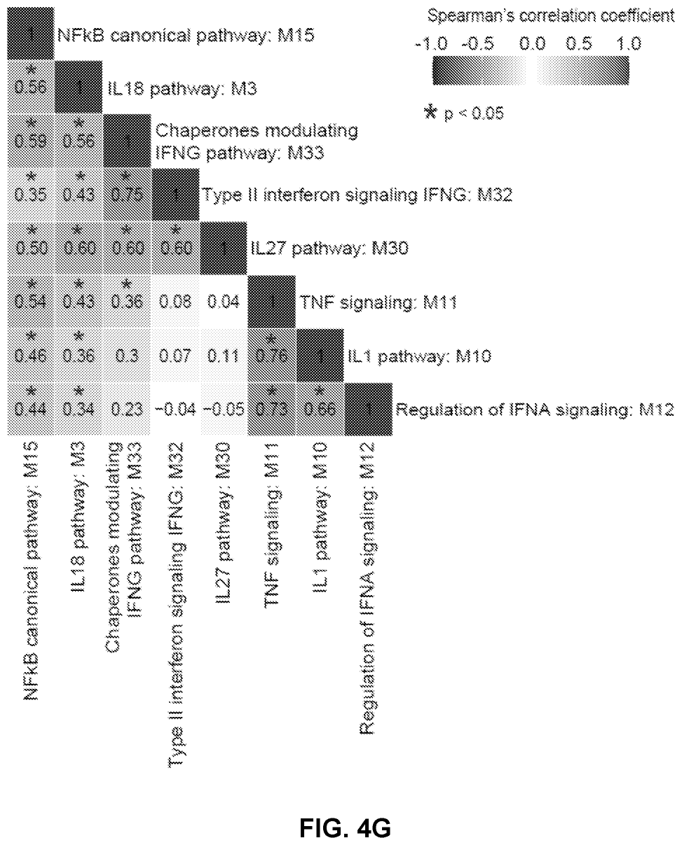

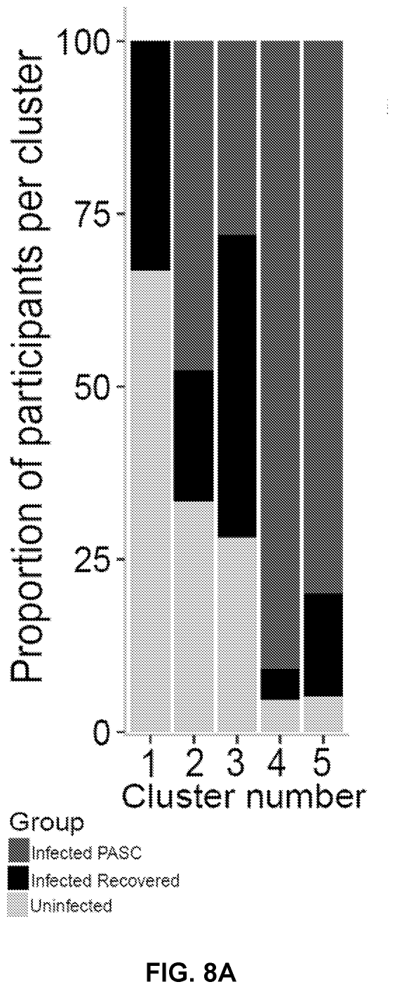

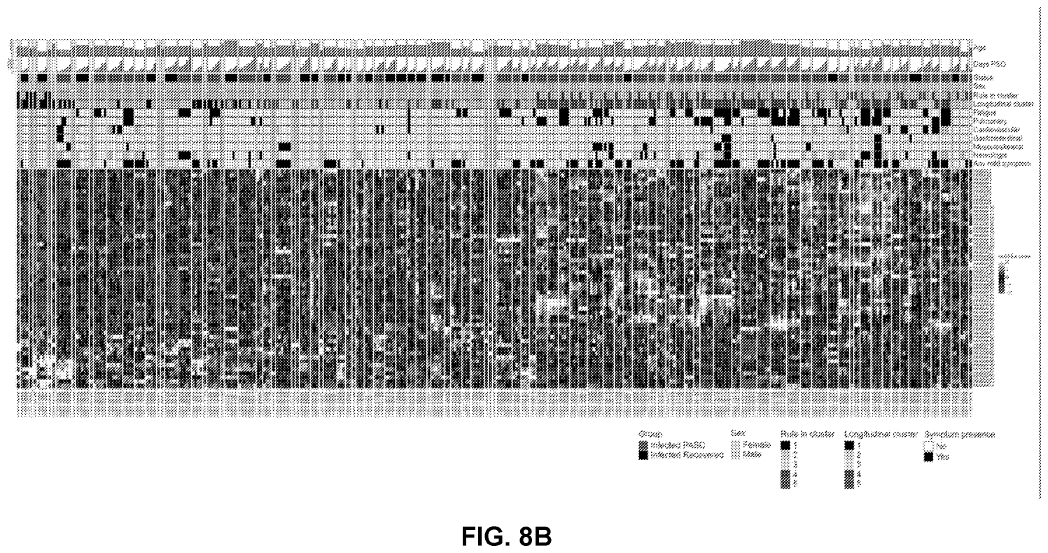

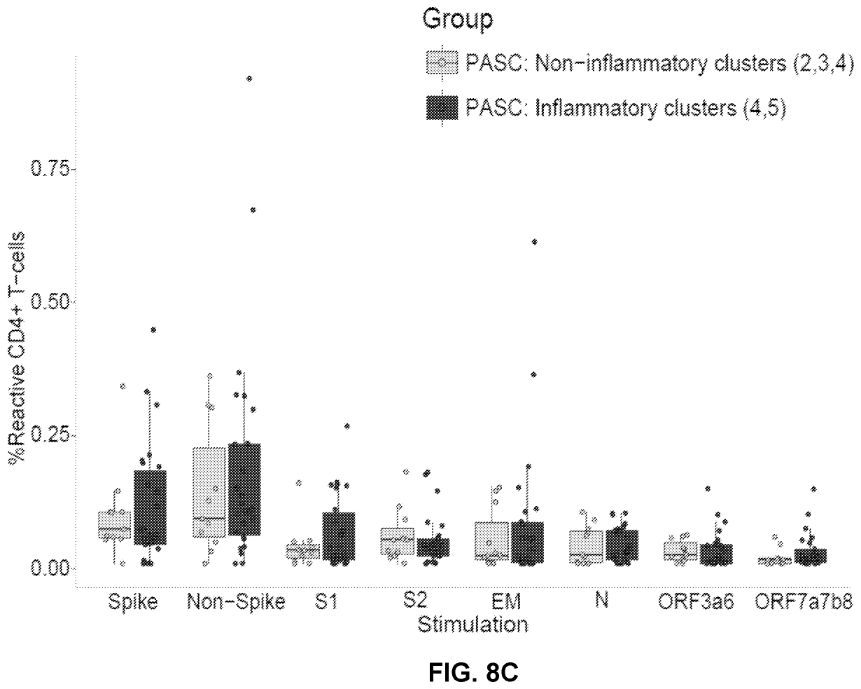

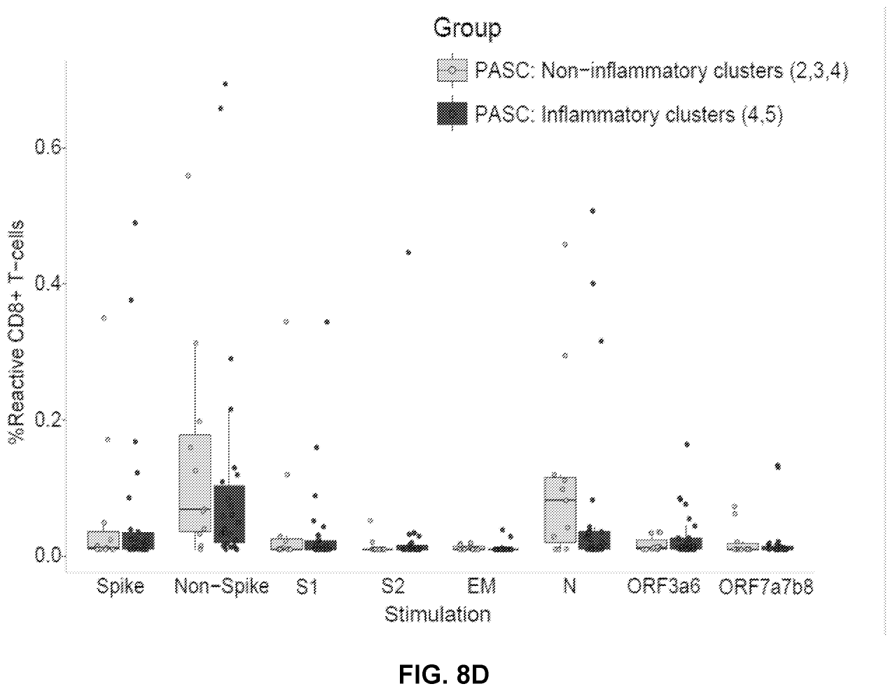

In some embodiments, canonical pathway enrichment is performed on the first post-60 day sample available for each PASC subject, the last available post-60 day sample for each recovered subject (to maximize the chance that they had returned to baseline), and on the solitary sample from the uninfected individuals. In some embodiments, curated canonical pathways from the Molecular Signatures Database (MSigDB) are used and a rule-in approach applied, resulting in 85 pathways that distinguish PASC from recovered and uninfected individuals with a significant rule-in performance (p<0.01). In some embodiments, these pathways are merged into 54 modules to avoid gene set redundancy using the enrichment map approach with a minimum Jaccard index threshold of 25% (Table 1, after REFERENCES section). In some embodiments, hierarchical clustering using the 54 proteomic modules identify 5 discrete clusters showing distinct expression patterns of the modules (FIG. 4A). In some embodiments, two of the clusters (4 & 5) show a marked enrichment for inflammatory modules while clusters 1, 2, and 3 lack a distinct inflammatory protein signature. In some embodiments, inflammatory clusters 4 and 5 include predominantly PASC subjects (91% and 80% respectively), whereas cluster 1 consists of only uninfected or recovered subjects. In some embodiments, clusters 2 and 3 consist of a mixture of PASC (48% and 28% respectively), recovered, and uninfected subjects (FIG. 8A). The distribution of PASC subjects across inflammatory (4 & 5; 65% of PASC) and non-inflammatory (2 & 3; 35% of PASC) proteomic clusters underscores the heterogeneity of PASC. In some embodiments, to determine whether the differential serum proteomic signatures discovered by comparing the first post-60 day PSO sample for PASC to the last post-60 day PSO sample for recovered are stable over time, the analysis is extended to include all longitudinal samples available for each subject. In some embodiments, PASC subjects that have an inflammatory protein signature continue to have that signature over time and most subjects remain in the same cluster throughout the study period (FIG. 8B).

In some embodiments, subsets of PASC with distinct signatures of persistent inflammation are identified. In some embodiments, Type II interferon signaling and canonical NF-κB signaling, particularly associated with TNF, are the most differentially enriched pathways. In some embodiments, the heterogeneity of PASC, identifying patients with molecular evidence of persistent inflammation, and highlighting dominant pathways that may have diagnostic or therapeutic relevance are resolved.

In some embodiments, an inflammatory plasma protein signature may also correlate with being more symptomatic. In some embodiments, because a cohort consists primarily of patients with only mild to moderate COVID-19 (WHO ordinal scale 2 or 3), commonly used COVID severity indices do not capture a range of heterogeneity in symptomatology. Disclosed herein is a clinical activity index that accounts for both symptoms and their impact on activities of daily living. In some embodiments, inflammatory PASC subjects in clusters 4 & 5 have a significantly higher clinical activity score (p=0.003) compared to non-inflammatory PASC subjects in clusters 2 & 3 (FIG. 4B).

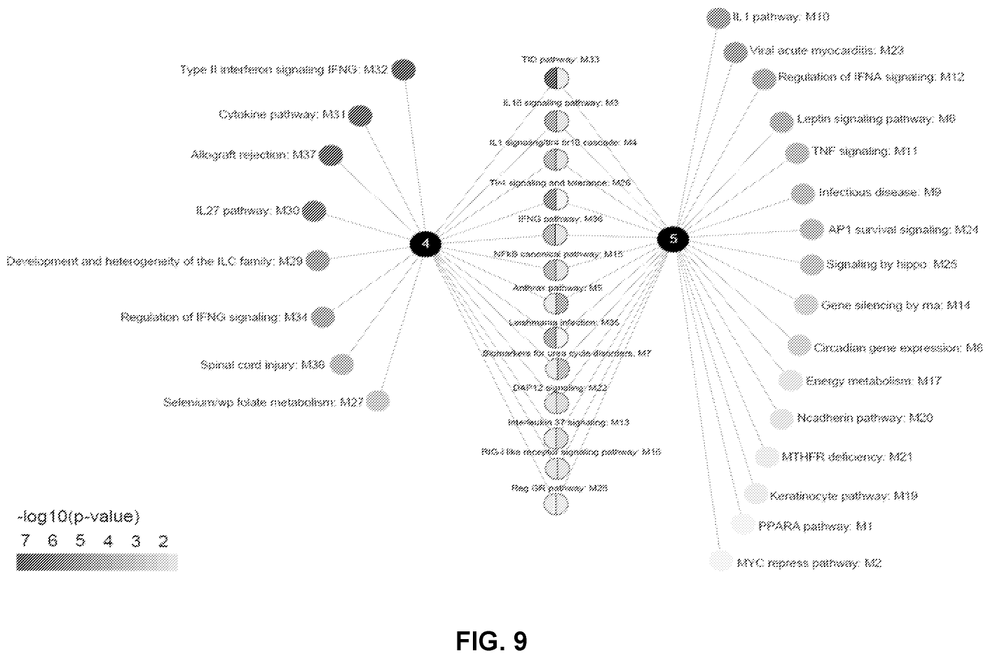

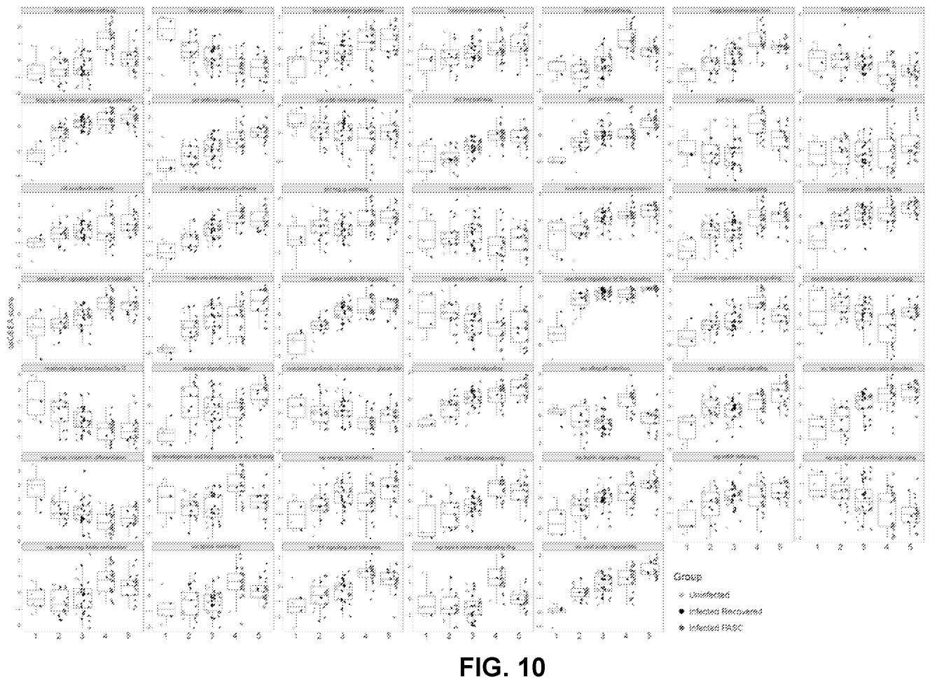

In some embodiments, among the 54 modules that define the 5 clusters (FIG. 4A), those that significantly distinguish each cluster are identified by calculating the single-sample-Gene Set Enrichment Analysis (ssGSEA) score per module across samples. In some embodiments, ranking modules by adjusted p-value identify those most significantly associated with clusters 4 and 5 (FIG. 9, FIG. 10, Table 2, after REFERENCES section). In some embodiments, within cluster 4, multiple pathways associated with type II interferon (IFN-γ) signaling (Type II IFN signaling, IL-27, TID, etc.) are among those most highly enriched (FIG. 4D). In some embodiments, canonical NF-κB signaling and NF-κB activating cytokine pathways (IL-18, TNF, IL-1) are enriched in both clusters 4 and 5 (FIG. 4E). In some embodiments, cluster 5 is also enriched for proteins associated with regulation of IFN-α signaling (FIG. 4F). In some embodiments, the expression scores of these modules across all samples are significantly correlated with each other. In some embodiments, the expression scores being significantly correlated with each other indicates that patients with higher IFN-γ signaling have higher IL27, IL18, and NF-κB signaling, and patients with higher TNF signaling have higher IL1, NF-κB, and IFN-α signaling, suggesting a global activation of immune cascades that drive inflammation (FIG. 4G).

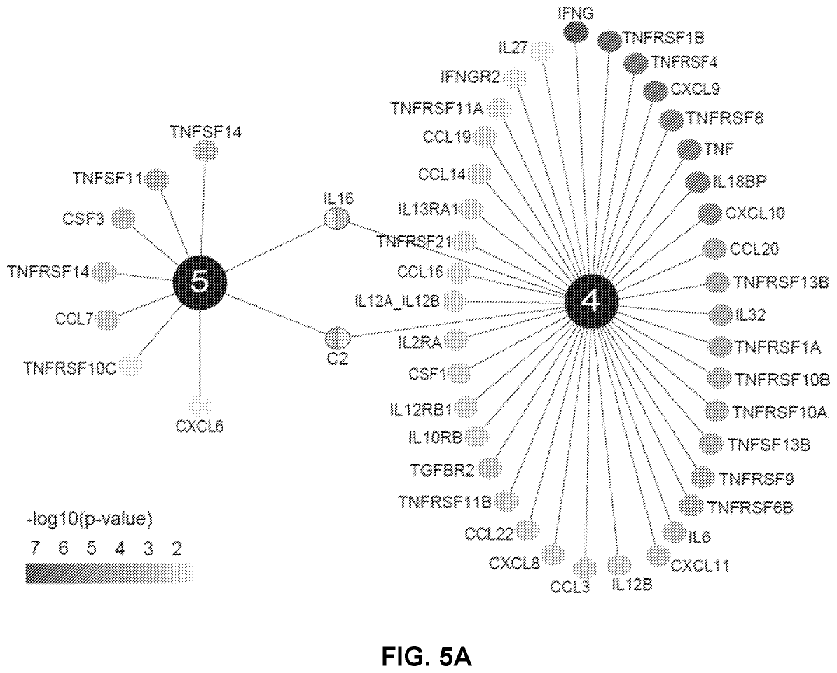

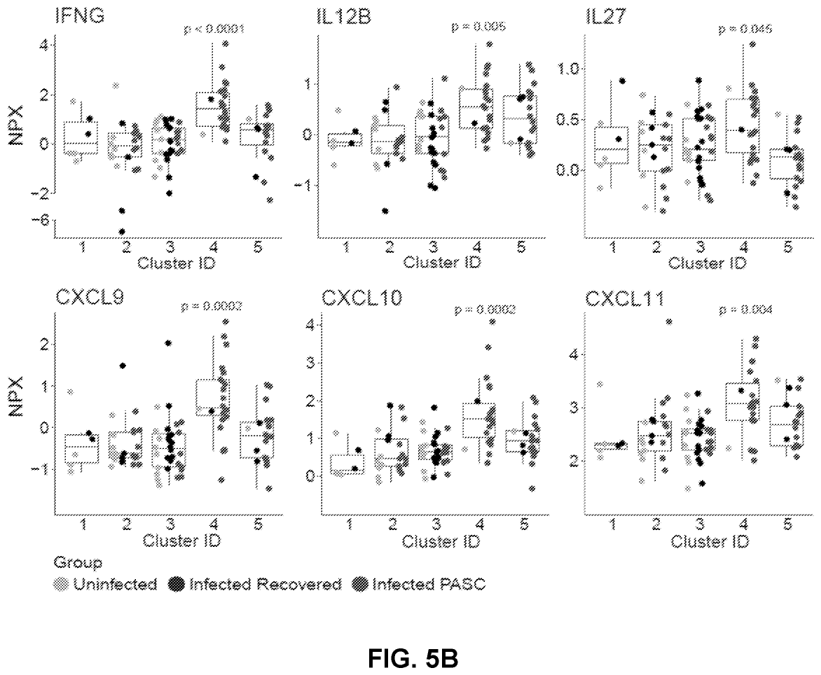

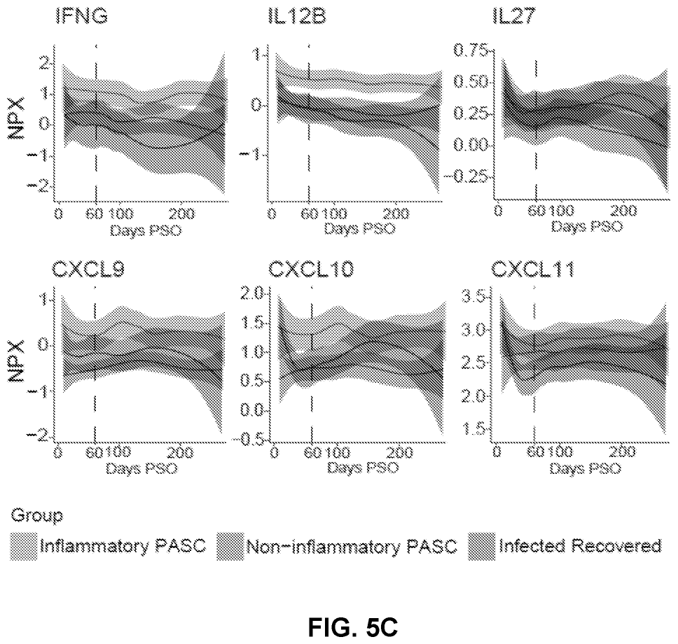

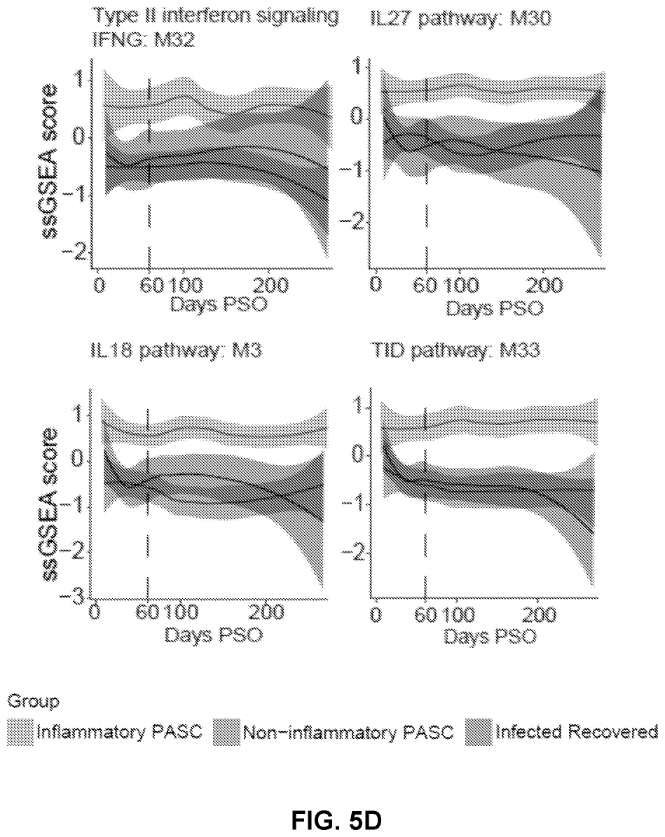

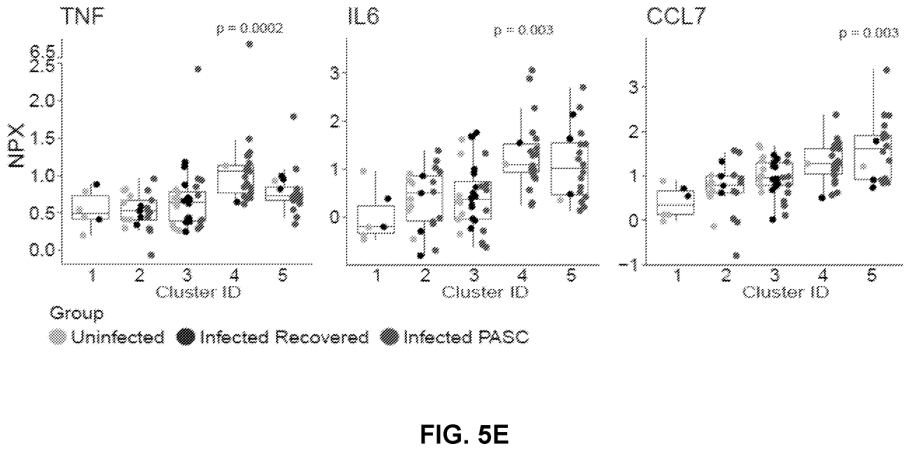

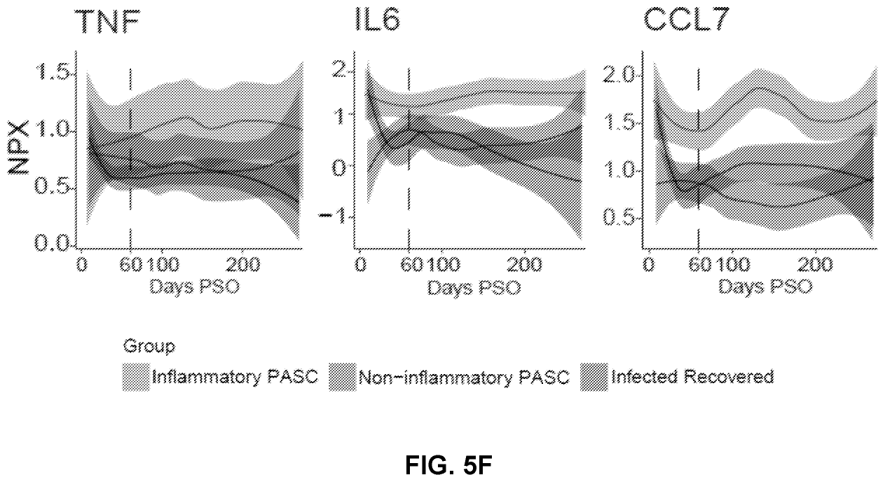

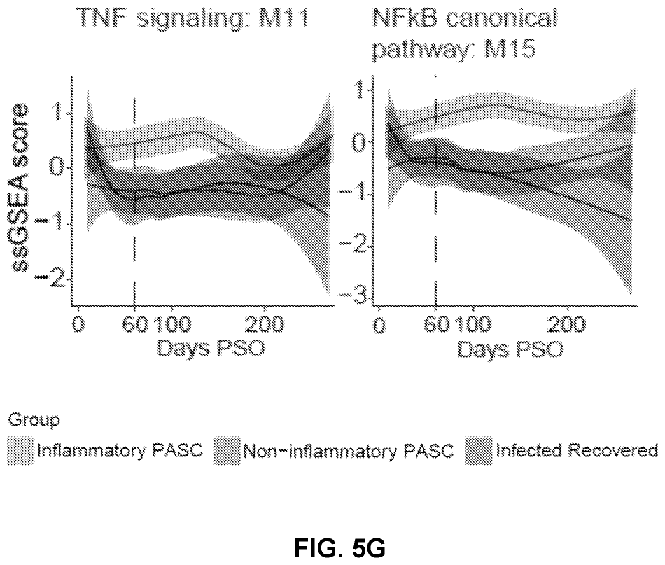

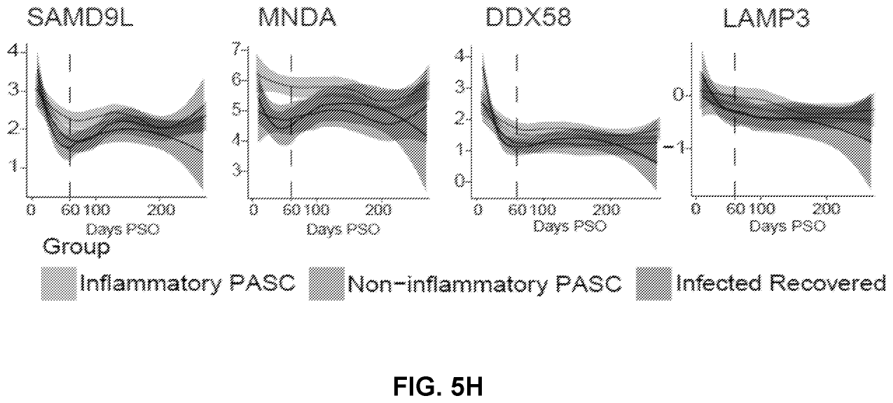

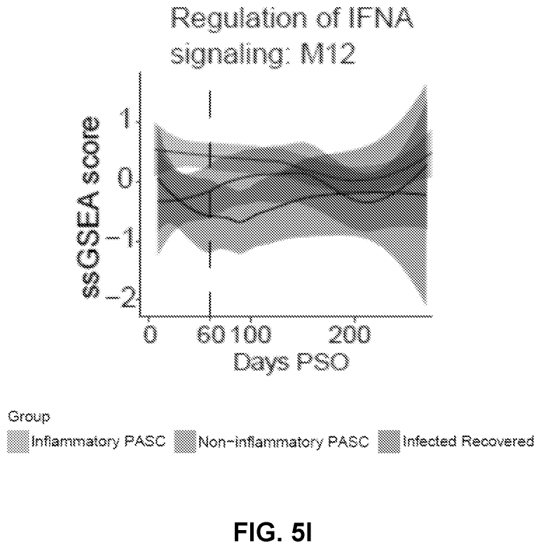

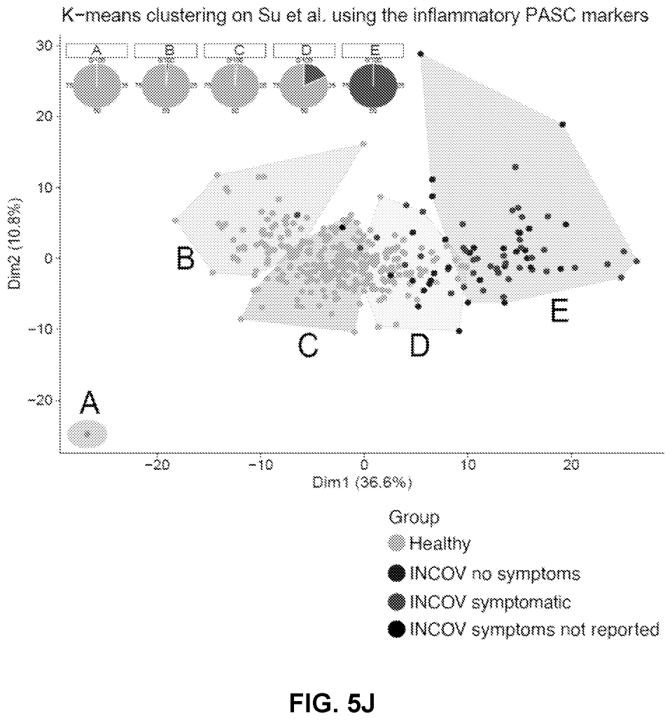

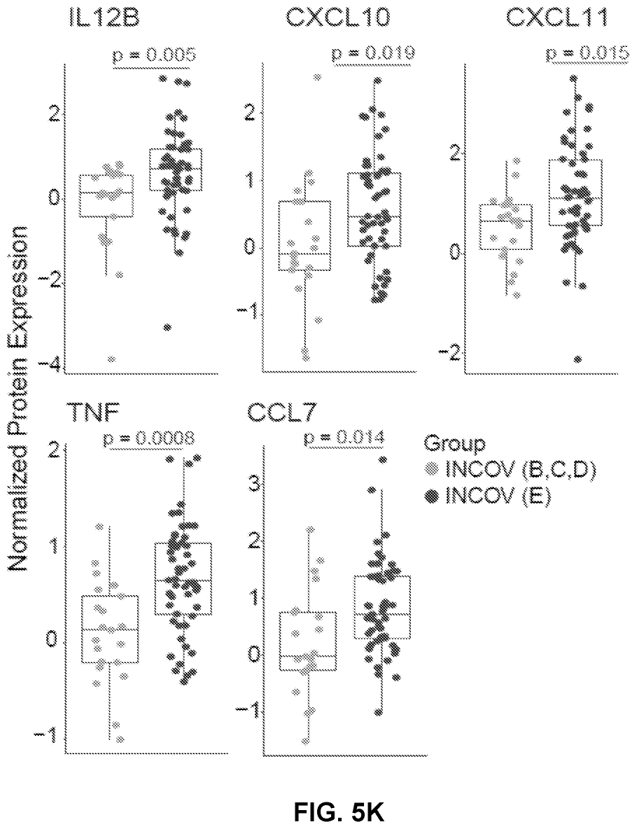

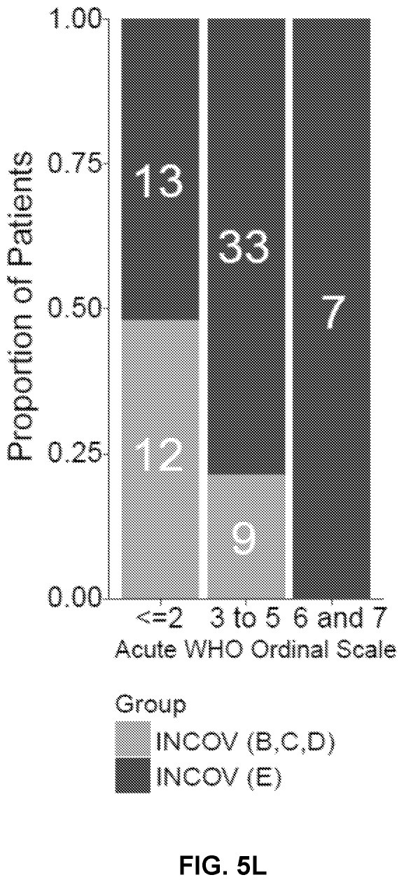

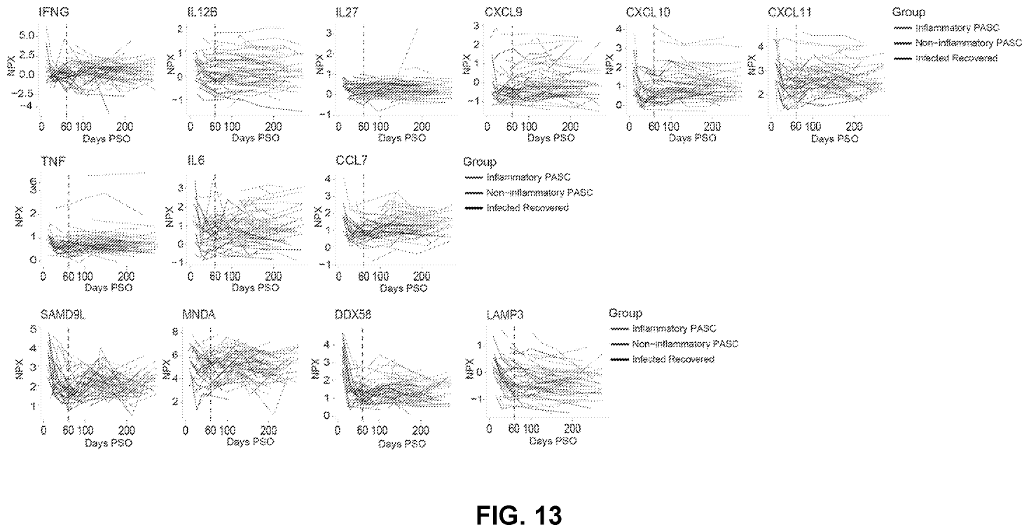

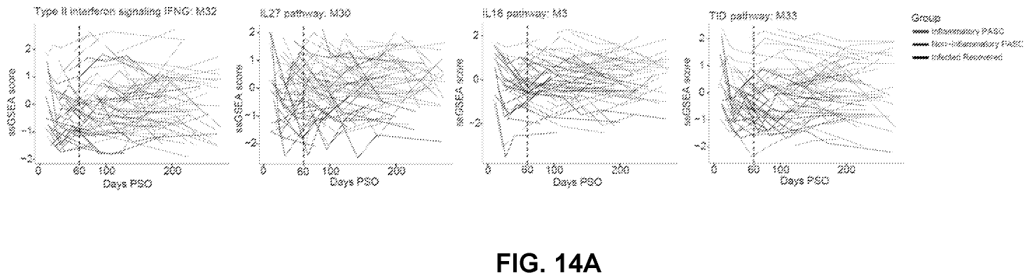

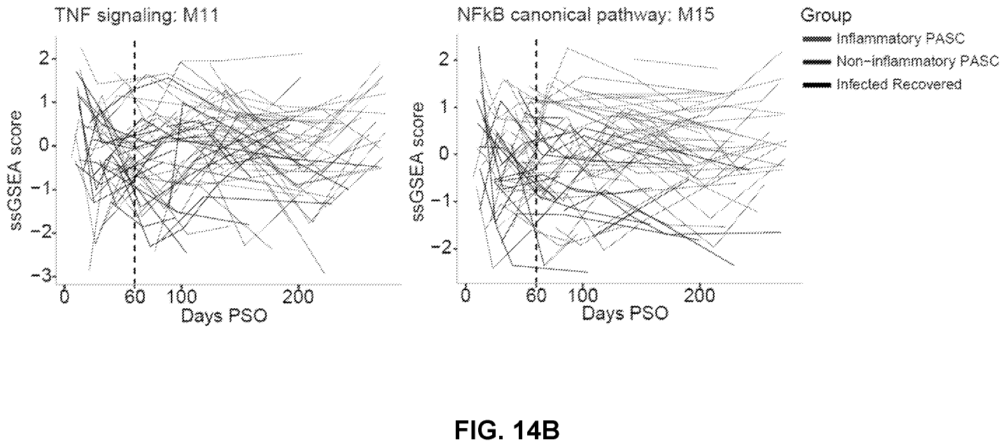

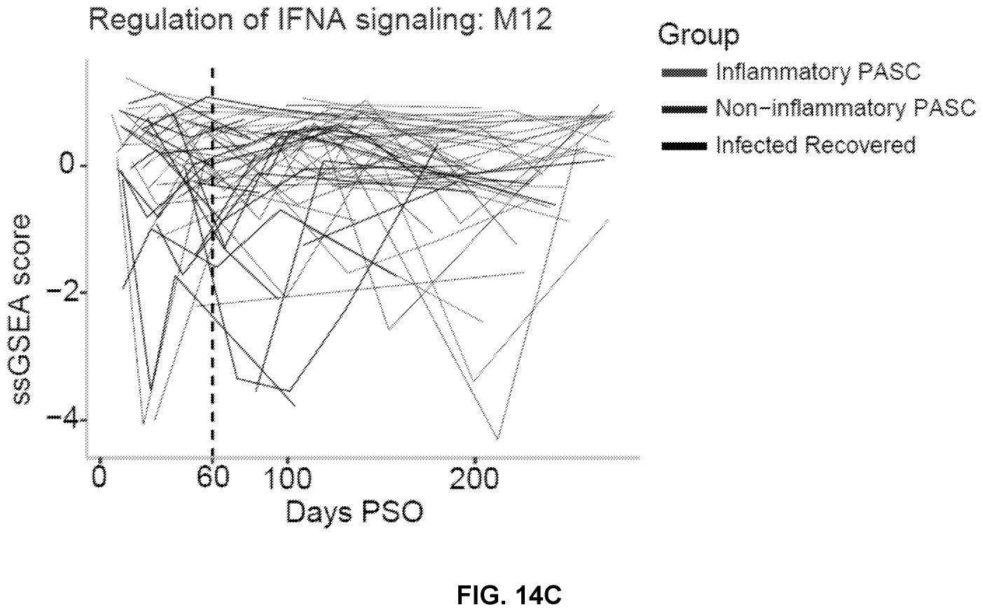

In some embodiments, IFN-γ, IL-12 p40, and IFN-γ-driven chemokines are consistently elevated within inflammatory PASC from clusters 4 & 5 compared to non-inflammatory PASC from clusters 1, 2, and 3, extending to at least 275 days after initial SARS-CoV-2 infection (FIG. 5C, FIG. 13). In some embodiments, IFN-γ related signaling modules also show persistent enrichment over the same time (FIG. 5D, FIG. 14). In some embodiments, in addition to IFN-γ, TNF, TNF-driven cytokines and chemokines (including IL-6 and CCL7 (MCP3)), and several TNF receptor superfamily members are also increased in clusters 4 and 5 (FIG. 5A, FIG. 5E, FIG. 13). TNF, IL-6, and CCL7 remain persistently elevated in inflammatory PASC over time compared to non-inflammatory PASC (FIG. 5F, FIG. 13). In addition, TNF signaling and canonical NF-κB signaling pathways previously found to be enriched at early time points in inflammatory PASC remain elevated over time (FIG. 5G, FIG. 14).

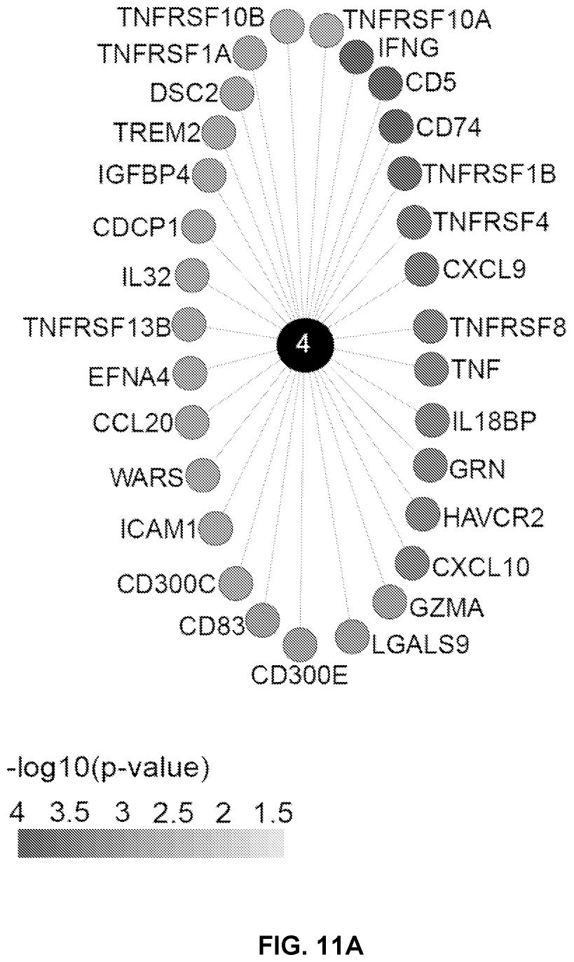

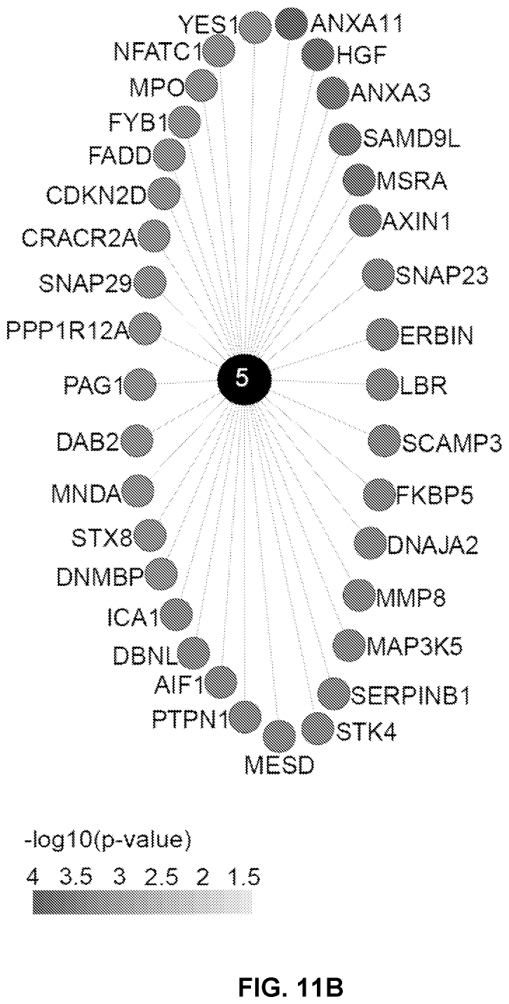

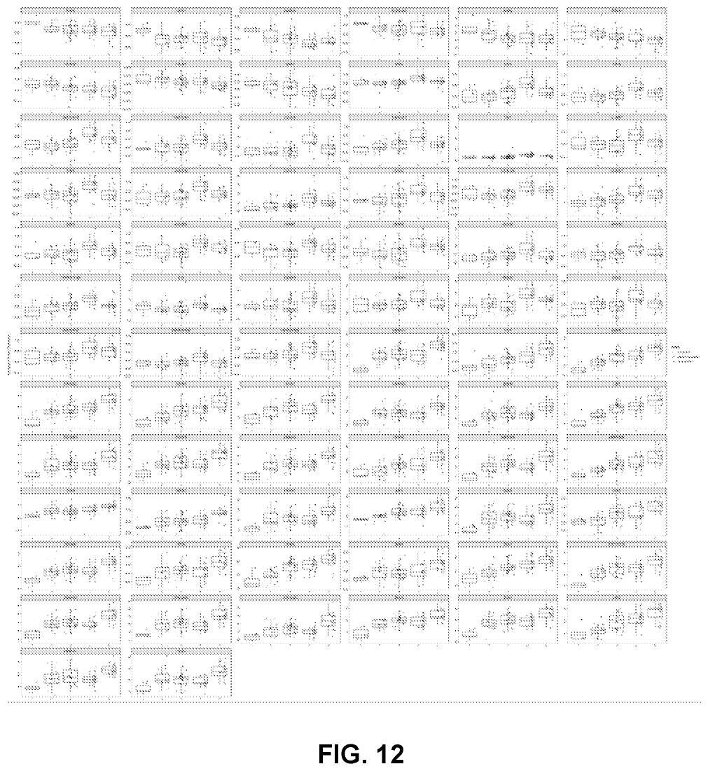

In some embodiments, the pathway related to expression of IFNA signaling is found to be enriched at the first post-60 day PSO timepoint in cluster 5 (FIG. 4F). In some embodiments, proteins associated with type I IFN activation including SAMD9L, MNDA, DDX58, LAMP3, and others are characterized by increased expression (FIG. 11, FIG. 12). In some embodiments, said proteins are found to be highly increased early after acute infection but in inflammatory PASC, remain elevated over time compared to non-inflammatory PASC. In some embodiments, longitudinal assessment show that said proteins trend toward levels seen in non-inflammatory PASC and recovered subjects by approximately 180 days post infection (FIG. 5H), similar to the kinetic observed for the expression of IFNA signaling pathway over time (FIG. 5I).

In some embodiments, there is increased expression of IFN-γ, IFN-β, IFN-λ1/2/3, TNF, IL-6, IL-1β, and PTX3 in plasma from PASC patients using targeted cytokine panels. In some embodiments, plasma proteomic profiling can identify subjects with PASC who have an ongoing inflammatory signature, offering a first opportunity to subset PASC patients for further mechanistic studies, clinical trials, or development of diagnostics based on an underlying molecular signature. In some embodiments, in PASC subjects with inflammatory protein signatures, IL-12/IFN-γ axis is highly active and is combined with a NF-κB driven protein signature. In some embodiments, the high activity of IL-12/IFN-γ axis combined with the NF-κB driven protein signature is possibly driven by TNF, leading to excess IL-6 expression. In some embodiments, there is a persistent type I IFN driven protein signature that is present in PASC subjects with an inflammatory protein signature early in the PASC period (>60 days post-symptom onset) and extending to approximately 6 months post-infection that then trends toward normal.

In some aspects, the disclosure provides methods for diagnosing or classifying a subject as having a long-term infection of a virus and/or long-term symptoms caused by infection of a virus. The long-term symptoms caused by infection of a virus can be direct infection effects or symptoms, as well as secondary and tertiary effects that may be initiated by the infection but persist by other mechanisms in the body. In some embodiments, the virus is SARS-CoV-2. In some embodiments, the long-term viral infection is post-acute sequelae to SARS-CoV-2 infection (PASC).

In some embodiments, the methods for diagnosing or classifying a subject as having a long-term viral infection and/or long-term symptoms associate thereof (e.g., PASC) comprise determining the level of one or more biomarkers in a biological sample obtained from the subject. In some embodiments, the biological sample can be a blood sample or a serum sample. These samples comprise peripheral blood mononuclear cells (PBMCs). As known to a person of ordinary skill in the art, the levels of one or more biomarkers can be determined from the biological sample using various laboratory techniques, non-limiting examples of which include immunoassays, flow cytometry, proteomics, and nucleotide (e.g., DNA, RNA) sequencing techniques including those at a single-cell resolution.

In some embodiments, the one or more biomarkers comprise proteins associated with an inflammatory response, including those associated with respiratory burst, immune cell antigen processing and presentation, germinal center formation, immune cell proliferation, T cell cytokine production, and acute inflammatory responses. In some embodiments, the proteins associated with an inflammatory response comprise one or more of IFNLR1, BCAM, S100A16, and IL5. In some embodiments, an upregulation of one or more of these proteins indicate that the subject has PASC.

In some embodiments, the one or more biomarkers comprise cytokines, chemokines, and/or immunomodulatory proteins. In some embodiments, the cytokines, chemokines, and/or immunomodulatory proteins comprise one or more of IL5, IL11, IL1B, CXCL1, CXCL8, CCL3, CCL11, IL1RL2, CD28, HLA-DRA, LAG3, and PDCD1. An elevation of one or more of these cytokines, chemokines, and/or immunomodulatory proteins indicate that the subject has PASC. In some embodiments, the cytokines and chemokines comprise IL15, which is a unique cytokine biomarker in PASC patients. IL15 is consistently lower in PASC patients compared to recovered subjects and correlates with disease severity and mortality. In some embodiments, the cytokines and chemokines comprise IL13, which is also a unique cytokine biomarker in PASC patients and is elevated in PASC patients compared to recovered subjects.

In some embodiments, the one or more biomarkers comprise hormones and hormone receptors. In some embodiments, the hormones and hormone receptors comprise one or more CRH, CRHR1, and PTH1R. In some embodiments, PASC patients have elevated levels of one or more of CRH, CRHR1, and PTH1R.

In some embodiments, the one or more biomarkers comprise transcription factors and motifs thereof that might be associated with aberrant cell phenotypes. In some embodiments, the transcription factors and motifs thereof comprise AP-1 family transcription factor motifs. In some embodiments, the transcription factors and motifs thereof comprise one or more of BACH, BATF, IRF, and STAT. In some embodiments, PASC patients have increased activation of signaling pathways driving one or more of these transcription factor motifs.

In one aspect, disclosed herein is a molecular signature for use in determining whether a subject infected with or previously infected with a virus or other pathogen is likely to suffer from a chronic inflammatory syndrome with or without a chronic or long-term infection of the virus or other pathogen, the molecular signature comprising one or more inflammatory proteins or biomarkers that are enriched in the subject relative to an uninfected or recovered control subject. In some embodiments, the molecular signature comprises two or more inflammatory proteins or biomarkers that are enriched in the subject relative to an uninfected or recovered control subject. In some embodiments, the molecular signature comprises three or more inflammatory proteins or biomarkers that are enriched in the subject relative to an uninfected or recovered control subject. In some embodiments, the molecular signature comprises four or more inflammatory proteins or biomarkers that are enriched in the subject relative to an uninfected or recovered control subject. In some embodiments, the molecular signature comprises five or more inflammatory proteins or biomarkers that are enriched in the subject relative to an uninfected or recovered control subject. In some embodiments, the molecular signature comprises six or more inflammatory proteins or biomarkers that are enriched in the subject relative to an uninfected or recovered control subject. In some embodiments, the molecular signature comprises seven or more inflammatory proteins or biomarkers that are enriched in the subject relative to an uninfected or recovered control subject. In some embodiments, the molecular signature comprises eight or more inflammatory proteins or biomarkers that are enriched in the subject relative to an uninfected or recovered control subject. In some embodiments, the molecular signature comprises nine or more inflammatory proteins or biomarkers that are enriched in the subject relative to an uninfected or recovered control subject. In some embodiments, the molecular signature comprises ten or more inflammatory proteins or biomarkers that are enriched in the subject relative to an uninfected or recovered control subject. In some embodiments, the molecular signature comprises eleven or more inflammatory proteins or biomarkers that are enriched in the subject relative to an uninfected or recovered control subject. In some embodiments, the molecular signature comprises twelve or more inflammatory proteins or biomarkers that are enriched in the subject relative to an uninfected or recovered control subject. In some embodiments, the molecular signature comprises thirteen or more inflammatory proteins or biomarkers that are enriched in the subject relative to an uninfected or recovered control subject. In some embodiments, the molecular signature comprises fourteen or more inflammatory proteins or biomarkers that are enriched in the subject relative to an uninfected or recovered control subject. In some embodiments, the molecular signature comprises fifteen or more inflammatory proteins or biomarkers that are enriched in the subject relative to an uninfected or recovered control subject. In some embodiments, the molecular signature comprises sixteen or more inflammatory proteins or biomarkers that are enriched in the subject relative to an uninfected or recovered control subject. In some embodiments, the molecular signature comprises seventeen or more inflammatory proteins or biomarkers that are enriched in the subject relative to an uninfected or recovered control subject. In some embodiments, the molecular signature comprises eighteen or more inflammatory proteins or biomarkers that are enriched in the subject relative to an uninfected or recovered control subject. In some embodiments, the molecular signature comprises nineteen or more inflammatory proteins or biomarkers that are enriched in the subject relative to an uninfected or recovered control subject. In some embodiments, the molecular signature comprises twenty or more inflammatory proteins or biomarkers that are enriched in the subject relative to an uninfected or recovered control subject. In some embodiments, the subject is likely to suffer from a chronic inflammatory syndrome with a chronic or long-term infection of the virus or other pathogen. In some embodiments, the subject is likely to suffer from a chronic inflammatory syndrome without a chronic or long-term infection of the virus or other pathogen.

In some embodiments, the molecular signature comprises one or more inflammatory proteins that are enriched in the subject relative to an uninfected or recovered control subject. In some embodiments, the molecular signature comprises two or more inflammatory proteins that are enriched in the subject relative to an uninfected or recovered control subject. In some embodiments, the molecular signature comprises three or more inflammatory proteins that are enriched in the subject relative to an uninfected or recovered control subject. In some embodiments, the molecular signature comprises four or more inflammatory proteins that are enriched in the subject relative to an uninfected or recovered control subject. In some embodiments, the molecular signature comprises five or more inflammatory proteins that are enriched in the subject relative to an uninfected or recovered control subject. In some embodiments, the molecular signature comprises six or more inflammatory proteins that are enriched in the subject relative to an uninfected or recovered control subject. In some embodiments, the molecular signature comprises seven or more inflammatory proteins that are enriched in the subject relative to an uninfected or recovered control subject. In some embodiments, the molecular signature comprises eight or more inflammatory proteins that are enriched in the subject relative to an uninfected or recovered control subject. In some embodiments, the molecular signature comprises nine or more inflammatory proteins that are enriched in the subject relative to an uninfected or recovered control subject. In some embodiments, the molecular signature comprises ten or more inflammatory proteins that are enriched in the subject relative to an uninfected or recovered control subject.

In some embodiments, the molecular signature comprises one or more biomarkers that are enriched in the subject relative to an uninfected or recovered control subject. In some embodiments, the molecular signature comprises two or more biomarkers that are enriched in the subject relative to an uninfected or recovered control subject. In some embodiments, the molecular signature comprises three or more biomarkers that are enriched in the subject relative to an uninfected or recovered control subject. In some embodiments, the molecular signature comprises four or more biomarkers that are enriched in the subject relative to an uninfected or recovered control subject. In some embodiments, the molecular signature comprises five or more biomarkers that are enriched in the subject relative to an uninfected or recovered control subject. In some embodiments, the molecular signature comprises six or more biomarkers that are enriched in the subject relative to an uninfected or recovered control subject. In some embodiments, the molecular signature comprises seven or more biomarkers that are enriched in the subject relative to an uninfected or recovered control subject. In some embodiments, the molecular signature comprises eight or more biomarkers that are enriched in the subject relative to an uninfected or recovered control subject. In some embodiments, the molecular signature comprises nine or more biomarkers that are enriched in the subject relative to an uninfected or recovered control subject. In some embodiments, the molecular signature comprises ten or more biomarkers that are enriched in the subject relative to an uninfected or recovered control subject.

In some embodiments, the one or more inflammatory proteins or biomarkers is selected from the group consisting of: TNF, IFNLR1, BCAM, S100A16, and IL5. In some embodiments, the one or more inflammatory proteins or biomarkers is TNF. In some embodiments, the one or more inflammatory proteins or biomarkers is IFNLR1. In some embodiments, the one or more inflammatory proteins or biomarkers is BCAM. In some embodiments, the one or more inflammatory proteins or biomarkers is S100A16. In some embodiments, the one or more inflammatory proteins or biomarkers is IL5.

In some embodiments, the one or more inflammatory proteins or biomarkers is selected from the group consisting of: TNF, IL5, IL11, IL13, IL15, IL1B, CXCL1, CXCL8, CCL3, CCL11, IL1RL2, CD28, HLA-DRA, LAG3, and PDCD1. In some embodiments, the one or more inflammatory proteins or biomarkers is TNF. In some embodiments, the one or more inflammatory proteins or biomarkers is IL5. In some embodiments, the one or more inflammatory proteins or biomarkers is IL11. In some embodiments, the one or more inflammatory proteins or biomarkers is IL13. In some embodiments, the one or more inflammatory proteins or biomarkers is IL15. In some embodiments, the one or more inflammatory proteins or biomarkers is IL1B. In some embodiments, the one or more inflammatory proteins or biomarkers is CXCL1. In some embodiments, the one or more inflammatory proteins or biomarkers is CXCL8. In some embodiments, the one or more inflammatory proteins or biomarkers is CCL3. In some embodiments, the one or more inflammatory proteins or biomarkers is CCL11. In some embodiments, the one or more inflammatory proteins or biomarkers is IL1RL2. In some embodiments, the one or more inflammatory proteins or biomarkers is CD28. In some embodiments, the one or more inflammatory proteins or biomarkers is HLA-DRA. In some embodiments, the one or more inflammatory proteins or biomarkers is LAG3. In some embodiments, the one or more inflammatory proteins or biomarkers is PDCD1.

In some embodiments, the one or more inflammatory proteins or biomarkers is selected from the group consisting of: CRH, CRHR1, and PTH1R. In some embodiments, the one or more inflammatory proteins or biomarkers is CRH. In some embodiments, the one or more inflammatory proteins or biomarkers is CRHR1. In some embodiments, the one or more inflammatory proteins or biomarkers is PTH1R. In some embodiments, the one or more inflammatory proteins or biomarkers is selected from the group consisting of: AP-1, BACH, BATF, IRF, and STAT. In some embodiments, the one or more inflammatory proteins or biomarkers is AP-1. In some embodiments, the one or more inflammatory proteins or biomarkers is BACH. In some embodiments, the one or more inflammatory proteins or biomarkers is BATF. In some embodiments, the one or more inflammatory proteins or biomarkers is IRF. In some embodiments, the one or more inflammatory proteins or biomarkers is STAT.

In some embodiments, the one or more inflammatory proteins is selected from the group consisting of: type II interferon (IFN), NF-κB, NF-κB-activating cytokine, IL-12, p40, IFN-γ-driven chemokine, TNF-driven cytokine and chemokine, Type I IFN, cytokine, IFNλ, and IL-12. In some embodiments, the enrichment of the type II IFN is associated with the enrichment of one or more of Type II IFN-γ, IL-27, and TID. In some embodiments, the enrichment of the type II IFN is associated with the enrichment of Type II IFN-γ. In some embodiments, the enrichment of the type II IFN is associated with the enrichment of IL-27. In some embodiments, the enrichment of the type II IFN is associated with the enrichment of TID. In some embodiments, the enrichment of the Type II IFN-γ is associated with the enrichment of one or more of IL-27, IL-18, and NF-κB. In some embodiments, the enrichment of the Type II IFN-γ is associated with the enrichment of IL-27. In some embodiments, the enrichment of the Type II IFN-γ is associated with the enrichment of IL-18. In some embodiments, the enrichment of the Type II IFN-γ is associated with the enrichment of NF-κB. In some embodiments, the enrichment of the NF-κB is associated with the enrichment of TNF. In some embodiments, the enrichment of the TNF is associated with the enrichment of one or more of IL-1 and IL-18. In some embodiments, the enrichment of the TNF is associated with the enrichment of IL-1. In some embodiments, the enrichment of the TNF is associated with the enrichment of IL-18. In some embodiments, the enrichment of the NF-κB-activating cytokine is associated with the enrichment of one or more of IL-18, TNF, and IL-1. In some embodiments, the enrichment of the NF-κB-activating cytokine is associated with the enrichment of IL-18. In some embodiments, the enrichment of the NF-κB-activating cytokine is associated with the enrichment of TNF. In some embodiments, the enrichment of the NF-κB-activating cytokine is associated with the enrichment of IL-1. In some embodiments, the enrichment of the TNF-driven cytokine and chemokine is associated with the enrichment of one or more of IL-6, CCL7, and MCP3. In some embodiments, the enrichment of the TNF-driven cytokine and chemokine is associated with the enrichment of IL-6. In some embodiments, the enrichment of the TNF-driven cytokine and chemokine is associated with the enrichment of CCL7. In some embodiments, the enrichment of the TNF-driven cytokine and chemokine is associated with the enrichment of MCP3. In some embodiments, the enrichment of the Type I IFN is associated with the enrichment of one or more of SAMD9L, MNDA, DDX58, and LAMP3. In some embodiments, the enrichment of the Type I IFN is associated with the enrichment of SAMD9L. In some embodiments, the enrichment of the Type I IFN is associated with the enrichment of MNDA. In some embodiments, the enrichment of the Type I IFN is associated with the enrichment of DDX58. In some embodiments, the enrichment of the Type I IFN is associated with the enrichment of LAMP3. In some embodiments, the enrichment of the cytokine is associated with the enrichment of one or more of IFN-γ, IFN-β, IFN-λ1/2/3, TNF, IL-6, IL-1β, and PTX3. In some embodiments, the enrichment of the cytokine is associated with the enrichment of IFN-γ. In some embodiments, the enrichment of the cytokine is associated with the enrichment of IFN-β. In some embodiments, the enrichment of the cytokine is associated with the enrichment of IFN-λ1/2/3. In some embodiments, the enrichment of the cytokine is associated with the enrichment of TNF. In some embodiments, the enrichment of the cytokine is associated with the enrichment of IL-6. In some embodiments, the enrichment of the cytokine is associated with the enrichment of IL-1β. In some embodiments, the enrichment of the cytokine is associated with the enrichment of PTX3. In some embodiments, the molecular signature is a serum proteome signature.

In some embodiments, the enrichment is between 1.5-fold and 10-fold as compared to an uninfected or recovered control subject. In some embodiments, the enrichment is 1.5-fold. In some embodiments, the enrichment is 1.6-fold. In some embodiments, the enrichment is 1.7-fold. In some embodiments, the enrichment is 1.8-fold. In some embodiments, the enrichment is 1.9-fold. In some embodiments, the enrichment is 2.0-fold. In some embodiments, the enrichment is 2.1-fold. In some embodiments, the enrichment is 2.2-fold. In some embodiments, the enrichment is 2.3-fold. In some embodiments, the enrichment is 2.4-fold. In some embodiments, the enrichment is 2.5-fold. In some embodiments, the enrichment is 2.6-fold. In some embodiments, the enrichment is 2.7-fold. In some embodiments, the enrichment is 2.8-fold. In some embodiments, the enrichment is 2.9-fold. In some embodiments, the enrichment is 3.0-fold. In some embodiments, the enrichment is 3.1-fold. In some embodiments, the enrichment is 3.2-fold. In some embodiments, the enrichment is 3.3-fold. In some embodiments, the enrichment is 3.4-fold. In some embodiments, the enrichment is 3.5-fold. In some embodiments, the enrichment is 3.6-fold. In some embodiments, the enrichment is 3.7-fold. In some embodiments, the enrichment is 3.8-fold. In some embodiments, the enrichment is 3.9-fold. In some embodiments, the enrichment is 4.0-fold. In some embodiments, the enrichment is 4.5-fold. In some embodiments, the enrichment is 5.0-fold. In some embodiments, the enrichment is 5.5-fold. In some embodiments, the enrichment is 6.0-fold. In some embodiments, the enrichment is 6.5-fold. In some embodiments, the enrichment is 7.0-fold. In some embodiments, the enrichment is 7.5-fold. In some embodiments, the enrichment is 8.0-fold. In some embodiments, the enrichment is 8.5-fold. In some embodiments, the enrichment is 9.0-fold. In some embodiments, the enrichment is 9.5-fold. In some embodiments, the enrichment is 10.0-fold. In some embodiments, the enrichment is between 1.1-fold and 1.5-fold. In some embodiments, the enrichment is between 1.5-fold and 2.0-fold. In some embodiments, the enrichment is between 2.0-fold and 2.5-fold. In some embodiments, the enrichment is between 2.5-fold and 3.0-fold. In some embodiments, the enrichment is between 3.0-fold and 3.5-fold. In some embodiments, the enrichment is between 3.5-fold and 4.0-fold. In some embodiments, the enrichment is between 4.0-fold and 4.5-fold. In some embodiments, the enrichment is between 4.5-fold and 5.0-fold. In some embodiments, the enrichment is between 5.0-fold and 5.5-fold. In some embodiments, the enrichment is between 5.5-fold and 6.0-fold. In some embodiments, the enrichment is between 6.0-fold and 6.5-fold. In some embodiments, the enrichment is between 6.5-fold and 7.0-fold. In some embodiments, the enrichment is between 7.0-fold and 7.5-fold. In some embodiments, the enrichment is between 7.5-fold and 8.0-fold. In some embodiments, the enrichment is between 8.0-fold and 8.5-fold. In some embodiments, the enrichment is between 8.5-fold and 9.0-fold. In some embodiments, the enrichment is between 9.0-fold and 9.5-fold. In some embodiments, the enrichment is between 9.5-fold and 10.0-fold. In some embodiments, the enrichment is 10.0-fold or more.

In some embodiments, the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV). In some embodiments, the virus is MERS-CoV. In some embodiments, the virus is Epstein Barr virus (EBV). In some embodiments, the virus is Ross River virus (RRV). In some embodiments, the virus is human immunodeficiency virus (HIV). In some embodiments, the virus is Ebolavirus. In some embodiments, the virus is chikungunya virus (CHIKV). In some embodiments, the virus is SARS-CoV-2. In some embodiments, the chronic inflammatory syndrome is post-acute sequelae of SARS-CoV-2 infection (PASC). In some embodiments, the subject is likely to have persistent symptoms lasting a specific period after onset of the infection. In some embodiments, the specific period is between 30 days and 2 years. In some embodiments, the specific period is at least 30 days. In some embodiments, the specific period is at least 45 days. In some embodiments, the specific period is at least 60 days. In some embodiments, the specific period is at least 75 days. In some embodiments, the specific period is at least 90 days. In some embodiments, the specific period is between 30 days and 90 days. In some embodiments, the specific period is between 90 days and 180 days. In some embodiments, the specific period is between 180 days and 1 year. In some embodiments, the specific period is between 1 year and 2 years. In some embodiments, the specific period is 2 years or more. In some embodiments, the specific period is between 30 days and 60 days. In some embodiments, the specific period is between 60 days and 90 days. In some embodiments, the specific period is between 90 days and 120 days. In some embodiments, the specific period is between 120 days and 150 days. In some embodiments, the specific period is between 150 days and 180 days. In some embodiments, the specific period is between 180 days and 210 days. In some embodiments, the specific period is between 210 days and 240 days. In some embodiments, the specific period is between 240 days and 270 days. In some embodiments, the specific period is between 270 days and 300 days. In some embodiments, the specific period is between 300 days and 330 days. In some embodiments, the specific period is between 330 days and 1 year.

In another aspect, disclosed herein is a molecular signature for use in diagnosing a subject as having a chronic inflammatory syndrome with or without a chronic or long-term infection with a virus or other pathogen, the molecular signature comprising one or more inflammatory proteins or biomarkers that are enriched in the subject relative to an uninfected or recovered control subject. In some embodiments, the molecular signature comprises two or more inflammatory proteins or biomarkers that are enriched in the subject relative to an uninfected or recovered control subject. In some embodiments, the molecular signature comprises three or more inflammatory proteins or biomarkers that are enriched in the subject relative to an uninfected or recovered control subject. In some embodiments, the molecular signature comprises four or more inflammatory proteins or biomarkers that are enriched in the subject relative to an uninfected or recovered control subject. In some embodiments, the molecular signature comprises five or more inflammatory proteins or biomarkers that are enriched in the subject relative to an uninfected or recovered control subject. In some embodiments, the molecular signature comprises six or more inflammatory proteins or biomarkers that are enriched in the subject relative to an uninfected or recovered control subject. In some embodiments, the molecular signature comprises seven or more inflammatory proteins or biomarkers that are enriched in the subject relative to an uninfected or recovered control subject. In some embodiments, the molecular signature comprises eight or more inflammatory proteins or biomarkers that are enriched in the subject relative to an uninfected or recovered control subject. In some embodiments, the molecular signature comprises nine or more inflammatory proteins or biomarkers that are enriched in the subject relative to an uninfected or recovered control subject. In some embodiments, the molecular signature comprises ten or more inflammatory proteins or biomarkers that are enriched in the subject relative to an uninfected or recovered control subject. In some embodiments, the molecular signature comprises eleven or more inflammatory proteins or biomarkers that are enriched in the subject relative to an uninfected or recovered control subject. In some embodiments, the molecular signature comprises twelve or more inflammatory proteins or biomarkers that are enriched in the subject relative to an uninfected or recovered control subject. In some embodiments, the molecular signature comprises thirteen or more inflammatory proteins or biomarkers that are enriched in the subject relative to an uninfected or recovered control subject. In some embodiments, the molecular signature comprises fourteen or more inflammatory proteins or biomarkers that are enriched in the subject relative to an uninfected or recovered control subject. In some embodiments, the molecular signature comprises fifteen or more inflammatory proteins or biomarkers that are enriched in the subject relative to an uninfected or recovered control subject. In some embodiments, the molecular signature comprises sixteen or more inflammatory proteins or biomarkers that are enriched in the subject relative to an uninfected or recovered control subject. In some embodiments, the molecular signature comprises seventeen or more inflammatory proteins or biomarkers that are enriched in the subject relative to an uninfected or recovered control subject. In some embodiments, the molecular signature comprises eighteen or more inflammatory proteins or biomarkers that are enriched in the subject relative to an uninfected or recovered control subject. In some embodiments, the molecular signature comprises nineteen or more inflammatory proteins or biomarkers that are enriched in the subject relative to an uninfected or recovered control subject. In some embodiments, the molecular signature comprises twenty or more inflammatory proteins or biomarkers that are enriched in the subject relative to an uninfected or recovered control subject.

In some embodiments, the molecular signature comprises one or more inflammatory proteins that are enriched in the subject relative to an uninfected or recovered control subject. In some embodiments, the molecular signature comprises two or more inflammatory proteins that are enriched in the subject relative to an uninfected or recovered control subject. In some embodiments, the molecular signature comprises three or more inflammatory proteins that are enriched in the subject relative to an uninfected or recovered control subject. In some embodiments, the molecular signature comprises four or more inflammatory proteins that are enriched in the subject relative to an uninfected or recovered control subject. In some embodiments, the molecular signature comprises five or more inflammatory proteins that are enriched in the subject relative to an uninfected or recovered control subject. In some embodiments, the molecular signature comprises six or more inflammatory proteins that are enriched in the subject relative to an uninfected or recovered control subject. In some embodiments, the molecular signature comprises seven or more inflammatory proteins that are enriched in the subject relative to an uninfected or recovered control subject. In some embodiments, the molecular signature comprises eight or more inflammatory proteins that are enriched in the subject relative to an uninfected or recovered control subject. In some embodiments, the molecular signature comprises nine or more inflammatory proteins that are enriched in the subject relative to an uninfected or recovered control subject. In some embodiments, the molecular signature comprises ten or more inflammatory proteins that are enriched in the subject relative to an uninfected or recovered control subject.

In some embodiments, the molecular signature comprises one or more biomarkers that are enriched in the subject relative to an uninfected or recovered control subject. In some embodiments, the molecular signature comprises two or more biomarkers that are enriched in the subject relative to an uninfected or recovered control subject. In some embodiments, the molecular signature comprises three or more biomarkers that are enriched in the subject relative to an uninfected or recovered control subject. In some embodiments, the molecular signature comprises four or more biomarkers that are enriched in the subject relative to an uninfected or recovered control subject. In some embodiments, the molecular signature comprises five or more biomarkers that are enriched in the subject relative to an uninfected or recovered control subject. In some embodiments, the molecular signature comprises six or more biomarkers that are enriched in the subject relative to an uninfected or recovered control subject. In some embodiments, the molecular signature comprises seven or more biomarkers that are enriched in the subject relative to an uninfected or recovered control subject. In some embodiments, the molecular signature comprises eight or more biomarkers that are enriched in the subject relative to an uninfected or recovered control subject. In some embodiments, the molecular signature comprises nine or more biomarkers that are enriched in the subject relative to an uninfected or recovered control subject. In some embodiments, the molecular signature comprises ten or more biomarkers that are enriched in the subject relative to an uninfected or recovered control subject.

In some embodiments, the one or more inflammatory proteins or biomarkers is selected from the group consisting of: TNF, IFNLR1, BCAM, S100A16, and IL5. In some embodiments, the one or more inflammatory proteins or biomarkers is TNF. In some embodiments, the one or more inflammatory proteins or biomarkers is IFNLR1. In some embodiments, the one or more inflammatory proteins or biomarkers is BCAM. In some embodiments, the one or more inflammatory proteins or biomarkers is S100A16. In some embodiments, the one or more inflammatory proteins or biomarkers is IL5.

In some embodiments, the one or more inflammatory proteins or biomarkers is selected from the group consisting of: TNF, IL5, IL11, IL13, IL15, IL1B, CXCL1, CXCL8, CCL3, CCL11, IL1RL2, CD28, HLA-DRA, LAG3, and PDCD1. In some embodiments, the one or more inflammatory proteins or biomarkers is TNF. In some embodiments, the one or more inflammatory proteins or biomarkers is IL5. In some embodiments, the one or more inflammatory proteins or biomarkers is IL11. In some embodiments, the one or more inflammatory proteins or biomarkers is IL13. In some embodiments, the one or more inflammatory proteins or biomarkers is IL15. In some embodiments, the one or more inflammatory proteins or biomarkers is IL1B. In some embodiments, the one or more inflammatory proteins or biomarkers is CXCL1. In some embodiments, the one or more inflammatory proteins or biomarkers is CXCL8. In some embodiments, the one or more inflammatory proteins or biomarkers is CCL3. In some embodiments, the one or more inflammatory proteins or biomarkers is CCL11. In some embodiments, the one or more inflammatory proteins or biomarkers is IL1RL2. In some embodiments, the one or more inflammatory proteins or biomarkers is CD28. In some embodiments, the one or more inflammatory proteins or biomarkers is HLA-DRA. In some embodiments, the one or more inflammatory proteins or biomarkers is LAG3. In some embodiments, the one or more inflammatory proteins or biomarkers is PDCD1.

In some embodiments, the one or more inflammatory proteins or biomarkers is selected from the group consisting of: CRH, CRHR1, and PTH1R. In some embodiments, the one or more inflammatory proteins or biomarkers is CRH. In some embodiments, the one or more inflammatory proteins or biomarkers is CRHR1. In some embodiments, the one or more inflammatory proteins or biomarkers is PTH1R. In some embodiments, the one or more inflammatory proteins or biomarkers is selected from the group consisting of: AP-1, BACH, BATF, IRF, and STAT. In some embodiments, the one or more inflammatory proteins or biomarkers is AP-1. In some embodiments, the one or more inflammatory proteins or biomarkers is BACH. In some embodiments, the one or more inflammatory proteins or biomarkers is BATF. In some embodiments, the one or more inflammatory proteins or biomarkers is IRF. In some embodiments, the one or more inflammatory proteins or biomarkers is STAT.

In some embodiments, the one or more inflammatory proteins is selected from the group consisting of: type II interferon (IFN), NF-κB, NF-κB-activating cytokine, IL-12, p40, IFN-γ-driven chemokine, TNF-driven cytokine and chemokine, Type I IFN, cytokine, IFNλ, and IL-12. In some embodiments, the enrichment of the type II IFN is associated with the enrichment of one or more of Type II IFN-γ, IL-27, and TID. In some embodiments, the enrichment of the type II IFN is associated with the enrichment of Type II IFN-γ. In some embodiments, the enrichment of the type II IFN is associated with the enrichment of IL-27. In some embodiments, the enrichment of the type II IFN is associated with the enrichment of TID. In some embodiments, the enrichment of the Type II IFN-γ is associated with the enrichment of one or more of IL-27, IL-18, and NF-κB. In some embodiments, the enrichment of the Type II IFN-γ is associated with the enrichment of IL-27. In some embodiments, the enrichment of the Type II IFN-γ is associated with the enrichment of IL-18. In some embodiments, the enrichment of the Type II IFN-γ is associated with the enrichment of NF-κB. In some embodiments, the enrichment of the NF-κB is associated with the enrichment of TNF. In some embodiments, the enrichment of the TNF is associated with the enrichment of one or more of IL-1 and IL-18. In some embodiments, the enrichment of the TNF is associated with the enrichment of IL-1. In some embodiments, the enrichment of the TNF is associated with the enrichment of IL-18. In some embodiments, the enrichment of the NF-κB-activating cytokine is associated with the enrichment of one or more of IL-18, TNF, and IL-1. In some embodiments, the enrichment of the NF-κB-activating cytokine is associated with the enrichment of IL-18. In some embodiments, the enrichment of the NF-κB-activating cytokine is associated with the enrichment of TNF. In some embodiments, the enrichment of the NF-κB-activating cytokine is associated with the enrichment of IL-1. In some embodiments, the enrichment of the TNF-driven cytokine and chemokine is associated with the enrichment of one or more of IL-6, CCL7, and MCP3. In some embodiments, the enrichment of the TNF-driven cytokine and chemokine is associated with the enrichment of IL-6. In some embodiments, the enrichment of the TNF-driven cytokine and chemokine is associated with the enrichment of CCL7. In some embodiments, the enrichment of the TNF-driven cytokine and chemokine is associated with the enrichment of MCP3. In some embodiments, the enrichment of the Type I IFN is associated with the enrichment of one or more of SAMD9L, MNDA, DDX58, and LAMP3. In some embodiments, the enrichment of the Type I IFN is associated with the enrichment of SAMD9L. In some embodiments, the enrichment of the Type I IFN is associated with the enrichment of MNDA. In some embodiments, the enrichment of the Type I IFN is associated with the enrichment of DDX58. In some embodiments, the enrichment of the Type I IFN is associated with the enrichment of LAMP3. In some embodiments, the enrichment of the cytokine is associated with the enrichment of one or more of IFN-γ, IFN-β, IFN-λ1/2/3, TNF, IL-6, IL-1β, and PTX3. In some embodiments, the enrichment of the cytokine is associated with the enrichment of IFN-γ. In some embodiments, the enrichment of the cytokine is associated with the enrichment of IFN-β. In some embodiments, the enrichment of the cytokine is associated with the enrichment of IFN-λ1/2/3. In some embodiments, the enrichment of the cytokine is associated with the enrichment of TNF. In some embodiments, the enrichment of the cytokine is associated with the enrichment of IL-6. In some embodiments, the enrichment of the cytokine is associated with the enrichment of IL-1β. In some embodiments, the enrichment of the cytokine is associated with the enrichment of PTX3. In some embodiments, the molecular signature is a serum proteome signature.

In some embodiments, the enrichment is between 1.5-fold and 10-fold as compared to an uninfected or recovered control subject. In some embodiments, the enrichment is 1.5-fold. In some embodiments, the enrichment is 1.6-fold. In some embodiments, the enrichment is 1.7-fold. In some embodiments, the enrichment is 1.8-fold. In some embodiments, the enrichment is 1.9-fold. In some embodiments, the enrichment is 2.0-fold. In some embodiments, the enrichment is 2.1-fold. In some embodiments, the enrichment is 2.2-fold. In some embodiments, the enrichment is 2.3-fold. In some embodiments, the enrichment is 2.4-fold. In some embodiments, the enrichment is 2.5-fold. In some embodiments, the enrichment is 2.6-fold. In some embodiments, the enrichment is 2.7-fold. In some embodiments, the enrichment is 2.8-fold. In some embodiments, the enrichment is 2.9-fold. In some embodiments, the enrichment is 3.0-fold. In some embodiments, the enrichment is 3.1-fold. In some embodiments, the enrichment is 3.2-fold. In some embodiments, the enrichment is 3.3-fold. In some embodiments, the enrichment is 3.4-fold. In some embodiments, the enrichment is 3.5-fold. In some embodiments, the enrichment is 3.6-fold. In some embodiments, the enrichment is 3.7-fold. In some embodiments, the enrichment is 3.8-fold. In some embodiments, the enrichment is 3.9-fold. In some embodiments, the enrichment is 4.0-fold. In some embodiments, the enrichment is 4.5-fold. In some embodiments, the enrichment is 5.0-fold. In some embodiments, the enrichment is 5.5-fold. In some embodiments, the enrichment is 6.0-fold. In some embodiments, the enrichment is 6.5-fold. In some embodiments, the enrichment is 7.0-fold. In some embodiments, the enrichment is 7.5-fold. In some embodiments, the enrichment is 8.0-fold. In some embodiments, the enrichment is 8.5-fold. In some embodiments, the enrichment is 9.0-fold. In some embodiments, the enrichment is 9.5-fold. In some embodiments, the enrichment is 10.0-fold. In some embodiments, the enrichment is between 1.1-fold and 1.5-fold. In some embodiments, the enrichment is between 1.5-fold and 2.0-fold. In some embodiments, the enrichment is between 2.0-fold and 2.5-fold. In some embodiments, the enrichment is between 2.5-fold and 3.0-fold. In some embodiments, the enrichment is between 3.0-fold and 3.5-fold. In some embodiments, the enrichment is between 3.5-fold and 4.0-fold. In some embodiments, the enrichment is between 4.0-fold and 4.5-fold. In some embodiments, the enrichment is between 4.5-fold and 5.0-fold. In some embodiments, the enrichment is between 5.0-fold and 5.5-fold. In some embodiments, the enrichment is between 5.5-fold and 6.0-fold. In some embodiments, the enrichment is between 6.0-fold and 6.5-fold. In some embodiments, the enrichment is between 6.5-fold and 7.0-fold. In some embodiments, the enrichment is between 7.0-fold and 7.5-fold. In some embodiments, the enrichment is between 7.5-fold and 8.0-fold. In some embodiments, the enrichment is between 8.0-fold and 8.5-fold. In some embodiments, the enrichment is between 8.5-fold and 9.0-fold. In some embodiments, the enrichment is between 9.0-fold and 9.5-fold. In some embodiments, the enrichment is between 9.5-fold and 10.0-fold. In some embodiments, the enrichment is 10.0-fold or more.

In some embodiments, the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV). In some embodiments, the virus is MERS-CoV. In some embodiments, the virus is Epstein Barr virus (EBV). In some embodiments, the virus is Ross River virus (RRV). In some embodiments, the virus is human immunodeficiency virus (HIV). In some embodiments, the virus is Ebolavirus. In some embodiments, the virus is chikungunya virus (CHIKV). In some embodiments, the virus is SARS-CoV-2. In some embodiments, the chronic inflammatory syndrome is post-acute sequelae of SARS-CoV-2 infection (PASC). In some embodiments, the subject is likely to have persistent symptoms lasting a specific period after onset of the infection. In some embodiments, the specific period is between 30 days and 2 years. In some embodiments, the specific period is at least 30 days. In some embodiments, the specific period is at least 45 days. In some embodiments, the specific period is at least 60 days. In some embodiments, the specific period is at least 75 days. In some embodiments, the specific period is at least 90 days. In some embodiments, the specific period is between 30 days and 90 days. In some embodiments, the specific period is between 90 days and 180 days. In some embodiments, the specific period is between 180 days and 1 year. In some embodiments, the specific period is between 1 year and 2 years. In some embodiments, the specific period is 2 years or more. In some embodiments, the specific period is between 30 days and 60 days. In some embodiments, the specific period is between 60 days and 90 days. In some embodiments, the specific period is between 90 days and 120 days. In some embodiments, the specific period is between 120 days and 150 days. In some embodiments, the specific period is between 150 days and 180 days. In some embodiments, the specific period is between 180 days and 210 days. In some embodiments, the specific period is between 210 days and 240 days. In some embodiments, the specific period is between 240 days and 270 days. In some embodiments, the specific period is between 270 days and 300 days. In some embodiments, the specific period is between 300 days and 330 days. In some embodiments, the specific period is between 330 days and 1 year.

In another aspect, disclosed herein are methods of identifying whether a subject infected or previously infected with a virus or other pathogen is likely or not likely to suffer from a chronic inflammatory syndrome with or without a chronic or long-term infection of the virus or other pathogen, comprising: (a) determining an expression level of one or more inflammatory proteins or biomarkers of a molecular signature disclosed herein in a first sample obtained from the subject; (b) comparing the first expression level to a control expression level obtained from an uninfected or recovered control subject; and (c) classifying the subject as likely to suffer from a chronic or long-term infection of the virus or other pathogen when the expression level corresponds to a molecular signature disclosed herein. In some embodiments, the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV). In some embodiments, the virus is SARS-CoV. In some embodiments, the virus is MERS-CoV. In some embodiments, the virus is Epstein Barr virus (EBV). In some embodiments, the virus is Ross River virus (RRV). In some embodiments, the virus is human immunodeficiency virus (HIV). In some embodiments, the virus is Ebolavirus. In some embodiments, the virus is chikungunya virus (CHIKV). In some embodiments, the virus is SARS-CoV-2. In some embodiments, the chronic inflammatory syndrome is post-acute sequelae of SARS-CoV-2 infection (PASC). In some embodiments, the sample is obtained within the first 15 days of post-symptom onset. In some embodiments, the sample is obtained within the first 2 days of post-symptom onset. In some embodiments, the sample is obtained within the first 3 days of post-symptom onset. In some embodiments, the sample is obtained within the first 4 days of post-symptom onset. In some embodiments, the sample is obtained within the first 5 days of post-symptom onset. In some embodiments, the sample is obtained within the first 6 days of post-symptom onset. In some embodiments, the sample is obtained within the first 7 days of post-symptom onset. In some embodiments, the sample is obtained within the first 8 days of post-symptom onset. In some embodiments, the sample is obtained within the first 9 days of post-symptom onset. In some embodiments, the sample is obtained within the first 10 days of post-symptom onset. In some embodiments, the sample is obtained within the first 11 days of post-symptom onset. In some embodiments, the sample is obtained within the first 12 days of post-symptom onset. In some embodiments, the sample is obtained within the first 13 days of post-symptom onset. In some embodiments, the sample is obtained within the first 14 days of post-symptom onset. In some embodiments, the sample is obtained within the first 3 weeks of post-symptom onset. In some embodiments, the sample is obtained within the first 4 weeks of post-symptom onset. In some embodiments, the sample is obtained within the first 1 month of post-symptom onset. In some embodiments, the sample is obtained within the first 2 months of post-symptom onset. In some embodiments, the sample is obtained within the first 3 months of post-symptom onset. In some embodiments, the sample is obtained within the first 4 months of post-symptom onset. In some embodiments, the sample is obtained within the first 5 months of post-symptom onset. In some embodiments, the sample is obtained within the first 6 months of post-symptom onset. In some embodiments, the sample is obtained within the first 7 months of post-symptom onset. In some embodiments, the sample is obtained within the first 8 months of post-symptom onset. In some embodiments, the sample is obtained within the first 9 months of post-symptom onset. In some embodiments, the sample is obtained within the first 10 months of post-symptom onset. In some embodiments, the sample is obtained within the first 11 months of post-symptom onset. In some embodiments, the sample is obtained within the first year of post-symptom onset. In some embodiments, the sample is obtained within the first 2 years of post-symptom onset. In some embodiments, the sample is obtained within the first 3 years of post-symptom onset. In some embodiments, the sample is obtained within the first 4 years of post-symptom onset. In some embodiments, the sample is obtained within the first 5 years or more of post-symptom onset. In some embodiments, the subject is placed into a cohort for a clinical trial to test investigational drugs to treat PASC. In some embodiments, the subject is administered a drug for treating PASC. In some embodiments, the subject is likely to suffer from a chronic inflammatory syndrome with a chronic or long-term infection of the virus or other pathogen. In some embodiments, the subject is likely to suffer from a chronic inflammatory syndrome without a chronic or long-term infection of the virus or other pathogen.

In some embodiments, at least due to shared immune response pathways and mechanisms underlying infections and diseases caused by foreign antigens (e.g., viruses, bacteria, fungi, or parasites), the methods for diagnosing or classifying a subject as having a long-term infection and/or long-term symptoms associated thereof according to various embodiments disclosed therein can be applied to other types of diseases or illnesses that can induce changes in immunity, such as those caused by viruses, bacteria, fungi, or parasites. Non-limiting examples of such diseases or illnesses caused by a virus or bacterium include Severe Acute Respiratory Syndrome (SARS) caused by SARS-CoV, Middle East Respiratory Syndrome (MERS) caused by MERS-CoV, mononucleosis caused by Epstein Barr virus (EBV), Ross River fever caused by Ross River virus (RRV), human immunodeficiency virus (HIV) infection, Ebola (also known as Ebola Virus Disease (EVD)) caused by Ebolavirus, or chikungunya caused by chikungunya virus (CHIKV), Lyme disease caused by the bacterium Borrelia burgdorferi, and myalgic encephalomyelitis/chronic fatigue syndrome (ME/CFS).

The methods for diagnosing or classifying a subject as having a long-term infection and/or long-term symptoms associated thereof according to various embodiments disclosed therein can also be applied to autoimmune diseases and disorders. Therefore, in some embodiments, the disclosure provides methods for diagnosing or classifying a subject as having an autoimmune disease or disorder, and/or symptoms associated thereof. Non-limiting examples of autoimmune diseases include rheumatoid arthritis, inflammatory arthritis, lupus or systemic lupus erythematosus (SLE), inflammatory bowel disease (IBD), celiac disease, multiple sclerosis (MS), Type 1 diabetes, psoriasis, vasculitis, allergic inflammation (e.g., allergic asthma, atopic dermatitis, contact hypersensitivity), Graves' disease (i.e., overactive thyroid), Hashimoto's thyroiditis (i.e., underactive thyroid), chronic graft versus host disease, hemophilia with antibodies to coagulation factors, Crohn's disease, ulcerative colitis, Guillain-Barre syndrome, primary biliary sclerosis or cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis, giant cell arteritis, chronic fatigue syndrome (CFS), autoimmune Addison's Disease, ankylosing spondylitis, acute disseminated encephalomyelitis, antiphospholipid antibody syndrome, aplastic anemia, idiopathic thrombocytopenia purpura, myasthenia gravis, opsoclonus myoclonus syndrome, optic neuritis, chronic inflammatory demyelinating polyneuropathy, Ord's thyroiditis, pemphigus, pernicious anemia, Reiter's syndrome, Takayasu's arteritis, warm autoimmune hemolytic anemia, fibromyalgia, and drug-induced autoimmunity or immune related adverse events (IRAEs) (e.g., CAR-T cells, check point blockade, drug-induced autoimmune liver disease, drug-induced hemolytic anemia).

### Methods of Treatment or Prevention

In some aspects, the disclosure provides methods for treatment or prevention of one or more symptoms in a subject diagnosed or classified as having a long-term infection of a virus according to various embodiments disclosed herein. In some embodiments, the virus is SARS-CoV-2, and the viral infection is COVID-19. In some embodiments, the subject is diagnosed or classified as having PASC.

The term “treatment” in relation a given disease, disorder or viral infection, includes, but is not limited to, inhibiting the disease, disorder or viral infection, for example, arresting the development of the disease, disorder, or viral infection; relieving the disease, disorder, or viral infection for example, causing regression of the disease, disorder, or viral infection; or relieving a condition caused by or resulting from the disease, disorder, or viral infection for example, relieving or treating symptoms of the disease, disorder, or viral infection. The term “prevention” in relation to a given disease, disorder, or viral infection means: preventing the onset of disease, disorder, or viral infection development if none had occurred, preventing the disease, disorder, or viral infection from occurring in a subject that may be predisposed to the disorder, disease, or viral infection but has not yet been diagnosed as having the disorder, disease, or viral infection and/or preventing further disease/disorder/infection development if already present.

In some embodiments, the methods comprising administering to the subject a therapeutically effective amount one or more therapeutic agents. A “therapeutically effective amount” as used herein is an amount that produces a desired effect in a subject for treating and/or preventing a long-term viral infection. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including but not limited to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject's response to administration and adjusting the dosage accordingly. For additional guidance, see, e.g., Remington: The Science and Practice of Pharmacy, 22nd Edition, Pharmaceutical Press, London, 2012, and Goodman & Gilman's The Pharmacological Basis of Therapeutics, 12th Edition, McGraw-Hill, New York, NY, 2011, the entire disclosures of which are incorporated by reference herein.

In some embodiments, one or more therapeutic agents can be small molecule drugs, antibodies (e.g., monoclonal antibodies, polyclonal antibodies, one-antigen antibodies, multi-antigen antibodies), or siRNAs.

In some embodiments, the one or more therapeutic agents comprise an anti-inflammatory agent. Non-limiting examples of an anti-inflammatory agent include nonsteroidal anti-inflammatory drugs (NSAIDs) including aspirin, ibuprofen, naproxen, meloxicam, celecoxib, and indomethacin. An anti-inflammatory agent may also be an agent that targets an inflammatory pathway, including, but not limited to, agents targeting IL15, IL1b, CXCL5, vascular endothelial growth factor (VEGF), and inflammasome. An anti-inflammatory agent may also be an inhibitor a transcription factor, for example, an inhibitor targeting STAT1.

In some embodiments, the one or more therapeutic agents comprise an agent that induces hormonal and/or metabolic changes.

In some embodiments, the one or more therapeutic agents comprise an agent that treats or prevents tissue damage or fibrosis.

In some embodiments, the one or more therapeutic agents comprise an antagonist of a secreted factor or a receptor selected from the group consisting of TNF, IFNG, IL12A, IL1B, MDK.NOTCH2, HMGB2, LTA, GZMB, TGFB1, IL10, NAMPT, CD28, TRAIL, and PTH1R.

In some embodiments, the one or more therapeutic agents comprise an agent targeting a transcription factor selected from the group consisting of STAT, IRF, AP1, CEBPC, BACH, DDIT4, and mTOR.

In some embodiments, the one or more therapeutic agents comprise an agent targeting an immune cell, such as an agent targeting monocytes, an agent targeting DCs, an agent targeting adaptive effector immune cells (e.g., CD4+ T cells, CD8+ T cells).

In some embodiments, the methods comprising administering to the subject an agent to supplement or enhance IL15 function. As described herein, L-15 is a unique cytokine biomarker in PASC patients and is consistently lower in PASC patients compared to recovered subjects. In some embodiments, the methods comprising administering to the subject recombinant IL15. In some embodiments, the methods comprising administering to the subject an IL15 agonist, a non-limiting example of which is ALT803.

In some embodiments, the methods comprising administering to the subject an agent to inhibit or reduce IL13 function. As described herein, IL13 is another unique cytokine biomarker in PASC patients but is elevated in PASC patients compared to recovered subjects. In some embodiments, the methods comprising administering to the subject an antagonist of IL13, for example, an inhibiting antibody of IL13, a loss-of-function mutant of wild-type IL13, or a binding domain of IL13 that blocks its normal function.

In some embodiments, the methods comprising administering to the subject an agonist of Toll-like receptors (TLRs) and/or retinoic acid-inducible gene-l (RIG-I)-like receptors (RLR)s. TLRs and RLRs are distinct families of pattern-recognition receptors that sense nucleic acids derived from viruses and trigger antiviral innate immune responses. Without being bound to a particular theory, activating TLRs and RLRs through an agonist may enhance the innate immune system's abilities to fight against viral infections, which may alleviate symptoms in scenarios of long-term infections such as PASC.

In some embodiments, the methods comprising administering to the subject IFNs, including Type I, Type II, and/or Type III IFNs. IFNs are a group of signaling proteins made and released by host cells in response to the presence of viruses. In a typical scenario, a virus-infected cell will release interferons causing nearby cells to heighten their anti-viral defenses. All three classes of IFNs (i.e., Type I, Type II, and Type III IFNs) are important for fighting viral infections and for the regulation of the immune system. Without being bound to a particular theory, administration of IFNs may also contribute to the immune system's abilities to fight against viral infections, which may alleviate symptoms in scenarios of long-term infections such as PASC.

In some embodiments, the methods comprising administering to the subject an agent that treats and/or inhibit hypoxia. Hypoxia is a condition in which the body or a region of the body is deprived of adequate oxygen supply at the tissue level, and sometimes occurs in COVID-19 patients due to damages to the respiratory system.

In some embodiments, the methods comprising administering to the subject the one or more therapeutic agents for a period of time of about 3 days up to about 5 years. In some embodiments, the subject is administered the one or more therapeutic agents for about 3 days, about 4 days, about 5 days, about 6 days, about 1 week, about 1.5 weeks, about 2 weeks, about 2.5 weeks, about 3 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, or about 5 years. In some embodiments, the one or more therapeutic agents may be administered to the subject for longer than 5 years for chronic or prolonged treatment. A single dose or multiple doses of the one or more therapeutic agents may be administered to a subject once or multiple times in a period of time, e.g., a day, a week, or a month. It is within the purview of one of ordinary skill in the art to select a suitable administration route, such as oral administration, subcutaneous administration, intravenous administration, intramuscular administration, intradermal administration, intrathecal administration, or intraperitoneal administration, for the one or more therapeutic agents.

In some embodiments, the one or more therapeutic agents may be administered over a pre-determined time period. Alternatively, the one or more therapeutic agents may be administered until a particular therapeutic benchmark is reached. In certain embodiments, the methods provided herein include a step of evaluating one or more therapeutic benchmarks of the viral infection, such as the intensity of one or more symptoms associated with the viral infection, or the level of one or more biomarkers according to various embodiments disclosed herein.

In some embodiments, the methods further comprise monitoring the subject for evidence of SARS-CoV-2 infection, PASC, and/or symptoms thereof, including coughing, wheezing, fever, fatigue, anxiety, and difficulty in breathing.

### EXAMPLES

### Example 1: Biomarkers for Diagnosis, Monitoring, and Treatment of Post-Acute Sequelae to SARS-CoV-2 Infection (PASC)

The present study sought to deeply phenotype mild COVID-19 to define events coordinating innate and adaptive immunity that enable convalescent adaptive immunity and drive heterogeneity in outcomes. Participants with a WHO ordinal severity score of 2 or 3 who did not require hospitalization were recruited and analyzed longitudinally by scRNAseq, scATACseq, serum proteomics, serology, and flow cytometry for at least 3 visits spanning early acute infection to convalescent recovery or chronic post-COVID-19 up to 100 days. Proteomic profiling of serum uncovered unique protein signatures that defined early acute COVID-19 and PASC. These data provide a unique resource for integration with existing and future studies and enable mechanistic hypothesis generation on disease progression and identified multiple targets for future validation studies in immunomonitoring natural infections and vaccine-induced immunity.

SARS-CoV-2 infection results in clinically heterogeneous manifestations that are partially driven by complex longitudinal interactions with the immune system. The public health burden of mild COVID-19 is massive given the more than 100 million infected worldwide, but most research has focused on severe and fatal COVID-19. This study recruited a mild COVID-19 cohort for multimodal immunophenotyping of single immune cells (scRNAseq, scATACseq, flow cytometry), serum proteins, virus-specific cellular and humoral immune responses, and clinical annotation from early acute infection to convalescence. Samples were acquired longitudinally from early acute infection before 15 days to more than 100 days post-symptom onset (PSO). Comparison to uninfected controls revealed marked immune activation consistent with acute response to viral infection at 15 days post-symptom onset with stronger inflammatory responses in older (>=40) compared to younger (<40) participants. Type I, II, and Ill interferon responses were most consistently upregulated in nearly all PBMC subsets. Plasmablasts were expanded in early infection with signatures of active IFNL and IFNG signaling, linking early inflammation and IFN responses to control of viral replication via antibodies. Longitudinal models of scRNAseq confirmed most inflammatory pathways decreased over time, including type I and II IFN signaling, as well as signaling by innate danger sensors (TLR, RIG-I) that detect viral replication. By contrast, genes with increasing expression over time were enriched for epithelial-to-mesenchymal transition (EMT) and wound healing pathways. Most participants showed consistent decay of inflammatory signatures by convalescence at more than 30-day PSO except those presenting with PASC. Despite similar virus-specific adaptive immune responses between post-acute sequelae PASC and recovered convalescent participants, PASC participants showed persistent serum cytokine and chemokine differences including elevated IL5, TNF, IL-12p70, and soluble CD28 at more than 30-day PSO. Integrative network analyses defined the most enriched ligand-receptor pathways across PBMC subsets including LTA/TNFβ, TNF, and IFNg in early infection, and these also showed signs of persistent activation in PASC. Common downstream targets of these pathways in PASC point to novel therapeutic targets and mechanistic hypotheses, including IL-1β, AP-1, ZFP36/TTP, and CEBP/β. Serum protein signatures predicting antibody responses and correlating with PASC may provide opportunities for enhanced immunomonitoring and personalized treatment.

The present study sought to deeply phenotype mild COVID-19 to define events coordinating innate and adaptive immunity that enable convalescent responses and drive heterogeneity in outcomes. Participants with a WHO ordinal severity score of 2 or 3 who did not require hospitalization were recruited. Peripheral blood was sampled and analyzed longitudinally by (1) scRNAseq, (2) scATACseq, (3) serum proteomics, (4) serology, and (5) flow cytometry. At least 3 visits were collected for every participant at: (1) early acute infection <=15 days PSO, (2) late acute infection 16-30 days PSO, and (3) convalescent recovery or chronic post-COVID-19 from 31 to 100 days PSO. Explicit links were established between multimodal immune features over time that drive interpatient heterogeneity of SARS-CoV-2-specific immune responses, and enabled hypothesis generation and predictive modeling of convalescent outcomes including levels of virus-specific T cells, memory B cells, and antibodies, as well as occurrence of PASC. Integrative multi-omic analysis provided new insights into the key immune regulatory nodes in mild COVID-19 infection and the priming of adaptive immune responses to natural infection. These data provide a unique resource for integration with existing and future studies and enable mechanistic hypothesis generation on mild disease progression as well as identifying multiple targets for future validation studies in immunomonitoring natural infections and vaccine-induced immunity.

As discussed further below, broad and deep multi-omic immunophenotyping identified molecular and cellular differences in the serum, PBMCs, and SARS COV2-specific immune responses of PASC patients compared to recovered controls. Computational and statistical modeling was performed to construct classification models and to reconstruct key cell-cell interactions that identify potential causal mechanisms and therapeutic targets. Olink proteomics identified a set of dysregulated proteins including but not limited to: enriched (IL5, CXCL8, CCL16, LGALS3, IFNLR1, CXCL1, TNFRSF9, TNFSF10, IL24, NRP2, CCL11, IL11, IL1B, LAG3, CCL3, FASLG) and depleted (IGFBP3, IL15, CCN2, CA14) proteins. These suggest persistent cytokine and chemokine upregulation in the blood are major contributors to at least 1 subset of PASC patients, driving immune hyperactivation to create a self-perpetuating proinflammatory cascade in a broad array of immune cells including subsets of monocytes (TNF, IFNG), CD8+ T cells (IL12A, IFNG, GHRL, TNF, IL10), dendritic cells, natural killer cells, and B cells. scATACseq data indicate increased activation of signaling pathways driving AP1 family transcription factors, and other motifs such as NFE2, MAF, MYC, PRDM1, BATF, IRF, STAT, BACH2. Motifs depleted in B cells (ZEB, ID4, MESP1, and TCF3/4/12) suggest reduced signals essential for germinal center formation and differentiation. Cumulatively, these effects may drive long-term impairments of adaptive immune function and antibodies as well as a state of chronic inflammation driven by innate immune and immune effector cells. The dynamics of SARS-CoV-2-specific immunity in PASC were also dysregulated. These changes are exemplified by early rapid declines in CD8+ T cells, B cells, and plasmablasts, as well as early peaks of receptor-binding domain (RBD)-specific antibodies (IgG/A/M). Metaclustering analysis identified a non-antigen specific CD4+T effector memory cell population (metacluster 41) with CXCR3+ CCR6-GZMB− that demonstrates broader disruption of homeostatic adaptive immune repertoire, such as circulating Th1 cells, during acute inflammation that precedes PASC. In addition to facilitating diagnosis and monitoring of PASC by molecular or cellular analyses (ELISA, flow cytometry, sequencing), these characteristic signatures can be targeted by either non-specific immunosuppressive regimens or targeted therapies, such as cytokine/chemokine blocking antibodies. Changes in acute early infection (<30 days) may be useful cellular and molecular biomarkers of PASC risk stratification.

A second PASC signature was observed that was less inflammatory and immune-centric, suggesting existence of multiple causal pathogenic mechanisms in PASC and demonstrating the importance of subtype classification in monitoring and treatment. Finally, some shared elements of PASC signatures were observed in fully recovered COVID19 patients, which suggests the development and severity of PASC may lie along a spectrum and indicates some early soluble mediators that may be effective targets to identify higher-risk acute infections and prevent progression to PASC.

### Results

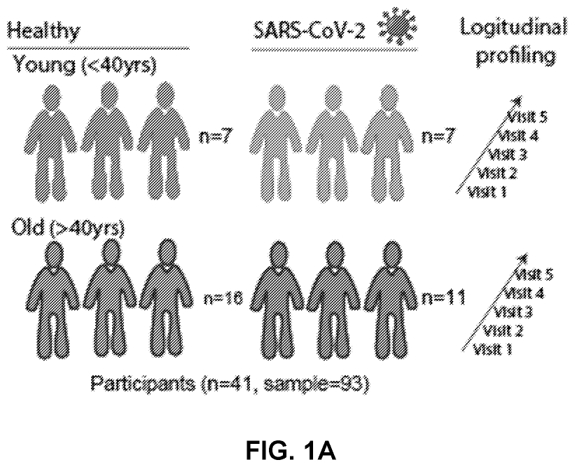

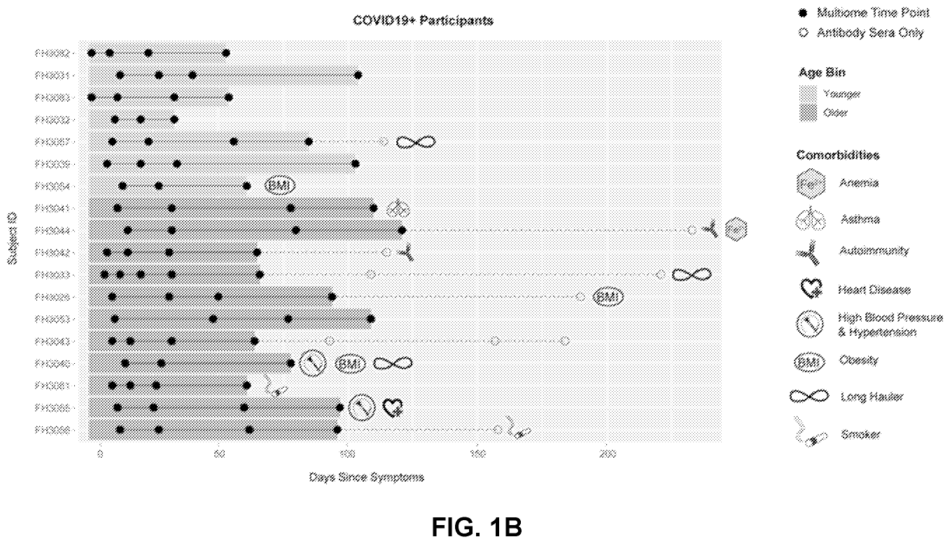

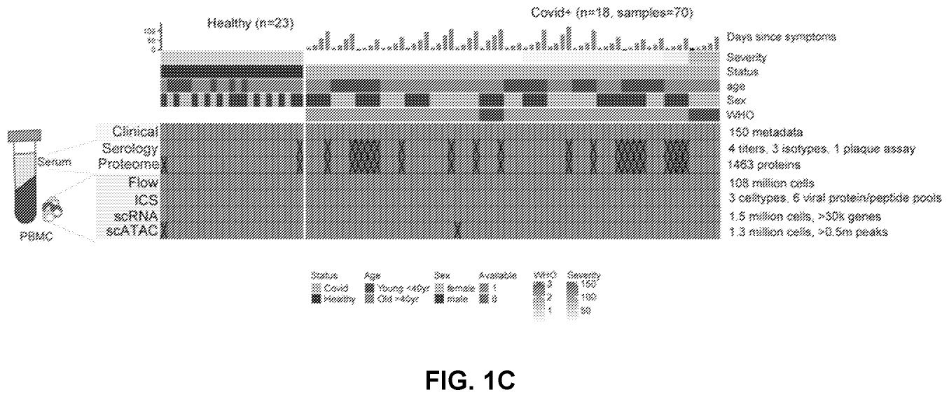

COVID-19 cases exhibited heterogeneity in clinical presentation and immune magnitude. 20 participants positive by PCR test for COVID-19 and 23 PCR-negative uninfected controls were recruited from first responders and other healthcare workers in the Seattle metropolitan area. Of the COVID-19 participants, 18 were selected for downstream analysis after filtering for inclusion criteria and quality control. Participants were split between younger and older age groups (median age 29 vs. 57 years, respectively) and were predominantly non-hispanic (n=21 vs. n=17, respectively). Peripheral blood was collected longitudinally for 3-5 visits from mild COVID-19 participants or at a single visit for uninfected controls recruited (FIG. 1A). Longitudinal samples for all COVID-19 participants included a minimum of 3 timepoints: (1) early acute infection within the first 15 days post-symptom onset (PSO), (2) late acute infection from 16-30 days PSO, and (3) post-acute COVID-19 at least 60 days PSO with a median follow-up of 81.5 (33-121) days PSO (FIG. 1B). Infection status was diagnosed by either COVID-19 PCR test or blood antibody test. All participants were either WHO ordinal severity score 2 or 3 (mild disease, no hospitalization). Each sample was processed in parallel by a multi-omic immunophenotyping pipeline, including PBMCs analyzed by scRNAseq, scATACseq, and flow cytometry, while serum was analyzed by O-link proteomics (FIG. 1C). Virus-specific adaptive immune responses were evaluated by antibody titering (IgG, IgM, IgA to spike receptor binding domain, RBD; IgG to nucleocapsid, N) (Stamatatos et al. 2021), focus reduction neutralization assays against an infectious clone (Vanderheiden et al. 2020), and intracellular cytokine stimulation (ICS) of CD4+ and CD8+ T cells using viral peptide pools covering structural proteins and memory B cells using whole proteins (S, RBD). Detailed symptom surveys were also collected at each visit during acute infection and longitudinal follow-up.

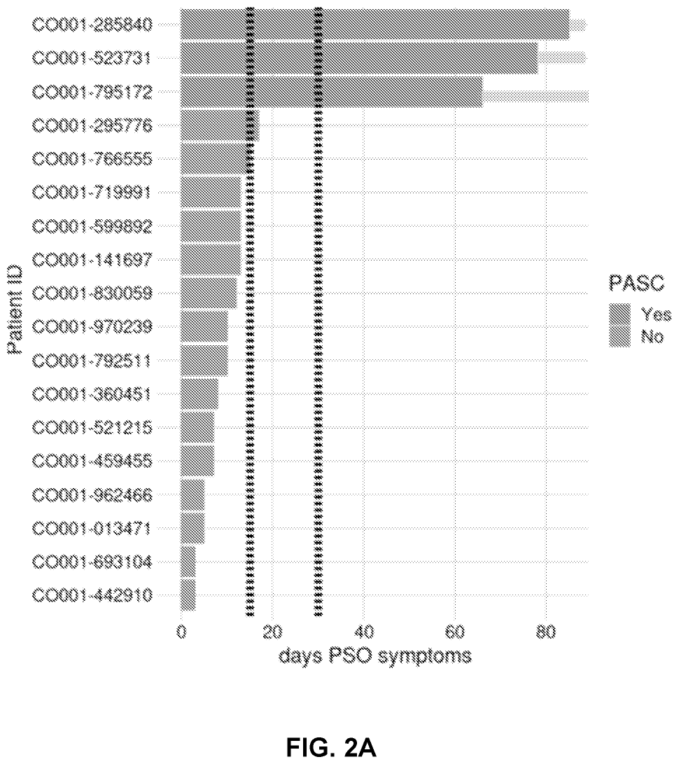

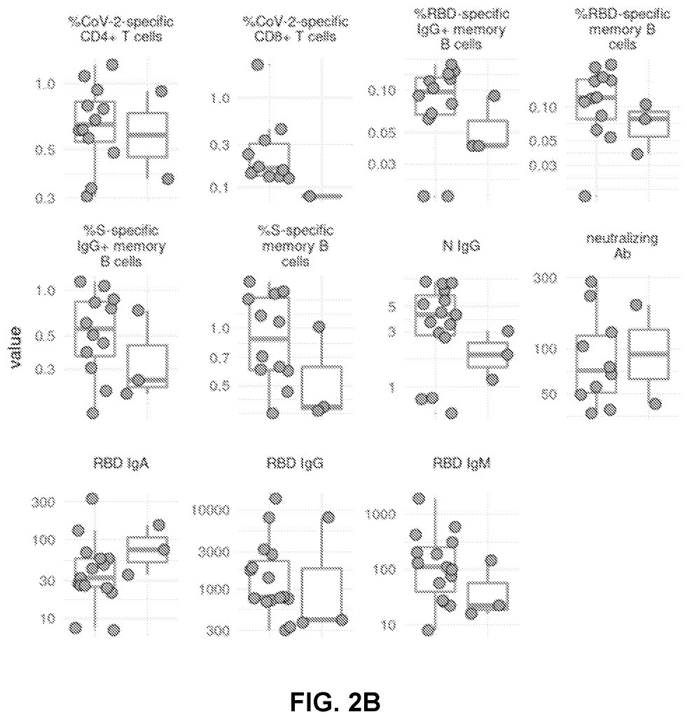

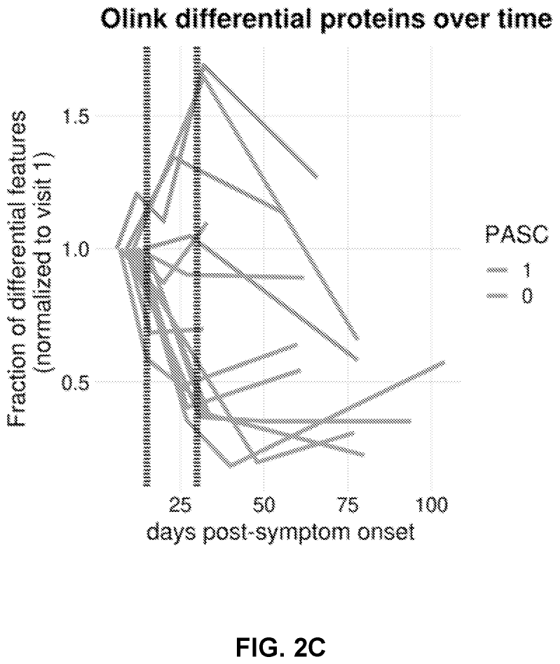

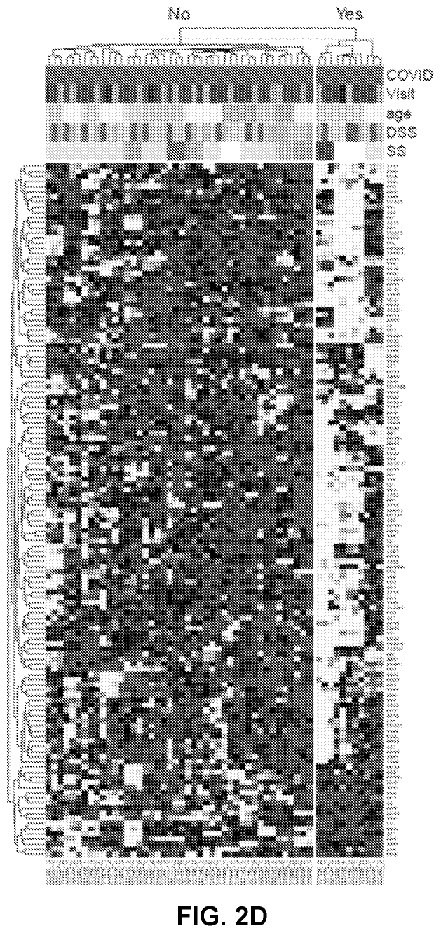

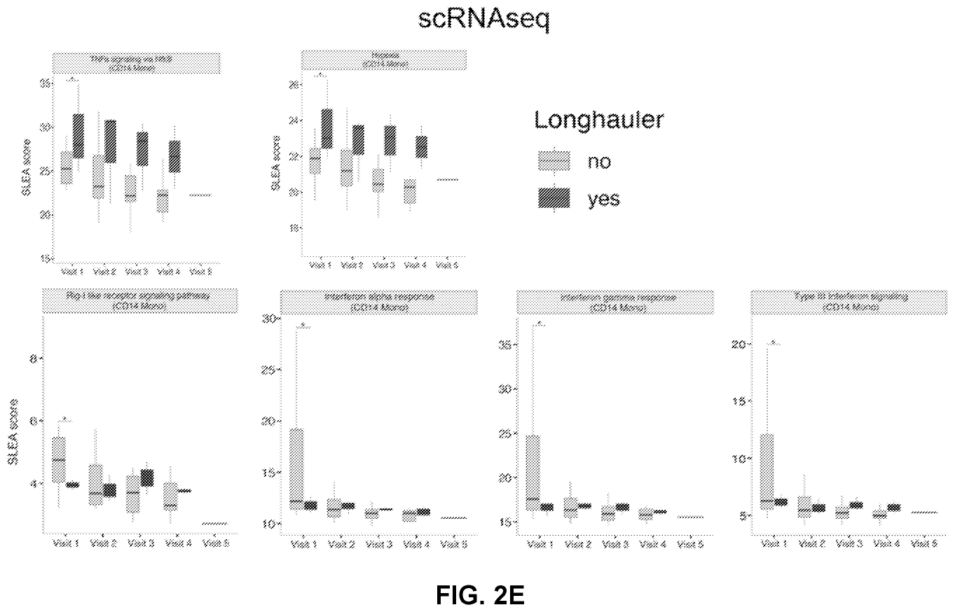

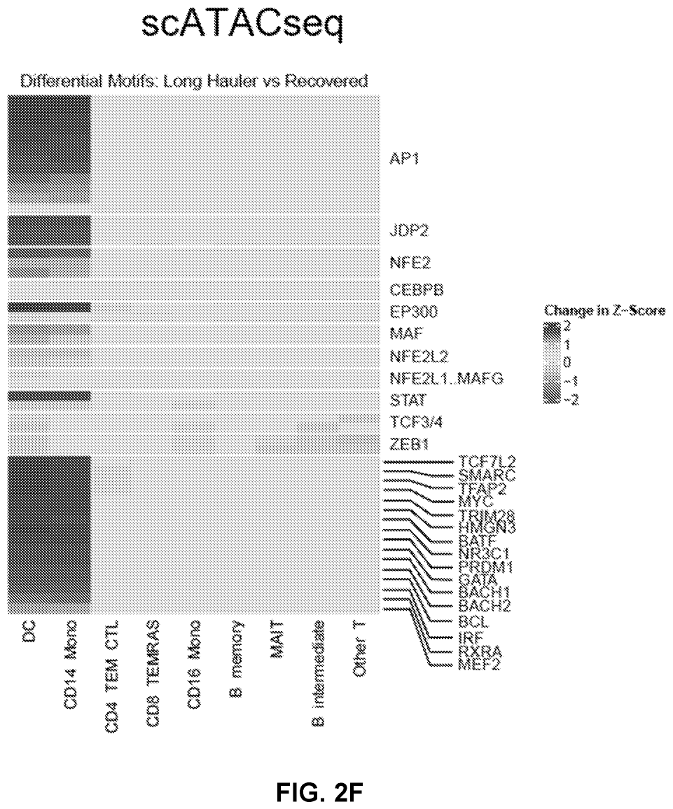

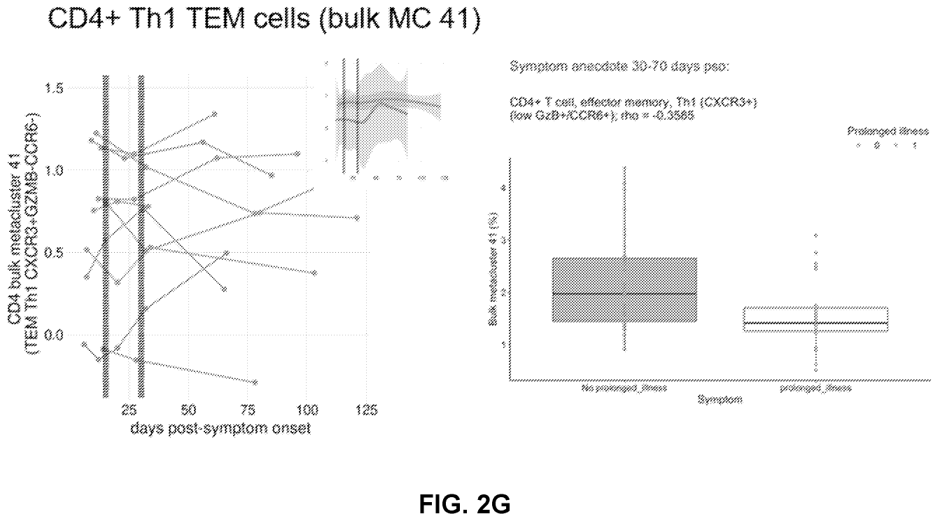

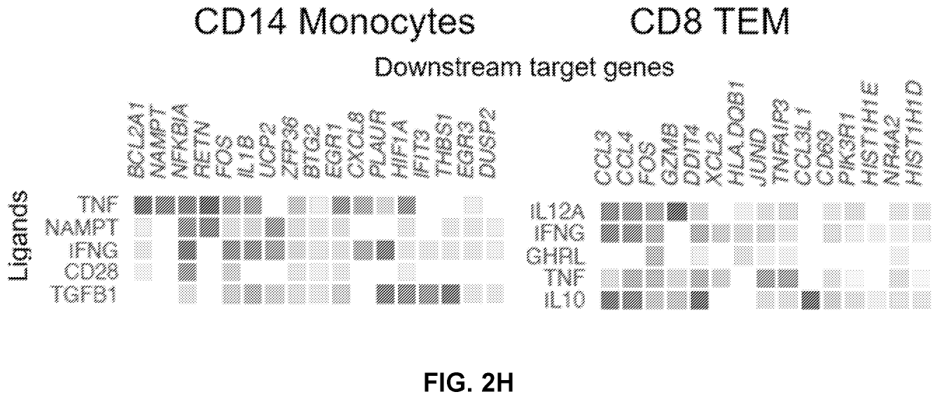

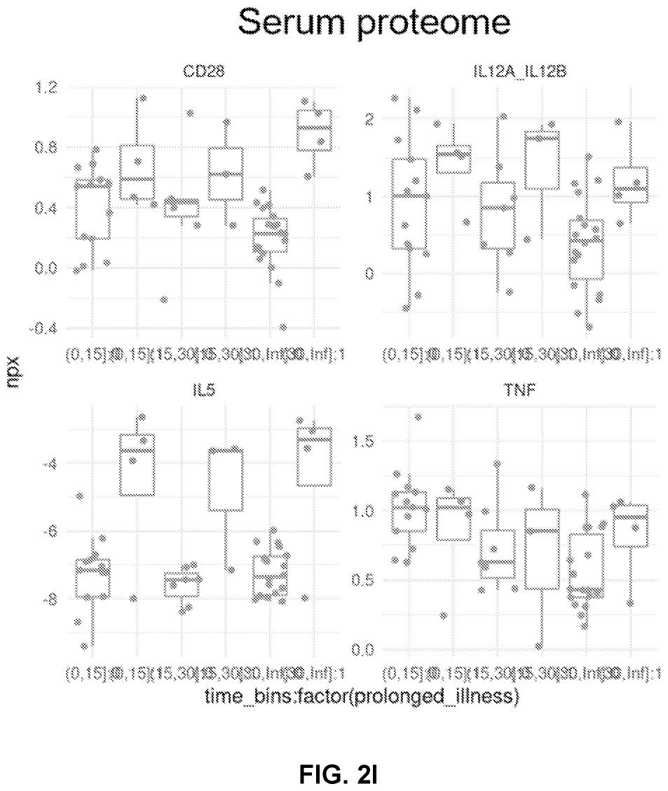

PASC participants were exceptions to resolution, sustained persistent inflammation. Recovered participants had no symptoms after day 20 PSO, while 3 participants continued to present symptoms throughout the study, termed PASC aka long COVID (FIG. 2A). These PASC participants were all female, consistent with published reports of PASC skewing female. Two participants had more severe courses of infection and persistent symptoms including cognitive impairment (PTID 795172; severity score 71, WHO score 3) and cardiovascular abnormalities (PTID 523731; severity score 141, WHO score 3). The third PASC participant (PTID 285840, severity score 3, WHO score 2) showed mild symptoms during acute infection, but presented myriad symptoms during PASC including joint swelling, tingling, chest pain, abdominal pain, and loss of smell. PASC persisted in these participants as of final follow-up (233 days PSO). PASC participants had qualitative differences in SARS-CoV-2-specific responses, but no statistical differences were identified due to the small sample size. Qualitatively, SARS-CoV-2-specific CD8+ T cells were low or absent in all PASC participants after 30 days PSO. RBD- and S-specific memory B cells, RBD IgG, and neutralizing antibodies were low in 2 of 3 PASC participants compared to recovered COVID-19 participants. RBD IgA titers of PASC participants were among the highest quantile of recovered participants. RBD IgA titers for PASC participants were among the upper quartile of recovered COVID-19 levels.

To interrogate differences in longitudinal serum proteome between PASC and recovered COVID-19 participants, outlier analysis was performed and showed most COVID-19 participants (14/15) had a decreasing number of differential proteins over time, while PASC participants had stable or increasing numbers of differential proteins over time (FIG. 2C). A signature of 132 differentially expressed proteins (p<0.05) were identified that were enriched in functional pathways for respiratory burst involved in inflammatory response (p=5.67×10⁻⁴), T cell antigen processing and presentation (p=5.67×10⁻⁴), germinal center formation (p=6×10⁻⁴), innate response in mucosa (p=3.69×10⁻³), NK proliferation (p=1.9×10⁻²), T cell cytokine production (p=1.9×10⁻²), and acute inflammatory response (p=3.86×10⁻³) (FIG. 2D). Significantly upregulated proteins included IFNLR1 (adj p=1.45×10⁻⁹), BCAM (adj p=1.03×10⁻⁷), S100A16 (adj p=9.68×10⁻⁴), IL5 (adj p=0.015), and PTH1R (adj p=0.07). A signature of increased inflammatory signals in PASC participants, including IL5, IL11, IL1B, CXCL1, CCL3, CCL11, IL1RL2, CXCL8, CD28, and HLA-DRA, suggested a hyperinflammatory state consistent with an earlier study (Ren et al. 2021). Select immunosuppressive proteins were also significantly upregulated, including checkpoint molecules LAG3 and PDCD1, which may limit adaptive immune responses to viral infection and increase symptom severity (Saheb Sharif-Askari et al. 2021). Hierarchical clustering of differentially expressed proteins showed that the 2 more severe PASC participants clustered together, while the third showed a distinct signature. A systemic inflammatory signature was more prominent in the 2 severe PASC participants including TNFRSF4/OX40, TNFRSF9/4-1BB, IL11, and IL1B, and shared elevated hormones and hormone receptors (CRH, CRHR1, PTH1R) with 523731. These signatures suggested both inflammatory and hormonal components of PASC, which may provide a molecular classification of disease subsets to better deal with heterogeneity. scRNAseq data provided evidence of dysregulated signaling in immune cells, including CD14+ monocytes (FIG. 2E). TNF signaling and hypoxia pathways were significantly higher in CD14+ monocytes from PASC participants compared to uninfected controls. Early infection in PASC was characterized by significantly lower scores for RIG-I signaling and IFN responses. These signatures persisted at similar levels throughout disease, while levels in uninfected participants dropped. These data paint a complex picture of PASC that include potential pathogenic roles for chronic inflammation and cellular stress coupled with poor early innate immune responses. This latter finding parallels results from other studies showing SARS-CoV-2 dysregulates early innate immune responses and IFNs in more severe acute COVID-19.

Transcription factor motif analysis of scATACseq revealed key motifs correlated with aberrant cell phenotypes in PASC participants compared to COVID-19+ recovered participants at >30 days PSO. A set of AP-1 family motifs were significantly enriched in dendritic cells (DCs) and CD14+ monocytes, providing further evidence of persistent immune activation in key innate immune phagocytes (FIG. 2F). Other prominent enriched motifs were BACH, BATF, IRF, and STAT families, suggesting ongoing inflammatory cytokine signaling in innate immune cells. The same motifs were also enriched, albeit to a lesser extent, in CD4+ and CD8+ effector memory T cells. PASC-specific motif enrichments were not observed across all innate immune cells or for most adaptive immune cell types, suggesting innate inflammation is a major contributor to pathogenesis. Many transcription factor motifs, such as Myc, were broadly enriched in acute infection persisted of PASC participants, supporting hypotheses of prolonged unresolved inflammation >30 days PSO. Metaclustering analysis revealed a non-CoV-2-specific CD4+ T cell subset that was low in PASC participants (FIG. 2G). In a broader cohort of COVID-19 participants, these cells were validated. These cells were CD4+ T cells with a Th1 and TEM-like phenotype (CXCR3+GZMB− CCR6−), termed bulk metacluster 41. The two more severe PASC participants showed depletion of this subset at 30-70 days PSO and together with high IL-5 suggested potential Th2 skewing in PASC.

scRNAseq data was analyzed by NicheNet to identify predicted ligand-receptor interactions that were associated with persistent innate immune activation. DEGs for PASC participants compared to recovered COVID-19+ participants were used as the input and CD14+ monocytes were set as receiver cells with all other celltypes set as senders (FIG. 2H). Olink data showed that these signals were elevated in serum of PASC participants (FIG. 2I). This analysis identified TNF and IFNG as key inflammatory cytokines contributing to the gene expression signature of CD14+ monocytes in PASC participants. These cytokines may be driving AP-1 and STAT/IRF enrichment observed in motif analyses, and motivating therapeutic targeting of these cytokines with therapeutic blockade. NAMPT is a major regulator of NAD metabolism as well as a known myeloid cell modulator. In other inflammatory disorders, it has been identified as upregulated and a potential therapeutic target based on preclinical models. Serum proteome data confirmed upregulation of a subset of predicted ligands, including increased CD28, IL-12p70, IL-5, and TNF. Overall, these data suggested a key role for the inflammatory cytokine milieu driving a persistently proinflammatory state in CD14+ monocytes, identifying biomarkers for diagnosis of PASC and revealing multiple therapeutic strategies.

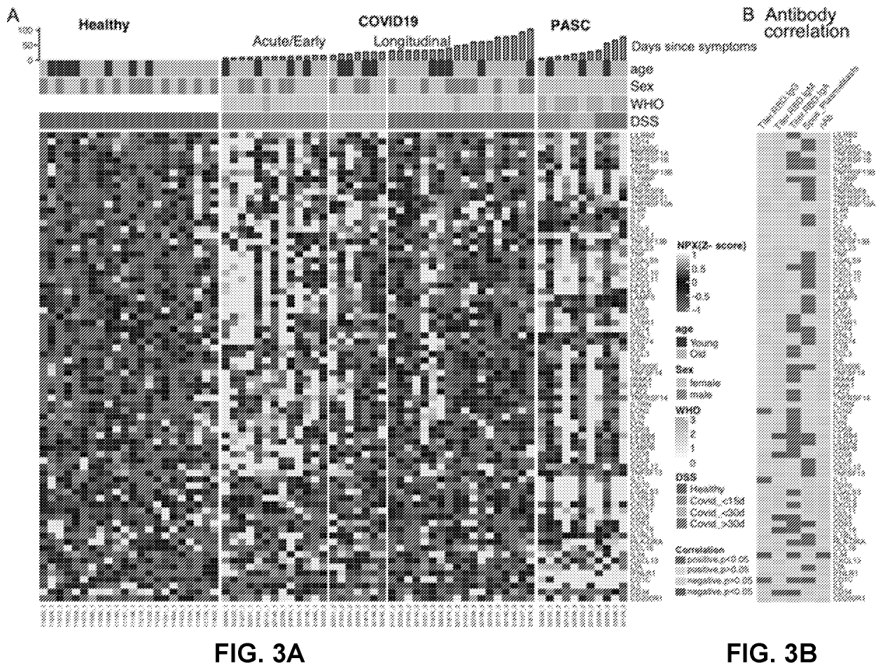

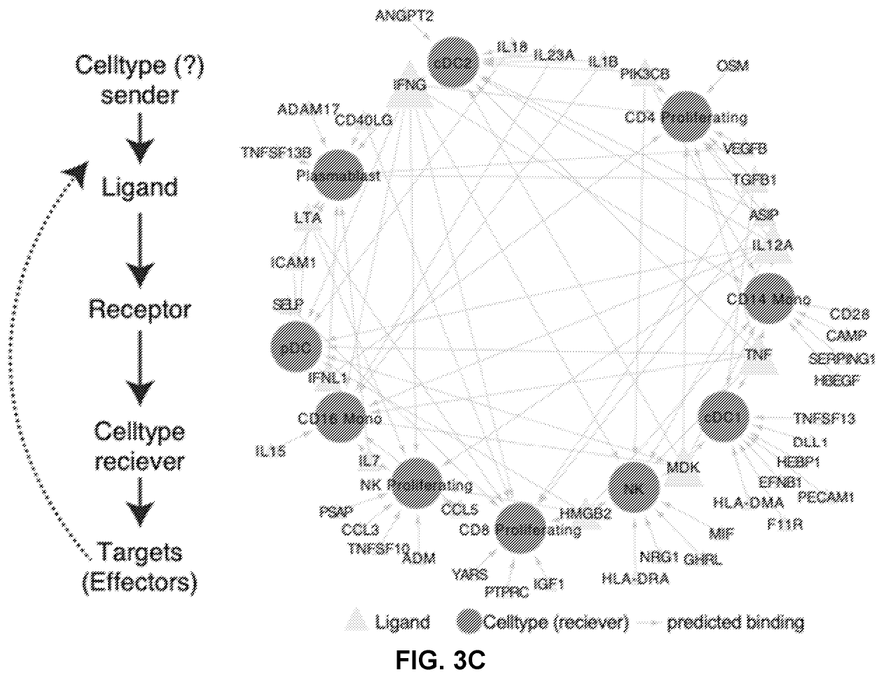

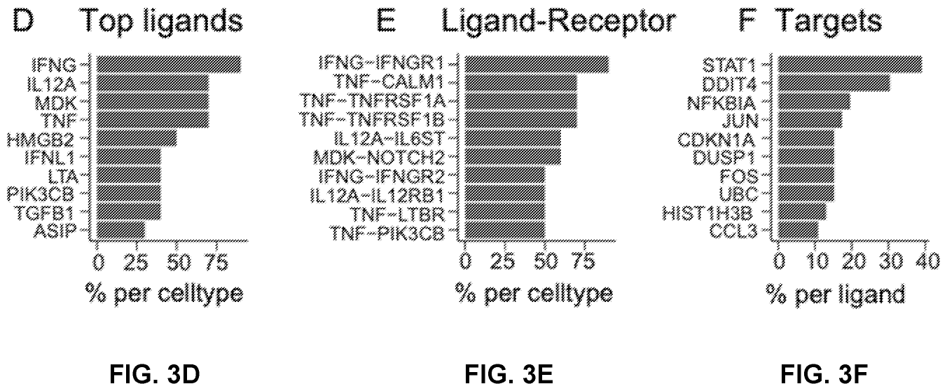

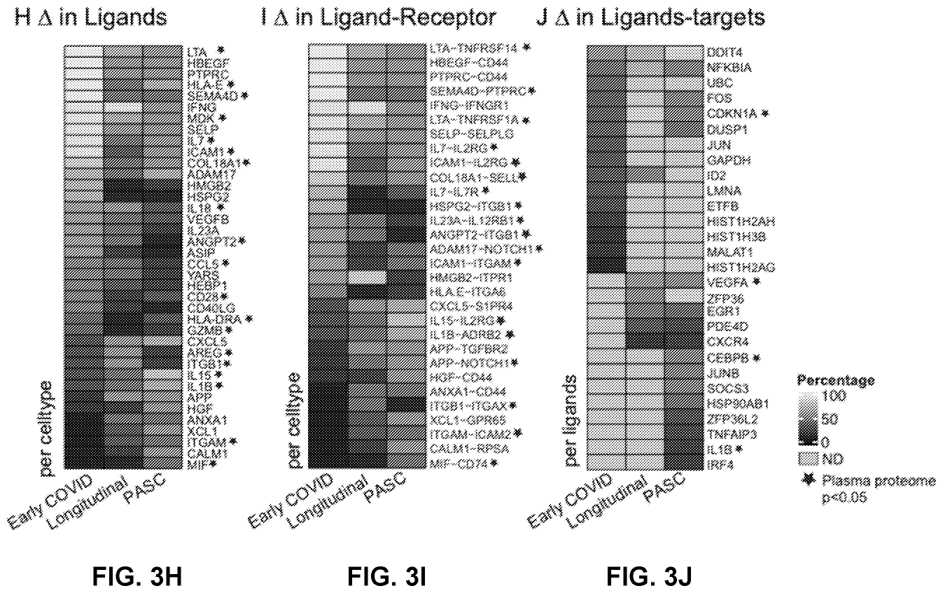

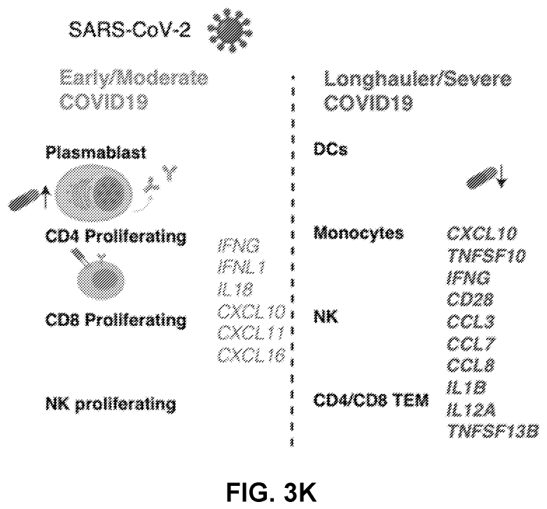

Integrative analysis reveals key network nodes for immunomonitoring and therapeutic targeting in early COVID-19+ infection and PASC. It was sought to provide context for the findings at the network level to identify potential nodes to focus efforts for monitoring immune responses and therapeutic targeting. Serum proteomics of COVID-19 subjects identified proteins differentially expressed in early acute infection (<15 days PSO), longitudinally and in PASC. The differential chemokines and cytokines like IFNG, IL7, IL18, CCL5, CXCL10 observed at early infection were significantly upregulated and the signal decayed substantially over the visits in recovered COVID-19 subjects. These findings coincided with single cell immune cell activation of plasmablast, CD4 & CD8 proliferating cells, NKs and TEMRAs suggesting a strong immune response from immune cell types towards viral infection in recovered participants. However, PASC participants showed persistent activation of IL11, CCL3, CXCL8 and upregulation of IL7R, IL5, CD1C, CD33, CCL11 after >30 days since symptoms (FIG. 3A). These proteins were significantly correlated with both IgA RBD titer and S-specific plasmablasts suggesting a complex relationship between PASC and antigen-specific response to SARS-CoV-2 (FIG. 3B).

To infer the origin of the activated key proteins, intracellular communication analyses of single cell RNA data were performed from early acute COVID-19 infection subjects, longitudinal, and PASC subjects respectively using ligand-receptor-target model incorporated in NicheNet (Methods). At single cell RNA level, the protein corresponding genes can be seen relatively expressed in immune cell types tracking their probable source. We retrieved top 10 inferred ligands expressed in sender cell types (n=31, >10% of cells) influencing the expression in receiver cell types. We focused on 10 cell types as potential receivers of signals based on their changes in early acute infection: plasmablasts, CD14 monocytes, CD16 monocytes, NK cells, proliferating cells (CD4, CD8, NK), and dendritic cells (cDC1, cDC2, pDC). IFNG, IFNL1, IL12A, MDK, LTA, TNF, and TGFB1 were high-confidence predicted ligands signaling into receiver immune cell types during early acute COVID-19 infection by interacting with cognate receptors (FIGS. 3C-3E), and these paralleled signals identified in analysis of early acute plasmablasts. Signaling from predicted ligands converged onto two regulators of inflammation: DDIT4 (mTOR inhibitor and Th17 enhancer (Zhang et al. 2018), 85% of predicted ligands) and ZFP36 (RNA binding protein, 83% of predicted ligands), as well as multiple pro-inflammatory mediators including transcription factors CEBP/β, and AP-1 subunits JUNB and FOS (50% of predicted ligands) (FIG. 3F). These results suggested PASC drives a complex dysregulation of immunity including both pro- and anti-inflammatory mechanisms.

Differential network analysis of intercellular communication was performed for longitudinal samples and PASC participants with controls as COVID-19 at <=15 days PSO and non-PASC recovered COVID-19 participants, respectively. To identify longitudinal changes in ligand-receptor usage, we focused on differentially expressed target genes from early acute infection. There was significant overlap (27%) between ligand-receptor pairs identified in early acute infection (<=15 days PSO), longitudinal timepoints (>15 days PSO), and PASC participants independent of their direction of change (FIG. 3G). Shared LR interactions were the most common (567 pairs), indicating common signals driving each phase of mild COVID-19. Early acute infection had the most unique LR interactions (461 pairs, 22.2%) followed by PASC (291 pairs, 14%), and longitudinal timepoints (175 pairs, 8.4%). Notably, a large number of interactions (644, 31%) were shared between early acute infection and PASC, suggesting that many early signals persist into PASC. We visualized individual LR interactions for each receiver cell type to identify key cell types involved in PASC. Predicted ligand activity increased in CD14 monocytes (IL15-IL2RG, HBEGF-CD44, CXCL16-C3AR1) and DCs (cDC2, pDC; II1B-ADRB2, HMGB2-AR, LTA-LTBR), while they decreased in plasmablasts (IFNG-IFNGR1, IL7-IL2RG, IL23A-IL12RB1). The top predicted ligands (e.g., LTA, HBEGF, HLA-E, IFNG) increased in early acute infection and persisted in PASC later timepoints (FIG. 3H). IFNG-IFNGR1 predicted interactions per celltype (22% vs 66%), LTA-TNFRSF14/1B (55.55% vs 100%), HBEGF-CD44 (44% vs 100%), IL12A-IL6ST (44% vs 70%) were lower in PASC compared to (FIG. 3I). Increased ligand activity of CXCL5, IL1B, IL15, and MIF suggest pro-inflammatory milieu in some PASC patients. These results were consistent with our scRNA analysis which showed reduced early IFN signaling and increased inflammatory signaling in PASC participants. Early acute infection showed broad prediction of inflammatory cytokine activity that correlated with Olink serum proteomics (CCL5, IL7, IL18, LTA, MDK). In particular, we identified a large number of early acute signals in innate immune cells from PASC patients, such as TNF and IFN signaling, that persist in PASC while resolving in recovered COVID-19 participants. The low level of IFN signaling in acute infection may implicate early innate immune responses against SARS-CoV-2 as a risk factor for PASC and persistence of this signaling as a pathogenic mechanism. Based on these results, a subset of PASC patients may benefit from multiple therapeutic options, such as inhibiting inflammatory cytokine signaling by TNF and IFNs in innate immune cells, as well as convergent downstream targets which were primarily intracellular, including CEBP/beta, CDKN1A/p21, IL1beta, ZFP36/TTP, and DDIT4 (FIG. 3J). Overall, network analyses provide a platform for mechanistic hypothesis generation on key active pathways throughout mild COVID-19 infection and resolution that may prime adaptive immune responses, influence disease outcome, and diagnose and treat complications such as PASC (FIG. 3K).

### CONCLUSION

The present study provided an in-depth longitudinal analysis of the immune response to SARS-CoV-2 natural infection, integrating serum proteomics, single-cell transcriptomics and epigenomics, and cellular immunophenotype by flow cytometry with comprehensive analysis of the CoV-2-specific adaptive immune response in T cells, memory B cells, and antibodies. To our knowledge, this is the deepest longitudinal systems immunology study to-date in mild COVID-19 infection. We defined immune responses to early acute infection, including age-enhanced IFN responses across all circulating immune celltypes and a potential IFN-plasmablast regulatory circuit, confirmed the longitudinal resolution of these inflammatory pathways and re-establishment of homeostasis in most participants, identified acute infection correlates of convalescent antibody and memory B cell responses, defined a subset of PASC participants with innate immune hyperactivation, and integrated these data to identify potential regulatory nodes in early infection and PASC.

A defining characteristic in our mild COVID-19 cohort was robust immune activation in the first 2 weeks of acute infection that resolved over time. This included inflammatory cytokine responses (IFNs, TNF) and innate immune sensor (TLR, RLR, NLR) signaling pathways, along with cellular activation in both adaptive and innate immune compartments. The key innate immune sensors triggered in natural SARS-CoV-2 infection are not confirmed, but our data support involvement of the expected RNA-sensing TLRs (TLR3, TLR7) and RLRs (RIG-I, MDA5), and potentially downstream activation of inflammasomes through cell death or stress released ligands. The implications of increased serum RIG-I during acute infection are unclear, perhaps facilitating capture of extracellular viral nucleic acids. Innate danger sensors are key drivers of the IFN response, which was also robustly induced in our cohort along with signaling by other inflammatory cytokines such as TNF. As these pathways waned over time, activation marker positive cells and inflammatory proteins largely returned to baseline levels by ˜day 30, and this temporal control is likely one key factor to successful resolution of mild disease. This contrasts with the persistent CRS characterizing severe COVID-19 and mechanisms of inflammatory damage to tissue such as TNF/IFNγ-mediated cell death (Karki et al. 2021). Proteins involved in homeostatic functions (EMT, coagulation, angiogenesis) increased from acute infection to convalescence. In particular, the increase over time of coagulation proteins raises questions about the risk of immune thrombocytopenia, a common complication typically associated with more severe COVID-19 infection (Guan et al. 2020). Timing of increased coagulation parallels reports of late-onset mild thrombocytopenia at 3-4 weeks (Chen et al. 2020). We observed significantly increasing levels of thrombomodulin (THBD) in convalescence, a factor that was strongly correlated with duration of hospitalization and risk of mortality in COVID-19 (Goshua et al. 2020). These results suggest a direct link between the inflammatory response in acute infection, the dynamics of inflammatory resolution, and long-term coagulopathy risk.

PASC or long COVID is one of the most enigmatic consequences of the ongoing pandemic. The scale of impact on global health is difficult to overstate given conservative estimates of ˜2% to higher estimates of ˜two-thirds of all outpatient COVID-19 cases progressing to PASC after 30 days post-infection (Hernandez-Romieu 2021; Sudre et al. 2021), suggesting there are over 3 million PASC sufferers globally. The involvement of many organ systems coupled with the highly subjective nature of symptoms has made it difficult to define consensus, objective criteria for diagnosis or clear therapeutic options. In our cohort, a subset of 3 COVID-19 participants progressed to PASC. All 3 PASC participants in our study were female, consistent with prior reports of female-biased presentation. Females are known to have stronger inflammatory responses in autoimmune disease and HIV infection, and this predisposition to hyperinflammatory responses may be a risk factor for PASC. Significant correlation was observed between PASC and number of initial symptoms, as previously reported, but correlation with age was not reproduced, likely due to small sample size (Sudre et al. 2021).

Inflammatory and hormonal proteins in serum were signatures of PASC participants compared to recovered COVID-19 participants. These signatures were coupled with evidence of persistent activation in innate immune cells based on gene expression and chromatin accessibility after 30 days PSO. DCs and CD14+ monocytes in PASC showed strongest evidence of transcription factor motif enrichment including many AP-1 family motifs, along with inflammatory cytokine and IFN-driven motifs such as STAT and IRF family motifs. AP-1 is pleiotropically activated by diverse signals including innate immune sensors, inflammatory cytokines, and cellular stress. Gene expression signatures showed stronger TNF and hypoxia signaling in CD14 monocytes over time. Early infection signaling and dynamics were unique in PASC participants, including lower RLR and IFN pathway scores that did not wane longitudinally. This combination of changes is reminiscent of risk factors for severe COVID-19: early innate immune activation is persistently dysregulated in both, with dampened IFN responses that may not control viral replication. Prolonged viral replication drives an over-exuberant innate immune response that persists beyond acute infection and drives pathology. While 2 participants had strong inflammatory serum protein signatures, one participant had fewer inflammatory proteins and elevated hormones and hormone receptors. Hormonal changes suggest a non-inflammatory contributor to PASC.

Multi-omic data integration identified multiple soluble proteins for potential therapeutic neutralization, including LTA/TNFβ, IL-1β, and TRAIL. Persistently elevated TNF may be an appealing target given the potential for TNF-driven pathogenic cell death and correlations with disease severity and death (Karki et al. 2021; Del Valle et al. 2020). STAT1 was another key downstream target that was activated by multiple predicted ligands. A previous study also linked STAT1 with JAK1/2 to IFN-driven complement hyperactivation in SARS-CoV-2 as a major mechanism of tissue damage (Yan et al. 2020). These parallels suggest mechanisms driving PASC may be shared with those driving severe COVID-19. Most differential proteins were elevated in PASC, but IL-15 was uniquely low in PASC participants throughout COVID-19 infection and convalescence. This may contribute to poor innate immune responses to infection via impaired NK cells. This scenario contrasts with elevated IL-15 reported in other studies to correlate with disease severity and an exhausted NK cell phenotype in severe COVID-19 (Liu et al. 2021).

Overall, our study results provide a comprehensive longitudinal roadmap for immune activation and resolution in mild COVID-19, including a key age-dependent effect on immune responses. We observed a robust plasmablast response that may be tightly regulated by early IFN responses, and identified key early correlates of antibody and B cell responses, both findings which should be broadly tested as potential shared features in diverse natural infections. A subset of participants who progressed to PASC revealed novel inflammatory and non-inflammatory signatures in serum proteins, and innate immune-centric hyperactivation. Multiple potential therapeutic targets in PASC are nominated by our analyses, and serum protein biomarkers may provide objective diagnosis of inflammatory and non-inflammatory PASC patients after validation in larger cohorts. A more personalized approach to immunomonitoring and therapy will result in improved outcomes across the spectrum of COVID-19 and PASC.

### Example 2: Persistent Serum Protein Signatures Define an Inflammatory Subset of Long COVID

The serum proteome may provide insights into potential drivers of PASC symptomatology and may offer a clinically accessible tool to help define subgroups of PASC. Therefore the serum proteome was analyzed using the Olink Explore 1536 panel in 55 subjects (21 men and 34 women, age 22-82 years) with persistent symptoms lasting ≥60 days after an acute, PCR-confirmed SARS-CoV-2 infection (termed “PASC”), 24 subjects (9 men and 15 women, age 20-79 years) who had a PCR-confirmed SARS-CoV-2 infection but symptomatically recovered (termed “Recovered”), and 22 subjects (12 men and 10 women, age 29-77) that had a negative nasopharyngeal PCR test (termed “Uninfected”) (FIG. 6A). The uninfected individuals had blood drawn once at baseline while the PASC and recovered subjects had one or more blood draws collected at timepoints ≥60 days and up to 379 days post-symptom onset (PSO) of acute COVID (FIG. 6B). Most patients had mild to moderate symptoms during acute infection (World Health Organization (WHO) ordinal scale 2 or 3) but 3 subjects were hospitalized and required oxygen (WHO ordinal scale 5). None required mechanical ventilation.

Previous studies have tried to subset PASC patients by either type, number, or severity of clinical features. For the cohort used in this example, hierarchical clustering on PASC symptomatology alone at ≥60 days post symptom onset (PSO) did not clearly drive significant patient clustering (FIG. 6A, FIG. 7A). Subsequently, symptoms were attempted to be used to drive clustering of significantly associated serum protein signatures, but no single symptom or combination of symptoms was able to clearly distinguish patient groups (FIG. 7B, C, D) suggesting that symptoms alone are unable to differentiate subsets of PASC.

Thus, an alternative approach was used, using unbiased clustering of the serum proteome across the entire cohort (PASC+recovered+uninfected) to find clusters of individuals that had similar serum proteome signatures regardless of their status or symptomatology. Canonical pathway enrichment was performed on the first post-60 day sample available for each PASC subject, the last available post-60 day sample for each recovered subject (to maximize the chance that they had returned to baseline) and on the solitary sample from the uninfected individuals Curated canonical pathways from the Molecular Signatures Database (MSigDB) were used and a rule-in approach applied, which resulted in 85 pathways that distinguished PASC from recovered and uninfected individuals with a significant rule-in performance (p<0.01). These pathways were merged into 54 modules to avoid gene set redundancy using the enrichment map approach with a minimum Jaccard index threshold of 25% (Table 1, after REFERENCES section). Hierarchical clustering using the 54 proteomic modules identified 5 discrete clusters that showed distinct expression patterns of the modules (FIG. 4A). Two of the clusters (4 & 5) showed a marked enrichment for inflammatory modules while clusters 1, 2, and 3 lacked a distinct inflammatory protein signature. Inflammatory clusters 4 and 5 included predominantly PASC subjects (91% and 80% respectively) whereas cluster 1 consisted of only uninfected or recovered subjects. Clusters 2 and 3 consisted of a mixture of PASC (48% and 28% respectively), recovered, and uninfected subjects (FIG. 8A). The distribution of PASC subjects across inflammatory (4 & 5; 65% of PASC) and non-inflammatory (2 & 3; 35% of PASC) proteomic clusters underscores the heterogeneity of PASC. To determine whether the differential serum proteomic signatures discovered by comparing the first post-60 day PSO sample for PASC to the last post-60 day PSO sample for recovered are stable over time, the analysis was extended to include all longitudinal samples available for each subject. It was observed that PASC subjects that have an inflammatory protein signature continue to have that signature over time and that most subjects remained in the same cluster throughout the study period (FIG. 8B).

An inflammatory plasma protein signature may also correlate with being more symptomatic but because the cohort used in this example consisted primarily of patients with only mild to moderate COVID-19 (WHO ordinal scale 2 or 3), commonly used COVID severity indices did not capture a range of heterogeneity in symptomatology. Thus, a clinical activity index was developed that accounted for both symptoms and their impact on activities of daily living. Inflammatory PASC subjects in clusters 4 & 5 had a significantly higher clinical activity score (p=0.003) compared to non-inflammatory PASC subjects in clusters 2 & 3 (FIG. 4B). There was a possibility that subjects with an inflammatory protein signature may have had less robust immune responses to SARS-CoV-2, thus increasing the chance that they might have delayed viral clearance or an increased risk for viral persistence. However, comparison of SARS-CoV-2 receptor binding domain (RBD)-specific IgG titers in infected subjects (PASC+Recovered) 90 days PSO identified no significant difference between the inflammatory (4 & 5) and non-inflammatory (1, 2 & 3) clusters (FIG. 4C).

Among the 54 modules that defined the 5 clusters (FIG. 4A), those were identified that significantly distinguished each cluster by calculating the single-sample-Gene Set Enrichment Analysis (ssGSEA) score per module across samples. Ranking modules by adjusted p-value identified those most significantly associated with clusters 4 and 5 (FIG. 9, FIG. 10, Table 2, after REFERENCES section). Within cluster 4, multiple pathways associated with type II interferon (IFN-γ) signaling (Type II IFN signaling, IL-27, TID, etc.) were among those most highly enriched (FIG. 4D). Canonical NF-κB signaling and NF-κB activating cytokine pathways (IL-18, TNF, IL-1 were enriched in both clusters 4 and 5 (FIG. 4E). In addition, cluster 5 was also enriched for proteins associated with regulation of IFN-α signaling (FIG. 4F). The expression scores of these modules across all samples were significantly correlated with each other, indicating, patients with higher IFN-γ signaling have higher IL27, IL18, and NF-κB signaling, and patients with higher TNF signaling have higher IL1, NF-κB, and IFN-α signaling, suggesting a global activation of immune cascades that drive inflammation (FIG. 4G).

Subsequently, the individual proteins differentially expressed in the serum of subjects within each cluster were investigated. Clusters 1-5 were individually compared to all other clusters. Cluster 4 had 234 differentially expressed proteins (DEPs) whereas cluster 5 had 296 DEPs (Table 3, after REFERENCES section; adj. p-value <0.05). Since cytokines, chemokines, and cytokine/chemokine receptors are major drivers of inflammation and potential targets for therapeutic intervention, these were the focus and individual DEPs were ranked by adjusted p-value (FIG. 5A). IFN-γ was found to be the cytokine that most significantly defines cluster 4. Moreover, IFN-γ was the top DEP enriched in cluster 4 among all 1463 analytes in the Olink protein panel (FIG. 11, FIG. 12, Table 3, after REFERENCES section). Increased expression of chemokines and cytokines known to be regulated by IFN-γ including CXCL9, CXCL10, CXCL11, and IL-27 in cluster 4 suggests that it is functionally active (FIG. 5A, FIG. 5B). Increased expression of IL-12 p40 (IL12B) and the IL-12 p40/p70 heterodimer (IL12A_IL12B) in cluster 4 was also observed, which may drive expression of IFN-γ and an overall Th1 signature.

To determine whether IFN-γ and IFN-γ driven cytokines, chemokines, and pathways remained persistently elevated over time in inflammatory PASC, these signatures were evaluated longitudinally in available samples beginning from early acute infection to 275 days PSO. IFN-γ, IL-12 p40, and IFN-γ-driven chemokines were consistently elevated within inflammatory PASC from clusters 4 & 5 compared to non-inflammatory PASC from clusters 1, 2, and 3, extending to at least 275 days after initial SARS-CoV-2 infection (FIG. 5C, FIG. 13). IFN-γ related signaling modules also showed persistent enrichment over the same time (FIG. 5D, FIG. 14). In addition to IFN-γ, TNF, TNF-driven cytokines and chemokines (including IL-6 and CCL7 (MCP3)), and several TNF receptor superfamily members were also increased in clusters 4 and 5 (FIG. 5A, FIG. 2E, FIG. 13). TNF, IL-6, and CCL7 remained persistently elevated in inflammatory PASC over time compared to non-inflammatory PASC (FIG. 5F, FIG. 13). In addition, TNF signaling and canonical NF-κB signaling pathways previously found to be enriched at early time points in inflammatory PASC remained elevated over time (FIG. 5G, FIG. 14).

Finally, the pathway related to expression of IFNA signaling was found to be enriched at the first post-60 day PSO timepoint in cluster 5 (FIG. 4F). The Olink assay only quantifies IFN-γ and IFNλ1 but increased expression of proteins associated with type I IFN activation including SAMD9L, MNDA, DDX58, LAMP3, and others was observed (FIG. 11, FIG. 12). These proteins were found to be highly increased early after acute infection but in inflammatory PASC, remained elevated over time compared to non-inflammatory PASC. Longitudinal assessment showed that they trended toward the levels seen in non-inflammatory PASC and recovered subjects by approximately 180 days post infection (FIG. 5H), similar to the kinetic observed for the expression of IFNA signaling pathway over time (FIG. 5I). This is notable in light of recent studies reporting detection of SARS-CoV-2 RNA and protein in gastrointestinal and hepatic tissue of convalescent patients up to 180 days after acute infection and in diverse extrapulmonary tissues including brain up to 230 days after acute symptom onset (Cheung et al. 2022, Chertow et al. 2021). Whether residual viral RNA and/or protein may serve as a driver of the phenotype in inflammatory PASC remains to be investigated more thoroughly.

To determine whether the observations could be extended to an independent cohort of PASC patients collected across a broader range of acute COVID severities, a similar analysis approach was applied to the recently published INCOV cohort that included Olink plasma proteomic data from 204 SARS-CoV-2 patients and 289 healthy controls (Su et al. 2022, Su et al. 2020). Of the 204 INCOV patients, 125 had a blood sample obtained and clinical data collected within a PASC time window of 2-3 months after onset of acute disease. Seventy-five (60%) of these had at least 1 PASC symptom. The Olink panel employed in this example measured only 443 of the 1472 proteins measured in our study but 163 proteins overlapped with the inflammatory signatures that significantly defined clusters 4 & 5 in our cohort. To be consistent with our cohort, k-means unsupervised clustering of the Olink proteomic data from the INCOV cohort was performed with k=5 using the 163 overlapping proteins on the sample available at the first timepoint ≥60 days PSO per INCOV patient (74 INCOV patients).

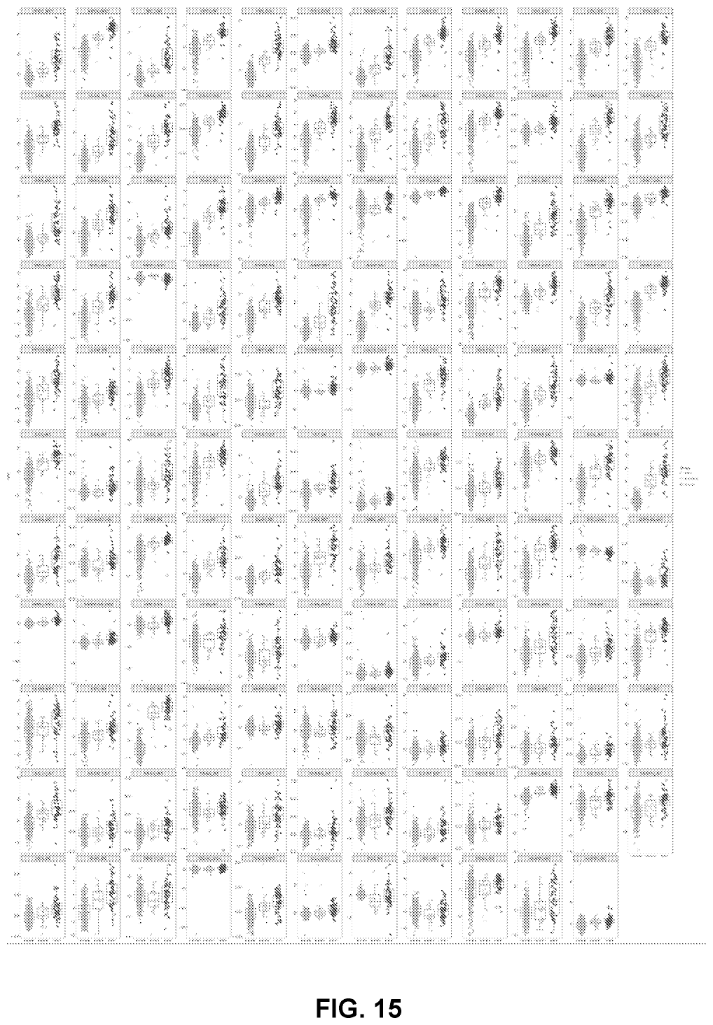

Similar to the previous cohort's clustering, the 5 identified clusters were cluster E, consisting of only INCOV patients, a second cluster with a mix of INCOV and healthy individuals (cluster D), and clusters A, B, C with predominantly healthy individuals. Compared to patients from clusters B, C, and D, patients from Cluster E showed significant enrichment of 128 of the 163 proteins that defined our inflammatory PASC (78.5%) (FIG. 15, Table 4, after REFERENCES section). Among the cytokines and chemokines observed in our inflammatory PASC subjects, the proteins that were also significantly higher in cluster E INCOV were IL12, CXCL10, CXCL11, TNF and CCL7 (FIG. 5K) along with several other proteins (like DDX58, LAMP3, etc, FIG. 15, Table 4, after REFERENCES section). Lastly, the broader diversity of disease severity in the INCOV cohort compared to the mild cohort, allowing making an association between the clinical measure of acute disease severity (WHO ordinal scale score) and proteomic inflammatory signatures. Interestingly, INCOV patients from cluster E predominantly exhibited an acute WHO ordinal score of ≥3 reflecting the association between more severe acute disease and persistent inflammation (FIG. 5L).

These findings substantially confirm and extend previous observations that have variably reported increased expression of IFN-γ, IFN-β, IFN-λ1/2/3, TNF, IL-6, IL-1B, and PTX3 in plasma from PASC patients using targeted cytokine panels (Phetsouphanh et al. 2022, Schultheiß et al. 2021, Peluso et al 2021). The results in this example demonstrate that plasma proteomic profiling can identify subjects with PASC who have an ongoing inflammatory signature and that this offers the first opportunity to subset PASC patients for further mechanistic studies, clinical trials, or development of diagnostics based on an underlying molecular signature. It is shown that in PASC subjects with inflammatory protein signatures, the IL-12/IFN-γ axis is highly active and is combined with a NF-κB driven protein signature, possibly driven by TNF and leading to excess IL-6 expression. Furthermore, evidence is shown of a persistent type I IFN driven protein signature that is present in PASC subjects with an inflammatory protein signature early in the PASC period (>60 days post-symptom onset) and extending to approximately 6 months post-infection that then trends toward normal. The timing of the type I IFN response may be related to persistent viral RNA and protein, which was observed in non-pulmonary tissues for 6-8 months after infection. Whether the two clusters of inflammatory PASC described here represents two distinct subtypes with different molecular drivers or a continuum of disease requires testing in future large validation cohorts. It is shown that these findings can be applied to another PASC proteomic dataset to identify PASC subjects with persistent inflammatory disease. These data also highlight potential targets (TNF, IL-6, IFN-γ, etc.) The approach disclosed herein provides proof-of-concept that serum protein profiling could be used to guide patient selection in investigator-initiated trials.

### Study Conduct

Serum was collected from participants enrolled in the longitudinal study, “Seattle COVID-19 Cohort Study to Evaluate Immune Responses in Persons at Risk and with SARS-CoV-2 Infection”. Eligibility criteria included adults in the greater Seattle area at risk for SARS-CoV2 infection or those diagnosed with COVID-19 by a commercially available SARS COV-2 PCR assay. Study data were collected and managed using REDCap electronic data capture tools hosted at Fred Hutchinson Cancer Research Center, including detailed information on symptoms during acute infection and longitudinal follow-up ranging from 33-233 days post symptom onset. Plasma from pre-pandemic controls used for ELISA controls were blindly selected at random from the study, “Establishing Immunologic Assays for Determining HIV-1 Prevention and Control”, with no considerations made for age, or sex. Informed consent was obtained from all participants at the Seattle Vaccine Trials Unit and the Fred Hutchinson Cancer Research Center Institutional Review Board approved the studies and procedures.

Regulatory Approvals from FH and AIFI

COVID19 FH samples and healthy controls: FH RG: 1007696 IR File: 10440 Main Consent 04/05/2020 and 6/04/2020 Seattle COVID-19 Cohort Study to Evaluate Immune Responses in Persons at Risk and with SARSCOV-2 Infection.

### Olink Serum Protein Measurement

Serum samples were inactivated with 1% Triton X-100 for 2 h at room temperature according to the Olink COVID-19 inactivation protocol. Inactivated samples were then run on the Olink Explore 1536 platform, which uses paired antibody proximity extension assays (PEA) and a next generation sequencing (NGS) readout to measure the relative expression of 1472 protein analytes per sample. Analytes from the inflammation, oncology, cardiometabolic, and neurology panels were measured.

For plate setup, samples were randomized across plates to achieve a balanced distribution of age and gender. Longitudinal samples from the same participant were run on the same plate. To facilitate comparisons with future batches, sera from 15 donors was commercially purchased (BiolVT) and randomly interspersed amongst the above study samples. Commercial samples included serum from COVID-19 serology-negative, serology-positive, PCR-positive, and recovered (no longer symptomatic) participants.

Data were first normalized to an extension control that was included in each sample well. Plates were then standardized by normalizing to inter-plate controls run in triplicate on each plate. Data were then intensity normalized across all samples. Final normalized relative protein quantities were reported as log 2 normalized protein expression (NPX) values.

### Olink Data Preprocessing:

Olink results and QC flags were reviewed for overall quality. Results for TNF, IL6 and CXCL8, which were measured on all 4 Olink panels, were reviewed prior to averaging to a single NPX value for analysis. Two samples had discrepant cross-panel measurements on these proteins. The results that trended most consistently with the participant's longitudinal measurements were kept and averaged. Serum samples were analyzed in two batches. Following the method recommended by Olink, results of the later batch were bridged to those of the earlier batch using a set of 42 cohort samples that were tested in both batches. A batch offset for each analyte was calculated as the median difference on the 42 samples as measured between the two batches, excluding samples with QC warning flags. The analyte-specific offsets were then added to the raw NPX values of the later batch.

### Symptom Activity Metrics and Scoring

Symptom activity was classified by participant report of impact on Activities of Daily Living (ADLs) for each day of illness. Days hospitalized were recorded as were any treatment or therapies received. Participants were scored according to their maximum symptom activity for each day: 0, no symptoms; 1, mild impact on ADLs reported; 2, moderate impact on ADLs reported; 3, severe illness without hospitalization; 4, severe illness with hospitalization; 5, life threatening illness hospitalized with ICU care. Durations were assigned for days spent at each level of symptom activity. A cumulative symptom activity score was calculated for each subject by multiplying the symptom activity score by the number of days spent at each level, then summing all values.

### Antibody ELISAs for RBD

Half-well area plates (Greiner) were coated with purified RBD protein at 16.25 ng/well in PBS (Gibco) for 14-24 h at room temperature. After 4 150 ul washes with 1×PBS, 0.02% Tween-2 (Sigma) using the BioTek ELx405 plate washer, the IgA and IgG plates were blocked at 37ºC for 1-2 hours with 1×PBS, 10% non-fat milk (Lab Scientific), 0.02% Tween-20 (Sigma); IgM plates were blocked with 1×PBS, 10% non-fat milk, 0.05% Tween-20. Serum samples were heat inactivated by incubating at 56° C. for 30 minutes, then centrifuged at 10,000×g/5 minutes, and stored at 4° C. previous to use in the assay. For IgG ELISAs, serum was diluted into blocking buffer in 7-12 1:4 serial dilutions starting at 1:50. For IgM and IgA ELISAs, serum was diluted into 7 1:4 serial dilutions starting at 1:12.5 to account for their lower concentration. A qualified pre-pandemic sample (negative control) and a standardized mix of seropositive serums (positive control) was run in each plate and using to define passing criteria for each plate. All controls and test serums at multiple dilutions were plated in duplicate and incubated at 37ºC for 1 hour, followed by 4 washes in the automated washer. 8 wells in each plate did not receive any serum and served as blocking controls. Plates then were plated with secondary antibodies (all from Jackson ImmunoResearch) diluted in blocking buffer for 1 h at 37 C. IgG plates used donkey anti-human IgG HRP diluted at 1:7500; IgM plates used goat anti-human IgM HRP diluted at 1:10,000; IgA plates used goat anti-human IgA HRP at 1:5000. After 4 washes, plates were developed with 25 ul of SureBlock Reserve TMB Microwell Peroxide Substrate (Seracare) for 4 min, and the reaction stopped by the addition of 50 ml 1N sulfuric acid (Fisher) to all wells. Plates were read at OD450 nm on SpectraMax i3X ELISA plate reader within 20 min of adding the stop solution.

OD450 nm measurements for each dilution of each sample were used to extrapolate RBD endpoint titers when CVs were less than 20%. Using Excel, endpoint titers were determined by calculating the point in the curve at which the dilution of the sample surpassed that of 5 times the average OD450 nm of blocking controls+1 standard deviation of blocking controls.

### Symptoms Category Clustering

Symptoms data were collected from each donor over multiple visits. The symptoms were merged together into six major categories such as Fatigue/malaise, Pulmonary, Cardiovascular, Gastrointestinal, Musculoskeletal, and Neurologic. Other mild symptoms were combined into a single category “Any mild symptoms”. The symptoms information was converted to binary format such as yes corresponds to 1 and no corresponds to 0. The missing symptom information is denoted by NA. The binary information was used to perform principal component analysis (PCA) and visualize sample clustering using factoextra (v1.0.7). The contribution of variation for each symptom category was retrieved and shown in bar plot. For each symptom category we identified symptom specific differential plasma proteins using linear mixed model. The Ime4 package (v1.1) was used to carry out linear mixed model analysis where age, sex were fixed variable and donor information is a random variable.

$$NPX \sim Symptom status + Age + Sex + ( 1 \vert Donor ) \qquad ( 1 )$$

The p value is obtained from chi-square statistics. The specific symptom category associated with differential plasma proteins selected using p<0.05. The identified differential proteins from six symptom specific categories were merged together and their expression visualized in a heatmap using package ComplexHeatmap (v2.4).

### Symptom Activity Metrics and Scoring

Symptom activity was classified by participant report of impact on Activities of Daily Living (ADLs) for each day of illness. Days hospitalized were recorded as were any treatment or therapies received. Participants were scored according to their maximum symptom activity for each day: 0, no symptoms; 1, mild impact on ADLs reported; 2, moderate impact on ADLs reported; 3, severe illness without hospitalization; 4, severe illness with hospitalization; 5, life threatening illness hospitalized with ICU care. Durations were assigned for days spent at each level of symptom activity. A cumulative symptom activity score was calculated for each subject by multiplying the symptom activity score by the number of days spent at each level, then summing all values.

Identification of Pathways with High Rule-In Performance

Partial area under the receiver operating characteristic curve (pAUC) was used to evaluate the rule-in performance of individual pathways in identifying PASC subjects with respect to recovered and uninfected subjects. The pAUC bounded by a specificity between 90-100% and the corresponding 99% confidence interval (two-sided) of each pathway were calculated using the “ci.auc” function in the R package PROC with the following parameters: partial.auc=c(0.9, 1), conf.level=0.99, boot.n=1000. A pathway was identified as significant with p<0.01 if its pAUC lower confidence bound was above the corresponding pAUC of a random, non-performing classifier, i.e. 0.005.

The canonical pathway “c2.cp.v7.2.symbols” geneset and associated gene information from MsigDB (v7.2) were collected. The canonical pathway consists of 2871 pathways used to perform single sample GSEA (ssGSEA) using GSVA (v1.40) R package (Hänzelmann et al., 2013 PMID:23323831). Among 2871 pathways, 1960 pathways with overlapping plasma proteins were used as input for GSVA with min.size 2 and max.size 2000 genes as parameters. The ssGSEA resulted in a normalized enrichment score (NES) for each pathway. One sample for each PASC donor was selected as the last time point of infected recovered with >60 days PSO (n=24) and first time point with >60 days PSO for infected PASC donors (n=55). Total 101 donors with one sample including uninfected (n=22) were considered for biomarker analysis. Rule-in approach was implemented to identify pathways significantly associated with PASC donors. Parameters such as confidence interval (CI), pAUC and bootstrap (boot.n) of 200 were used. Bootsrtrap analysis was performed using random seed over multiple processors using function mcapply. Range of CI 0.8-0.99 and pAUC 0.8-0.95 was used to identify pathways associated with the PASC group. These pathways were used to differentiate the uninfected and PASC donors into separate clusters incorporating >50% of cluster size. The clustering was performed by the k-means approach implemented in ComplexHeatmap (v2.4) and visualized. The bootstrap analysis resulted in CI of 0.99 and pAUC of 0.95 which can differentiate uninfected and PASC donors in clusters. These parameters were used to identify pathways associated with PASC with a bootstrap of 1000 as mentioned before. The analysis resulted in 85 pathways. These 85 pathways then collapsed into 54 modules.

A module is defined if pairwise genests had an overlap of at least 25% (jaccard index 0.25) genes between them (Bader et al., 2010). The 54 modules then used to perform module enrichment at single sample level using GSVA. The normalized enrichment score for each module was scaled and clustered using K-means clustering implemented in ComplexHeatmap (v2.4) with parameter row_km and column_km. The identified clusters are then visualized in heatmap.

### Pathway Enrichment Analysis

Gene Set Enrichment Analysis (GSEA) was performed among genes that defined early acute infection status and genes that defined longitudinal changes. A custom collection of genesets that included the Hallmark v7.2 genesets, KEGG v7.2 and Reactomev7.2 from the Molecular Signatures Database (MSigDB, v4.0) was used as the pathway database. The “Type Ill interferon signaling” gene set was manually curated from the Interferome database. Genes were pre-ranked by the decreasing order of their log fold changes or coefficients. The running sum statistics and Normalized Enrichment Scores (NES) were calculated for each comparison. The pathway enrichment p-values were adjusted using the BH method and pathways with p-values <0.05 were considered significantly enriched.

### Sample-Level Enrichment (SLEA)

Sample-level enrichment analysis (SLEA) was used to represent the GSEA pathway expression results on a per-sample basis. The SLEA score was calculated by first calculating the mean expression value of genes (averaged across single cells) enriched in a pathway, then comparing it to the mean expression of random sets of genes (averaged across single cells) of the same size for 1,000 permutations per sample. The difference between the observed and expected mean expression values for each pathway was determined as the SLEA pathway score per sample.

### Statistical Analysis

All statistical analyses were performed using the corresponding functions in RStudio (version 4.1). Comparisons of single protein olink NPX or module ssGSEA scores between groups were tested using the Wilcoxon rank sum test and when appropriate, the Benjamini-Hochberg method was applied to adjust p values in multi-testing correction. Unless specified, an adjusted p-value of 0.05 was considered as significant.

### Analysis of Su Y et al (2022) Olink Data

The Olink proteomic data consisted of 204 SARS-CoV-2 (INCOV) patients and 289 healthy controls. The INCOV patients were studied at clinical diagnosis (T1), acute disease (acute, T2), and 2-3 months post onset of initial symptoms (convalescent, T3). Olink plasma proteomic data was available for a total of 443 proteins. Among these, 163 proteins overlapped with the differentially expressed proteins found in inflammatory signatures that significantly defined clusters 4 & 5 in our cohort. K-means unsupervised clustering of the INCOV Olink proteomic data was performed on the 163 protein overlap. To remain consistent with our cohort, we used samples available at the first timepoint ≥60 days PSO per INCOV patient (which made a total 74 INCOV patients). The kmeans function of the stats R package was used with k=5, allowing 100 iterations.

### REFERENCES

1. Groff, D. et al. Short-term and Long-term Rates of Postacute Sequelae of SARS-CoV-2 Infection: A Systematic Review. JAMA Netw Open 4, e2128568 (2021).
2. Nalbandian, A. et al. Post-acute COVID-19 syndrome. Nat. Med. 27, 601-615 (2021).
3. Wang, L. et al. PASCLex: A comprehensive post-acute sequelae of COVID-19 (PASC) symptom lexicon derived from electronic health record clinical notes. J. Biomed. Inform. 125, 103951 (2022).
4. Munblit, D. et al. Studying the post-COVID-19 condition: research challenges, strategies, and importance of Core Outcome Set development. BMC Med. 20, 50 (2022).
5. Davis, H. E. et al. Characterizing long COVID in an international cohort: 7 months of symptoms and their impact. EClinicalMedicine 38, 101019 (2021).
6. Evans, R. A. et al. Physical, cognitive, and mental health impacts of COVID-19 after hospitalisation (PHOSP-COVID): a UK multicentre, prospective cohort study. Lancet Respir Med 9, 1275-1287 (2021).
7. Cheung, C. C. L. et al. Residual SARS-CoV-2 viral antigens detected in GI and hepatic tissues from five recovered patients with COVID-19. Gut vol. 71 226-229 (2022).
8. Chertow, D. et al. SARS-CoV-2 infection and persistence throughout the human body and brain. Research Square (2021) doi:10.21203/rs.3.rs-1139035/v1.
9. Su, Y. et al. Multiple early factors anticipate post-acute COVID-19 sequelae. Cell 185, 881-895.e20 (2022).
10. Su, Y. et al. Multi-Omics Resolves a Sharp Disease-State Shift between Mild and Moderate COVID-19. Cell 183, 1479-1495.e20 (2020).
11. Phetsouphanh, C. et al. Immunological dysfunction persists for 8 months following initial mild-to-moderate SARS-CoV-2 infection. Nat. Immunol. 23, 210-216 (2022).
12. Schultheis, C. et al. From online data collection to identification of disease mechanisms: The IL-1s, IL-6 and TNF-α cytokine triad is associated with post-acute sequelae of COVID-19 in a digital research cohort. bioRxiv (2021) doi:10.1101/2021.11.16.21266391.
13. Peluso, M. J. et al. Markers of Immune Activation and Inflammation in Individuals With Postacute Sequelae of Severe Acute Respiratory Syndrome Coronavirus 2 Infection. J. Infect. Dis. 224, 1839-1848 (2021).
14. Allenspach, E. J. et al. (2021). Germline SAMD9L truncation variants trigger global translational repression. J. Exp. Med. 218.
15. Arunachalam, P. S. et al. (2020). Systems biological assessment of immunity to mild versus severe COVID-19 infection in humans. Science 369, 1210-1220.
16. Bastard, P. et al. (2020). Autoantibodies against type I IFNs in patients with life-threatening COVID-19. Science 370.
17. Benjamini, Y. and Hochberg, Y. (1995). Controlling the false discovery rate: A practical and powerful approach to multiple testing. J. R. Stat. Soc. 57, 289-300.
18. Bernardes, J. P. et al. (2020). Longitudinal Multi-omics Analyses Identify Responses of Megakaryocytes, Erythroid Cells, and Plasmablasts as Hallmarks of Severe COVID-19. Immunity 53, 1296-1314.e9.
19. Blanco-Melo, D. et al. (2020). Imbalanced Host Response to SARS-CoV-2 Drives Development of COVID-19. Cell 181, 1036-1045.e9.
20. Blomberg, B. et al. (2021). Long COVID in a prospective cohort of home-isolated patients. Nat. Med. 1-7.
21. Bosteels, C. et al. (2020). Inflammatory Type 2 cDCs Acquire Features of cDC1s and Macrophages to Orchestrate Immunity to Respiratory Virus Infection.

Immunity 52, 1039-1056.e9.
22. Browaeys, R. et al. (2020a). NicheNet: modeling intercellular communication by linking ligands to target genes. Nat. Methods 17, 159-162.
23. Browaeys, R. et al. (2020b). NicheNet: modeling intercellular communication by linking ligands to target genes. Nat. Methods 17, 159-162.
24. Carvalho, T. et al. (2021). The first 12 months of COVID-19: a timeline of immunological insights. Nat. Rev. Immunol. 21, 245-256.
25. Channappanavar, R. and Perlman, S. (2020). Age-related susceptibility to coronavirus infections: role of impaired and dysregulated host immunity. J. Clin. Invest. 130, 6204-6213.
26. Chen, P. et al. (2021). SARS-CoV-2 Neutralizing Antibody LY-CoV555 in Outpatients with Covid-19. N. Engl. J. Med. 384, 229-237.
27. Cohen, K. W. et al. (2021). Longitudinal analysis shows durable and broad immune memory after SARS-CoV-2 infection with persisting antibody responses and memory B and T cells. Cell Rep Med 2, 100354.
28. Consiglio, C. R. et al. (2020). The Immunology of Multisystem Inflammatory Syndrome in Children with COVID-19. Cell 183, 968-981.e7.
29. Dan, J. M. et al. (2020). Immunological memory to SARS-CoV-2 assessed for up to eight months after infection. BioRxiv.
30. Davis, H. E. et al. (2020a). Characterizing Long COVID in an International Cohort: 7 Months of Symptoms and Their Impact. MedRxiv.
31. Davis, H. E. et al. (2020b). Characterizing Long COVID in an International Cohort: 7 Months of Symptoms and Their Impact. MedRxiv.
32. Del Valle, D. M. et al. (2020). An inflammatory cytokine signature predicts COVID-19 severity and survival. Nat. Med. 26, 1636-1643.
33. Euser, S. et al. (2021). SARS-CoV-2 viral load distribution reveals that viral loads increase with age: a retrospective cross-sectional cohort study (medRxiv).
34. Evans, R. A. et al. (2021). Physical, cognitive and mental health impacts of COVID-19 following hospitalisation—a multi-centre prospective cohort study. MedRxiv.
35. Fajgenbaum, D. C. and June, C. H. (2020). Cytokine Storm. N. Engl. J. Med. 383, 2255-2273.
36. Filbin, M. R. et al. (2020). Plasma proteomics reveals tissue-specific cell death and mediators of cell-cell interactions in severe COVID-19 patients. BioRxiv.
37. Files, J. K. et al. (2021). Duration of post-COVID-19 symptoms are associated with sustained SARS-CoV-2 specific immune responses. JCI Insight.
38. Finak, G. et al. (2014a). Mixture models for single-cell assays with applications to vaccine studies. Biostatistics 15, 87-101.
39. Finak, G. et al. (2014b). OpenCyto: an open source infrastructure for scalable, robust, reproducible, and automated, end-to-end flow cytometry data analysis. PLOS Comput. Biol. 10, e1003806.
40. Finak, G. et al. (2015). MAST: a flexible statistical framework for assessing transcriptional changes and characterizing heterogeneity in single-cell RNA sequencing data. Genome Biol. 16, 278.
41. Finak, G. et al. (2018a). CytoML for cross-platform cytometry data sharing. Cytometry A 93, 1189-1196.
42. Finak, G. et al. (2018b). CytoML for cross-platform cytometry data sharing. Cytometry A 93, 1189-1196.
43. Fischer, M. B. et al. (1998). Dependence of germinal center B cells on expression of CD21/CD35 for survival. Science 280, 582-585.
44. Forero, A. et al. (2019). Differential Activation of the Transcription Factor IRF1 Underlies the Distinct Immune Responses Elicited by Type I and Type III Interferons. Immunity 51, 451-464.e6.
45. Galani, I.-E. et al. (2021). Untuned antiviral immunity in COVID-19 revealed by temporal type I/III interferon patterns and flu comparison. Nat. Immunol. 22, 32-40.
46. García-Abellán, J. et al. (2021). Antibody response to SARS-CoV-2 is associated with long-term clinical outcome in patients with COVID-19: A longitudinal study. J. Clin. Immunol.
47. Gill, M. A. et al. (2008). Differential recruitment of dendritic cells and monocytes to respiratory mucosal sites in children with influenza virus or respiratory syncytial virus infection. J. Infect. Dis. 198, 1667-1676.
48. Goshua, G. et al. (2020). Endotheliopathy in COVID-19-associated coagulopathy: evidence from a single-centre, cross-sectional study. The Lancet Haematology 7, e575-e582.
49. Granja, J. M. et al. (2021). Author Correction: ArchR is a scalable software package for integrative single-cell chromatin accessibility analysis. Nat. Genet.
50. de Groen, R. A. et al. (2015). IFN-A is able to augment TLR-mediated activation and subsequent function of primary human B cells. J. Leukoc. Biol. 98, 623-630.
51. Guan, W.-J. et al. (2020). Clinical Characteristics of Coronavirus Disease 2019 in China. N. Engl. J. Med. 382, 1708-1720.
52. Gundem, G. and Lopez-Bigas, N. (2012). Sample-level enrichment analysis unravels shared stress phenotypes among multiple cancer types. Genome Med. 4, 28.
53. Hadjadj, J. et al. (2020). Impaired type I interferon activity and inflammatory responses in severe COVID-19 patients. Science 369, 718-724.
54. Hahne, F. et al. (2009). flowCore: a Bioconductor package for high throughput flow cytometry. BMC Bioinformatics 10, 106.
55. Hao, W. et al. (2020a). FKBP5 Regulates RIG-I-Mediated NF-κB Activation and Influenza A Virus Infection. Viruses 12.
56. Hao, Y. et al. (2020b). Integrated analysis of multimodal single-cell data. BioRxiv.
57. Hao, Y. et al. (2020c). Integrated analysis of multimodal single-cell data. BioRxiv.
58. Hao, Y. et al. (2020d). Integrated analysis of multimodal single-cell data. BioRxiv.
59. Huang, Y. et al. (2021). COVID Symptoms, Symptom Clusters, and Predictors for Becoming a Long-Hauler: Looking for Clarity in the Haze of the Pandemic. MedRxiv.
60. Kaneko, N. et al. (2020). Loss of Bcl-6-Expressing T Follicular Helper Cells and Germinal Centers in COVID-19. Cell 183, 143-157.e13.
61. Karki, R. et al. (2021). Synergism of TNF-α and IFN-γ Triggers Inflammatory Cell Death, Tissue Damage, and Mortality in SARS-CoV-2 Infection and Cytokine Shock Syndromes. Cell 184, 149-168.e17.
62. Katzelnick, L. C. et al. (2018). Viridot: An automated virus plaque (immunofocus) counter for the measurement of serological neutralizing responses with application to dengue virus. PLOS Negl. Trop. Dis. 12, e0006862.
63. Kim, J. H. et al. (2016). High-dose influenza vaccine favors acute plasmablast responses rather than long-term cellular responses. Vaccine 34, 4594-4601.
64. Klein, S. L. and Flanagan, K. L. (2016). Sex differences in immune responses. Nat. Rev. Immunol. 16, 626-638.
65. Kuri-Cervantes, L. et al. (2020). Comprehensive mapping of immune perturbations associated with severe COVID-19. Sci Immunol 5.
66. Lareau, C. A. et al. (2019). Droplet-based combinatorial indexing for massive-scale single-cell chromatin accessibility. Nature Biotechnology 37, 916-924.
67. Lau, A. W. Y. et al. (2021). BAFFR controls early memory B cell responses but is dispensable for germinal center function. J. Exp. Med. 218.
68. Lawrence, M. et al. (2009). rtracklayer: an R package for interfacing with genome browsers. Bioinformatics 25, 1841-1842.
69. Liu, C. et al. (2021). Time-resolved systems immunology reveals a late juncture linked to fatal COVID-19. Cell 0.
70. Logue, J. K. et al. (2021). Sequelae in Adults at 6 Months After COVID-19 Infection. JAMA Netw Open 4, e210830.
71. Lucas, C. et al. (2020). Longitudinal analyses reveal immunological misfiring in severe COVID-19. Nature 584, 463-469.
72. Lyubchenko, T. et al. (2005). Coligation of the B Cell Receptor with Complement Receptor Type 2 (CR2/CD21) Using Its Natural Ligand C3dg: Activation without Engagement of an Inhibitory Signaling Pathway. The Journal of Immunology 174, 3264-3272.
73. Mahallawi, W. H. et al. (2021). Association of viral load in SARS-CoV-2 patients with age and gender. Front. Med. (Lausanne) 8, 608215.
74. Manni, M. et al. (2018). Regulation of age-associated B cells by IRF5 in systemic autoimmunity. Nat. Immunol. 19, 407-419.
75. Marshall, J. C. et al. (2020). A minimal common outcome measure set for COVID-19 clinical research. Lancet Infect. Dis. 20, e192-e197.
76. Mathew, D. et al. (2020). Deep immune profiling of COVID-19 patients reveals distinct immunotypes with therapeutic implications. Science 369.
77. McElroy, A. K. et al. (2015). Human Ebola virus infection results in substantial immune activation. Proc. Natl. Acad. Sci. U.S.A 112, 4719-4724.
78. Molony, R. D. et al. (2017). Aging impairs both primary and secondary RIG-I signaling for interferon induction in human monocytes. Sci. Signal. 10.
79. Morris, S. B. et al. (2020). Case Series of Multisystem Inflammatory Syndrome in Adults Associated with SARS-CoV-2 Infection—United Kingdom and United States, March-August 2020. MMWR Morb. Mortal. Wkly. Rep. 69, 1450-1456.
80. Myall, K. J. et al. (2021). Persistent Post-COVID-19 Interstitial Lung Disease. An Observational Study of Corticosteroid Treatment. Annals ATS 18, 799-806.
81. Panda, A. et al. (2010). Age-associated decrease in TLR function in primary human dendritic cells predicts influenza vaccine response. J. Immunol. 184, 2518-2527.
82. Pappas, D. J. et al. (2009). Longitudinal system-based analysis of transcriptional responses to type I interferons. Physiol. Genomics 38, 362-371.
83. Phan, T. G. et al. (2007). Subcapsular encounter and complement-dependent transport of immune complexes by lymph node B cells. Nat. Immunol. 8, 992-1000.
84. Pillai, P. S. et al. (2016). Mx1 reveals innate pathways to antiviral resistance and lethal influenza disease. Science 352, 463-466.
85. Quinlan, A. R. and Hall, I. M. (2010). BEDTools: a flexible suite of utilities for comparing genomic features. Bioinformatics 26, 841-842.
86. Rao, D. A. et al. (2017). Pathologically expanded peripheral T helper cell subset drives B cells in rheumatoid arthritis. Nature 542, 110-114.
87. Ren, X. et al. (2021). COVID-19 immune features revealed by a large-scale single-cell transcriptome atlas. Cell.
88. Richardson, S. et al. (2020). Presenting Characteristics, Comorbidities, and Outcomes Among 5700 Patients Hospitalized With COVID-19 in the New York City Area. JAMA 323, 2052-2059.
89. Rusinova, I. et al. (2013). Interferome v2.0: an updated database of annotated interferon-regulated genes. Nucleic Acids Res. 41, D1040-6.
90. Schep, A. N. et al. (2017). chromVAR: inferring transcription-factor-associated accessibility from single-cell epigenomic data. Nat. Methods 14, 975-978.
91. Schulte-Schrepping, J. et al. (2020). Severe COVID-19 Is Marked by a Dysregulated Myeloid Cell Compartment. Cell 182, 1419-1440.e23.
92. Schultze, J. L. and Aschenbrenner, A. C. (2021). COVID-19 and the human innate immune system. Cell 184, 1671-1692.
93. Sette, A. and Crotty, S. (2021). Adaptive immunity to SARS-CoV-2 and COVID-19. Cell 184, 861-880.
94. Shaigany, S. et al. (2020). An adult with Kawasaki-like multisystem inflammatory syndrome associated with COVID-19. Lancet 396, e8-e10.
95. Sigfrid, L. et al. (2021). Long Covid in adults discharged from UK hospitals after Covid-19: A prospective, multicentre cohort study using the ISARIC WHO Clinical Characterisation Protocol. MedRxiv.
96. Soni, C. et al. (2020). Plasmacytoid Dendritic Cells and Type I Interferon Promote Extrafollicular B Cell Responses to Extracellular Self-DNA. Immunity 52, 1022-1038.e7.
97. Stamatatos, L. et al. (2021). mRNA vaccination boosts cross-variant neutralizing antibodies elicited by SARS-CoV-2 infection. Science.
98. Stephenson, E. et al. (2021). The cellular immune response to COVID-19 deciphered by single cell multi-omics across three UK centres (medRxiv).
99. Subramanian, A. et al. (2005). Gene set enrichment analysis: a knowledge-based approach for interpreting genome-wide expression profiles. Proc. Natl. Acad. Sci. U.S.A 102, 15545-15550.
100. Sudre, C. H. et al. (2021). Attributes and predictors of long COVID. Nat. Med.
101. Swanson, E. et al. (2021a). Simultaneous trimodal single-cell measurement of transcripts, epitopes, and chromatin accessibility using TEA-seq. Elife 10.
102. Swanson, E. et al. (2021b). Simultaneous trimodal single-cell measurement of transcripts, epitopes, and chromatin accessibility using TEA-seq. Elife 10.
103. Syedbasha, M. et al. (2020). Interferon-A Enhances the Differentiation of Naive B Cells into Plasmablasts via the mTORC1 Pathway. Cell Rep. 33, 108211.
104. Takahashi, T. et al. (2020). Sex differences in immune responses that underlie COVID-19 disease outcomes. Nature 588, 315-320.
105. U S Department, Human Services National Institutes of Health, National Institute of Allergy, and Infectious Diseases, Division of Aids (2017a). Division of AIDS (DAIDS) table for grading the severity of adult and pediatric adverse events, corrected version 2.1 (US Department of Health and Human Services, National Institutes of Health . . . ).
106. U S Department, Human Services National Institutes of Health, National Institute of Allergy, and Infectious Diseases, Division of Aids (2017b). Division of AIDS (DAIDS) table for grading the severity of adult and pediatric adverse events, corrected version 2.1 (US Department of Health and Human Services, National Institutes of Health . . . ).
107. Van, P. et al. (2018a). ggCyto: next generation open-source visualization software for cytometry. Bioinformatics 34, 3951-3953.
108. Van, P. et al. (2018b). ggCyto: next generation open-source visualization software for cytometry. Bioinformatics 34, 3951-3953.
109. Vanderheiden, A. et al. (2020a). Type I and Type III Interferons Restrict SARS-CoV-2 Infection of Human Airway Epithelial Cultures. J. Virol. 94.
110. Vanderheiden, A. et al. (2020b). Development of a Rapid Focus Reduction Neutralization Test Assay for Measuring SARS-CoV-2 Neutralizing Antibodies. Curr. Protoc. Immunol. 131, e116.
111. Vella, L. et al. (2020). Deep Immune Profiling of MIS-C demonstrates marked but transient immune activation compared to adult and pediatric COVID-19. MedRxiv.
112. Vierstra, J. et al. (2020). Global reference mapping of human transcription factor footprints. Nature 583, 729-736.
113. Wang, E. Y. et al. (2020a). Diverse Functional Autoantibodies in Patients with COVID-19. MedRxiv.
114. Wang, N. et al. (2020b). Retrospective multicenter cohort study shows early interferon therapy is associated with favorable clinical responses in COVID-19 patients. Cell Host Microbe 28, 455-464.e2.
115. Wickham, H. (2017). The tidyverse. R Package Ver 1, 1.
116. Williamson, E. J. et al. (2020). OpenSAFELY: factors associated with COVID-19 death in 17 million patients. Nature.
117. Woodruff, M. C. et al. (2020). Extrafollicular B cell responses correlate with neutralizing antibodies and morbidity in COVID-19. Nat. Immunol. 21, 1506-1516.
118. Wrammert, J. et al. (2012). Rapid and massive virus-specific plasmablast responses during acute dengue virus infection in humans. J. Virol. 86, 2911-2918.
119. Wu, Z. and McGoogan, J. M. (2020). Characteristics of and important lessons from the coronavirus disease 2019 (COVID-19) outbreak in China: summary of a report of 72 314 cases from the Chinese Center for Disease Control and Prevention. JAMA 323, 1239-1242.
120. Xie, X. et al. (2020). An Infectious cDNA Clone of SARS-CoV-2. Cell Host Microbe 27, 841-848.e3.
121. Yamada, T. et al. (2021). RIG-I triggers a signaling-abortive anti-SARS-CoV-2 defense in human lung cells. Nat. Immunol.
122. Yates, A. D. et al. (2020). Ensembl 2020. Nucleic Acids Res. 48, D682-D688.
123. Zhang, Q. et al. (2020). Inborn errors of type I IFN immunity in patients with life-threatening COVID-19. Science 370.
124. Zhang, Y. et al. (2019). Extracellular Vesicles with Exosome-like Features Transfer TLRs between Dendritic Cells. Immunohorizons 3, 186-193.

TABLE 1

| | | | | | Number of |
|---|---|---|---|---|---|
| | | | | Number of | genesets |
| Module | | Genesets of module | | genes in | occurance |
| number | Module name | (JI >= 25%) | Genes in module | module | per gene |
| module1 | BIOCARTA_{—} | BIOCARTA_PPARA_PATHWAY | ACOX1, APOA1, APOA2, CITED2, CREBBP, DUSP1, | 52 | 1, 1, 1, 1, 1, 1, |
| | PPARA_PATHWAY | | DUT, EHHADH, EP300, FABP1, HSD17B4, | | 1, 1, 1, 1, 1, 1, |
| | | | HSP90AA1, HSPA1A, JUN, LPL, MAPK1, MAPK3, | | 1, 1, 1, 1, 1, 1, |
| | | | ME1, MED1, MRPL11, MYC, NCOA1, NCOR1, | | 1, 1, 1, 1, 1, 1, |
| | | | NCOR2, NFKBIA, NOS2, NROB2, NR1H3, NRIP1, | | 1, 1, 1, 1, 1, 1, |
| | | | PDGFA, PIK3CA, PIK3CG, PIK3R1, PPARA, PPARGC1A, | | 1, 1, 1, 1, 1, 1, |
| | | | PRKACB, PRKACG, PRKAR1A, PRKAR1B, | | 1, 1, 1, 1, 1, 1, |
| | | | PRKAR2A, PRKAR2B, PRKCA, PRKCB, | | 1, 1, 1, 1, 1, 1, |
| | | | PTGS2, RB1, RELA, RXRA, SP1, SRA1, STAT5A, | | 1, 1, 1, 1 |
| | | | STAT5B, TNF | | |
| module2 | PID_MYC_{—} | PID_MYC_REPRESS_PATHWAY | ALDH9A1, BCL2, BRCA1, CCL5, CCND1, CDKN1A, | 63 | 1, 1, 1, 1, 1, 1, |
| | REPRESS_PATHWAY | | CDKN1B, CDKN2B, CEBPA, CEBPD, CFLAR, | | 1, 1, 1, 1, 1, 1, |
| | | | CLU, COL1A2, CREB1, CSDE1, DDIT3, DKK1, | | 1, 1, 1, 1, 1, 1, |
| | | | DNMT3A, DNTT, EP300, ERBB2, FOXO3, FTH1, | | 1, 1, 1, 1, 1, 1, |
| | | | GADD45A, GFI1, GTF2H2, HDAC1, HDAC3, HMGCS2, | | 1, 1, 1, 1, 1, 1, |
| | | | ID2, IRF8, ITGA6, ITGB1, ITGB4, LGALS1, | | 1, 1, 1, 1, 1, 1, |
| | | | MAX, MXD4, MYC, NDRG1, NDRG2, NFYA, NFYB, | | 1, 1, 1, 1, 1, 1, |
| | | | NFYC, PDGFRB, PTPA, RBL1, S100A7, SFRP1, | | 1, 1, 1, 1, 1, 1, |
| | | | SFXN3, SLC11A1, SMAD2, SMAD3, SMAD4, | | 1, 1, 1, 1, 1, 1, |
| | | | SP1, SPI1, TBP, TJP2, TMEFF2, TMEM126A, | | 1, 1, 1, 1, 1, 1, |
| | | | TSC2, WNT5A, ZBTB17, ZFP36L1 | | 1, 1, 1 |
| module3 | WP_IL18_{—} | WP_IL18_SIGNALING_PATHWAY | AARS1, ABCF1, ABHD16A, ACACB, ACADS, ACOD1, | 279 | 1, 1, 1, 1, 1, 1, |
| | SIGNALING_PATHWAY | | ACTA1, ACTA2, ADAMTS5, ADIPOQ, ALS2, | | 1, 1, 1, 1, 1, 1, |
| | | | ANP32A, APBA2, ARF6, ARFGAP1, ARFGAP2, | | 1, 1, 1, 1, 1, 1, |
| | | | ARG1, ARL4D, ATF3, B2M, BAD, BAX, BAZ1B, | | 1, 1, 1, 1, 1, 1, |
| | | | BCL2, BCL2L1, BID, BIN1, BIRC3, BMP2, BPGM, | | 1, 1, 1, 1, 1, 1, |
| | | | BSG, BTG2, CA11, CASP3, CASP8, CCDC9, | | 1, 1, 1, 1, 1, 1, |
| | | | CCL1, CCL18, CCL19, CCL2, CCL20, CCL3, CCL4, | | 1, 1, 1, 1, 1, 1, |
| | | | CCL5, CCN4, CCNA2, CCNB2, CD36, CD81, CD83, | | 1, 1, 1, 1, 1, 1, |
| | | | CDK5R2, CEBPB, CENPB, CETP, CFLAR, | | 1, 1, 1, 1, 1, 1, |
| | | | CHUK, CLDN1, CLDN12, CLDN15, CLDN3, CLDN4, | | 1, 1, 1, 1, 1, 1, |
| | | | CNTN2, COL1A1, COL1A2, COL3A1, COX17, | | 1, 1, 1, 1, 1, 1, |
| | | | CPT1A, CRYGC, CSN2, CTNNB1, CXCL16, CXCL2, | | 1, 1, 1, 1, 1, 1, |
| | | | CXCL3, CXCL8, CYCS, DEK, DES, ECH1, | | 1, 1, 1, 1, 1, 1, |
| | | | EEF2, ELAVL1, ENO1, EPB41, EPS8, FADD, FAM110A, | | 1, 1, 1, 1, 1, 1, |
| | | | FAM186B, FAS, FASLG, FBXW7, FN1, FOS, | | 1, 1, 1, 1, 1, 1, |
| | | | FOXN3, FUT1, GATA1, GPAT4, GRIN2B, GRM7, | | 1, 1, 1, 1, 1, 1, |
| | | | GRN, GSK3B, HADH, HCAR2, HDAC3, HDGF, | | 1, 1, 1, 1, 1, 1, |
| | | | HMOX1, HOXD8, HPS1, HSPB1, HSPB8, ICAM1, | | 1, 1, 1, 1, 1, 1, |
| | | | IER3, IFNG, IKBKB, IL10, IL12B, IL13, IL17RC, | | 1, 1, 1, 1, 1, 1, |
| | | | IL18, IL18BP, IL18R1, IL18RAP, IL1B, IL2RA, | | 1, 1, 1, 1, 1, 1, |
| | | | IL37, IL6, IL9, IMP3, IRAK1, IRF1, IRF6, ITGA2B, ITM2C, | | 1, 1, 1, 1, 1, 1, |
| | | | JUN, KCNH2, KIFC3, KITLG, KLC1, KLF2, | | 1, 1, 1, 1, 1, 1, |
| | | | KRT31, LARS2, LCK, LMNB2, LONP2, LRRFIP1, | | 1, 1, 1, 1, 1, 1, |
| | | | LTB, MAP2K7, MAP3K7, MAPK1, MAPK3, MAPK9, | | 1, 1, 1, 1, 1, 1, |
| | | | MBTPS1, MEF2A, MEPCE, MIR3606, MIR3917, | | 1, 1, 1, 1, 1, 1, |
| | | | MIR6732, MIR7108, MIR7114, MIR718, MMP1, | | 1, 1, 1, 1, 1, 1, |
| | | | MMP13, MMP14, MMP2, MMP3, MMP8, MMP9, | | 1, 1, 1, 1, 1, 1, |
| | | | MTCH1, MYD88, MYH6, MYH7, NACA, NCAPH2, | | 1, 1, 1, 1, 1, 1, |
| | | | NCF1, NCF2, NDUFC1, NFATC4, NFKB1, NFKB2, | | 1, 1, 1, 1, 1, 1, |
| | | | NFKBIA, NFKBIE, NFKBIZ, NOS2, NOX1, NPPA, | | 1, 1, 1, 1, 1, 1, |
| | | | NPPB, NROB2, NR1H3, NR4A1, NRN1, NSMF, | | 1, 1, 1, 1, 1, 1, |
| | | | PARP1, PHF20, PIGT, PIK3R1, PKN1, PLA2G7, | | 1, 1, 1, 1, 1, 1, |
| | | | PLCG1, PLD1, PLOD3, PPP1R13L, PPT2, PRCC, | | 1, 1, 1, 1, 1, 1, |
| | | | PRKAA1, PRKCA, PRKCB, PRKCD, PRM1, | | 1, 1, 1, 1, 1, 1, |
| | | | PTEN, PTGS2, PTMS, PTPN7, PTPRZ1, PTX3, PWWP3A, | | 1, 1, 1, 1, 1, 1, |
| | | | PYGB, RAE1, RANGAP1, RASA3, REL, | | 1, 1, 1, 1, 1, 1, |
| | | | RELA, RFX5, RGS16, RND2, RPS11, RPS6KB1, | | 1, 1, 1, 1, 1, 1, |
| | | | RPTOR, RUNX2, RUSC1, RXRB, S1PR4, SDC4, | | 1, 1, 1, 1, 1, 1, |
| | | | SEMA6C, SEMA6D, SLC12A3, SLC4A7, SNTB1, | | 1, 1, 1, 1, 1, 1, |
| | | | SOCS3, SP1, SPON1, SPP1, STK40, STMN1, | | 1, 1, 1, 1, 1, 1, |
| | | | STOML1, SYT10, TACR1, TBX21, TF, TGM2, TICAM2, | | 1, 1, 1, 1, 1, 1, |
| | | | TIMP1, TIMP3, TMEM165, TMSB4X, TNF, | | 1, 1, 1, 1, 1, 1, |
| | | | TNFAIP2, TNFAIP3, TNFRSF11B, TNFRSF1A, | | 1, 1, 1, 1, 1, 1, |
| | | | TNFSF11, TNIP3, TOMM40, TP53, TRAF1, TRAF6, | | 1, 1, 1, 1, 1, 1, |
| | | | TRPC2, TRPC4AP, TRPM7, TSHZ1, UCK1, UGGT1, | | 1, 1, 1, 1, 1, 1, |
| | | | UGT2B10, ULBP2, USP5, VEGFA, ZBTB7A, | | 1, 1, 1, 1, 1, 1, |
| | | | ZC3H12A, ZDHHC7, ZNF143, ZNF219, ZNF444 | | 1, 1, 1 |
| module4 | REACTOME_{—} | REACTOME_SIGNALING_{—} | AGER, APP, BTRC, CHUK, CUL1, FBXW11, HMGB1, | 501 | 5, 5, 5, 5, 5, 5, |
| | IL1_SIGNALING/ | BY_INTERLEUKINS, | IKBKB, IKBKG, IRAK1, IRAK2, MAP2K1, MAP2K4, | | 5, 5, 5, 5, 5, 5, |
| | TLR4_TLR10_{—} | REACTOME_INTERLEUKIN_{—} | MAP2K6, MAP3K7, MAP3K8, MAPK8, NFKB1, | | 5, 5, 5, 5, 5, 5, |
| | CASCADE | 1_FAMILY_SIGNALING, | NFKB2, NFKBIA, NFKBIB, NKIRAS1, NKIRAS2, | | 5, 5, 5, 5, 5, 5, |
| | | REACTOME_TOLL_LIKE_{—} | NOD1, NOD2, RELA, RIPK2, RPS27A, S100A12, | | 5, 5, 5, 5, 5, 5, |
| | | RECEPTOR_4_TLR4_CASCADE, | S100B, SAA1, SKP1, TAB1, TAB2, TAB3, | | 5, 5, 5, 5, 5, 5, |
| | | REACTOME_MYD88_{—} | TNIP2, TRAF6, UBA52, UBB, UBC, UBE2N, UBE2V1, | | 5, 5, 5, 5, 5, 5, |
| | | INDEPENDENT_TLR4_CASCADE_, | ATF1, ATF2, CREB1, DUSP3, DUSP4, DUSP6, | | 4, 4, 4, 4, 4, 4, |
| | | REACTOME_TOLL_LIKE_{—} | DUSP7, ELK1, FOS, IRAK4, JUN, MAP2K3, MAP2K7, | | 4, 4, 4, 4, 4, 4, |
| | | RECEPTOR_10_TLR10_CASCADE | MAPK1, MAPK10, MAPK11, MAPK14, MAPK3, | | 4, 4, 4, 4, 4, 4, |
| | | | MAPK7, MAPK9, MAPKAPK2, MAPKAPK3, | | 4, 4, 4, 4, 4, 4, |
| | | | MEF2A, MEF2C, MYD88, PELI1, PELI2, PELI3, | | 4, 4, 4, 4, 4, 4, |
| | | | PPP2CA, PPP2CB, PPP2R1A, PPP2R1B, PPP2R5D, | | 4, 4, 4, 4, 4, 4, |
| | | | PTPN11, RPS6KA1, RPS6KA2, RPS6KA3, | | 4, 4, 4, 4, 3, 3, |
| | | | RPS6KA5, TBK1, VRK3, IRAK3, PTPN4, SIGIRR, | | 3, 2, 2, 2, 2, 2, |
| | | | ALOX5, BIRC2, BIRC3, CASP1, CASP8, CD14, | | 2, 2, 2, 2, 2, 2, |
| | | | CD36, CTSG, ECSIT, FADD, IKBKE, IL13, IL18, IL18BP, | | 2, 2, 2, 2, 2, 2, |
| | | | IL18R1, IL18RAP, IL1A, IL1B, IL1F10, IL1R1, | | 2, 2, 2, 2, 2, 2, |
| | | | IL1R2, IL1RAP, IL1RAPL1, IL1RL1, IL1RL2, IL1RN, | | 2, 2, 2, 2, 2, 2, |
| | | | IL33, IL36A, IL36B, IL36G, IL36RN, IL37, IL4, | | 2, 2, 2, 2, 2, 2, |
| | | | IRF3, IRF7, ITGAM, ITGB2, LBP, LY96, MAP3K1, | | 2, 2, 2, 2, 2, 2, |
| | | | MAP3K3, PSMA1, PSMA2, PSMA3, PSMA4, PSMA5, | | 2, 2, 2, 2, 2, 2, |
| | | | PSMA6, PSMA7, PSMA8, PSMB1, PSMB10, | | 2, 2, 2, 2, 2, 2, |
| | | | PSMB11, PSMB2, PSMB3, PSMB4, PSMB5, | | 2, 2, 2, 2, 2, 2, |
| | | | PSMB6, PSMB7, PSMB8, PSMB9, PSMC1, PSMC2, | | 2, 2, 2, 2, 2, 2, |
| | | | PSMC3, PSMC4, PSMC5, PSMC6, PSMD1, | | 2, 2, 2, 2, 2, 2, |
| | | | PSMD10, PSMD11, PSMD12, PSMD13, PSMD14, | | 2, 2, 2, 2, 2, 2, |
| | | | PSMD2, PSMD3, PSMD4, PSMD5, PSMD6, PSMD7, | | 2, 2, 2, 2, 2, 2, |
| | | | PSMD8, PSMD9, PSME1, PSME2, PSME3, | | 2, 2, 2, 2, 2, 2, |
| | | | PSME4, PSMF1, PTPN12, PTPN13, PTPN14, | | 2, 2, 2, 2, 2, 2, |
| | | | PTPN18, PTPN2, PTPN20, PTPN23, PTPN5, PTPN6, | | 2, 2, 2, 2, 2, 2, |
| | | | PTPN7, PTPN9, RBX1, RIPK1, RIPK3, SARM1, | | 2, 2, 2, 2, 2, 2, |
| | | | SEM1, SMAD3, SOCS1, SQSTM1, STAT3, TANK, | | 2, 2, 2, 2, 2, 2, |
| | | | TICAM1, TICAM2, TLR4, TOLLIP, TRAF3, | | 2, 1, 1, 1, 1, 1, |
| | | | UBE2D1, UBE2D2, UBE2D3, AIP, AKT1, ALOX15, | | 1, 1, 1, 1, 1, 1, |
| | | | ANXA1, ANXA2, ARF1, BATF, BCL2, BCL2L1, | | 1, 1, 1, 1, 1, 1, |
| | | | BCL6, BIRC5, BLNK, BOLA2, BOLA2B, BPI, BRWD1, | | 1, 1, 1, 1, 1, 1, |
| | | | BTK, CA1, CANX, CAPZA1, CASP3, CBL, CCL11, | | 1, 1, 1, 1, 1, 1, |
| | | | CCL19, CCL2, CCL20, CCL22, CCL3, CCL3L1, | | 1, 1, 1, 1, 1, 1, |
| | | | CCL3L3, CCL4, CCL5, CCND1, CCR1, CCR2, | | 1, 1, 1, 1, 1, 1, |
| | | | CCR5, CD180, CD4, CD80, CD86, CDC42, CDKN1A, | | 1, 1, 1, 1, 1, 1, |
| | | | CEBPD, CFL1, CISH, CLCF1, CNN2, CNTF, | | 1, 1, 1, 1, 1, 1, |
| | | | CNTFR, COL1A2, CRK, CRKL, CRLF1, CRLF2, | | 1, 1, 1, 1, 1, 1, |
| | | | CSF1, CSF1R, CSF2, CSF2RA, CSF2RB, CSF3, | | 1, 1, 1, 1, 1, 1, |
| | | | CSF3R, CTF1, CXCL1, CXCL10, CXCL2, CXCL8, | | 1, 1, 1, 1, 1, 1, |
| | | | DNM1, DNM2, DNM3, EBI3, F13A1, FASLG, FCER2, | | 1, 1, 1, 1, 1, 1, |
| | | | FGF2, FN1, FOXO1, FOXO3, FPR1, FSCN1, | | 1, 1, 1, 1, 1, 1, |
| | | | FYN, GAB2, GATA3, GRB2, GSTA2, GSTO1, | | 1, 1, 1, 1, 1, 1, |
| | | | H3C1, H3C10, H3C11, H3C12, H3C13, H3C14, H3C15, | | 1, 1, 1, 1, 1, 1, |
| | | | H3C2, H3C3, H3C4, H3C6, H3C7, H3C8, HAVCR2, | | 1, 1, 1, 1, 1, 1, |
| | | | HCK, HGF, HIF1A, HMOX1, HNRNPA2B1, | | 1, 1, 1, 1, 1, 1, |
| | | | HNRNPDL, HNRNPF, HSP90AA1, HSP90B1, | | 1, 1, 1, 1, 1, 1, |
| | | | HSPA8, HSPA9, ICAM1, IFNG, IFNL1, IFNL2, IFNL3, | | 1, 1, 1, 1, 1, 1, |
| | | | IFNLR1, IGHE, IGHG1, IGHG4, IL10, IL10RA, | | 1, 1, 1, 1, 1, 1, |
| | | | IL10RB, IL11, IL11RA, IL12A, IL12B, IL12RB1, | | 1, 1, 1, 1, 1, 1, |
| | | | IL12RB2, IL13RA1, IL13RA2, IL15, IL15RA, IL16, | | 1, 1, 1, 1, 1, 1, |
| | | | IL17A, IL17C, IL17F, IL17RA, IL17RB, IL17RC, IL17RE, | | 1, 1, 1, 1, 1, 1, |
| | | | IL19, IL2, IL20, IL20RA, IL20RB, IL21, IL21R, | | 1, 1, 1, 1, 1, 1, |
| | | | IL22, IL22RA1, IL22RA2, IL23A, IL23R, IL24, IL25, | | 1, 1, 1, 1, 1, 1, |
| | | | IL26, IL27, IL27RA, IL2RA, IL2RB, IL2RG, IL3, | | 1, 1, 1, 1, 1, 1, |
| | | | IL31, IL31RA, IL32, IL34, IL3RA, IL4R, IL5, IL5RA, | | 1, 1, 1, 1, 1, 1, |
| | | | IL6, IL6R, IL6ST, IL7, IL7R, IL9, IL9R, INPP5D, INPPL1, | | 1, 1, 1, 1, 1, 1, |
| | | | IRF4, IRS1, IRS2, ITGAX, ITGB1, JAK1, JAK2, | | 1, 1, 1, 1, 1, 1, |
| | | | JAK3, JUNB, LAMA5, LCK, LCN2, LCP1, LGALS9, | | 1, 1, 1, 1, 1, 1, |
| | | | LIF, LIFR, LMNB1, LY86, LYN, MAOA, MCL1, | | 1, 1, 1, 1, 1, 1, |
| | | | MIF, MMP1, MMP2, MMP3, MMP9, MSN, MTAP, | | 1, 1, 1, 1, 1, 1, |
| | | | MUC1, MYC, NANOG, NDN, NOS2, OPRD1, OPRM1, | | 1, 1, 1, 1, 1, 1, |
| | | | OSM, OSMR, P4HB, PAK2, PDCD4, PIK3CA, | | 1, 1, 1, 1, 1, 1, |
| | | | PIK3CB, PIK3CD, PIK3R1, PIK3R2, PIK3R3, PIM1, | | 1, 1, 1, 1, 1, 1, |
| | | | PITPNA, PLCG2, POMC, POU2F1, PPIA, PRKACA, | | 1, 1, 1, 1, 1, 1, |
| | | | PRTN3, PTAFR, PTGS2, PTK2B, PTPRZ1, | | 1, 1, 1, 1, 1, 1, |
| | | | RAG1, RAG2, RALA, RAP1B, RAPGEF1, RHOU, | | 1, 1, 1, 1, 1, 1, |
| | | | RORA, RORC, RPLPO, S1PR1, SDC1, SERPINB2, | | 1, 1, 1, 1, 1, 1, |
| | | | SFTPA1, SFTPA2, SFTPD, SHC1, SMARCA4, | | 1, 1, 1, 1, 1, 1, |
| | | | SNAP25, SNRPA1, SOCS2, SOCS3, SOCS5, | | 1, 1, 1, 1, 1, 1, |
| | | | SOD1, SOD2, SOS1, SOS2, SOX2, STAT1, STAT2, | | 1, 1, 1, 1, 1, 1, |
| | | | STAT4, STAT5A, STAT5B, STAT6, STX1A, | | 1, 1, 1, 1, 1, 1, |
| | | | STX3, STX4, STXBP2, SYK, TALDO1, TCP1, TEC, | | 1, 1, 1, 1, 1, 1, |
| | | | TGFB1, TIMP1, TIRAP, TLR1, TLR10, TLR2, | | 1, 1, 1, 1, 1, 1, |
| | | | TLR5, TLR6, TNF, TNFRSF1A, TNFRSF1B, TP53, | | 1, 1, 1, 1, 1, 1, |
| | | | TSLP, TWIST1, TXLNA, TYK2, VAMP2, VAMP7, | | 1, 1, 1, 1, 1, 1, |
| | | | VAV1, VCAM1, VEGFA, VIM, YES1, YWHAZ, ZEB1 | | 1, 1, 1, 1, 1, 1, |
| | | | | | 1, 1, 1, 1, 1, 1, |
| | | | | | 1, 1, 1 |
| module5 | PID_ANTHRAX_{—} | PID_ANTHRAX_PATHWAY, | ANTXR1, ANTXR2, MAP2K1, MAP2K2, MAP2K3, | 21 | 2, 2, 2, 2, 2, 2, |
| | PATHWAY | REACTOME_UPTAKEAND_{—} | MAP2K4, MAP2K6, MAP2K7, CALM1, CASP1, | | 2, 2, 1, 1, 1, 1, |
| | | FUNCTION_OF_ANTHRAX_TOXINS | DEFA1, FURIN, IL18, IL1B, MAPK1, MAPK3, NLRP1, | | 1, 1, 1, 1, 1, 1, |
| | | | PDCD6IP, PGR, TNF, VCAM1 | | 1, 1, 1 |
| module6 | WP_LEPTIN_{—} | WP_LEPTIN_SIGNALING_PATHWAY | ACACA, ACACB, AKT1, BAD, BAX, BCL2L1, CCND1, | 76 | 1, 1, 1, 1, 1, 1, |
| | SIGNALING_PATHWAY | | CDC42, CFL2, CHUK, CISH, CREB1, EIF4E, | | 1, 1, 1, 1, 1, 1, |
| | | | EIF4EBP1, ELK1, ERBB2, ESR1, FOXO1, FYN, | | 1, 1, 1, 1, 1, 1, |
| | | | GRB2, GSK3A, GSK3B, HRAS, IKBKB, IKBKG, | | 1, 1, 1, 1, 1, 1, |
| | | | IL1B, IL1RN, IRS1, JAK1, JAK2, KHDRBS1, KPNA4, | | 1, 1, 1, 1, 1, 1, |
| | | | LEP, LEPR, MAP2K1, MAP2K2, MAPK1, MAPK14, | | 1, 1, 1, 1, 1, 1, |
| | | | MAPK3, MAPK8, MTOR, NCOA1, NFKB1, | | 1, 1, 1, 1, 1, 1, |
| | | | NOS3, PDE3B, PIK3R1, PIK3R2, PLCG1, PLCG2, | | 1, 1, 1, 1, 1, 1, |
| | | | PRKAA1, PRKAA2, PTEN, PTK2, PTPN1, PTPN11, | | 1, 1, 1, 1, 1, 1, |
| | | | RAC1, RAF1, REL, RELA, RHOA, ROCK1, ROCK2, | | 1, 1, 1, 1, 1, 1, |
| | | | RPS6, RPS6KA1, RPS6KB1, SH2B1, SHC1, | | 1, 1, 1, 1, 1, 1, |
| | | | SOCS2, SOCS3, SOCS7, SOS1, SP1, SRC, | | 1, 1, 1, 1, 1, 1, |
| | | | STAT1, STAT3, STAT5B | | 1, 1, 1, 1 |
| module7 | WP_BIOMARKERS_{—} | WP_BIOMARKERS_FOR_UREA_{—} | ARG1, ASL, ASS1, F10, F7, GAMT, GATM, GLS2, | 12 | 1, 1, 1, 1, 1, 1, |
| | FOR_UREA_CYCLE_{—} | CYCLE_DISORDERS | GOT1, GPT, NAGS, OTC | | 1, 1, 1, 1, 1, 1 |
| | DISORDERS | | | | |
| module8 | REACTOME_{—} | REACTOME_BMAL1_CLOCK_{—} | ARNTL, ARNTL2, AVP, BHLHE40, BHLHE41, CARM1, | 27 | 1, 1, 1, 1, 1, 1, |
| | CIRCADIAN_{—} | NPAS2_ACTIVATES_{—} | CHD9, CLOCK, CREBBP, DBP, F7, HELZ2, | | 1, 1, 1, 1, 1, 1, |
| | GENE_{—} | CIRCADIAN_GENE_EXPRESSION | KLF15, MED1, NAMPT, NCOA1, NCOA2, NCOA6, | | 1, 1, 1, 1, 1, 1, |
| | EXPRESSION | | NOCT, NPAS2, PPARA, RXRA, SERPINE1, | | 1, 1, 1, 1, 1, 1, |
| | | | SMARCD3, TBL1X, TBL1XR1, TGS1 | | 1, 1, 1 |
| module9 | REACTOME_{—} | REACTOME_INFECTIOUS_DISEASE | AAAS, ABI1, ABI2, ABL1, ACE2, ACTB, ACTG1, ACTR2, | 866 | 1, 1, 1, 1, 1, 1, |
| | INFECTIOUS_DISEASE | | ACTR3, ADAM17, ADCY1, ADCY2, ADCY3, | | 1, 1, 1, 1, 1, 1, |
| | | | ADCY4, ADCY5, ADCY6, ADCY7, ADCY8, ADCY9, | | 1, 1, 1, 1, 1, 1, |
| | | | ADCYAP1, ADCYAP1R1, ADM, ADM2, ADORA2A, | | 1, 1, 1, 1, 1, 1, |
| | | | ADORA2B, ADRB1, ADRB2, ADRB3, | | 1, 1, 1, 1, 1, 1, |
| | | | AHCYL1, ANTXR1, ANTXR2, AP1B1, AP1G1, AP1M1, | | 1, 1, 1, 1, 1, 1, |
| | | | AP1M2, AP1S1, AP1S2, AP1S3, AP2A1, AP2A2, | | 1, 1, 1, 1, 1, 1, |
| | | | AP2B1, AP2M1, AP2S1, APOBEC3G, APP, | | 1, 1, 1, 1, 1, 1, |
| | | | ARF1, ARPC1A, ARPC1B, ARPC2, ARPC3, ARPC4, | | 1, 1, 1, 1, 1, 1, |
| | | | ARPC5, ATP1A1, ATP6V1H, AVP, AVPR2, | | 1, 1, 1, 1, 1, 1, |
| | | | B2M, BAIAP2, BANF1, BECN1, BRD4, BRK1, BTK, | | 1, 1, 1, 1, 1, 1, |
| | | | BTRC, C3, C3AR1, CALCA, CALCB, CALCR, | | 1, 1, 1, 1, 1, 1, |
| | | | CALCRL, CALM1, CALR, CANX, CASP1, CBL, CBLL1, | | 1, 1, 1, 1, 1, 1, |
| | | | CBX1, CCNH, CCNK, CCNT1, CCNT2, CCR5, | | 1, 1, 1, 1, 1, 1, |
| | | | CD163, CD247, CD28, CD3G, CD4, CD8B, CD9, | | 1, 1, 1, 1, 1, 1, |
| | | | CDC42, CDH1, CDK7, CDK9, CEBPD, CGA, | | 1, 1, 1, 1, 1, 1, |
| | | | CHMP1A, CHMP2A, CHMP2B, CHMP3, CHMP4A, | | 1, 1, 1, 1, 1, 1, |
| | | | CHMP4B, CHMP4C, CHMP5, CHMP6, CHMP7, | | 1, 1, 1, 1, 1, 1, |
| | | | CLTA, CLTC, COMT, CORO1A, CPSF4, CRBN, | | 1, 1, 1, 1, 1, 1, |
| | | | CREB1, CRH, CRHR1, CRHR2, CRK, CTDP1, CTNNB1, | | 1, 1, 1, 1, 1, 1, |
| | | | CTNND1, CTSG, CTSL, CUL5, CXCR4, | | 1, 1, 1, 1, 1, 1, |
| | | | CYBA, CYFIP1, CYFIP2, CYSLTR1, CYSLTR2, | | 1, 1, 1, 1, 1, 1, |
| | | | DAXX, DDX5, DNAJC3, DOCK1, DOCK2, DPEP1, | | 1, 1, 1, 1, 1, 1, |
| | | | DPEP2, DPEP3, DRD1, DRD5, DUSP16, DVL1, | | 1, 1, 1, 1, 1, 1, |
| | | | DVL2, DVL3, DYNC1H1, DYNC111, DYNC112, DYNC1LI1, | | 1, 1, 1, 1, 1, 1, |
| | | | DYNC1LI2, DYNLL1, DYNLL2, EED, EEF2, | | 1, 1, 1, 1, 1, 1, |
| | | | EGFR, EIF2AK2, ELK1, ELL, ELMO1, ELMO2, | | 1, 1, 1, 1, 1, 1, |
| | | | ELOA, ELOA2, ELOB, ELOC, ENO1, ENTPD1, | | 1, 1, 1, 1, 1, 1, |
| | | | ENTPD5, EPS15, ERCC2, ERCC3, EZH2, FAU, | | 1, 1, 1, 1, 1, 1, |
| | | | FCGR1A, FCGR2A, FCGR3A, FEN1, FGR, FKBP1A, | | 1, 1, 1, 1, 1, 1, |
| | | | FSHB, FSHR, FURIN, FYN, FZD7, GALNT1, | | 1, 1, 1, 1, 1, 1, |
| | | | GANAB, GCG, GGT1, GGT5, GHRH, GHRHR, GIP, | | 1, 1, 1, 1, 1, 1, |
| | | | GIPR, GLP1R, GLP2R, GNAI1, GNAI2, GNAI3, | | 1, 1, 1, 1, 1, 1, |
| | | | GNAS, GNAT3, GNAZ, GNB1, GNB2, GNB3, GNB4, | | 1, 1, 1, 1, 1, 1, |
| | | | GNB5, GNG10, GNG11, GNG12, GNG13, GNG2, | | 1, 1, 1, 1, 1, 1, |
| | | | GNG3, GNG4, GNG5, GNG7, GNG8, GNGT1, | | 1, 1, 1, 1, 1, 1, |
| | | | GNGT2, GPBAR1, GPHA2, GPHB5, GPR15, GPR150, | | 1, 1, 1, 1, 1, 1, |
| | | | GPR176, GPR20, GPR25, GPR27, GPR32, | | 1, 1, 1, 1, 1, 1, |
| | | | GPR39, GPR45, GPR83, GPR84, GPS2, GRB2, | | 1, 1, 1, 1, 1, 1, |
| | | | GRSF1, GSK3A, GSK3B, GTF2A1, GTF2A2, GTF2B, | | 1, 1, 1, 1, 1, 1, |
| | | | GTF2E1, GTF2E2, GTF2F1, GTF2F2, GTF2H1, | | 1, 1, 1, 1, 1, 1, |
| | | | GTF2H2, GTF2H3, GTF2H4, GTF2H5, GUCY2C, | | 1, 1, 1, 1, 1, 1, |
| | | | H2AC1, H2AC11, H2AC12, H2AC13, H2AC14, | | 1, 1, 1, 1, 1, 1, |
| | | | H2AC15, H2AC16, H2AC17, H2AC18, H2AC19, | | 1, 1, 1, 1, 1, 1, |
| | | | H2AC20, H2AC21, H2AC4, H2AC6, H2AC7, | | 1, 1, 1, 1, 1, 1, |
| | | | H2AC8, H2AW, H2BC1, H2BC10, H2BC11, H2BC12, | | 1, 1, 1, 1, 1, 1, |
| | | | H2BC13, H2BC14, H2BC15, H2BC17, H2BC18, | | 1, 1, 1, 1, 1, 1, |
| | | | H2BC21, H2BC3, H2BC4, H2BC5, H2BC6, | | 1, 1, 1, 1, 1, 1, |
| | | | H2BC7, H2BC8, H2BC9, H2BU1, H3C1, H3C10, | | 1, 1, 1, 1, 1, 1, |
| | | | H3C11, H3C12, H3C13, H3C14, H3C15, H3C2, H3C3, | | 1, 1, 1, 1, 1, 1, |
| | | | H3C4, H3C6, H3C7, H3C8, H4-16, | | 1, 1, 1, 1, 1, 1, |
| | | | H4C1, H4C11, H4C12, H4C13, H4C14, H4C15, | | 1, 1, 1, 1, 1, 1, |
| | | | H4C2, H4C3, H4C4, H4C5, H4C6, H4C8, H4C9, | | 1, 1, 1, 1, 1, 1, |
| | | | HBEGF, HCK, HDAC2, HDAC3, HGS, HLA-A, | | 1, 1, 1, 1, 1, 1, |
| | | | HMGA1, HNRNPK, HRH2, HSP90AA1, HSP90AB1, | | 1, 1, 1, 1, 1, 1, |
| | | | HSPA1A, HTR4, HTR6, HTR7, IAPP, IGHG1, | | 1, 1, 1, 1, 1, 1, |
| | | | IGHG2, IGHG4, IGHV1-2, IGHV1-46, IGHV1-69, | | 1, 1, 1, 1, 1, 1, |
| | | | IGHV2-5, IGHV2-70, IGHV3-11, IGHV3-13, | | 1, 1, 1, 1, 1, 1, |
| | | | IGHV3-23, IGHV3-30, IGHV3-33, IGHV3-48, | | 1, 1, 1, 1, 1, 1, |
| | | | IGHV3-53, IGHV3-7, IGHV4-34, IGHV4-39, | | 1, 1, 1, 1, 1, 1, |
| | | | IGHV4-59, IGKV1-12, IGKV1-16, IGKV1-17, | | 1, 1, 1, 1, 1, 1, |
| | | | IGKV1-33, IGKV1-39, IGKV1-5, IGKV1D-12, | | 1, 1, 1, 1, 1, 1, |
| | | | IGKV1D-16, IGKV1D-33, IGKV1D-39, IGKV2-28, | | 1, 1, 1, 1, 1, 1, |
| | | | IGKV2-30, IGKV2D-28, IGKV2D-30, IGKV2D-40, | | 1, 1, 1, 1, 1, 1, |
| | | | IGKV3-11, IGKV3-15, IGKV3-20, IGKV3D-20, | | 1, 1, 1, 1, 1, 1, |
| | | | IGKV4-1, IGKV5-2, IGLC2, IGLC3, IGLV1-40, | | 1, 1, 1, 1, 1, 1, |
| | | | IGLV1-44, IGLV1-47, IGLV1-51, IGLV2-11, | | 1, 1, 1, 1, 1, 1, |
| | | | IGLV2-14, IGLV2-23, IGLV2-8, IGLV3-1, | | 1, 1, 1, 1, 1, 1, |
| | | | IGLV3-19, IGLV3-21, IGLV3-25, IGLV3-27, | | 1, 1, 1, 1, 1, 1, |
| | | | IGLV6-57, IGLV7-43, IL10, IL18, IL1A, IL1B, | | 1, 1, 1, 1, 1, 1, |
| | | | IL1R1, IL1RAP, IL6, IL6R, IMPDH1, IMPDH2, INSL3, | | 1, 1, 1, 1, 1, 1, |
| | | | IPO5, ISG15, ITGA4, | | 1, 1, 1, 1, 1, 1, |
| | | | ITGB1, ITPR1, ITPR2, ITPR3, JAK1, JAK2, JAK3, | | 1, 1, 1, 1, 1, 1, |
| | | | JUN, KPNA1, KPNA2, KPNA3, KPNA4, KPNA5, | | 1, 1, 1, 1, 1, 1, |
| | | | KPNA7, KPNB1, LCK, LHB, LHCGR, LIG1, LIG4, | | 1, 1, 1, 1, 1, 1, |
| | | | LTF, LYN, MAP1LC3B, MAP2K1, MAP2K2, MAP2K3, | | 1, 1, 1, 1, 1, 1, |
| | | | MAP2K4, MAP2K6, MAP2K7, MAPK1, MAPK14, | | 1, 1, 1, 1, 1, 1, |
| | | | MAPK3, MAPK8, MC1R, MC2R, MC3R, MC4R, | | 1, 1, 1, 1, 1, 1, |
| | | | MC5R, MEFV, MET, MGAT1, MNAT1, MOGS, | | 1, 1, 1, 1, 1, 1, |
| | | | MRC1, MVB12A, MVB12B, MYH2, MYH9, MYO10, | | 1, 1, 1, 1, 1, 1, |
| | | | MYO1C, MYO5A, MYO9B, NCBP1, NCBP2, | | 1, 1, 1, 1, 1, 1, |
| | | | NCK1, NCKAP1, NCKAP1L, NCKIPSD, NCOR1, | | 1, 1, 1, 1, 1, 1, |
| | | | NCOR2, NDC1, NEDD4L, NELFA, NELFB, NELFCD, | | 1, 1, 1, 1, 1, 1, |
| | | | NELFE, NFKB1, NFKB2, NLRP3, NMT1, NMT2, | | 1, 1, 1, 1, 1, 1, |
| | | | NOS2, NOX1, NOXA1, NOXO1, NPM1, NPS, | | 1, 1, 1, 1, 1, 1, |
| | | | NPSR1, NR3C1, NT5E, NUP107, NUP133, NUP153, | | 1, 1, 1, 1, 1, 1, |
| | | | NUP155, NUP160, NUP188, NUP205, NUP210, | | 1, 1, 1, 1, 1, 1, |
| | | | NUP214, NUP35, NUP37, NUP42, NUP43, | | 1, 1, 1, 1, 1, 1, |
| | | | NUP50, NUP54, NUP58, NUP62, NUP85, NUP88, | | 1, 1, 1, 1, 1, 1, |
| | | | NUP93, NUP98, P2RX4, P2RX7, P2RY11, PABPN1, | | 1, 1, 1, 1, 1, 1, |
| | | | PACS1, PAK2, PARP1, PARP10, PARP14, | | 1, 1, 1, 1, 1, 1, |
| | | | PARP16, PARP4, PARP6, PARP8, PARP9, PDCD1, | | 1, 1, 1, 1, 1, 1, |
| | | | PDCD6IP, PDZD3, PGK1, PIK3C3, PIK3R4, | | 1, 1, 1, 1, 1, 1, |
| | | | PLCG1, PLCG2, PLK2, PML, POLR2A, POLR2B, | | 1, 1, 1, 1, 1, 1, |
| | | | POLR2C, POLR2D, POLR2E, POLR2F, POLR2G, | | 1, 1, 1, 1, 1, 1, |
| | | | POLR2H, POLR2I, POLR2J, POLR2K, POLR2L, | | 1, 1, 1, 1, 1, 1, |
| | | | POM121, POM121C, POMC, PPIA, PRKACA, | | 1, 1, 1, 1, 1, 1, |
| | | | PRKACB, PRKACG, PRKAR1A, PRKAR1B, PRKAR2A, | | 1, 1, 1, 1, 1, 1, |
| | | | PRKAR2B, PRKCSH, PRKX, PSIP1, PSMA1, | | 1, 1, 1, 1, 1, 1, |
| | | | PSMA2, PSMA3, PSMA4, PSMA5, PSMA6, | | 1, 1, 1, 1, 1, 1, |
| | | | PSMA7, PSMA8, PSMB1, PSMB10, PSMB11, PSMB2, | | 1, 1, 1, 1, 1, 1, |
| | | | PSMB3, PSMB4, PSMB5, PSMB6, PSMB7, | | 1, 1, 1, 1, 1, 1, |
| | | | PSMB8, PSMB9, PSMC1, PSMC2, PSMC3, PSMC4, | | 1, 1, 1, 1, 1, 1, |
| | | | PSMC5, PSMC6, PSMD1, PSMD10, PSMD11, | | 1, 1, 1, 1, 1, 1, |
| | | | PSMD12, PSMD13, PSMD14, PSMD2, PSMD3, | | 1, 1, 1, 1, 1, 1, |
| | | | PSMD4, PSMD5, PSMD6, PSMD7, PSMD8, | | 1, 1, 1, 1, 1, 1, |
| | | | PSMD9, PSME1, PSME2, PSME3, PSME4, PSMF1, | | 1, 1, 1, 1, 1, 1, |
| | | | PSTPIP1, PTGDR, PTGER2, PTGER4, PTGIR, | | 1, 1, 1, 1, 1, 1, |
| | | | PTH, PTH1R, PTH2, PTH2R, PTHLH, PTK2, | | 1, 1, 1, 1, 1, 1, |
| | | | PYCARD, RAB5A, RAB7A, RAC1, RAE1, RAMP1, | | 1, 1, 1, 1, 1, 1, |
| | | | RAMP2, RAMP3, RAN, RANBP1, RANBP2, RANGAP1, | | 1, 1, 1, 1, 1, 1, |
| | | | RB1, RBBP4, RBBP7, RBX1, RCC1, RELA, | | 1, 1, 1, 1, 1, 1, |
| | | | RHBDF2, RIPK1, RLN2, RLN3, RNF213, RNGTT, | | 1, 1, 1, 1, 1, 1, |
| | | | RNMT, ROCK1, ROCK2, RPL10, RPL10A, | | 1, 1, 1, 1, 1, 1, |
| | | | RPL10L, RPL11, RPL12, RPL13, RPL13A, RPL14, | | 1, 1, 1, 1, 1, 1, |
| | | | RPL15, RPL17, RPL18, RPL18A, RPL19, RPL21, | | 1, 1, 1, 1, 1, 1, |
| | | | RPL22, RPL22L1, RPL23, RPL23A, RPL24, RPL26, | | 1, 1, 1, 1, 1, 1, |
| | | | RPL26L1, RPL27, RPL27A, RPL28, RPL29, | | 1, 1, 1, 1, 1, 1, |
| | | | RPL3, RPL30, RPL31, RPL32, RPL34, RPL35, RPL35A, | | 1, 1, 1, 1, 1, 1, |
| | | | RPL36, RPL36A, RPL36AL, RPL37, RPL37A, | | 1, 1, 1, 1, 1, 1, |
| | | | RPL38, RPL39, RPL39L, RPL3L, RPL4, RPL41, | | 1, 1, 1, 1, 1, 1, |
| | | | RPL5, RPL6, RPL7, RPL7A, RPL8, RPL9, RPLPO, | | 1, 1, 1, 1, 1, 1, |
| | | | RPLP1, RPLP2, RPS10, RPS11, RPS12, RPS13, | | 1, 1, 1, 1, 1, 1, |
| | | | RPS14, RPS15, RPS15A, RPS16, RPS17, RPS18, | | 1, 1, 1, 1, 1, 1, |
| | | | RPS19, RPS2, RPS20, RPS21, RPS23, RPS24, | | 1, 1, 1, 1, 1, 1, |
| | | | RPS25, RPS26, RPS27, RPS27A, RPS27L, | | 1, 1, 1, 1, 1, 1, |
| | | | RPS28, RPS29, RPS3, RPS3A, RPS4X, RPS4Y1, | | 1, 1, 1, 1, 1, 1, |
| | | | RPS4Y2, RPS5, RPS6, RPS7, RPS8, RPS9, | | 1, 1, 1, 1, 1, 1, |
| | | | RPSA, RXFP1, RXFP2, S1PR1, SCT, SCTR, SEC13, | | 1, 1, 1, 1, 1, 1, |
| | | | SEH1L, SEM1, SFPQ, SH3GL1, SH3GL2, SH3GL3, | | 1, 1, 1, 1, 1, 1, |
| | | | SH3KBP1, SIGMAR1, SKP1, SLC25A4, | | 1, 1, 1, 1, 1, 1, |
| | | | SLC25A5, SLC25A6, SNAP25, SNF8, SRC, SSRP1, | | 1, 1, 1, 1, 1, 1, |
| | | | ST3GAL1, ST3GAL2, ST3GAL3, ST3GAL4, | | 1, 1, 1, 1, 1, 1, |
| | | | ST6GAL1, ST6GALNAC2, ST6GALNAC3, ST6 | | 1, 1, 1, 1, 1, 1, |
| | | | GALNAC4, STAM, STAM2, STX1A, STX1B, SUGT1, | | 1, 1, 1, 1, 1, 1, |
| | | | SUMO1, SUPT16H, SUPT4H1, SUPT5H, SUZ12, | | 1, 1, 1, 1, 1, 1, |
| | | | SV2A, SV2B, SV2C, SYK, SYT1, SYT2, TAAR1, | | 1, 1, 1, 1, 1, 1, |
| | | | TAAR2, TAAR5, TAAR6, TAAR8, TAAR9, TAF1, | | 1, 1, 1, 1, 1, 1, |
| | | | TAF10, TAF11, TAF12, TAF13, TAF15, TAF1L, | | 1, 1, 1, 1, 1, 1, |
| | | | TAF2, TAF3, TAF4, TAF4B, TAF5, TAF6, TAF7, | | 1, 1, 1, 1, 1, 1, |
| | | | TAF7L, TAF9, TAF9B, TBK1, TBL1X, TBL1XR1, | | 1, 1, 1, 1, 1, 1, |
| | | | TBP, TCEA1, TGFB1, TLR2, TLR7, TLR9, TMPRSS2, | | 1, 1, 1, 1, 1, 1, |
| | | | TPR, TRIM27, TRIM28, TSG101, TSHB, TSHR, | | 1, 1, 1, 1, 1, 1, |
| | | | TUBA1A, TUBA1B, TUBA1C, TUBA3C, TUBA3D, | | 1, 1, 1, 1, 1, 1, |
| | | | TUBA3E, TUBA4A, TUBA4B, TUBA8, TUBAL3, | | 1, 1 |
| | | | TUBB, TUBB1, TUBB2A, TUBB2B, TUBB3, | | |
| | | | TUBB4A, TUBB4B, TUBB6, TUBB8, TUBB8B, TXN, | | |
| | | | TXNIP, TXNRD1, TYK2, UBA52, UBAP1, UBB, | | |
| | | | UBC, UBE21, UVRAG, VAMP1, VAMP2, VAV1, | | |
| | | | VAV2, VAV3, VCP, VEGFA, VHL, VIP, VIPR1, VIPR2, | | |
| | | | VPS25, VPS28, VPS33B, VPS36, VPS37A, VPS37B, | | |
| | | | VPS37C, VPS37D, VPS4A, VPS4B, VTA1, | | |
| | | | WAS, WASF1, WASF2, WASF3, WASL, WIPF1, | | |
| | | | WIPF2, WIPF3, WNT5A, XPO1, XRCC4, XRCC5, | | |
| | | | XRCC6, YES1, ZCRB1 | | |
| module10 | PID_IL1_PATHWAY | PID_IL1_PATHWAY | CASP1, CHUK, ERC1, IKBKB, IKBKG, IL1A, IL1B, | 34 | 1, 1, 1, 1, 1, 1, |
| | | | IL1R1, IL1R2, IL1RAP, IL1RN, IRAK1, IRAK3, IRAK4, | | 1, 1, 1, 1, 1, 1, |
| | | | JUN, MAP2K6, MAP3K3, MAP3K7, MAPK8, | | 1, 1, 1, 1, 1, 1, |
| | | | MYD88, NFKB1, PIK3CA, PIK3R1, PRKCI, PRKCZ, | | 1, 1, 1, 1, 1, 1, |
| | | | RELA, SQSTM1, TAB1, TAB2, TICAM2, TOLLIP, | | 1, 1, 1, 1, 1, 1, |
| | | | TRAF6, UBE2N, UBE2V1 | | 1, 1, 1, 1 |
| module11 | REACTOME_{—} | WP_FAS_LIGAND_FASL_PATHWAY_{—} | CASP8, RIPK1, TRADD, TRAF2, BIRC3, FADD, | 162 | 11, 11, 11, 11, |
| | TNF_SIGNALING | AND_STRESS_INDUCTION_OF_HEAT_{—} | TNF, TNFRSF1A, BIRC2, CFLAR, CHUK, XIAP, | | 10, 10, 10, 9, 8, |
| | | SHOCK_PROTEINS_HSP_REGULATION, | CASP3, TRAF1, APAF1, BAG4, BCL2, CASP6, CASP7, | | 7, 7, 7, 6, 6, 5, |
| | | PID_HIV_NEF_PATHWAY, | CASP9, DFFA, DFFB, FAS, FASLG, IKBKG, | | 5, 5, 5, 5, 5, 5, |
| | | BIOCARTA_HIVNEF_PATHWAY, | NFKB1, NFKBIA, TNFAIP3, BID, CYLD, IKBKB, | | 5, 5, 5, 5, 5, 5, |
| | | WP_APOPTOSIS, | MAP3K7, OTUD7B, RELA, RPS27A, TNFRSF1B, | | 5, 4, 4, 4, 4, 4, |
| | | BIOCARTA_DEATH_PATHWAY, | UBA52, UBB, UBC, USP2, USP21, USP4, CASP10, | | 4, 4, 4, 4, 4, 4, |
| | | REACTOME_RIPK1_MEDIATED_{—} | CASP2, CRADD, CYCS, DAXX, JUN, LMNA, | | 4, 4, 4, 3, 3, 3, |
| | | REGULATED_NECROSIS, | MAP2K4, MAP3K1, MAP3K14, MAPK8, RACK1, | | 3, 3, 3, 3, 3, 3, |
| | | REACTOME_TNFR1_INDUCED_{—} | RBCK1, RNF31, SHARPIN, SPTAN1, TNFSF10, | | 3, 3, 3, 3, 3, 3, |
| | | NFKAPPAB_SIGNALING_PATHWAY, | ACTG1, ARHGDIB, CLIP3, LMNB1, LMNB2, MADD, | | 3, 3, 2, 2, 2, 2, |
| | | REACTOME_TNF_SIGNALING, | MAP2K7, MAP3K5, MDM2, NFKBIB, NFKBIE, | | 2, 2, 2, 2, 2, 2, |
| | | REACTOME_REGULATION_OF_{—} | OTULIN, PAK2, PARP1, PRKDC, RB1, SPPL2A, | | 2, 2, 2, 2, 2, 2, |
| | | TNFR1_SIGNALING, | SPPL2B, TAB1, TAB2, TAB3, TAX1BP1, TNFRSF10B, | | 2, 2, 2, 2, 2, 2, |
| | | ST_TUMOR_NECROSIS_FACTOR_{—} | TNFRSF25, ACTA1, ACTB, ADAM17, AGFG1, | | 2, 2, 1, 1, 1, 1, |
| | | PATHWAY, | AKT1, BAD, BAK1, BAX, BBC3, BCL2L1, BCL2L11, | | 1, 1, 1, 1, 1, 1, |
| | | BIOCARTA_SODD_PATHWAY, | BCL2L2, BIRC5, BNIP3L, BOK, CASP1, | | 1, 1, 1, 1, 1, 1, |
| | | REACTOME_TNFR1_INDUCED_{—} | CASP4, CD247, CDK11A, CDK11B, CDKN2A, DIABLO, | | 1, 1, 1, 1, 1, 1, |
| | | PROAPOPTOTIC_SIGNALING | FAF1, GAS2, GSN, GZMB, HELLS, HRK, | | 1, 1, 1, 1, 1, 1, |
| | | | HSPB1, IGF1, IGF1R, IGF2, IL1A, IRF1, IRF2, IRF3, | | 1, 1, 1, 1, 1, 1, |
| | | | IRF4, IRF5, IRF6, IRF7, LTA, MAP3K3, MAPK10, | | 1, 1, 1, 1, 1, 1, |
| | | | MAPKAPK2, MAPKAPK3, MCL1, MIR3191, MIR34C, | | 1, 1, 1, 1, 1, 1, |
| | | | MIR7108, MIR7846, MLKL, MYC, NFKB2, | | 1, 1, 1, 1, 1, 1, |
| | | | NFKBIL1, NFX1, NR2C2, NSMAF, NUMA1, PAK1, | | 1, 1, 1, 1, 1, 1, |
| | | | PIK3R1, PMAIP1, PRF1, PRKCD, PSEN1, PSEN2, | | 1, 1, 1, 1, 1, 1, |
| | | | RALBP1, RASA1, RIPK2, RIPK3, SCAF11, SMPD2, | | 1, 1, 1, 1, 1, 1, |
| | | | SMPD3, TNFRSF10A, TNFRSF21, TONSL, | | 1, 1, 1, 1, 1, 1, |
| | | | TP53, TP63, TP73, TRAF3 | | 1, 1, 1 |
| module12 | REACTOME_{—} | REACTOME_REGULATION_OF_{—} | IFNA1, IFNA10, IFNA13, IFNA14, IFNA16, IFNA17, | 26 | 1, 1, 1, 1, 1, 1, |
| | REGULATION_{—} | IFNA_SIGNALING | IFNA2, IFNA21, IFNA4, IFNA5, IFNA6, IFNA7, | | 1, 1, 1, 1, 1, 1, |
| | OF_IFNA_{—} | | IFNA8, IFNAR1, IFNAR2, IFNB1, JAK1, PTPN1, | | 1, 1, 1, 1, 1, 1, |
| | SIGNALING | | PTPN11, PTPN6, SOCS1, SOCS3, STAT1, STAT2, | | 1, 1, 1, 1, 1, 1, |
| | | | TYK2, USP18 | | 1, 1 |
| module13 | REACTOME_{—} | REACTOME_INTERLEUKIN_{—} | CASP1, IL18BP, IL18R1, IL37, PTPN11, PTPN12, | 21 | 1, 1, 1, 1, 1, 1, |
| | INTERLEUKIN_{—} | 37_SIGNALING | PTPN13, PTPN14, PTPN18, PTPN2, PTPN20, | | 1, 1, 1, 1, 1, 1, |
| | 37_SIGNALING | | PTPN23, PTPN4, PTPN5, PTPN6, PTPN7, PTPN9, | | 1, 1, 1, 1, 1, 1, |
| | | | SIGIRR, SMAD3, STAT3, TBK1 | | 1, 1, 1 |
| module14 | REACTOME_GENE_{—} | REACTOME_GENE_{—} | AAAS, AGO1, AGO2, AGO3, AGO4, ASZ1, BCDIN3D, | 138 | 1, 1, 1, 1, 1, 1, |
| | SILENCING_BY_RNA | SILENCING_BY_RNA | DDX4, DGCR8, DICER1, DROSHA, FKBP6, | | 1, 1, 1, 1, 1, 1, |
| | | | H2AB1, H2AC14, H2AC18, H2AC19, H2AC20, | | 1, 1, 1, 1, 1, 1, |
| | | | H2AC4, H2AC6, H2AC7, H2AC8, H2AJ, H2AX, | | 1, 1, 1, 1, 1, 1, |
| | | | H2AZ1, H2AZ2, H2BC1, H2BC10, H2BC11, H2BC12, | | 1, 1, 1, 1, 1, 1, |
| | | | H2BC13, H2BC14, H2BC15, H2BC17, H2BC21, | | 1, 1, 1, 1, 1, 1, |
| | | | H2BC3, H2BC4, H2BC5, H2BC6, H2BC7, H2BC8, | | 1, 1, 1, 1, 1, 1, |
| | | | H2BC9, H2BS1, H2BU1, H3-3A, H3-3B, H3C1, | | 1, 1, 1, 1, 1, 1, |
| | | | H3C10, H3C11, H3C12, H3C13, H3C14, H3C15, | | 1, 1, 1, 1, 1, 1, |
| | | | H3C2, H3C3, H3C4, H3C6, H3C7, H3C8, H4-16, | | 1, 1, 1, 1, 1, 1, |
| | | | H4C1, H4C11, H4C12, H4C13, H4C14, H4C15, | | 1, 1, 1, 1, 1, 1, |
| | | | H4C2, H4C3, H4C4, H4C5, H4C6, H4C8, H4C9, | | 1, 1, 1, 1, 1, 1, |
| | | | HENMT1, HSP90AA1, IPO8, MAEL, MIR23B, MOV10L1, | | 1, 1, 1, 1, 1, 1, |
| | | | MYBL1, NDC1, NUP107, NUP133, NUP153, | | 1, 1, 1, 1, 1, 1, |
| | | | NUP155, NUP160, NUP188, NUP205, NUP210, | | 1, 1, 1, 1, 1, 1, |
| | | | NUP214, NUP35, NUP37, NUP42, NUP43, | | 1, 1, 1, 1, 1, 1, |
| | | | NUP50, NUP54, NUP58, NUP62, NUP85, NUP88, | | 1, 1, 1, 1, 1, 1, |
| | | | NUP93, NUP98, PIWIL1, PIWIL2, PIWIL4, PLD6, | | 1, 1, 1, 1, 1, 1, |
| | | | POLR2A, POLR2B, POLR2C, POLR2D, POLR2E, | | 1, 1, 1, 1, 1, 1, |
| | | | POLR2F, POLR2G, POLR2H, POLR2I, POLR2J, | | 1, 1, 1, 1, 1, 1, |
| | | | POLR2K, POLR2L, POM121, POM121C, PRKRA, | | 1, 1, 1, 1, 1, 1, |
| | | | RAE1, RAN, RANBP2, SEC13, SEH1L, TARBP2, | | 1, 1, 1, 1, 1, 1, |
| | | | TDRD1, TDRD12, TDRD6, TDRD9, TDRKH, | | 1, 1, 1, 1, 1, 1 |
| | | | TNRC6A, TNRC6B, TNRC6C, TPR, TSN, TSNAX, | | |
| | | | XPO5 | | |
| module15 | PID_NFKAPPAB_{—} | PID_NFKAPPAB_CANONICAL_PATHWAY, | CHUK, IKBKB, NFKB1, NFKBIA, RELA, IKBKG, | 46 | 4, 4, 4, 4, 4, 3, |
| | CANONICAL_{—} | BIOCARTA_TNFR2_PATHWAY, | MAP3K1, TNF, TNFAIP3, TRAF2, ATF2, ATM, BCL10, | | 2, 2, 2, 2, 1, 1, |
| | PATHWAY | BIOCARTA_41BB_PATHWAY, | BIRC2, CYLD, DUSP1, ELP1, ERC1, IFNG, | | 1, 1, 1, 1, 1, 1, |
| | | WP_ROLE_OF_ALTERED_{—} | IL2, IL4, IL6, JUN, MALT1, MAP3K14, MAP3K5, | | 1, 1, 1, 1, 1, 1, |
| | | GLYCOLYSATION_{—} | MAP4K5, MAPK14, MAPK8, MUC1, NOD2, PRKCA, | | 1, 1, 1, 1, 1, 1, |
| | | OF_MUC1_IN_TUMOUR_{—} | RAN, RIPK1, RIPK2, SSPOP, TANK, TNFRSF1A, | | 1, 1, 1, 1, 1, 1, |
| | | MICROENVIRONMENT | TNFRSF1B, TNFRSF9, TNFSF9, TRAF1, TRAF3, | | 1, 1, 1, 1, 1, 1, |
| | | | TRAF6, UBE2D3, XPO1 | | 1, 1, 1, 1 |
| module16 | KEGG_RIG_I_{—} | KEGG_RIG_I_LIKE_RECEPTOR_{—} | ATG12, ATG5, AZI2, CASP10, CASP8, CHUK, CXCL10, | 71 | 1, 1, 1, 1, 1, 1, |
| | LIKE_RECEPTOR_{—} | SIGNALING_PATHWAY | CXCL8, CYLD, DDX3X, DDX3Y, DDX58, | | 1, 1, 1, 1, 1, 1, |
| | SIGNALING_PATHWAY | | DHX58, FADD, IFIH1, IFNA1, IFNA10, IFNA13, IFNA14, | | 1, 1, 1, 1, 1, 1, |
| | | | IFNA16, IFNA17, IFNA2, IFNA21, IFNA4, IFNA5, | | 1, 1, 1, 1, 1, 1, |
| | | | IFNA6, IFNA7, IFNA8, IFNB1, IFNE, IFNK, IFNW1, | | 1, 1, 1, 1, 1, 1, |
| | | | IKBKB, IKBKE, IKBKG, IL12A, IL12B, IRF3, | | 1, 1, 1, 1, 1, 1, |
| | | | IRF7, ISG15, MAP3K1, MAP3K7, MAPK10, MAPK11, | | 1, 1, 1, 1, 1, 1, |
| | | | MAPK12, MAPK13, MAPK14, MAPK8, MAPK9, | | 1, 1, 1, 1, 1, 1, |
| | | | MAVS, NFKB1, NFKBIA, NFKBIB, NLRX1, | | 1, 1, 1, 1, 1, 1, |
| | | | OTUD5, PIN1, RELA, RIPK1, RNF125, SIKE1, | | 1, 1, 1, 1, 1, 1, |
| | | | STING1, TANK, TBK1, TBKBP1, TKFC, TNF, TRADD, | | 1, 1, 1, 1, 1, 1, |
| | | | TRAF2, TRAF3, TRAF6, TRIM25 | | 1, 1, 1, 1, 1 |
| module17 | WP_ENERGY_{—} | WP_ENERGY_METABOLISM | ATF2, CAMK2G, CAMK4, CREB1, EP300, ESRRA, | 48 | 1, 1, 1, 1, 1, 1, |
| | METABOLISM | | FOXO1, FOXO3, GABPA, GSK3B, HDAC1, MAPK14, | | 1, 1, 1, 1, 1, 1, |
| | | | MED1, MEF2A, MEF2B, MEF2C, MEF2D, | | 1, 1, 1, 1, 1, 1, |
| | | | MIR1281, MYBBP1A, NCOA1, NRF1, PPARA, | | 1, 1, 1, 1, 1, 1, |
| | | | PPARD, PPARG, PPARGC1A, PPARGC1B, PPP3CA, | | 1, 1, 1, 1, 1, 1, |
| | | | PPP3CB, PPP3CC, PPP3R1, PPP3R2, PPRC1, | | 1, 1, 1, 1, 1, 1, |
| | | | PRKAA1, PRKAA2, PRKAB1, PRKAB2, PRKAG1, | | 1, 1, 1, 1, 1, 1, |
| | | | PRKAG2, PRKAG3, PRMT1, RXRA, SIRT1, | | 1, 1, 1, 1, 1, 1 |
| | | | SIRT3, TFAM, TFB1M, TFB2M, UCP2, UCP3 | | |
| module18 | REACTOME_PD_{—} | REACTOME_PD_1_SIGNALING | CD247, CD274, CD3D, CD3E, CD3G, CD4, CSK, | 28 | 1, 1, 1, 1, 1, 1, |
| | 1_SIGNALING | | HLA-DPA1, HLA-DPB1, HLA-DQA1, HLA-DQA2, | | 1, 1, 1, 1, 1, 1, |
| | | | HLA-DQB1, HLA-DQB2, HLA-DRA, HLA-DRB1, | | 1, 1, 1, 1, 1, 1, |
| | | | HLA-DRB3, HLA-DRB4, HLA-DRB5, LCK, PDCD1, | | 1, 1, 1, 1, 1, 1, |
| | | | PDCD1LG2, PTPN11, PTPN6, TRAV19, TRAV29DV5, | | 1, 1, 1, 1 |
| | | | TRAV8-4, TRBV12-3, TRBV7-9 | | |
| module19 | BIOCARTA_{—} | BIOCARTA_KERATINOCYTE_PATHWAY | BCL2, CEBPA, CHUK, DAXX, EGF, EGFR, ETS1, | 46 | 1, 1, 1, 1, 1, 1, |
| | KERATINOCYTE_{—} | | ETS2, FAS, FASLG, FOS, HOXA7, HRAS, IKBKB, | | 1, 1, 1, 1, 1, 1, |
| | PATHWAY | | JUN, MAP2K1, MAP2K3, MAP2K4, MAP2K6, | | 1, 1, 1, 1, 1, 1, |
| | | | MAP2K7, MAP3K1, MAP3K14, MAP3K5, MAPK1, | | 1, 1, 1, 1, 1, 1, |
| | | | MAPK13, MAPK14, MAPK3, MAPK8, NFKB1, | | 1, 1, 1, 1, 1, 1, |
| | | | NFKBIA, PPP2CA, PRKCA, PRKCB, PRKCD, PRKCE, | | 1, 1, 1, 1, 1, 1, |
| | | | PRKCG, PRKCH, PRKCQ, RAF1, RELA, | | 1, 1, 1, 1, 1, 1, |
| | | | RIPK1, SP1, TNF, TNFRSF1A, TNFRSF1B, TRAF2 | | 1, 1, 1, 1 |
| module20 | PID_NCADHERIN_{—} | PID_NCADHERIN_PATHWAY | AXIN1, CAMK2G, CDC42, CDH2, CNR1, CTNNA1, | 33 | 1, 1, 1, 1, 1, 1, |
| | PATHWAY | | CTNNB1, CTNND1, CTTN, DAGLA, DAGLB, DCTN1, | | 1, 1, 1, 1, 1, 1, |
| | | | FER, FGFR1, GAP43, GJA1, GRIA2, GSN, | | 1, 1, 1, 1, 1, 1, |
| | | | JUP, KIF5B, LRP5, MAPK8, MAPRE1, MYL2, PIK3CA, | | 1, 1, 1, 1, 1, 1, |
| | | | PIK3R1, PIP5K1C, PLCG1, PTPN1, PTPN11, | | 1, 1, 1, 1, 1, 1, |
| | | | RAC1, RHOA, ROCK1 | | 1, 1, 1 |
| module21 | WP_MTHFR_{—} | WP_MTHFR_DEFICIENCY | ALDH7A1, ASMT, BHMT, CASP3, CASP9, CHDH, | 27 | 1, 1, 1, 1, 1, 1, |
| | DEFICIENCY | | CHKA, CHPT1, COMT, CYCS, DNMT1, DNMT3A, | | 1, 1, 1, 1, 1, 1, |
| | | | DNMT3B, EHMT1, EHMT2, GRIN1, GRIN2A, | | 1, 1, 1, 1, 1, 1, |
| | | | GRIN2D, HNMT, MARS1, MIR4761, MIR6758, MTHFR, | | 1, 1, 1, 1, 1, 1, |
| | | | NDUFAF7, PCYT1A, PEMT, SGMS1 | | 1, 1, 1 |
| module22 | REACTOME_{—} | REACTOME_DAP12_SIGNALING, | B2M, BTK, FYN, GRAP2, GRB2, HLA-E, HRAS, | 45 | 2, 2, 2, 2, 2, 2, |
| | DAP12_SIGNALING | REACTOME_DAP12_INTERACTIONS | KLRC2, KLRD1, KLRK1, KRAS, LAT, LCK, | | 2, 2, 2, 2, 2, 2, |
| | | | LCP2, NRAS, PIK3CA, PIK3CB, PIK3R1, PIK3R2, | | 2, 2, 2, 2, 2, 2, |
| | | | PLCG1, PLCG2, RAC1, SHC1, SOS1, SYK, | | 2, 2, 2, 2, 2, 2, |
| | | | TREM2, TYROBP, VAV2, VAV3, CD300E, CD300LB, | | 2, 2, 2, 2, 2, 1, |
| | | | CLEC5A, HLA-B, HLA-C, KIR2DS1, KIR2DS2, | | 1, 1, 1, 1, 1, 1, |
| | | | KIR2DS3, KIR2DS4, KIR2DS5, | | 1, 1, 1, 1, 1, 1, |
| | | | KIR3DS1, NCR2, SIGLEC14, SIGLEC15, | | 1, 1, 1 |
| | | | SIRPB1, TREM1 | | |
| module23 | WP_VIRAL_{—} | WP_VIRAL_ACUTE_MYOCARDITIS | ABL1, ABL2, ACTB, AIF1, AKT1, BAX, BCL2, BCL2L1, | 87 | 1, 1, 1, 1, 1, 1, |
| | ACUTE_MYOCARDITIS | | BID, BNIP2, CAAP1, CASP1, CASP2, CASP3, | | 1, 1, 1, 1, 1, 1, |
| | | | CASP6, CASP7, CASP8, CASP9, CAV1, CCND1, | | 1, 1,1, 1, 1, 1, |
| | | | CCR3, CCR5, CD4, CD40LG, CD55, CD80, | | 1, 1, 1, 1, 1, 1, |
| | | | CHRAC1, CREB1, CXADR, CXCR4, CYCS, DAG1, | | 1, 1, 1, 1, 1, 1, |
| | | | DFFA, DFFB, DMD, EDN1, EIF4G1, EIF4G2, ENDOG, | | 1, 1, 1, 1, 1, 1, |
| | | | FYN, GSK3B, HLA-DMA, | | 1, 1, 1, 1, 1, 1, |
| | | | IFNG, IL10, IL12A, IL12B, IL2, IL6, ILK, ITGAL, | | 1, 1, 1, 1, 1, 1, |
| | | | ITGB2, JAK1, JKAMP, KRT8, LAMA2, MAPK1, | | 1, 1, 1, 1, 1, 1, |
| | | | MAPK3, MICA, MIR7705, MMP9, MYH6, NFKB2, | | 1, 1, 1, 1, 1, 1, |
| | | | NOD2, NOS1, PABPC1, PARP1, PIK3R1, PTCRA, | | 1, 1, 1, 1, 1, 1, |
| | | | PYCARD, RAC2, RAC3, RASA1, SGCA, SGCB, | | 1, 1, 1, 1, 1, 1, |
| | | | SGCD, SGCG, SOCS1, SOS1, SRC, STAT1, STAT3, | | 1, 1, 1, 1, 1, 1, |
| | | | TGFB1, TICAM1, TLR3, TLR4, TLR5, TNF | | 1, 1, 1, 1, 1, 1, |
| | | | | | 1, 1, 1 |
| module24 | WP_AP1_{—} | WP_PHOTODYNAMIC_THERAPYINDUCED_{—} | ATF2, BAK1, BAX, BCL2, BCL2L1, BCL2L11, BCL3, | 51 | 1, 1, 1, 1, 1, 1, |
| | SURVIVAL_SIGNALING | AP1_SURVIVAL_SIGNALING | BID, BMF, CCNA2, CCND1, CCNE1, CDKN1A, | | 1, 1, 1, 1, 1, 1, |
| | | | CDKN2A, CFLAR, EGFR, ELK1, FAS, FASLG, | | 1, 1, 1, 1, 1, 1, |
| | | | FGF7, FOS, HBEGF, HSP90AA1, IFNG, IL2, IL6, | | 1, 1, 1, 1, 1, 1, |
| | | | JUN, JUNB, MAP2K3, MAP2K4, MAP2K6, MAP2K7, | | 1, 1, 1, 1, 1, 1, |
| | | | MAP3K5, MAPK11, MAPK12, MAPK13, MAPK14, | | 1, 1, 1, 1, 1, 1, |
| | | | MAPK8, MCL1, MIR8085, MMP2, NFE2L2, | | 1, 1, 1, 1, 1, 1, |
| | | | PDGFRA, RB1, TNF, TNFRSF1A, TNFSF10, TP53, | | 1, 1, 1, 1, 1, 1, |
| | | | TRAF2, TRAF5, TRAF6 | | 1, 1, 1 |
| module25 | REACTOME_{—} | REACTOME_SIGNALING_BY_HIPPO | AMOT, AMOTL1, AMOTL2, CASP3, DVL2, LATS1, | 20 | 1, 1, 1, 1, 1, 1, |
| | SIGNALINGBY_HIPPO | | LATS2, MOB1A, MOB1B, NPHP4, SAV1, STK3, | | 1, 1, 1, 1, 1, 1, |
| | | | STK4, TJP1, TJP2, WWC1, WWTR1, YAP1, YWHAB, | | 1, 1, 1, 1, 1, 1, |
| | | | YWHAE | | 1, 1 |
| module26 | WP_TLR4_{—} | WP_MIRNAS_INVOLVEMENT_IN_THE_{—} | CHUK, CXCL8, IKBKB, IKBKG, IL6, IRAK1, IRAK4, | 74 | 2, 2, 2, 2, 2, 2, |
| | SIGNALING_AND_{—} | IMMUNE_RESPONSE_IN_SEPSIS, | IRF7, MAP3K7, MIR718, MYD88, NFKB1, NFKBIA, | | 2, 2, 2, 2, 2, 2, |
| | TOLERANCE | WP_TLR4_SIGNALING_AND_TOLERANCE | TAB1, TAB2, TLR4, TNF, TRAF3, TRAF6, CCL3, | | 2, 2, 2, 2, 2, 2, |
| | | | CCL4, ELANE, GZMB, ICAM1, IFNB1, IKBKE, | | 2, 1, 1, 1, 1, 1, |
| | | | IL10, IL1A, INPP5D, IRAK3, IRF1, IRF3, IRF5, LCN2, | | 1, 1, 1, 1, 1, 1, |
| | | | MAPK14, MAPK8, MIR106B, MIR125B1, MIR125B2, | | 1, 1, 1, 1, 1, 1, |
| | | | MIR126, MIR145, MIR146B, MIR149, | | 1, 1, 1, 1, 1, 1, |
| | | | MIR155, MIR155HG, MIR16-1, MIR16-2, | | 1, 1, 1, 1, 1, 1, |
| | | | MIR187, MIR199A1, MIR199A2, MIR200B, MIR200C, | | 1, 1, 1, 1, 1, 1, |
| | | | MIR203A, MIR203B, MIR223, MIR29A, | | 1, 1, 1, 1, 1, 1, |
| | | | MIR29B1, MIR6502, MIR758, MIR9-1, | | 1, 1, 1, 1, 1, 1, |
| | | | MIRLET7E, MIRLET7I, NFKB2, REL, RELA, RELB, | | 1, 1, 1, 1, 1, 1, |
| | | | RIPK1, TBK1, TICAM1, TIRAP, TLR7, TLR8, | | 1, 1 |
| | | | TRAM1, VCAM1 | | |
| module27 | WP_SELENIUM/ | WP_SELENIUM_MICRONUTRIENT_NETWORK, | ABCA1, ALB, APOA1, APOB, CAT, CBS, CCL2, CRP, | 113 | 2, 2, 2, 2, 2, 2, |
| | WP_FOLATE_{—} | WP_FOLATE_METABOLISM | CTH, F2, F7, FGA, FGB, FGG, FLAD1, GPX1, | | 2, 2, 2, 2, 2, 2, |
| | METABOLISM | | GPX2, GPX3, GPX4, GPX6, HBA1, HBB, ICAM1, IFNG, | | 2, 2, 2, 2, 2, 2, |
| | | | IL1B, IL6, INS, INSR, LDLR, MIR6886, MPO, | | 2, 2, 2, 2, 2, 2, |
| | | | MTHFR, MTR, NFKB1, NFKB2, PLAT, PLG, RELA, | | 2, 2, 2, 2, 2, 2, |
| | | | RFK, SAA1, SAA2, SAA3P, SAA4, SCARB1, SERPINA3, | | 2, 2, 2, 2, 2, 2, |
| | | | SERPINE1, SOD1, SOD2, SOD2-OT1, | | 2, 2, 2, 2, 2, 2, |
| | | | SOD3, TNF, AHCY, ALOX15B, ALOX5, ALOX5AP, | | 2, 2, 2, 2, 2, 2, |
| | | | CSF1, DHFR, DIO1, DIO2, DIO3, FOLR1, | | 2, 2, 2, 1, 1, 1, |
| | | | FOLR2, FOLR3, GART, GGT1, GGT2, GGTLC1, | | 1, 1, 1, 1, 1, 1, |
| | | | GGTLC2, GSR, IL2, IL4, IZUMO1R, KMO, KYNU, | | 1, 1, 1, 1, 1, 1, |
| | | | MAT1A, MIR6778, MSRB1, MTHFD1, MTHFD2, | | 1, 1, 1, 1, 1, 1, |
| | | | MTHFS, MTRR, NOS1, PNPO, PRDX1, PRDX2, | | 1, 1, 1, 1, 1, 1, |
| | | | PRDX3, PRDX4, PRDX5, PTGS1, PTGS2, SELENOF, | | 1, 1, 1, 1, 1, 1, |
| | | | SELENOH, SELENOI, SELENOK, SELENOM, | | 1, 1, 1, 1, 1, 1, |
| | | | SELENON, SELENOO, SELENOP, SELENOS, | | 1, 1, 1, 1, 1, 1, |
| | | | SELENOT, SELENOV, SELENOW, SEPHS2, | | 1, 1, 1, 1, 1, 1, |
| | | | SHMT1, SHMT2, SLC19A1, SLC46A1, TP53, TXN, | | 1, 1, 1, 1, 1, 1, |
| | | | TXNRD1, TXNRD2, TXNRD3, XDH | | 1, 1, 1, 1, 1 |
| module28 | PID_REG_GR_{—} | PID_REG_GR_PATHWAY | AFP, AKT1, BAX, BGLAP, CDK5, CDK5R1, CDKN1A, | 82 | 1, 1, 1, 1, 1, 1, |
| | PATHWAY | | CGA, CREB1, CREBBP, CSF2, CSN2, CXCL8, | | 1, 1, 1, 1, 1, 1, |
| | | | EGR1, EP300, FGG, FKBP4, FKBP5, FOS, GATA3, | | 1, 1, 1, 1, 1, 1, |
| | | | GSK3B, HDAC1, HDAC2, HSP90AA1, ICAM1, | | 1, 1, 1, 1, 1, 1, |
| | | | IFNG, IL13, IL2, IL4, IL5, IL6, IRF1, JUN, KMT5B, | | 1, 1, 1, 1, 1, 1, |
| | | | KRT14, KRT17, KRT5, MAPK1, MAPK10, MAPK11, | | 1, 1, 1, 1, 1, 1, |
| | | | MAPK14, MAPK3, MAPK8, MAPK9, MDM2, | | 1, 1, 1, 1, 1, 1, |
| | | | MMP1, NCOA1, NCOA2, NFATC1, NFKB1, NR113, | | 1, 1, 1, 1, 1, 1, |
| | | | NR3C1, NR4A1, PBX1, PCK2, POMC, POU1F1, | | 1, 1, 1, 1, 1, 1, |
| | | | POU2F1, PPP5C, PRKACA, PRKACB, PRKACG, | | 1, 1, 1, 1, 1, 1, |
| | | | PRL, RELA, SELE, SFN, SGK1, SMARCA4, | | 1, 1, 1, 1, 1, 1, |
| | | | SMARCC1, SMARCC2, SMARCD1, SPI1, STAT1, | | 1, 1, 1, 1, 1, 1, |
| | | | STAT5A, STAT5B, SUMO2, TBP, TBX21, TP53, | | 1, 1, 1, 1, 1, 1, |
| | | | TSG101, VIPR1, YWHAH | | 1, 1, 1, 1 |
| module29 | WP_DEVELOPMENT_{—} | WP_DEVELOPMENT_AND_HETEROGENEITY_{—} | AHR, AREG, BCL11B, EOMES, GATA3, GFI1, HNF1A, | 32 | 1, 1, 1, 1, 1, 1, |
| | AND_{—} | OF_THE_ILC_FAMILY | ID2, IFNG, IL12A, IL12B, IL13, IL15, IL17A, | | 1, 1, 1, 1, 1, 1, |
| | HETEROGENEITY_{—} | | IL18, IL1B, IL22, IL23A, IL25, IL33, IL4, IL5, IL6, IL7, | | 1, 1, 1, 1, 1, 1, |
| | OF_THE_{—} | | IL9, NFIL3, RORA, TBX21, TNF, TOX, TSLP, ZBTB16 | | 1, 1, 1, 1, 1, 1, |
| | ILC_FAMILY | | | | 1, 1, 1, 1, 1, 1, |
| | | | | | 1, 1 |
| module30 | PID_IL27_PATHWAY | PID_IL27_PATHWAY | EBI3, GATA3, IFNG, IL12A, IL12B, IL12RB1, IL12RB2, | 26 | 1, 1, 1, 1, 1, 1, |
| | | | IL17A, IL18, IL1B, IL2, IL27, IL27RA, IL6, IL6ST, | | 1, 1, 1, 1, 1, 1, |
| | | | JAK1, JAK2, STAT1, STAT2, STAT3, STAT4, | | 1, 1, 1, 1, 1, 1, |
| | | | STAT5A, TBX21, TGFB1, TNF, TYK2 | | 1, 1, 1, 1, 1, 1, |
| | | | | | 1, 1 |
| module31 | BIOCARTA_CYTOKINE_{—} | BIOCARTA_CYTOKINE_PATHWAY | CXCL8, IFNA1, IFNB1, IFNG, IL10, IL13, IL15, IL16, | 19 | 1, 1, 1, 1, 1, 1, |
| | PATHWAY | | IL17A, IL18, IL1A, IL2, IL3, IL4, IL5, IL6, IL9, TNF, | | 1, 1, 1, 1, 1, 1, |
| | | | TXLNA | | 1, 1, 1, 1, 1, 1, |
| | | | | | 1 |
| module32 | WP_TYPE_II_{—} | WP_TYPE_II_INTERFERON_SIGNALING_IFNG | CIITA, CXCL10, CXCL9, CYBB, EIF2AK2, GBP1, | 37 | 1, 1, 1, 1, 1, 1, |
| | INTERFERON_{—} | | H4C14, HLA-B, | | 1, 1, 1, 1, 1, 1, |
| | SIGNALING_IFNG | | ICAM1, IFI6, IFIT2, IFNA2, IFNB1, IFNG, IFNGR1, | | 1, 1, 1, 1, 1, 1, |
| | | | IFNGR2, IL1B, IRF1, IRF2, IRF4, IRF8, IRF9, ISG15, | | 1, 1, 1, 1, 1, 1, |
| | | | JAK1, JAK2, NOS2, OAS1, PRKCD, PSMB9, | | 1, 1, 1, 1, 1, 1, |
| | | | PTPN11, REG1A, SOCS1, SOCS3, SPI1, STAT1, | | 1, 1, 1, 1, 1, 1, |
| | | | STAT2, TAP1 | | 1 |
| module33 | BIOCARTA_TID_{—} | BIOCARTA_TID_PATHWAY | DNAJA3, HSPA1A, IFNG, IFNGR1, IFNGR2, IKBKB, | 19 | 1, 1, 1, 1, 1, 1, |
| | PATHWAY | | JAK2, LIN7A, NFKB1, NFKBIA, RB1, RELA, TAX1BP3, | | 1, 1, 1, 1, 1, 1, |
| | | | TNF, TNFRSF1A, TNFRSF1B, TP53, USH1C, | | 1, 1, 1, 1, 1, 1, |
| | | | WT1 | | 1 |
| module34 | REACTOME_{—} | BIOCARTA_IFNG_PATHWAY, | IFNG, IFNGR1, IFNGR2, JAK1, JAK2, STAT1, PIAS1, | 14 | 2, 2, 2, 2, 2, 2, |
| | REGULATION_OF_{—} | REACTOME_REGULATION_OF_IFNG_{—} | PTPN1, PTPN11, PTPN2, PTPN6, SOCS1, | | 1, 1, 1, 1, 1, 1, |
| | IFNG_SIGNALING | SIGNALING | SOCS3, SUMO1 | | 1, 1 |
| module35 | KEGG_LEISHMANIA_{—} | KEGG_LEISHMANIA_INFECTION | C3, CR1, CYBA, ELK1, FCGR1A, FCGR2A, FCGR2C, | 72 | 1, 1, 1, 1, 1, 1, |
| | INFECTION | | FCGR3A, FCGR3B, FOS, HLA-DMA, HLA-DMB, | | 1, 1, 1, 1, 1, 1, |
| | | | HLA-DOA, HLA-DOB, HLA-DPA1, HLA-DPB1, | | 1, 1, 1, 1, 1, 1, |
| | | | HLA-DQA1, HLA-DQA2, HLA-DQB1, | | 1, 1, 1, 1, 1, 1, |
| | | | HLA-DRA, HLA-DRB1, HLA-DRB3, HLA-DRB4, | | 1, 1, 1, 1, 1, 1, |
| | | | HLA-DRB5, IFNG, IFNGR1, IFNGR2, IL10, IL12A, IL12B, | | 1, 1, 1, 1, 1, 1, |
| | | | IL1A, IL1B, IL4, IRAK1, IRAK4, ITGA4, ITGAM, ITGB1, | | 1, 1, 1, 1, 1, 1, |
| | | | ITGB2, JAK1, JAK2, JUN, MAP3K7, MAPK1, | | 1, 1, 1, 1, 1, 1, |
| | | | MAPK11, MAPK12, MAPK13, MAPK14, MAPK3, | | 1, 1, 1, 1, 1, 1, |
| | | | MARCKSL1, MYD88, NCF1, NCF2, NCF4, NFKB1, | | 1, 1, 1, 1, 1, 1, |
| | | | NFKBIA, NFKBIB, NOS2, PRKCB, PTGS2, | | 1, 1, 1, 1, 1, 1, |
| | | | PTPN6, RELA, STAT1, TAB1, TAB2, TGFB1, TGFB2, | | 1, 1, 1, 1, 1, 1 |
| | | | TGFB3, TLR2, TLR4, TNF, TRAF6 | | |
| module36 | PID_IFNG_PATHWAY | PID_IFNG_PATHWAY | AKT1, CAMK2A, CAMK2B, CAMK2D, CAMK2G, | 40 | 1, 1, 1, 1, 1, 1, |
| | | | CASP1, CBL, CEBPB, CREBBP, CRKL, DAPK1, | | 1, 1, 1, 1, 1, 1, |
| | | | EP300, IFNG, IFNGR1, IL1B, IRF1, IRF9, JAK1, JAK2, | | 1, 1, 1, 1, 1, 1, |
| | | | MAP2K1, MAP3K1, MAP3K11, MAPK1, MAPK3, | | 1, 1, 1, 1, 1, 1, |
| | | | MTOR, PIAS1, PIAS4, PIK3CA, PIK3R1, PRKCD, | | 1, 1, 1, 1, 1, 1, |
| | | | PTGES2, PTPN11, PTPN2, RAP1A, RAP1B, | | 1, 1, 1, 1, 1, 1, |
| | | | RAPGEF1, SMAD7, SOCS1, STAT1, STAT3 | | 1, 1, 1, 1 |
| module37 | WP_ALLOGRAFT_{—} | WP_ALLOGRAFT_REJECTION, | CD28, CD80, CD86, FAS, FASLG, GZMB, HLA-A, | 109 | 3, 3, 3, 3, 3, 3, |
| | REJECTION | KEGG_TYPE_I_DIABETES_MELLITUS, | HLA-B, HLA-C, HLA-DMA, HLA-DMB, HLA-DOA, | | 3, 3, 3, 3, 3, 3, |
| | | KEGG_GRAFT_VERSUS_HOST_DISEASE | HLA-DOB, HLA-DPA1, HLA-DPB1, HLA-DQA1, | | 3, 3, 3, 3, 3, 3, |
| | | | HLA-DQA2, HLA-DQB1, HLA-DRA, HLA-DRB1, | | 3, 3, 3, 3, 3, 3, |
| | | | HLA-DRB5, HLA-E, HLA-F, HLA-G, | | 3, 3, 3, 3, 3, 3, |
| | | | IFNG, IL1A, IL1B, IL2, PRF1, TNF, HLA-DRB3, | | 2, 2, 2, 2, 1, 1, |
| | | | HLA-DRB4, IL12A, IL12B, ABCB1, AGTR1, BHMT2, C1QA, | | 1, 1, 1, 1, 1, 1, |
| | | | C1QB, C1QC, C2, C3, C4A, C4B, C5, C6, C7, | | 1, 1, 1, 1, 1, 1, |
| | | | C8A, C8B, C9, CASP3, CASP7, CASP8, CASP9, | | 1, 1, 1, 1, 1, 1, |
| | | | CCL19, CCL21, CD40, CD40LG, CD55, COL5A1, | | 1, 1, 1, 1, 1, 1, |
| | | | CPE, CSNK2A2, CTLA4, CXCL11, CXCL12, CXCL13, | | 1, 1, 1, 1, 1, 1, |
| | | | CXCL8, CXCL9, CXCR5, FOXP3, GABPA, | | 1, 1, 1, 1, 1, 1, |
| | | | GAD1, GAD2, GDNF, GNLY, HARS1, HSPD1, ICA1, | | 1, 1, 1, 1, 1, 1, |
| | | | IL10, IL13, IL17A, IL21, IL22, IL2RA, IL4, IL5, IL6, | | 1, 1, 1, 1, 1, 1, |
| | | | INS, KIR2DL1, KIR2DL2, KIR2DL3, KIR2DL5A, | | 1, 1, 1, 1, 1, 1, |
| | | | KIR3DL1, KIR3DL2, KLRC1, KLRD1, LRRK2, LTA, | | 1, 1, 1, 1, 1, 1, |
| | | | MICA, PDGFRA, PECR, PRKCZ, PTPRN, PTPRN2, | | 1, 1, 1, 1, 1, 1, |
| | | | STAT1, TGFB1, TUBA1B, VEGFA, VIM | | 1, 1, 1, 1, 1, 1, |
| | | | | | 1 |
| module38 | WP_SPINAL_CORD_{—} | WP_SPINAL_CORD_INJURY | ACAN, AIF1, ANXA1, APEX1, AQP1, AQP4, ARG1, | 119 | 1, 1, 1, 1, 1, 1, |
| | INJURY | | BCAN, BDNF, BTG2, C1QB, C5, CASP3, CCL2, | | 1, 1, 1, 1, 1, 1, |
| | | | CCND1, CCNG1, CCR2, CD47, CDC42, CDK1, | | 1, 1, 1, 1, 1, 1, |
| | | | CDK2, CDK4, CDKN1B, CHST11, COL2A1, COL4A1, | | 1, 1, 1, 1, 1, 1, |
| | | | CSPG4, CXCL1, CXCL10, CXCL2, CXCL8, | | 1, 1, 1, 1, 1, 1, |
| | | | E2F1, E2F5, EFNB2, EGFR, EGR1, EPHA4, FCGR2A, | | 1, 1, 1, 1, 1, 1, |
| | | | FCGR2C, FKBP1A, FOS, FOXO3, GADD45A, | | 1, 1, 1, 1, 1, 1, |
| | | | GAP43, GDNF, GFAP, GJA1, GRIN1, ICAM1, | | 1, 1, 1, 1, 1, 1, |
| | | | IFNG, IL1A, IL1B, IL1R1, IL2, IL4, IL6, KLK8, LEP, | | 1, 1, 1, 1, 1, 1, |
| | | | LGALS3, LILRB2, LILRB3, LTB, LTB4R, MAG, MAPK1, | | 1, 1, 1, 1, 1, 1, |
| | | | MAPK3, MBP, MIF, MIR23B, MIR6869, MMP12, | | 1, 1, 1, 1, 1, 1, |
| | | | MMP9, MYC, NCAN, NGFR, NOS1, NOS2, | | 1, 1, 1, 1, 1, 1, |
| | | | NOX4, NR4A1, NTN1, OMG, PDYN, PLA2G2A, | | 1, 1, 1, 1, 1, 1, |
| | | | PLA2G5, PLA2G6, PLXNA2, PPP3CA, PRB1, PRKCA, | | 1, 1, 1, 1, 1, 1, |
| | | | PTGS2, PTPRA, PTPRZ1, RAC1, RB1, RGMA, | | 1, 1, 1, 1, 1, 1, |
| | | | RHOA, RHOB, RHOC, ROCK2, ROS1, RTN4, | | 1, 1, 1, 1, 1, 1, |
| | | | RTN4R, SELP, SEMA6A, SLIT1, SLIT2, SLIT3, | | 1, 1, 1, 1, 1, 1, |
| | | | SOX9, TACR1, TGFB1, TLR4, TNF, TNFSF13, | | 1, 1, 1, 1, 1, 1, |
| | | | TNFSF13B, TP53, VCAN, VIM, XYLT1, ZFP36 | | 1, 1, 1, 1, 1, 1, |
| | | | | | 1, 1, 1, 1, 1 |
| module39 | REACTOME_{—} | REACTOME_INTERLEUKIN_36_PATHWAY | IL1F10, IL1RAP, IL1RL2, IL36A, IL36B, IL36G, IL36RN | 7 | 1, 1, 1, 1, 1, 1, |
| | INTERLEUKIN_{—} | | | | 1 |
| | 36_PATHWAY | | | | |
| module40 | PID_HNF3A_{—} | PID_HNF3A_PATHWAY | AP1B1, APOB, AR, ATP5PF, BRCA1, C4BPB, CDKN1B, | 44 | 1, 1, 1, 1, 1, 1, |
| | PATHWAY | | CEBPB, COL18A1, CREBBP, CYP2C18, | | 1, 1, 1, 1, 1, 1, |
| | | | DSCAM, EP300, ESR1, FOS, FOXA1, FOXA2, | | 1, 1, 1, 1, 1, 1, |
| | | | FOXA3, GCG, INS, JUN, KLK3, NCOA3, NDUFV3, | | 1, 1, 1, 1, 1, 1, |
| | | | NFIA, NFIB, NFIC, NKX3-1, | | 1, 1, 1, 1, 1, 1, |
| | | | NR2F2, NRIP1, PISD, POU2F1, PRDM15, SCGB1A1, | | 1, 1, 1, 1, 1, 1, |
| | | | SERPINA1, SFTPA1, SFTPA2, SFTPD, | | 1, 1, 1, 1, 1, 1, |
| | | | SHH, SOD1, SP1, TFF1, VTN, XBP1 | | 1, 1 |
| module41 | KEGG_PROXIMAL_{—} | KEGG_PROXIMAL_TUBULE_BICARBONATE_{—} | AQP1, ATP1A1, ATP1A2, ATP1A3, ATP1A4, ATP1B1, | 23 | 1, 1, 1, 1, 1, 1, |
| | TUBULE_{—} | RECLAMATION | ATP1B2, ATP1B3, ATP1B4, CA2, CA4, FXYD2, | | 1, 1, 1, 1, 1, 1, |
| | BICARBONATE_{—} | | GLS, GLS2, GLUD1, GLUD2, MDH1, PCK1, | | 1, 1, 1, 1, 1, 1, |
| | RECLAMATION | | PCK2, SLC25A10, SLC38A3, SLC4A4, SLC9A3 | | 1, 1, 1, 1, 1 |
| module42 | REACTOME_TYPE_I_{—} | REACTOME_TYPE_I_HEMIDESMOSOME_{—} | CD151, COL17A1, DST, ITGA6, ITGB4, KRT14, | 11 | 1, 1, 1, 1, 1, 1, |
| | HEMIDESMOSOME_{—} | ASSEMBLY | KRT5, LAMA3, LAMB3, LAMC2, PLEC | | 1, 1, 1, 1, 1 |
| | ASSEMBLY | | | | |
| module43 | REACTOME_{—} | REACTOME_CILIUM_ASSEMBLY | ACTR1A, AHI1, AKAP9, ALMS1, ARF4, ARL13B, | 202 | 1, 1, 1, 1, 1, 1, |
| | CILIUM_ASSEMBLY | | ARL3, ARL6, ASAP1, ATAT1, B9D1, B9D2, BBIP1, | | 1, 1, 1, 1, 1, 1, |
| | | | BBS1, BBS10, BBS12, BBS2, BBS4, BBS5, BBS7, | | 1, 1, 1, 1, 1, 1, |
| | | | BBS9, C2CD3, CC2D2A, CCP110, CCT2, CCT3, | | 1, 1, 1, 1, 1, 1, |
| | | | CCT4, CCT5, CCT8, CDK1, CDK5RAP2, CENPJ, | | 1, 1, 1, 1, 1, 1, |
| | | | CEP131, CEP135, CEP152, CEP162, CEP164, | | 1, 1, 1, 1, 1, 1, |
| | | | CEP192, CEP250, CEP290, CEP41, CEP43, | | 1, 1, 1, 1, 1, 1, |
| | | | CEP57, CEP63, CEP70, CEP72, CEP76, CEP78, | | 1, 1, 1, 1, 1, 1, |
| | | | CEP83, CEP89, CEP97, CETN2, CKAP5, CLASP1, | | 1, 1, 1, 1, 1, 1, |
| | | | CLUAP1, CNGA2, CNGA4, CNGB1, CNTRL, | | 1, 1, 1, 1, 1, 1, |
| | | | CSNK1D, CSNK1E, CYS1, DCTN1, DCTN2, DCTN3, | | 1, 1, 1, 1, 1, 1, |
| | | | DYNC1H1, DYNC112, DYNC2H1, DYNC2L11, | | 1, 1, 1, 1, 1, 1, |
| | | | DYNLL1, DYNLL2, DYNLRB1, DYNLRB2, EXOC1, | | 1, 1, 1, 1, 1, 1, |
| | | | EXOC2, EXOC3, EXOC4, EXOC5, EXOC6, | | 1, 1, 1, 1, 1, 1, |
| | | | EXOC7, EXOC8, FBF1, GBF1, HAUS1, HAUS2, | | 1, 1, 1, 1, 1, 1, |
| | | | HAUS3, HAUS4, HAUS5, HAUS6, HAUS7, HAUS8, | | 1, 1, 1, 1, 1, 1, |
| | | | HDAC6, HSP90AA1, HSPB11, IFT122, IFT140, | | 1, 1, 1, 1, 1, 1, |
| | | | IFT172, IFT20, IFT22, IFT27, IFT43, IFT46, IFT52, | | 1, 1, 1, 1, 1, 1, |
| | | | IFT57, IFT74, IFT80, IFT81, IFT88, INPP5E, IQCB1, | | 1, 1, 1, 1, 1, 1, |
| | | | KIF17, KIF24, KIF3A, KIF3B, KIF3C, KIFAP3, | | 1, 1, 1, 1, 1, 1, |
| | | | LZTFL1, MAPRE1, MARK4, MCHR1, MKKS, | | 1, 1, 1, 1, 1, 1, |
| | | | MKS1, NDE1, NEDD1, NEK2, NINL, NPHP1, NPHP3, | | 1, 1, 1, 1, 1, 1, |
| | | | NPHP4, ODF2, OFD1, PAFAH1B1, PCM1, | | 1, 1, 1, 1, 1, 1, |
| | | | PCNT, PDE6D, PKD1, PKD2, PLK1, PLK4, PPP2R1A, | | 1, 1, 1, 1, 1, 1, |
| | | | PRKACA, PRKAR2B, RAB11A, RAB11FIP3, | | 1, 1, 1, 1, 1, 1, |
| | | | RAB3IP, RAB8A, RHO, RP2, RPGRIP1L, SCLT1, | | 1, 1, 1, 1, 1, 1, |
| | | | SDCCAG8, SEPTIN2, SFI1, SMO, SSNA1, SSTR3, | | 1, 1, 1, 1, 1, 1, |
| | | | TCP1, TCTE3, TCTEX1D1, TCTEX1D2, TCTN1, | | 1, 1, 1, 1, 1, 1, |
| | | | TCTN2, TCTN3, TMEM216, TMEM67, TNPO1, | | 1, 1, 1, 1, 1, 1, |
| | | | TRAF3IP1, TRIP11, TTBK2, TTC21B, TTC26, | | 1, 1, 1, 1, 1, 1, |
| | | | TTC30A, TTC30B, TTC8, TUBA1A, TUBA1B, | | 1, 1, 1, 1, 1, 1, |
| | | | TUBA1C, TUBA3C, TUBA3D, TUBA3E, TUBA4A, | | 1, 1, 1, 1, 1, 1, |
| | | | TUBA4B, TUBA8, TUBAL3, TUBB, TUBB1, TUBB2A, | | 1, 1, 1, 1, 1, 1, |
| | | | TUBB2B, TUBB3, TUBB4A, TUBB4B, TUBB6, | | 1, 1, 1, 1 |
| | | | TUBB8, TUBB8B, TUBG1, UNC119B, WDR19, | | |
| | | | WDR34, WDR35, WDR60, YWHAE, YWHAG | | |
| module44 | KEGG_GALACTOSE_{—} | KEGG_GALACTOSE_METABOLISM | AKR1B1, B4GALT1, B4GALT2, G6PC, G6PC2, | 26 | 1, 1, 1, 1, 1, 1, |
| | METABOLISM | | GAA, GALE, GALK1, GALK2, GALT, GANC, GCK, | | 1, 1, 1, 1, 1, 1, |
| | | | GLA, GLB1, HK1, HK2, HK3, LALBA, LCT, MGAM, | | 1, 1, 1, 1, 1, 1, |
| | | | PFKL, PFKM, PFKP, PGM1, PGM2, UGP2 | | 1, 1, 1, 1, 1, 1, |
| | | | | | 1, 1 |
| module45 | KEGG_OOCYTE_{—} | KEGG_OOCYTE_MEIOSIS | AC023512.1, ADCY1, ADCY2, ADCY3, ADCY4, | 113 | 1, 1, 1, 1, 1, 1, |
| | MEIOSIS | | ADCY5, ADCY6, ADCY7, ADCY8, ADCY9, ANAPC1, | | 1, 1, 1, 1, 1, 1, |
| | | | ANAPC10, ANAPC11, ANAPC13, ANAPC2, | | 1, 1, 1, 1, 1, 1, |
| | | | ANAPC4, ANAPC5, ANAPC7, AR, AURKA, BTRC, | | 1, 1, 1, 1, 1, 1, |
| | | | BUB1, CALM1, CALM2, CALM3, CALML3, CALML5, | | 1, 1, 1, 1, 1, 1, |
| | | | CALML6, CAMK2A, CAMK2B, CAMK2D, | | 1, 1, 1, 1, 1, 1, |
| | | | CAMK2G, CCNB1, CCNB2, CCNE1, CCNE2, CDC16, | | 1, 1, 1, 1, 1, 1, |
| | | | CDC20, CDC23, CDC25C, CDC26, CDC27, | | 1, 1, 1, 1, 1, 1, |
| | | | CDK1, CDK2, CHP1, CHP2, CPEB1, CUL1, ESPL1, | | 1, 1, 1, 1, 1, 1, |
| | | | FBXO43, FBXO5, FBXW11, IGF1, IGF1R, INS, | | 1, 1, 1, 1, 1, 1, |
| | | | ITPR1, ITPR2, ITPR3, MAD2L1, MAD2L2, MAP2K1, | | 1, 1, 1, 1, 1, 1, |
| | | | MAPK1, MAPK12, MAPK3, MOS, PGR, PKMYT1, | | 1, 1, 1, 1, 1, 1, |
| | | | PLCZ1, PLK1, PPP1CA, PPP1CB, PPP1CC, | | 1, 1, 1, 1, 1, 1, |
| | | | PPP2CA, PPP2CB, PPP2R1A, PPP2R1B, | | 1, 1, 1, 1, 1, 1, |
| | | | PPP2R5A, PPP2R5B, PPP2R5C, PPP2R5D, PPP2R5E, | | 1, 1, 1, 1, 1, 1, |
| | | | PPP3CA, PPP3CB, PPP3CC, PPP3R1, | | 1, 1, 1, 1, 1, 1, |
| | | | PPP3R2, PRKACA, PRKACB, PRKACG, PRKX, | | 1, 1, 1, 1, 1, 1, |
| | | | PTTG1, PTTG2, RBX1, REC8, RPS6KA1, RPS6KA2, | | 1, 1, 1, 1, 1, 1, |
| | | | RPS6KA3, RPS6KA6, SGO1, SKP1, SLK, SMC1A, | | 1, 1, 1, 1, 1 |
| | | | SMC1B, SMC3, SPDYA, SPDYC, STAG3, | | |
| | | | YWHAB, YWHAE, YWHAG, YWHAH, YWHAQ, | | |
| | | | YWHAZ | | |
| module46 | REACTOME_{—} | REACTOME_BIOSYNTHESIS_OF_THE_{—} | ALG5, AMDHD2, CMAS, CTSA, DHDDS, DOLK, | 78 | 2, 2, 2, 2, 2, 2, |
| | SYNTHESIS_OF_{—} | N_GLYCAN_PRECURSOR_DOLICHOL_{—} | DOLPP1, DPM1, DPM2, DPM3, FCSK, FPGT, FUOM, | | 2, 2, 2, 2, 2, 2, |
| | SUBSTRATES_INN_{—} | LIPID_LINKED_OLIGOSACCHARIDE_{—} | GFPT1, GFPT2, GLB1, GMDS, GMPPA, GMPPB, | | 2, 2, 2, 2, 2, 2, |
| | GLYCAN_BIO | LLO_AND_TRANSFER_TO_A_{—} | GNE, GNPNAT1, MPI, MVD, NAGK, NANP, | | 2, 2, 2, 2, 2, 2, |
| | | NASCENT_PROTEIN, | NANS, NEU1, NEU2, NEU3, NEU4, NPL, NUDT14, | | 2, 2, 2, 2, 2, 2, |
| | | REACTOME_SYNTHESIS_OF_{—} | NUS1, PGM3, PMM1, PMM2, RENBP, SLC17A5, | | 2, 2, 2, 2, 2, 2, |
| | | SUBSTRATES_IN_NGLYCAN_{—} | SLC35A1, SLC35C1, SRD5A3, ST3GAL1, ST3GAL2, | | 2, 2, 2, 2, 2, 2, |
| | | BIOSYTHESIS | ST3GAL3, ST3GAL4, ST3GAL5, ST3GAL6, | | 2, 2, 2, 2, 2, 2, |
| | | | ST6GAL1, ST6GAL2, ST6GALNAC1, ST6GALNAC2, | | 2, 2, 2, 2, 2, 2, |
| | | | ST6GALNAC3, ST6GALNAC4, ST6GALNAC5, | | 2, 2, 2, 2, 2, 2, |
| | | | ST6GALNAC6, ST8SIA1, ST8SIA2, ST8SIA3, | | 2, 2, 2, 1, 1, 1, |
| | | | ST8SIA4, ST8SIA5, ST8SIA6, TSTA3, UAP1, | | 1, 1, 1, 1, 1, 1, |
| | | | ALG1, ALG10, ALG10B, ALG11, ALG12, ALG13, | | 1, 1, 1, 1, 1, 1 |
| | | | ALG14, ALG2, ALG3, ALG6, ALG8, ALG9, DPAGT1, | | |
| | | | MPDU1, RFT1 | | |
| module47 | REACTOME_{—} | REACTOME_ACTIVATED_NOTCH1_{—} | ADAM10, ADAM17, APH1A, APH1B, ARRB1, ARRB2, | 31 | 1, 1, 1, 1, 1, 1, |
| | ACTIVATED_NOTCH1_{—} | TRANSMITS_SIGNAL_TO_THE_NUCLEUS | CNTN1, DLK1, DLL1, DLL4, DNER, DTX1, DTX2, | | 1, 1, 1, 1, 1, 1, |
| | TRANSMITSSIGNAL_{—} | | DTX4, ITCH, JAG1, JAG2, MIB1, MIB2, NCSTN, | | 1, 1, 1, 1, 1, 1, |
| | TO_TH | | NEURL1, NEURL1B, NOTCH1, NUMB, PSEN1, | | 1, 1, 1, 1, 1, 1, |
| | | | PSEN2, PSENEN, RPS27A, UBA52, UBB, UBC | | 1, 1, 1, 1, 1, 1, |
| | | | | | 1 |
| module48 | REACTOME_NETRIN_{—} | REACTOME_NETRIN_1_SIGNALING | ABLIM1, ABLIM2, ABLIM3, AGAP2, CDC42, DCC, | 50 | 1, 1, 1, 1, 1, 1, |
| | 1_SIGNALING | | DOCK1, DSCAM, DSCAML1, EZR, FYN, HJV, | | 1, 1, 1, 1, 1, 1, |
| | | | MAPK11, MAPK12, MAPK13, MAPK14, MAPK8, | | 1, 1, 1, 1, 1, 1, |
| | | | MYO10, NCK1, NEO1, NTN1, NTN4, PAK1, PITPNA, | | 1, 1, 1, 1, 1, 1, |
| | | | PLCG1, PRKCQ, PTK2, PTPN11, RAC1, RGMA, | | 1, 1, 1, 1, 1, 1, |
| | | | RGMB, ROBO1, SIAH1, SIAH2, SLIT1, SLIT2, | | 1, 1, 1, 1, 1, 1, |
| | | | SLIT3, SRC, TRIO, TRPC1, TRPC3, TRPC4, TRPC5, | | 1, 1, 1, 1, 1, 1, |
| | | | TRPC6, TRPC7, UNC5A, UNC5B, UNC5C, | | 1, 1, 1, 1, 1, 1, |
| | | | UNC5D, WASL | | 1, 1 |
| module49 | REACTOME_SIGNAL_{—} | REACTOME_SIGNAL_{—} | CSNK2A1, CSNK2A2, CSNK2B, EGFR, FGFR1, | 21 | 1, 1, 1, 1, 1, 1, |
| | TRANSDUCTION_{—} | TRANSDUCTION_BY_L1 | ITGA2B, ITGA5, ITGA9, ITGAV, ITGB1, ITGB3, L1CAM, | | 1, 1, 1, 1, 1, 1, |
| | BY_L1 | | MAP2K1, MAP2K2, MAPK1, MAPK3, NCAM1, | | 1, 1, 1, 1, 1, 1, |
| | | | NRP1, PAK1, RAC1, VAV2 | | 1, 1, 1 |
| module50 | BIOCARTA_EDG1_{—} | BIOCARTA_EDG1_PATHWAY | ADCY1, AKT1, ASAH1, ITGAV, ITGB3, MAPK1, | 22 | 1, 1, 1, 1, 1, 1, |
| | PATHWAY | | MAPK3, PDGFA, PDGFRA, PIK3CA, PIK3CG, PIK3R1, | | 1, 1, 1, 1, 1, 1, |
| | | | PLCB1, PRKCA, PRKCB, RAC1, RHOA, S1PR1, | | 1, 1, 1, 1, 1, 1, |
| | | | SMPD1, SMPD2, SPHK1, SPHKAP | | 1, 1, 1, 1 |
| module51 | WP_CARDIAC_{—} | WP_CARDIAC_PROGENITOR_{—} | ACTC1, ANPEP, BMP1, BMP4, CXCR4, DKK1, FGF2, | 53 | 1, 1, 1, 1, 1, 1, |
| | PROGENITOR_{—} | DIFFERENTIATION | FOXA2, GATA4, GSK3B, IGF1, IGF2, INHBA, | | 1, 1, 1, 1, 1, 1, |
| | DIFFERENTIATION | | INS, IRX4, ISL1, KDR, KIT, LIN28A, LIN28B, MAPK14, | | 1, 1, 1, 1, 1, 1, |
| | | | MEF2C, MESP1, MESP2, MIXL1, MYH6, | | 1, 1, 1, 1, 1, 1, |
| | | | MYL2, MYLK3, NANOG, NCAM1, NKX2-5, | | 1, 1, 1, 1, 1, 1, |
| | | | NODAL, NOG, NOTCH1, NRG1, PAX6, PDGFRA, | | 1, 1, 1, 1, 1, 1, |
| | | | POU5F1, ROR2, SCN5A, SIRPA, SOX1, SOX17, | | 1, 1, 1, 1, 1, 1, |
| | | | SOX2, TBX20, TBX5, TBXT, TGFB1, THY1, | | 1, 1, 1, 1, 1, 1, |
| | | | TNNI3, TNNT2, WNT3A, ZFP42 | | 1, 1, 1, 1, 1 |
| module52 | WP_REGULATION_OF_{—} | WP_REGULATION_OF_{—} | APC, AXIN1, CSNK1A1, CTNNB1, DKK3, DVL2, | 18 | 1, 1, 1, 1, 1, 1, |
| | WNTBCATENIN_{—} | WNTBCATENIN_SIGNALING_{—} | FZD1, FZD7, FZD8, GSK3B, LEF1, LRP1, MBOAT1, | | 1, 1, 1, 1, 1, 1, |
| | SIGNALING | BY_SMALL_MOLECULE_COMPOUNDS | MIR4683, SFRP4, TCF4, TNKS, WNT1 | | 1, 1, 1, 1, 1, 1 |
| module53 | REACTOME_{—} | REACTOME_SEMA4D_IN_{—} | ARHGAP35, ARHGEF11, ARHGEF12, ERBB2, | 24 | 1, 1, 1, 1, 1, 1, |
| | SEMA4D_IN_{—} | SEMAPHORIN_SIGNALING | LIMK1, LIMK2, MET, MYH10, MYH11, MYH14, MYH9, | | 1, 1, 1, 1, 1, 1, |
| | SEMAPHORIN_SIGNALING | | MYL12B, MYL6, MYL9, PLXNB1, RAC1, RHOA, | | 1, 1, 1, 1, 1, 1, |
| | | | RHOB, RHOC, RND1, ROCK1, ROCK2, RRAS, | | 1, 1, 1, 1, 1, 1 |
| | | | SEMA4D | | |
| module54 | PID_ERBB_NETWORK_{—} | REACTOME_ERBB2_ACTIVATES_{—} | BTC, EGF, EGFR, ERBB3, ERBB4, EREG, HBEGF, | 71 | 6, 6, 6, 6, 6, 6, |
| | PATHWAY | PTK6_SIGNALING, | NRG1, NRG2, NRG3, NRG4, ERBB2, GRB2, | | 6, 6, 6, 6, 6, 5, |
| | | REACTOME_ERBB2_REGULATES_{—} | HRAS, KRAS, NRAS, PIK3CA, PIK3R1, SHC1, SOS1, | | 3, 2, 2, 2, 2, 2, |
| | | CELL_MOTILITY, | ADAM17, ADAP1, APH1A, APH1B, APOE, | | 2, 2, 1, 1, 1, 1, |
| | | REACTOME_SHC1_EVENTS_IN_{—} | AREG, CSN2, CXCL12, DIAPH1, DLG4, ESR1, GAB1, | | 1, 1, 1, 1, 1, 1, |
| | | ERBB2_SIGNALING, | GABRA1, GABRB1, GABRB2, GABRB3, GABRG2, | | 1, 1, 1, 1, 1, 1, |
| | | REACTOME_PI3K_EVENTS_IN_{—} | GABRG3, GABRQ, GFAP, HSP90AA1, ITCH, | | 1, 1, 1, 1, 1, 1, |
| | | ERBB2_SIGNALING, | MEMO1, MXD4, NCOR1, NCSTN, NEDD4, | | 1, 1, 1, 1, 1, 1, |
| | | REACTOME_SIGNALING_BY_ERBB4, | PGR, PRKCA, PRKCD, PRKCE, PSEN1, PSEN2, | | 1, 1, 1, 1, 1, 1, |
| | | PID_ERBB_NETWORK_PATHWAY | PSENEN, PTK6, PTPN12, RHOA, RPS27A, S100B, | | 1, 1, 1, 1, 1, 1, |
| | | | SPARC, SRC, STAT5A, STMN1, TAB2, TGFA, | | 1, 1, 1, 1, 1, 1, |
| | | | UBA52, UBB, UBC, WWOX, WWP1, YAP1 | | 1, 1, 1, 1, 1 |

TABLE 2

| | | | Mean | Mean expression | | | |
|---|---|---|---|---|---|---|---|
| | | | expression | across other | | | Adjusted |
| Module | gsName | Cluster | in Cluster | clusters | Delta | P-value | P-value |
| module50 | biocarta edg1 pathway | 1 | 1.573245408 | −0.099362868 | 1.672608275 | 0.005682431 | 0.01334136 |
| module54 | pid erbb network pathway | 1 | 0.840619008 | −0.053091727 | 0.893710734 | 0.028458885 | 0.051225992 |
| module49 | reactome signal transduction | 1 | 1.454213032 | −0.091845034 | 1.546058065 | 0.004548292 | 0.011695609 |
| | by l1 | | | | | | |
| module51 | wp cardiac progenitor | 1 | 1.830401801 | −0.115604324 | 1.946006125 | 0.000794815 | 0.003065714 |
| | differentiation | | | | | | |
| module52 | wp regulation of wntbcatenin | 1 | 0.861550076 | −0.054413689 | 0.915963765 | 0.031729465 | 0.05527068 |
| | signaling | | | | | | |
| module50 | biocarta edg1 pathway | 2 | 0.477294504 | −0.125289807 | 0.602584312 | 0.005535758 | 0.012455455 |
| module45 | kegg oocyte meiosis | 2 | 0.448233641 | −0.117661331 | 0.565894972 | 0.019776344 | 0.035597419 |
| module48 | reactome netrin 1 signaling | 2 | 0.610135537 | −0.160160579 | 0.770296116 | 0.001494445 | 0.004483336 |
| module53 | reactome sema4d in | 2 | 0.583764748 | −0.153238246 | 0.737002995 | 0.002102622 | 0.005160981 |
| | semaphorin signaling | | | | | | |
| module49 | reactome signal transduction | 2 | 0.591918705 | −0.15537866 | 0.747297365 | 0.001219251 | 0.003872915 |
| | by l1 | | | | | | |
| module52 | wp regulation of wntbcatenin | 2 | 0.503191673 | −0.132087814 | 0.635279487 | 0.007692075 | 0.016614881 |
| | signaling | | | | | | |
| module43 | reactome cilium assembly | 3 | 0.338122111 | −0.156810254 | 0.494932366 | 0.01584471 | 0.071301194 |
| module46 | reactome synthesis of | 3 | 0.39317691 | −0.182342915 | 0.575519825 | 0.010963395 | 0.065780372 |
| | substrates in n glycan bio | | | | | | |
| module31 | biocarta cytokine pathway | 4 | 1.17499677 | −0.32721429 | 1.502211061 | 6.46E−09 | 8.72E−08 |
| module19 | biocarta keratinocyte pathway | 4 | 0.323926643 | −0.09020742 | 0.414134063 | 0.047849394 | 0.078299008 |
| module33 | biocarta tid pathway | 4 | 1.190026386 | −0.331399753 | 1.521426139 | 1.51E−09 | 4.30E−08 |
| module35 | kegg leishmania infection | 4 | 0.867259789 | −0.241515384 | 1.108775174 | 5.27E−06 | 2.84E−05 |
| module16 | kegg rig i like receptor | 4 | 0.590130807 | −0.164340225 | 0.754471032 | 0.001241036 | 0.003350797 |
| | signaling pathway | | | | | | |
| module5 | pid anthrax pathway | 4 | 0.564334917 | −0.157156559 | 0.721491476 | 0.00670913 | 0.014855334 |
| module36 | pid ifng pathway | 4 | 0.795876238 | −0.221636421 | 1.017512659 | 1.59E−05 | 6.62E−05 |
| module30 | pid il27 pathway | 4 | 1.0626822 | −0.295936815 | 1.358619015 | 4.79E−08 | 5.17E−07 |
| module15 | pid nfkappab canonical | 4 | 0.794032812 | −0.221123062 | 1.015155874 | 1.59E−05 | 6.62E−05 |
| | pathway | | | | | | |
| module28 | pid reg gr pathway | 4 | 0.503490767 | −0.140212619 | 0.643703386 | 0.014709998 | 0.028369281 |
| module22 | reactome dap12 signaling | 4 | 0.657234313 | −0.183027277 | 0.840261591 | 0.000689234 | 0.002189332 |
| module4 | reactome il1 signaling/tlr4 | 4 | 0.817787968 | −0.227738422 | 1.04552639 | 1.09E−05 | 5.37E−05 |
| | tlr10 cascade | | | | | | |
| module13 | reactome interleukin 37 | 4 | 0.541558644 | −0.150813799 | 0.692372443 | 0.004833823 | 0.011348975 |
| | signaling | | | | | | |
| module34 | reactome regulation of ifng | 4 | 0.950306791 | −0.264642397 | 1.214949188 | 2.10E−06 | 1.26E−05 |
| | signaling | | | | | | |
| module37 | wp allograft rejection | 4 | 1.155194982 | −0.321699868 | 1.47689485 | 6.15E−09 | 8.72E−08 |
| module7 | wp biomarkers for urea cycle | 4 | 0.471388721 | −0.131272808 | 0.60266153 | 0.006877469 | 0.014855334 |
| | disorders | | | | | | |
| module29 | wp development and | 4 | 1.000196825 | −0.278535825 | 1.278732649 | 5.79E−07 | 4.46E−06 |
| | heterogeneity of the ilc fami | | | | | | |
| module3 | wp il18 signaling pathway | 4 | 0.887135033 | −0.247050262 | 1.134185295 | 1.65E−06 | 1.11E−05 |
| module27 | wp selenium/wp folate | 4 | 0.741471167 | −0.206485642 | 0.947956809 | 9.46E−05 | 0.000319423 |
| | metabolism | | | | | | |
| module38 | wp spinal cord injury | 4 | 0.822265808 | −0.228985415 | 1.051251222 | 1.92E−05 | 7.40E−05 |
| module26 | wp tlr4 signaling and | 4 | 0.908230335 | −0.252924903 | 1.161155238 | 3.16E−07 | 2.85E−06 |
| | tolerance | | | | | | |
| module32 | wp type ii interferon signaling | 4 | 1.233016077 | −0.343371566 | 1.576387643 | 1.59E−09 | 4.30E−08 |
| | ifng | | | | | | |
| module19 | biocarta keratinocyte pathway | 5 | 0.564292483 | −0.139331477 | 0.70362396 | 0.006474579 | 0.012949158 |
| module1 | biocarta ppara pathway | 5 | 0.453857918 | −0.112063684 | 0.565921602 | 0.024194603 | 0.043550285 |
| module33 | biocarta tid pathway | 5 | 0.39892643 | −0.098500353 | 0.497426782 | 0.010966414 | 0.021149513 |
| module35 | kegg leishmania infection | 5 | 0.434379948 | −0.107254308 | 0.541634256 | 0.01932285 | 0.035980479 |
| module16 | kegg rig i like receptor | 5 | 0.550176058 | −0.13584594 | 0.686021998 | 0.001895032 | 0.00487294 |
| | signaling pathway | | | | | | |
| module5 | pid anthrax pathway | 5 | 0.885141919 | −0.21855356 | 1.103695479 | 1.86E−06 | 2.51E−05 |
| module36 | pid ifng pathway | 5 | 0.616671461 | −0.152264558 | 0.76893602 | 0.000622731 | 0.002040987 |
| module10 | pid il1 pathway | 5 | 0.968753336 | −0.239198355 | 1.207951691 | 8.92E−08 | 4.82E−06 |
| module30 | pid il27 pathway | 5 | 0.34950938 | −0.086298612 | 0.435807992 | 0.0429768 | 0.068257271 |
| module2 | pid myc repress pathway | 5 | 0.523272668 | −0.129203128 | 0.652475796 | 0.027007062 | 0.044193375 |
| module20 | pid ncadherin pathway | 5 | 0.610892018 | −0.150837535 | 0.761729553 | 0.00159216 | 0.00442618 |
| module15 | pid nfkappab canonical | 5 | 0.649141459 | −0.160281842 | 0.8094233 | 0.000930522 | 0.002791566 |
| | pathway | | | | | | |
| module28 | pid reg gr pathway | 5 | 0.551508786 | −0.136175009 | 0.687683795 | 0.003063065 | 0.006891897 |
| module8 | reactome circadian gene | 5 | 0.673702567 | −0.166346313 | 0.84004888 | 0.000103731 | 0.000466789 |
| | expression | | | | | | |
| module22 | reactome dap12 signaling | 5 | 0.638310395 | −0.157607505 | 0.7959179 | 0.00043933 | 0.001694558 |
| module14 | reactome gene silencing by | 5 | 0.683830199 | −0.168846963 | 0.852677162 | 7.03E−05 | 0.000344867 |
| | rna | | | | | | |
| module4 | reactome il1 signaling/tlr4 | 5 | 0.645297613 | −0.159332744 | 0.804630357 | 0.000531959 | 0.001915052 |
| | tlr10 cascade | | | | | | |
| module9 | reactome infectious disease | 5 | 0.914264834 | −0.225744403 | 1.140009237 | 8.48E−06 | 6.54E−05 |
| module13 | reactome interleukin 37 | 5 | 0.677602766 | −0.167309325 | 0.84491209 | 0.000199396 | 0.000828261 |
| | signaling | | | | | | |
| module12 | reactome regulation of ifna | 5 | 0.624806421 | −0.15427319 | 0.779079611 | 4.64E−07 | 8.35E−06 |
| | signaling | | | | | | |
| module25 | reactome signaling by hippo | 5 | 0.828496155 | −0.204566952 | 1.033063107 | 1.85E−05 | 0.000111016 |
| module11 | reactome tnf signaling | 5 | 0.862040598 | −0.21284953 | 1.074890129 | 4.83E−06 | 4.35E−05 |
| module24 | wp ap1 survival signaling | 5 | 0.920053653 | −0.227173742 | 1.147227395 | 1.07E−05 | 7.26E−05 |
| module7 | wp biomarkers for urea cycle | 5 | 0.773439826 | −0.190972797 | 0.964412623 | 2.24E−05 | 0.000120909 |
| | disorders | | | | | | |
| module17 | wp energy metabolism | 5 | 0.689899777 | −0.170345624 | 0.860245401 | 0.000642533 | 0.002040987 |
| module3 | wp il18 signaling pathway | 5 | 0.59958279 | −0.148045133 | 0.747627924 | 0.001639326 | 0.00442618 |
| module6 | wp leptin signaling pathway | 5 | 0.857428353 | −0.211710704 | 1.069139057 | 2.83E−06 | 3.06E−05 |
| module21 | wp mthfr deficiency | 5 | 0.525001912 | −0.129630102 | 0.654632013 | 0.00485552 | 0.010355308 |
| module26 | wp tlr4 signaling and | 5 | 0.423157588 | −0.104483355 | 0.527640943 | 0.025849897 | 0.043621701 |
| | tolerance | | | | | | |
| module23 | wp viral acute myocarditis | 5 | 1.021036053 | −0.252107668 | 1.273143721 | 1.79E−07 | 4.84E−06 |

TABLE 3

| | | Mean expression | Mean expression | | | Adjusted |
|---|---|---|---|---|---|---|
| Protein | Cluster | in Cluster | across other clusters | Delta | P-value | P-value |
| TNXB | 1 | 0.54266667 | 0.13158526 | 0.4110814 | 0.00161346 | 0.018298399 |
| GPC1 | 1 | 0.24831667 | −0.1247863 | 0.37310298 | 0.00249322 | 0.022797379 |
| MATN3 | 1 | 0.86065 | 0.48014316 | 0.38050684 | 0.0036232 | 0.028046219 |
| CLEC14A | 1 | 0.57923333 | 0.23620947 | 0.34302386 | 0.00593777 | 0.041764215 |
| GHRL | 1 | 2.58776667 | 1.40502947 | 1.18273719 | 0.00593777 | 0.041764215 |
| CPE | 1 | 0.85933333 | 0.27425895 | 0.58507439 | 0.00803413 | 0.052945617 |
| MSTN | 1 | 1.33146667 | 0.52357579 | 0.80789088 | 0.00803413 | 0.052945617 |
| MYOC | 1 | 0.90363333 | 0.22981474 | 0.6738186 | 0.00874413 | 0.056604706 |
| ART3 | 1 | 0.42696667 | 0.01299789 | 0.41396877 | 0.00950969 | 0.060754022 |
| SDC1 | 1 | 0.79015 | 0.38172842 | 0.40842158 | 0.01122229 | 0.067843858 |
| DPP4 | 1 | 0.1465 | −0.1544947 | 0.30099474 | 0.01217722 | 0.071835797 |
| BOC | 1 | 0.63838333 | 0.38581895 | 0.25256439 | 0.01430518 | 0.080495723 |
| ITGB6 | 1 | 0.2457 | −0.30516 | 0.55086 | 0.01430546 | 0.080495723 |
| COMP | 1 | 0.51753333 | 0.11860316 | 0.39893018 | 0.01956601 | 0.102232428 |
| GALNT10 | 1 | 1.33165 | 1.01514105 | 0.31650895 | 0.02032963 | 0.105098192 |
| THBS2 | 1 | 0.73888333 | 0.38661895 | 0.35226439 | 0.02033 | 0.105098192 |
| MMP13 | 1 | 1.71203333 | 1.21299368 | 0.49903965 | 0.02111988 | 0.108415372 |
| C4BPB | 1 | 0.78415 | 0.39204421 | 0.39210579 | 0.02193636 | 0.111048082 |
| IL7R | 1 | 2.08311667 | 1.61016526 | 0.4729514 | 0.02193636 | 0.111048082 |
| NOS1 | 1 | −0.96315 | −1.5604284 | 0.59727842 | 0.02193636 | 0.111048082 |
| ACP5 | 1 | 0.7069 | 0.41636105 | 0.29053895 | 0.02365204 | 0.117697049 |
| CCL15 | 1 | 0.8997 | 0.46826632 | 0.43143368 | 0.02365204 | 0.117697049 |
| TIMD4 | 1 | 0.45883333 | 0.08283158 | 0.37600175 | 0.02455272 | 0.120944858 |
| ANGPTL1 | 1 | 0.54651667 | 0.26123368 | 0.28528298 | 0.02845842 | 0.133875717 |
| SMPDL3A | 1 | 0.83786667 | 0.10879789 | 0.72906877 | 0.02845842 | 0.133875717 |
| NRP1 | 1 | 0.46081667 | 0.14030526 | 0.3205114 | 0.03060516 | 0.141694154 |
| AKR1C4 | 1 | 0.67658333 | 0.05812737 | 0.61845596 | 0.03172946 | 0.145975493 |
| ENPP5 | 1 | 0.37725 | −0.0861989 | 0.46344895 | 0.03408462 | 0.153906801 |
| BMP4 | 1 | −0.3654 | −0.7953516 | 0.42995158 | 0.03658708 | 0.160262567 |
| CD55 | 1 | 0.4064 | 0.21068632 | 0.19571368 | 0.03658708 | 0.160262567 |
| DKK3 | 1 | 0.90153333 | 0.61702421 | 0.28450912 | 0.03658763 | 0.160262567 |
| TNNI3 | 1 | 0.90861667 | −0.4300768 | 1.33869351 | 0.03658763 | 0.160262567 |
| CA14 | 1 | 0.3777 | −0.0132084 | 0.39090842 | 0.03789678 | 0.164519244 |
| IGFBP1 | 1 | 1.68298333 | 0.40191895 | 1.28106439 | 0.03789678 | 0.164519244 |
| PDGFRA | 1 | 0.52601667 | 0.24781579 | 0.27820088 | 0.03924442 | 0.169369484 |
| HSD11B1 | 1 | 0.60783333 | 0.23563789 | 0.37219544 | 0.04063489 | 0.171817463 |
| PLA2G7 | 1 | 0.33815 | 0.06104842 | 0.27710158 | 0.04063489 | 0.171817463 |
| RGMB | 1 | 0.291 | 0.05112842 | 0.23987158 | 0.04134445 | 0.173847736 |
| GALNT2 | 1 | 0.68483333 | 0.46342526 | 0.22140807 | 0.04206569 | 0.173847736 |
| LTO1 | 1 | 0.5521 | 0.32892211 | 0.22317789 | 0.04353889 | 0.17842406 |
| PROK1 | 1 | 1.0146 | 0.28639895 | 0.72820105 | 0.04822238 | 0.191710176 |
| DCTPP1 | 1 | 0.6464 | 0.35837474 | 0.28802526 | 0.04987472 | 0.195621225 |
| EGFR | 1 | 0.41898333 | 0.27359789 | 0.14538544 | 0.04987472 | 0.195621225 |
| PON3 | 1 | 0.22551667 | −0.0252832 | 0.25079982 | 0.04987472 | 0.195621225 |
| ITGA11 | 2 | 0.39685238 | 0.0833325 | 0.31351988 | 0.00141049 | 0.015996445 |
| NTRK3 | 2 | 0.20189524 | 0.03223625 | 0.16965899 | 0.00228655 | 0.023557898 |
| ITGAV | 2 | 0.15302381 | 0.03488625 | 0.11813756 | 0.00335599 | 0.031474881 |
| CA14 | 2 | 0.30838095 | −0.0683075 | 0.37668845 | 0.00373631 | 0.034163869 |
| LEPR | 2 | 0.71551429 | 0.49348375 | 0.22203054 | 0.01437633 | 0.088372122 |
| VEGFD | 2 | 0.47244286 | 0.2371375 | 0.23530536 | 0.01576777 | 0.094541974 |
| MCAM | 2 | 0.1851619 | −0.0222063 | 0.20736815 | 0.0186983 | 0.107699254 |
| KLK13 | 2 | 1.19971905 | 0.90332625 | 0.2963928 | 0.02185297 | 0.119282838 |
| CLUL1 | 2 | 0.90739524 | 0.65328125 | 0.25411399 | 0.02209545 | 0.119282838 |
| AMY2B | 2 | 0.79070952 | 0.5736225 | 0.21708702 | 0.02411765 | 0.125566268 |
| AMY2A | 2 | 0.76780952 | 0.55281375 | 0.21499577 | 0.02573738 | 0.131200083 |
| DPEP1 | 2 | 0.06170476 | −0.1591013 | 0.22080601 | 0.0262984 | 0.132214957 |
| CRTAC1 | 2 | 0.21272857 | −0.0126913 | 0.22541982 | 0.02864703 | 0.14158986 |
| ITM2A | 2 | 1.3142 | 1.127925 | 0.186275 | 0.028953 | 0.142620324 |
| CNTN5 | 2 | 0.24662381 | 0.06458375 | 0.18204006 | 0.03389121 | 0.158411635 |
| IGFBP1 | 2 | 1.02670952 | 0.33399125 | 0.69271827 | 0.03532432 | 0.163026742 |
| DCN | 2 | 0.38734762 | 0.18756 | 0.19978762 | 0.03834498 | 0.173144165 |
| IGFBP2 | 2 | 1.21163333 | 0.7360925 | 0.47554083 | 0.0399359 | 0.175982602 |
| EPCAM | 2 | 1.11063333 | 0.64150875 | 0.46912458 | 0.04116494 | 0.178707152 |
| ICAM4 | 2 | 0.64820952 | 0.38991875 | 0.25829077 | 0.04158247 | 0.178926931 |
| GHRL | 2 | 1.8746619 | 1.37045625 | 0.50420565 | 0.04504846 | 0.189930558 |
| PON3 | 2 | 0.13134762 | −0.0475888 | 0.17893637 | 0.04780511 | 0.197567431 |
| UBAC1 | 3 | 0.79066875 | 0.56065362 | 0.23001513 | 0.00868617 | 0.4448788 |
| BOC | 3 | 0.5142625 | 0.34821304 | 0.16604946 | 0.01191429 | 0.4448788 |
| AHSP | 3 | 1.220225 | 0.8435942 | 0.3766308 | 0.01347853 | 0.446841295 |
| PDGFB | 3 | 5.23215313 | 5.0193058 | 0.21284733 | 0.01584471 | 0.446841295 |
| HARS | 3 | 0.90307344 | 0.6997413 | 0.20333213 | 0.01750262 | 0.446841295 |
| TPMT | 3 | 1.41978125 | 1.12744783 | 0.29233342 | 0.01856875 | 0.446841295 |
| PROK1 | 3 | 0.63605313 | 0.18756232 | 0.44849081 | 0.02297902 | 0.508487646 |
| KIT | 3 | 0.16286094 | 0.06086594 | 0.101995 | 0.02432955 | 0.508487646 |
| ANKRD54 | 3 | 0.86005625 | 0.51834348 | 0.34171277 | 0.03335417 | 0.525438876 |
| ALDH3A1 | 3 | 1.09747031 | 0.89408333 | 0.20338698 | 0.03396437 | 0.525438876 |
| PSPN | 3 | −0.7394328 | −1.2983717 | 0.55893893 | 0.03396437 | 0.525438876 |
| ADAMTS13 | 3 | 0.31067344 | 0.17872681 | 0.13194663 | 0.03585252 | 0.525438876 |
| RANGAP1 | 3 | 0.184775 | 0.01148841 | 0.17328659 | 0.03782947 | 0.525438876 |
| SMPDL3A | 3 | 0.33153438 | 0.0688971 | 0.26263727 | 0.03850817 | 0.525438876 |
| ESM1 | 3 | 1.3497375 | 1.16596522 | 0.18377228 | 0.03850874 | 0.525438876 |
| CDH3 | 3 | 0.68172188 | 0.54315362 | 0.13856825 | 0.03919834 | 0.525438876 |
| CD34 | 3 | 0.34491094 | 0.22070217 | 0.12420876 | 0.03989724 | 0.525438876 |
| AARSD1 | 3 | 1.67175938 | 1.42754638 | 0.244213 | 0.04133038 | 0.525438876 |
| NXPH1 | 3 | 0.42248125 | 0.17661014 | 0.24587111 | 0.04206254 | 0.525438876 |
| INPPL1 | 3 | 1.12208438 | 0.87694203 | 0.24514235 | 0.04432458 | 0.525438876 |
| AK1 | 3 | 0.8292625 | 0.59678696 | 0.23247554 | 0.04510188 | 0.525438876 |
| VPS37A | 3 | 1.07519688 | 0.94348116 | 0.13171572 | 0.04588938 | 0.525438876 |
| KLB | 3 | 0.11240313 | −0.2256797 | 0.33808284 | 0.04668972 | 0.525438876 |
| MPI | 3 | −0.0246406 | −0.2029652 | 0.17832459 | 0.04750111 | 0.52647066 |
| IFNG | 4 | 1.52867273 | 0.01028481 | 1.51838792 | 6.62E−08 | 5.07E−05 |
| CD5 | 4 | 1.60585 | 1.12630127 | 0.47954873 | 6.93E−08 | 5.07E−05 |
| CD74 | 4 | 0.92448182 | 0.47630127 | 0.44818055 | 1.19E−07 | 5.82E−05 |
| TNFRSF1B | 4 | 0.64795909 | 0.1662443 | 0.48171479 | 2.33E−07 | 8.52E−05 |
| TNFRSF4 | 4 | 0.52605 | −0.0130595 | 0.53910949 | 6.58E−07 | 0.00016727 |
| CXCL9 | 4 | 0.68722273 | −0.3586696 | 1.04589235 | 6.86E−07 | 0.00016727 |
| TNFRSF8 | 4 | 0.46712273 | −0.0958342 | 0.5629569 | 9.61E−07 | 0.000200859 |
| TNF | 4 | 1.26400795 | 0.64659747 | 0.61741049 | 1.29E−06 | 0.000219063 |
| IL18BP | 4 | 0.36087273 | −0.0250127 | 0.38588539 | 1.40E−06 | 0.000219063 |
| GRN | 4 | 0.60463182 | 0.31301139 | 0.29162043 | 1.58E−06 | 0.000219063 |
| HAVCR2 | 4 | 0.74133182 | 0.23080253 | 0.51052929 | 1.65E−06 | 0.000219063 |
| CXCL10 | 4 | 1.64645455 | 0.71831392 | 0.92814062 | 1.94E−06 | 0.000236509 |
| GZMA | 4 | 0.47079091 | 0.07486329 | 0.39592762 | 5.27E−06 | 0.000550307 |
| LGALS9 | 4 | 0.49726364 | −0.0306063 | 0.52786997 | 5.27E−06 | 0.000550307 |
| CD300E | 4 | 0.75362273 | 0.21589367 | 0.53772906 | 6.40E−06 | 0.000624109 |
| CD83 | 4 | 0.55057727 | 0.13392658 | 0.41665069 | 6.91E−06 | 0.000632239 |
| CD300C | 4 | 0.49257727 | 0.11162278 | 0.38095449 | 8.71E−06 | 0.000735505 |
| ICAM1 | 4 | 0.48803636 | 0.1805962 | 0.30744016 | 9.05E−06 | 0.000735505 |
| WARS | 4 | 0.22369091 | −0.2350468 | 0.45873774 | 1.01E−05 | 0.000781268 |
| CCL20 | 4 | −0.2611773 | −1.1578278 | 0.89665058 | 1.23E−05 | 0.000854186 |
| EFNA4 | 4 | 0.93634091 | 0.58174177 | 0.35459914 | 1.23E−05 | 0.000854186 |
| TNFRSF13B | 4 | 0.36865455 | 0.01468608 | 0.35396847 | 1.32E−05 | 0.000879101 |
| IL32 | 4 | 0.59012273 | 0.14236076 | 0.44776197 | 1.59E−05 | 0.000965448 |
| CDCP1 | 4 | 0.5683 | −0.0492456 | 0.61754557 | 1.65E−05 | 0.000965448 |
| IGFBP4 | 4 | 1.18170909 | 0.60021646 | 0.58149264 | 1.65E−05 | 0.000965448 |
| TREM2 | 4 | 0.87697273 | 0.11623924 | 0.76073349 | 1.72E−05 | 0.000965448 |
| DSC2 | 4 | 0.82720455 | 0.44812025 | 0.37908429 | 1.78E−05 | 0.000965448 |
| TNFRSF1A | 4 | 0.68923636 | 0.30180759 | 0.38742877 | 1.99E−05 | 0.001040058 |
| TNFRSF10B | 4 | 0.70628636 | 0.27915823 | 0.42712814 | 2.77E−05 | 0.001395529 |
| TNFRSF10A | 4 | 1.02953182 | 0.5917 | 0.43783182 | 2.87E−05 | 0.001398873 |
| TMSB10 | 4 | 1.92526364 | 1.15447468 | 0.77078895 | 2.97E−05 | 0.001403619 |
| PDCD1 | 4 | 0.36791364 | −0.0779899 | 0.44590351 | 3.08E−05 | 0.001409756 |
| F7 | 4 | 0.55238636 | 0.15787215 | 0.39451421 | 3.20E−05 | 0.001417213 |
| LAIR1 | 4 | 0.38851818 | −0.1019519 | 0.49047008 | 3.56E−05 | 0.001488256 |
| PILRA | 4 | 0.66043182 | 0.17439494 | 0.48603688 | 3.56E−05 | 0.001488256 |
| TNFSF13B | 4 | 0.71959091 | 0.4116557 | 0.30793521 | 3.96E−05 | 0.001567068 |
| EFNA1 | 4 | 0.69425 | 0.3761519 | 0.3180981 | 3.96E−05 | 0.001567068 |
| TNFRSF9 | 4 | 0.43423636 | −0.0360949 | 0.4703313 | 4.41E−05 | 0.001697449 |
| TNFRSF6B | 4 | 0.75731364 | 0.19332278 | 0.56399085 | 4.57E−05 | 0.001713523 |
| ADM | 4 | 0.53176818 | 0.11476203 | 0.41700616 | 4.73E−05 | 0.001730776 |
| C2 | 4 | 0.66716818 | 0.38695823 | 0.28020995 | 5.26E−05 | 0.001876666 |
| ZBTB17 | 4 | 0.8578 | 0.47254177 | 0.38525823 | 5.64E−05 | 0.001964896 |
| CLMP | 4 | 0.66130455 | 0.33912532 | 0.32217923 | 5.84E−05 | 0.001987558 |
| DLL1 | 4 | 0.49007273 | 0.15853924 | 0.33153349 | 6.48E−05 | 0.002156259 |
| REG1B | 4 | 1.04776364 | 0.22790253 | 0.8198611 | 7.19E−05 | 0.002269902 |
| SIGLEC1 | 4 | 0.55296364 | 0.08486076 | 0.46810288 | 7.19E−05 | 0.002269902 |
| CD160 | 4 | 0.72095455 | 0.24220506 | 0.47874948 | 7.45E−05 | 0.002269902 |
| SIGLEC6 | 4 | 0.73185909 | 0.41569241 | 0.31616669 | 7.45E−05 | 0.002269902 |
| REG1A | 4 | 0.72992273 | 0.07258101 | 0.65734171 | 8.84E−05 | 0.002639615 |
| CSTB | 4 | 1.04041364 | 0.60058987 | 0.43982376 | 0.00010129 | 0.002963832 |
| IL6 | 4 | 1.29036477 | 0.56122563 | 0.72913914 | 0.00010838 | 0.00310906 |
| ACTA2 | 4 | 0.89573182 | 0.39465316 | 0.50107865 | 0.0001121 | 0.003153855 |
| ADA2 | 4 | 0.32223636 | −0.0638101 | 0.38604649 | 0.0001199 | 0.003248324 |
| VCAM1 | 4 | 0.39913182 | 0.11247089 | 0.28666093 | 0.0001199 | 0.003248324 |
| KLRD1 | 4 | 0.54678182 | 0.03452152 | 0.5122603 | 0.00013255 | 0.003463119 |
| CLEC7A | 4 | 0.18768182 | −0.3783177 | 0.56599954 | 0.00013256 | 0.003463119 |
| PIGR | 4 | 0.44965455 | 0.06338734 | 0.3862672 | 0.00014169 | 0.003636714 |
| AMBP | 4 | 0.47613182 | 0.25498608 | 0.22114574 | 0.00015141 | 0.003754472 |
| FABP4 | 4 | 1.19534545 | 0.4119962 | 0.78334925 | 0.00015141 | 0.003754472 |
| XG | 4 | 1.01906364 | 0.63562911 | 0.38343452 | 0.0001565 | 0.003816069 |
| MSR1 | 4 | 0.29424091 | −0.1921013 | 0.48634217 | 0.00016718 | 0.004009472 |
| KLRB1 | 4 | 0.97523636 | 0.68443165 | 0.29080472 | 0.00017562 | 0.004144005 |
| CXCL11 | 4 | 3.0943 | 2.53033291 | 0.56396709 | 0.00018448 | 0.004216985 |
| MMP10 | 4 | 0.47199545 | −0.0047582 | 0.47675368 | 0.00018448 | 0.004216985 |
| TFPI2 | 4 | 0.65668182 | 0.31152405 | 0.34515777 | 0.00020345 | 0.004579128 |
| NPDC1 | 4 | 1.05676818 | 0.67027722 | 0.38649097 | 0.00021016 | 0.004658535 |
| ADAM8 | 4 | 0.78625 | 0.5004443 | 0.2858057 | 0.00022424 | 0.004824481 |
| CST3 | 4 | 0.75449091 | 0.4330038 | 0.32148711 | 0.00022424 | 0.004824481 |
| IL12B | 4 | 0.56997273 | 0.03191139 | 0.53806133 | 0.0002316 | 0.00491069 |
| SCARB2 | 4 | 0.81484091 | 0.45444177 | 0.36039914 | 0.00023919 | 0.004950503 |
| CCL3 | 4 | 1.07642727 | 0.69849494 | 0.37793234 | 0.00024702 | 0.004950503 |
| CXCL8 | 4 | 2.08384545 | 1.77823228 | 0.30561318 | 0.00024702 | 0.004950503 |
| MMP7 | 4 | 0.28325 | −0.0076253 | 0.29087532 | 0.00027195 | 0.005304851 |
| RRM2 | 4 | 0.24312273 | −0.1197949 | 0.36291766 | 0.00027195 | 0.005304851 |
| SIT1 | 4 | 2.58423636 | 2.20659494 | 0.37764143 | 0.00028077 | 0.005404878 |
| BTN2A1 | 4 | 0.30483636 | 0.10033291 | 0.20450345 | 0.0002945 | 0.005523733 |
| GFAP | 4 | 0.73805909 | 0.36686709 | 0.371192 | 0.0002945 | 0.005523733 |
| VWC2 | 4 | 0.93489545 | 0.45481646 | 0.480079 | 0.00030888 | 0.005720064 |
| LEP | 4 | 2.44170455 | 1.11416329 | 1.32754125 | 0.00031881 | 0.005830313 |
| CCL22 | 4 | 2.40146364 | 1.98719747 | 0.41426617 | 0.0003396 | 0.006133681 |
| TNFRSF11B | 4 | 0.70274545 | 0.34384177 | 0.35890368 | 0.00035046 | 0.006252632 |
| CD48 | 4 | 0.24962273 | −0.0164089 | 0.26603159 | 0.00037314 | 0.006577172 |
| TGFBR2 | 4 | 0.49916364 | 0.10657342 | 0.39259022 | 0.00037903 | 0.006601386 |
| CTSS | 4 | 0.29142727 | 0.12235949 | 0.16906778 | 0.00038498 | 0.006626262 |
| METAP1 | 4 | 0.47573182 | 0.21044304 | 0.26528878 | 0.00040345 | 0.006812871 |
| LAMP3 | 4 | 0.04732727 | −0.4777101 | 0.5250374 | 0.00040978 | 0.006812871 |
| ITIH3 | 4 | 0.55888182 | 0.11901646 | 0.43986536 | 0.0004098 | 0.006812871 |
| FSTL3 | 4 | 0.50985 | 0.09441392 | 0.41543608 | 0.00042274 | 0.006949071 |
| IL10RB | 4 | 0.41878636 | 0.10674177 | 0.31204459 | 0.00044978 | 0.007311373 |
| EFEMP1 | 4 | 0.01458636 | −0.3365684 | 0.35115472 | 0.00046389 | 0.007376908 |
| IL12RB1 | 4 | 0.92392273 | 0.55998101 | 0.36394171 | 0.00046389 | 0.007376908 |
| LILRA2 | 4 | 0.39405 | 0.04439747 | 0.34965253 | 0.00049336 | 0.007753305 |
| CES1 | 4 | 0.7898 | 0.11501646 | 0.67478354 | 0.00050876 | 0.007753305 |
| GFRA1 | 4 | 0.55563636 | 0.26945949 | 0.28617687 | 0.00050876 | 0.007753305 |
| JAM2 | 4 | 0.43997273 | 0.18699367 | 0.25297906 | 0.00050876 | 0.007753305 |
| CSF1 | 4 | 0.35024091 | 0.08235949 | 0.26788142 | 0.00052459 | 0.007831446 |
| PIK3IP1 | 4 | 0.54852273 | 0.26268608 | 0.28583665 | 0.00052459 | 0.007831446 |
| IL2RA | 4 | 0.6429 | 0.24922658 | 0.39367342 | 0.00055765 | 0.008240802 |
| SEMA3F | 4 | 0.50315 | 0.2213962 | 0.2817538 | 0.00056617 | 0.008245771 |
| RNASET2 | 4 | 0.92645455 | 0.68377089 | 0.24268366 | 0.00057487 | 0.008245771 |
| KIR2DL3 | 4 | 0.66205 | −0.0817354 | 0.74378544 | 0.00057489 | 0.008245771 |
| ADGRE2 | 4 | 0.42730909 | 0.1628 | 0.26450909 | 0.00059263 | 0.008257361 |
| FCRL6 | 4 | 1.37310909 | 0.57435443 | 0.79875466 | 0.00059263 | 0.008257361 |
| RBP5 | 4 | 0.95542273 | 0.39513924 | 0.56028349 | 0.00059263 | 0.008257361 |
| GDF15 | 4 | 0.41330909 | −0.0928987 | 0.50620783 | 0.00064896 | 0.008956864 |
| SORCS2 | 4 | 0.75760455 | 0.37848608 | 0.37911847 | 0.00066881 | 0.009144638 |
| CD27 | 4 | 0.42082273 | 0.0100557 | 0.41076703 | 0.00073182 | 0.00973323 |
| FGF21 | 4 | 2.15905909 | 0.96954051 | 1.18951858 | 0.00073182 | 0.00973323 |
| SNCG | 4 | 1.31121818 | 0.53832785 | 0.77289033 | 0.00073182 | 0.00973323 |
| CLEC6A | 4 | 0.95164545 | 0.50927975 | 0.44236571 | 0.00075402 | 0.009849394 |
| CRTAM | 4 | 1.08805455 | 0.50269494 | 0.58535961 | 0.00075402 | 0.009849394 |
| HYOU1 | 4 | 0.66179545 | 0.50418861 | 0.15760685 | 0.00080031 | 0.010361483 |
| CTSO | 4 | 0.45577727 | 0.23738608 | 0.2183912 | 0.00082442 | 0.010580117 |
| LGALS4 | 4 | 0.58054091 | 0.04569747 | 0.53484344 | 0.00083668 | 0.010643984 |
| CTSL | 4 | 0.55351364 | 0.24242911 | 0.31108452 | 0.00084922 | 0.010710376 |
| CD97 | 4 | 0.68882727 | 0.39079241 | 0.29803487 | 0.00086184 | 0.010776681 |
| WFDC2 | 4 | 0.39755909 | −0.0588165 | 0.45637555 | 0.0009278 | 0.01150316 |
| FABP1 | 4 | 0.86339545 | −0.1889241 | 1.05231951 | 0.00095546 | 0.011648632 |
| INHBC | 4 | 0.46212273 | 0.03592785 | 0.42619488 | 0.00095546 | 0.011648632 |
| CA5A | 4 | −0.3785227 | −1.2549835 | 0.87646082 | 0.00098388 | 0.011702549 |
| GPNMB | 4 | 0.56837273 | 0.33221266 | 0.23616007 | 0.00098388 | 0.011702549 |
| OGN | 4 | 1.05992273 | 0.50993418 | 0.54998855 | 0.00098388 | 0.011702549 |
| GZMH | 4 | 0.19912727 | −0.5805608 | 0.77968803 | 0.00099835 | 0.011778865 |
| SFRP1 | 4 | −0.0751864 | −0.6242038 | 0.54901743 | 0.00101308 | 0.01185707 |
| COLEC12 | 4 | 0.50479545 | 0.16439494 | 0.34040052 | 0.00104308 | 0.01192207 |
| IL12A_IL12B | 4 | 1.19082727 | 0.47715316 | 0.71367411 | 0.00104308 | 0.01192207 |
| MLN | 4 | 1.87589091 | 1.06306709 | 0.81282382 | 0.00104308 | 0.01192207 |
| HAVCR1 | 4 | −0.0453955 | −0.6353203 | 0.5899248 | 0.00105835 | 0.011993255 |
| EZR | 4 | 0.32507273 | 0.12968354 | 0.19538918 | 0.0010739 | 0.011993255 |
| TFF3 | 4 | 0.92423636 | 0.40524177 | 0.51899459 | 0.0010739 | 0.011993255 |
| CD28 | 4 | 0.63628182 | 0.33147468 | 0.30480713 | 0.00108959 | 0.012076271 |
| CST5 | 4 | 0.41491364 | −0.0751696 | 0.49008326 | 0.00110556 | 0.012161184 |
| FCRL3 | 4 | 0.27260909 | −0.1937747 | 0.46638377 | 0.00113809 | 0.012333471 |
| TYMP | 4 | 1.47066818 | 1.14465316 | 0.32601502 | 0.00113809 | 0.012333471 |
| REG3A | 4 | 1.15624091 | 0.47750506 | 0.67873585 | 0.00131438 | 0.014139211 |
| IL1RN | 4 | 0.75115 | 0.29691899 | 0.45423101 | 0.00143191 | 0.015180351 |
| SPINK1 | 4 | 0.78599091 | 0.29163165 | 0.49435926 | 0.00143191 | 0.015180351 |
| SIGLEC10 | 4 | 0.69076818 | 0.37844304 | 0.31232514 | 0.00147315 | 0.015505158 |
| CCL16 | 4 | 1.53732273 | 1.14761013 | 0.3897126 | 0.00151557 | 0.015614677 |
| DTX3 | 4 | 0.58335909 | 0.31263291 | 0.27072618 | 0.00151557 | 0.015614677 |
| TNFRSF21 | 4 | 0.42678636 | 0.22628734 | 0.20049902 | 0.00151557 | 0.015614677 |
| FGFR2 | 4 | 0.42169091 | 0.1909 | 0.23079091 | 0.00155907 | 0.015950501 |
| CRIM1 | 4 | 0.47601818 | 0.32632658 | 0.1496916 | 0.00160372 | 0.016293313 |
| ITGB2 | 4 | 0.61369545 | 0.36192532 | 0.25177014 | 0.00164954 | 0.016416805 |
| LILRB4 | 4 | 0.30405 | −0.0379051 | 0.34195506 | 0.00164954 | 0.016416805 |
| FAM3C | 4 | 1.04061818 | 0.78232785 | 0.25829033 | 0.00167279 | 0.016535748 |
| ADAMTS16 | 4 | 0.329 | 0.0433038 | 0.2856962 | 0.00174481 | 0.016904986 |
| DPY30 | 4 | 0.01415 | −0.4348861 | 0.44903608 | 0.00174481 | 0.016904986 |
| IL13RA1 | 4 | 0.51565909 | 0.29183924 | 0.22381985 | 0.00179421 | 0.017157423 |
| LAG3 | 4 | 0.99341818 | 0.55881392 | 0.43460426 | 0.00179432 | 0.017157423 |
| CCL14 | 4 | 1.88425909 | 1.65360253 | 0.23065656 | 0.00184506 | 0.017415506 |
| LTA | 4 | 0.72502727 | 0.44343797 | 0.2815893 | 0.00184512 | 0.017415506 |
| MARCO | 4 | 0.35072273 | 0.10542405 | 0.24529868 | 0.00189723 | 0.017792627 |
| CD33 | 4 | 0.20437727 | −0.3615481 | 0.56592537 | 0.00200554 | 0.018570308 |
| SMOC1 | 4 | 0.08061818 | −0.1743392 | 0.25495742 | 0.00200554 | 0.018570308 |
| CCL19 | 4 | 2.17577727 | 1.6233962 | 0.55238107 | 0.0020618 | 0.018852582 |
| VSIG4 | 4 | 0.64779545 | 0.2290519 | 0.41874356 | 0.0020618 | 0.018852582 |
| AGRN | 4 | 0.57449545 | 0.26718734 | 0.30730811 | 0.0021195 | 0.019259819 |
| REG4 | 4 | 0.87479091 | 0.34434304 | 0.53044787 | 0.00217862 | 0.019317637 |
| LTA4H | 4 | 0.75775 | 0.39995316 | 0.35779684 | 0.00217868 | 0.019317637 |
| PILRB | 4 | 0.73495 | 0.24987722 | 0.48507278 | 0.00217868 | 0.019317637 |
| BST2 | 4 | 0.54708636 | 0.08483291 | 0.46225345 | 0.00220877 | 0.019466442 |
| LGALS1 | 4 | 1.0798 | 0.69999747 | 0.37980253 | 0.00223937 | 0.019617964 |
| P4HB | 4 | 0.20455455 | −0.0184861 | 0.22304062 | 0.00230161 | 0.019924563 |
| TNFRSF11A | 4 | 0.85748182 | 0.48320886 | 0.37427296 | 0.00230161 | 0.019924563 |
| CD6 | 4 | 0.14274091 | −0.1961519 | 0.33889281 | 0.00236542 | 0.020356557 |
| THY1 | 4 | 0.54715909 | 0.27317215 | 0.27398694 | 0.00256672 | 0.02195969 |
| S100A11 | 4 | 1.67932273 | 1.27772911 | 0.40159361 | 0.00263722 | 0.0224317 |
| ASGR1 | 4 | 0.48652273 | 0.22227595 | 0.26424678 | 0.00270941 | 0.022781504 |
| IL16 | 4 | 1.41395 | 1.02853418 | 0.38541582 | 0.00270949 | 0.022781504 |
| PLAUR | 4 | 0.77555909 | 0.5223557 | 0.25320339 | 0.00278356 | 0.023270584 |
| SIGLEC5 | 4 | 0.96773636 | 0.20117848 | 0.76655788 | 0.00285948 | 0.023611272 |
| ALCAM | 4 | 0.51240909 | 0.35011772 | 0.16229137 | 0.0029372 | 0.023611272 |
| EPHA2 | 4 | 0.62239545 | 0.37410759 | 0.24828786 | 0.0029372 | 0.023611272 |
| CHRDL1 | 4 | 0.16026818 | −0.207743 | 0.36801122 | 0.00293729 | 0.023611272 |
| LAYN | 4 | 0.68707273 | 0.41643797 | 0.27063475 | 0.00293729 | 0.023611272 |
| OSCAR | 4 | 0.48906364 | 0.16731139 | 0.32175224 | 0.00293729 | 0.023611272 |
| PRSS2 | 4 | 1.02147727 | 0.5253 | 0.49617727 | 0.00293729 | 0.023611272 |
| LY6D | 4 | 0.30518636 | −0.0089658 | 0.31415219 | 0.00318235 | 0.025441401 |
| FOLR2 | 4 | 0.4247 | 0.13146962 | 0.29323038 | 0.00331179 | 0.026332314 |
| IFNGR2 | 4 | 0.75385 | 0.24877468 | 0.50507532 | 0.003446 | 0.027105609 |
| PGF | 4 | 0.36356818 | 0.14391772 | 0.21965046 | 0.0034461 | 0.027105609 |
| IGSF8 | 4 | 0.73723636 | 0.50747595 | 0.22976041 | 0.00363266 | 0.028269835 |
| LTBP2 | 4 | 0.60765909 | 0.20690127 | 0.40075783 | 0.00363276 | 0.028269835 |
| RARRES2 | 4 | 1.93439091 | 1.5749 | 0.35949091 | 0.0037295 | 0.028869082 |
| KLK4 | 4 | 1.02663182 | 0.38066835 | 0.64596346 | 0.00377864 | 0.029021769 |
| FOLR1 | 4 | 0.50620455 | 0.24801519 | 0.25818936 | 0.00382857 | 0.029021769 |
| PLA2G15 | 4 | 0.43827727 | 0.24233165 | 0.19594563 | 0.00382857 | 0.029021769 |
| TFF1 | 4 | 0.30378636 | −0.1122316 | 0.41601801 | 0.00382857 | 0.029021769 |
| CCN3 | 4 | 0.59151818 | 0.21561519 | 0.37590299 | 0.00392993 | 0.029185973 |
| VAMP5 | 4 | 1.48532727 | 1.10681266 | 0.37851461 | 0.00392993 | 0.029185973 |
| NECTIN2 | 4 | 0.69987727 | 0.45549114 | 0.24438613 | 0.00393003 | 0.029185973 |
| GUCA2A | 4 | 0.38676818 | 0.10023418 | 0.286534 | 0.00403393 | 0.02980624 |
| CD70 | 4 | 0.94213636 | 0.56119367 | 0.38094269 | 0.00436063 | 0.03173933 |
| HS6ST1 | 4 | 0.99762273 | 0.75563671 | 0.24198602 | 0.00447491 | 0.032409849 |
| FLT3LG | 4 | 0.32112273 | 0.01039494 | 0.31072779 | 0.00459165 | 0.033091555 |
| ADGRG1 | 4 | 0.61574545 | −0.1095443 | 0.72528976 | 0.00495913 | 0.03539124 |
| ANGPTL4 | 4 | 0.25721364 | −0.2020367 | 0.45925035 | 0.00508723 | 0.035782753 |
| CKAP4 | 4 | 0.8459 | 0.60805823 | 0.23784177 | 0.00508723 | 0.035782753 |
| BTN3A2 | 4 | 1.06839545 | 0.76870633 | 0.29968913 | 0.00508736 | 0.035782753 |
| NCR1 | 4 | 0.44698182 | 0.16965823 | 0.27732359 | 0.00515247 | 0.036067281 |
| DDAH1 | 4 | 0.53816364 | 0.22281646 | 0.31534718 | 0.00549012 | 0.037887927 |
| EPHB6 | 4 | 0.79914545 | 0.59995696 | 0.19918849 | 0.00549012 | 0.037887927 |
| TXNRD1 | 4 | 0.58085 | 0.38649747 | 0.19435253 | 0.00549025 | 0.037887927 |
| CD59 | 4 | 0.37226364 | 0.21583797 | 0.15642566 | 0.00563082 | 0.038315742 |
| CDHR5 | 4 | 0.31666364 | −0.0271481 | 0.34381174 | 0.00563082 | 0.038315742 |
| INPP1 | 4 | 0.06441818 | −0.2003215 | 0.2647397 | 0.00570217 | 0.038621627 |
| ADA | 4 | 0.57742727 | 0.3142 | 0.26322727 | 0.00577462 | 0.038932103 |
| TP53INP1 | 4 | 0.80100455 | 0.61702785 | 0.1839767 | 0.00584761 | 0.039243364 |
| RSPO3 | 4 | 0.38302727 | 0.10927089 | 0.27375639 | 0.00592172 | 0.039559272 |
| PPP1R2 | 4 | 1.21926818 | 0.92006709 | 0.29920109 | 0.0060722 | 0.040380107 |
| SPINK4 | 4 | 1.38998636 | 0.82546076 | 0.5645256 | 0.00622611 | 0.041030597 |
| ULBP2 | 4 | 0.62991364 | 0.36102911 | 0.26888452 | 0.00622611 | 0.041030597 |
| SCARF2 | 4 | 0.39079091 | 0.08835443 | 0.30243648 | 0.00638352 | 0.041879314 |
| COL6A3 | 4 | 0.81292727 | 0.48988228 | 0.32304499 | 0.00654435 | 0.042178888 |
| GNLY | 4 | −0.0401818 | −0.4602443 | 0.42006249 | 0.0065445 | 0.042178888 |
| NBL1 | 4 | 0.30521364 | 0.05728228 | 0.24793136 | 0.0065445 | 0.042178888 |
| POLR2F | 4 | 0.90982727 | 0.61831899 | 0.29150829 | 0.0065445 | 0.042178888 |
| OSMR | 4 | 0.32432273 | 0.17279114 | 0.15153159 | 0.00670897 | 0.042862255 |
| CLEC1A | 4 | 1.21929545 | 1.01266582 | 0.20662963 | 0.00670913 | 0.042862255 |
| PDCD5 | 4 | 1.12924091 | 0.82669241 | 0.3025485 | 0.00687747 | 0.043557306 |
| SCARA5 | 4 | 0.38676818 | 0.0601038 | 0.32666438 | 0.00687747 | 0.043557306 |
| F9 | 4 | 0.12989545 | −0.0583177 | 0.18821318 | 0.00704959 | 0.044264192 |
| FCGR3B | 4 | 0.32409091 | 0.08625949 | 0.23783142 | 0.00704959 | 0.044264192 |
| CASP1 | 4 | 0.69454091 | 0.3042481 | 0.39029281 | 0.00740549 | 0.045714083 |
| EBI3_IL27 | 4 | 0.43923636 | 0.20551772 | 0.23371864 | 0.00740549 | 0.045714083 |
| IGF2R | 4 | 0.44777727 | 0.26756329 | 0.18021398 | 0.00740549 | 0.045714083 |
| PTS | 4 | 0.63629091 | 0.25736076 | 0.37893015 | 0.00740549 | 0.045714083 |
| SIRPB1 | 4 | 0.58335 | 0.30666456 | 0.27668544 | 0.00758924 | 0.046651521 |
| NOS3 | 4 | 1.98990455 | 1.71133165 | 0.2785729 | 0.00777725 | 0.047213179 |
| SH2D1A | 4 | 0.64169091 | 0.17467595 | 0.46701496 | 0.00777725 | 0.047213179 |
| RTN4R | 4 | 0.69312727 | 0.42521772 | 0.26790955 | 0.00777743 | 0.047213179 |
| TINAGL1 | 4 | 0.49503636 | 0.31957215 | 0.17546421 | 0.00796923 | 0.047981576 |
| CPB1 | 4 | 1.07088636 | 0.65990253 | 0.41098383 | 0.00816601 | 0.048962602 |
| CD163 | 4 | 0.25440909 | −0.0683861 | 0.32279517 | 0.00857188 | 0.050978306 |
| CD46 | 4 | 0.48159545 | 0.31452152 | 0.16707394 | 0.00857188 | 0.050978306 |
| PPCDC | 4 | 0.74752727 | 0.47809367 | 0.2694336 | 0.00878131 | 0.051595741 |
| IL15 | 4 | 0.39077273 | 0.23090253 | 0.1598702 | 0.0087815 | 0.051595741 |
| ROBO2 | 4 | 0.12562727 | 0.00857975 | 0.11704753 | 0.0087815 | 0.051595741 |
| NOTCH1 | 4 | 0.4127 | 0.33598481 | 0.07671519 | 0.00888763 | 0.052010383 |
| CD300LF | 4 | 0.56657273 | 0.15358987 | 0.41298285 | 0.00899569 | 0.052224996 |
| IL15RA | 4 | 1.05255909 | 0.78249367 | 0.27006542 | 0.00899569 | 0.052224996 |
| B4GALT1 | 4 | 0.32916818 | 0.16897215 | 0.16019603 | 0.00921454 | 0.052454732 |
| CFC1 | 4 | 0.92356364 | 0.63766835 | 0.28589528 | 0.00921454 | 0.052454732 |
| LBP | 4 | 1.80717727 | 1.47294684 | 0.33423044 | 0.00921454 | 0.052454732 |
| SIGLEC7 | 4 | 0.43675 | 0.23728101 | 0.19946899 | 0.00921454 | 0.052454732 |
| MSLN | 4 | 0.29403182 | −0.0316076 | 0.32563941 | 0.00943812 | 0.053312612 |
| TAFA5 | 4 | 0.93089091 | 0.60123038 | 0.32966053 | 0.00943812 | 0.053312612 |
| MUC13 | 4 | 1.04898182 | 0.73716962 | 0.3118122 | 0.00966631 | 0.053772349 |
| IL18R1 | 4 | 0.39093182 | 0.17036456 | 0.22056726 | 0.00966653 | 0.053772349 |
| PDCD6 | 4 | 0.33288636 | −0.1474658 | 0.48035219 | 0.00966653 | 0.053772349 |
| SLAMF8 | 4 | −0.0799182 | −0.4624291 | 0.38251093 | 0.00966653 | 0.053772349 |
| CXCL1 | 4 | 3.71925 | 3.42393924 | 0.29531076 | 0.00989985 | 0.054449169 |
| RELT | 4 | 0.85219091 | 0.60803544 | 0.24415547 | 0.00989985 | 0.054449169 |
| SCG2 | 4 | 0.35350909 | 0.13935823 | 0.21415086 | 0.00989985 | 0.054449169 |
| CGA | 4 | 1.84225909 | 1.01870759 | 0.8235515 | 0.01013796 | 0.054933895 |
| CCL2 | 4 | 1.13984091 | 0.88065823 | 0.25918268 | 0.01013818 | 0.054933895 |
| ENAH | 4 | 0.96654545 | 0.72646456 | 0.2400809 | 0.01013818 | 0.054933895 |
| GSTA3 | 4 | 0.44943182 | −0.1239089 | 0.57334068 | 0.01013818 | 0.054933895 |
| HSPG2 | 4 | 0.46198636 | 0.21720253 | 0.24478383 | 0.0103816 | 0.056045308 |
| CPA2 | 4 | 0.83651818 | 0.34475949 | 0.49175869 | 0.01050502 | 0.056503124 |
| NRP2 | 4 | 0.36869091 | 0.19872152 | 0.16996939 | 0.01062998 | 0.056759102 |
| DUSP3 | 4 | 1.42420909 | 0.64208734 | 0.78212175 | 0.01063021 | 0.056759102 |
| CCL5 | 4 | 4.85892273 | 4.50101772 | 0.35790501 | 0.0108841 | 0.0579034 |
| TRIAP1 | 4 | 0.34273636 | 0.01348101 | 0.32925535 | 0.01114313 | 0.059066651 |
| NOMO1 | 4 | 0.32155455 | 0.17407089 | 0.14748366 | 0.0114081 | 0.059820965 |
| VWA1 | 4 | 0.71411818 | 0.42745696 | 0.28666122 | 0.0114081 | 0.059820965 |
| VWF | 4 | −0.1958727 | −0.551638 | 0.35576525 | 0.0116784 | 0.060802507 |
| CXCL14 | 4 | −0.6678727 | −0.8505772 | 0.18270449 | 0.01181543 | 0.061297774 |
| GCNT1 | 4 | 1.19317727 | 0.95103418 | 0.2421431 | 0.01195437 | 0.061799471 |
| ITGA5 | 4 | −0.4513455 | −0.5982354 | 0.14688999 | 0.01223611 | 0.062109146 |
| IFNGR1 | 4 | 0.19295455 | 0.08349241 | 0.10946214 | 0.01237841 | 0.062109146 |
| CCDC80 | 4 | 0.78463182 | 0.5195443 | 0.26508751 | 0.01237892 | 0.062109146 |
| NEFL | 4 | 0.7039 | 0.2911038 | 0.4127962 | 0.01237892 | 0.062109146 |
| GPKOW | 4 | 0.65789545 | 0.47946709 | 0.17842837 | 0.01252346 | 0.062109146 |
| CD79B | 4 | 0.53334545 | 0.31349114 | 0.21985432 | 0.01252372 | 0.062109146 |
| CEP85 | 4 | 1.39654545 | 1.06898354 | 0.32756191 | 0.01252372 | 0.062109146 |
| COL18A1 | 4 | 0.6589 | 0.49681392 | 0.16208608 | 0.01252372 | 0.062109146 |
| ENTPD6 | 4 | 0.77338182 | 0.60210506 | 0.17127675 | 0.01252372 | 0.062109146 |
| HNMT | 4 | 0.56802273 | 0.17816709 | 0.38985564 | 0.01252372 | 0.062109146 |
| SORD | 4 | −0.1881682 | −0.7418089 | 0.55364068 | 0.01252372 | 0.062109146 |
| MDK | 4 | 0.55788182 | 0.29707089 | 0.26081093 | 0.01281729 | 0.06292516 |
| PRSS8 | 4 | 0.07458636 | −0.2667329 | 0.34131928 | 0.01281729 | 0.06292516 |
| MAD1L1 | 4 | 1.17426818 | 0.80164177 | 0.37262641 | 0.01311694 | 0.063966941 |
| NTproBNP | 4 | 1.03816364 | 0.41076329 | 0.62740035 | 0.01311694 | 0.063966941 |
| LY9 | 4 | 0.36341364 | 0.18027089 | 0.18314275 | 0.01357776 | 0.065775685 |
| PTGDS | 4 | 0.31445 | 0.06687215 | 0.24757785 | 0.01357776 | 0.065775685 |
| CCL27 | 4 | 2.20810455 | 1.89984304 | 0.30826151 | 0.01373488 | 0.066099091 |
| FGF23 | 4 | −0.8374 | −1.1651114 | 0.32771139 | 0.01373488 | 0.066099091 |
| CD4 | 4 | 0.26260909 | 0.1500557 | 0.11255339 | 0.01405338 | 0.06675355 |
| DEFA1_DEFA1B | 4 | 0.60982273 | 0.34507722 | 0.26474551 | 0.01405338 | 0.06675355 |
| FCGR2A | 4 | 0.08221818 | −0.1859025 | 0.26812071 | 0.01405338 | 0.06675355 |
| ACVRL1 | 4 | 0.84393636 | 0.65544937 | 0.188487 | 0.01437838 | 0.06785669 |
| PCDH17 | 4 | 0.33575455 | 0.13046582 | 0.20528872 | 0.01437838 | 0.06785669 |
| LDLR | 4 | −0.1367636 | −0.5399304 | 0.40316674 | 0.01471 | 0.069198478 |
| CPM | 4 | 0.41665909 | 0.13038987 | 0.28626922 | 0.01504834 | 0.070337747 |
| TFF2 | 4 | 0.59058182 | 0.11899114 | 0.47159068 | 0.01504834 | 0.070337747 |
| LRRC25 | 4 | 0.68970909 | 0.47346329 | 0.2162458 | 0.01521976 | 0.070912468 |
| PCSK9 | 4 | 0.77193182 | 0.47596203 | 0.29596979 | 0.01539291 | 0.07126805 |
| PTK7 | 4 | 0.59205909 | 0.34319114 | 0.24886795 | 0.01539351 | 0.07126805 |
| CDH2 | 4 | 0.49336364 | 0.15190759 | 0.34145604 | 0.01574533 | 0.072439814 |
| GPR37 | 4 | 1.02376364 | 0.66292532 | 0.36083832 | 0.01610482 | 0.073629234 |
| LILRB2 | 4 | 0.68128636 | 0.45800127 | 0.2232851 | 0.01610482 | 0.073629234 |
| IL1RL2 | 4 | 0.48238636 | 0.26102152 | 0.22136484 | 0.01647119 | 0.074836513 |
| PSME2 | 4 | 0.91333182 | 0.69993671 | 0.21339511 | 0.01665679 | 0.075445479 |
| THOP1 | 4 | 0.19084091 | 0.02572785 | 0.16511306 | 0.01722564 | 0.076834088 |
| FURIN | 4 | 0.66341364 | 0.38504304 | 0.2783706 | 0.01722596 | 0.076834088 |
| CALB1 | 4 | 0.48252727 | 0.23821266 | 0.24431461 | 0.0176146 | 0.078091377 |
| SERPINB8 | 4 | 1.24231364 | 0.85373924 | 0.3885744 | 0.0176146 | 0.078091377 |
| CPA1 | 4 | 1.20900455 | 0.82562911 | 0.38337543 | 0.01841464 | 0.080181907 |
| CHI3L1 | 4 | 1.10017273 | 0.80705823 | 0.2931145 | 0.01841498 | 0.080181907 |
| KLK11 | 4 | 0.11470909 | −0.1042582 | 0.21896732 | 0.01841498 | 0.080181907 |
| SHMT1 | 4 | 0.13254091 | −0.3269481 | 0.45948901 | 0.01841498 | 0.080181907 |
| KYNU | 4 | 0.49017273 | 0.15498734 | 0.33518539 | 0.01882698 | 0.081732575 |
| CCL4 | 4 | 1.64624545 | 1.32863671 | 0.31760875 | 0.01924667 | 0.082094451 |
| CXCL17 | 4 | 0.46255909 | 0.12406203 | 0.33849707 | 0.01924667 | 0.082094451 |
| BCAM | 4 | 0.37405455 | 0.20766329 | 0.16639125 | 0.01924702 | 0.082094451 |
| MMP12 | 4 | 0.15049091 | −0.3311582 | 0.48164914 | 0.01924702 | 0.082094451 |
| MVK | 4 | 0.93046364 | 0.60255063 | 0.327913 | 0.01967523 | 0.08343439 |
| EPS8L2 | 4 | 0.38646364 | 0.15802911 | 0.22843452 | 0.02011101 | 0.084793864 |
| IL1R1 | 4 | 0.59337273 | 0.48704051 | 0.10633222 | 0.02011174 | 0.084793864 |
| DPT | 4 | 0.21310455 | −0.037838 | 0.25094252 | 0.02055667 | 0.085926875 |
| LTBR | 4 | 0.22547727 | 0.01628608 | 0.2091912 | 0.02055667 | 0.085926875 |
| TIMP1 | 4 | 1.29070909 | 1.14467342 | 0.14603567 | 0.02055667 | 0.085926875 |
| PSME1 | 4 | 1.19845909 | 0.9714481 | 0.22701099 | 0.02100979 | 0.086830125 |
| CCL8 | 4 | −2.02495 | −2.2675937 | 0.24264367 | 0.02101016 | 0.086830125 |
| GBP2 | 4 | −0.3237955 | −0.5266494 | 0.20285391 | 0.02101016 | 0.086830125 |
| GRPEL1 | 4 | 0.90726818 | 0.57280253 | 0.33446565 | 0.02101016 | 0.086830125 |
| FMNL1 | 4 | 2.25702727 | 1.89426076 | 0.36276651 | 0.02147234 | 0.087994509 |
| SERPINA9 | 4 | 0.79493636 | 0.37562152 | 0.41931484 | 0.02147234 | 0.087994509 |
| SLITRK2 | 4 | 0.68026818 | 0.45293165 | 0.22733654 | 0.02147234 | 0.087994509 |
| BSG | 4 | 0.27007273 | 0.12246582 | 0.1476069 | 0.02170636 | 0.088705023 |
| TIGAR | 4 | 0.95062727 | 0.72721899 | 0.22340829 | 0.02194297 | 0.088928338 |
| ANXA5 | 4 | 0.33185 | 0.17542785 | 0.15642215 | 0.02194336 | 0.088928338 |
| IL18 | 4 | 0.02471818 | −0.2347633 | 0.25948147 | 0.02242294 | 0.090372822 |
| LCN2 | 4 | 0.77870455 | 0.52052658 | 0.25817796 | 0.02242333 | 0.090372822 |
| SPON2 | 4 | 0.98952273 | 0.71859114 | 0.27093159 | 0.02291241 | 0.092090257 |
| LILRA5 | 4 | 0.41300455 | 0.23859367 | 0.17441087 | 0.02341072 | 0.093578936 |
| TNFRSF19 | 4 | 0.91015909 | 0.69057215 | 0.21958694 | 0.02341072 | 0.093578936 |
| EIF5A | 4 | 0.00636818 | −0.1290722 | 0.13544033 | 0.02366299 | 0.094319814 |
| BRK1 | 4 | 0.68538182 | 0.48057468 | 0.20480713 | 0.02391842 | 0.094319814 |
| EDA2R | 4 | 0.59440455 | 0.16942278 | 0.42498176 | 0.02391842 | 0.094319814 |
| ELOA | 4 | 1.5389 | 1.17159241 | 0.36730759 | 0.02391842 | 0.094319814 |
| XCL1 | 4 | 0.73665 | 0.39074684 | 0.34590316 | 0.02391842 | 0.094319814 |
| CREG1 | 4 | 0.67128182 | 0.47642911 | 0.1948527 | 0.02417542 | 0.094822087 |
| LXN | 4 | 0.3759 | 0.20576835 | 0.17013165 | 0.02417542 | 0.094822087 |
| CCL18 | 4 | 1.04149091 | 0.60172532 | 0.43976559 | 0.02496252 | 0.096870483 |
| CD99L2 | 4 | 0.60784091 | 0.44843671 | 0.1594042 | 0.02496252 | 0.096870483 |
| CES3 | 4 | −0.1914 | −0.4412 | 0.2498 | 0.02496252 | 0.096870483 |
| GSTA1 | 4 | 0.1727 | −0.5487418 | 0.72144177 | 0.02496252 | 0.096870483 |
| CAPG | 4 | 0.51766818 | 0.04016582 | 0.47750236 | 0.02549922 | 0.09843101 |
| TFPI | 4 | 0.15455455 | 0.01718987 | 0.13736467 | 0.02549922 | 0.09843101 |
| CCL7 | 4 | 1.29004091 | 1.00856962 | 0.28147129 | 0.02604586 | 0.099232023 |
| CRELD2 | 4 | 0.78821818 | 0.57328481 | 0.21493337 | 0.02604586 | 0.099232023 |
| GAS6 | 4 | 0.54651818 | 0.42737975 | 0.11913843 | 0.02604586 | 0.099232023 |
| IL6R | 4 | 0.19050909 | −0.0131051 | 0.20361415 | 0.02604586 | 0.099232023 |
| TNFRSF12A | 4 | 0.675 | 0.4176038 | 0.2573962 | 0.02604586 | 0.099232023 |
| PAM | 4 | 1.33281364 | 1.22261772 | 0.11019591 | 0.02660173 | 0.100567492 |
| IL4 | 4 | 0.62466818 | 0.1600962 | 0.46457198 | 0.02660261 | 0.100567492 |
| NT5E | 4 | 0.15443182 | −0.1711367 | 0.32556853 | 0.02660261 | 0.100567492 |
| APBB1IP | 4 | 2.14053636 | 1.76379367 | 0.37674269 | 0.02716961 | 0.102182872 |
| TIE1 | 4 | 0.3915 | 0.17287595 | 0.21862405 | 0.02716961 | 0.102182872 |
| CLPS | 4 | 1.70096818 | 1.33845823 | 0.36250995 | 0.02774701 | 0.103820638 |
| CTSZ | 4 | 0.22967727 | 0.00945063 | 0.22022664 | 0.02774701 | 0.103820638 |
| FCRL2 | 4 | 0.15329545 | −0.1549722 | 0.30826761 | 0.02833495 | 0.105213292 |
| OGFR | 4 | 0.93560909 | 0.76662911 | 0.16897998 | 0.02833495 | 0.105213292 |
| EIF4EBP1 | 4 | 1.13867273 | 0.86103165 | 0.27764108 | 0.02863247 | 0.10604886 |
| FST | 4 | 0.43654545 | 0.18054304 | 0.25600242 | 0.0289336 | 0.106624338 |
| PAG1 | 4 | 1.52495909 | 1.25976076 | 0.26519833 | 0.0289336 | 0.106624338 |
| IL17A | 4 | −0.0997955 | −0.4801759 | 0.38038049 | 0.02954311 | 0.108324736 |
| QDPR | 4 | 0.13733182 | −0.058543 | 0.19587486 | 0.02985151 | 0.109181881 |
| FLT4 | 4 | 0.52595455 | 0.32751013 | 0.19844442 | 0.03016363 | 0.109774593 |
| LHB | 4 | −2.4676636 | −2.8373139 | 0.36965029 | 0.03016363 | 0.109774593 |
| FLT1 | 4 | 0.15772727 | 0.08205443 | 0.07567284 | 0.03143783 | 0.113847186 |
| MMP1 | 4 | 3.49542727 | 3.19626962 | 0.29915765 | 0.03143832 | 0.113847186 |
| CD300LG | 4 | 0.65366818 | 0.38383544 | 0.26983274 | 0.03209281 | 0.114796541 |
| CHIT1 | 4 | −0.1379409 | −0.423043 | 0.28510213 | 0.03209281 | 0.114796541 |
| CNTN2 | 4 | 0.72643182 | 0.40898101 | 0.31745081 | 0.03209281 | 0.114796541 |
| MOG | 4 | 0.73425909 | 0.45572911 | 0.27852998 | 0.03209281 | 0.114796541 |
| FCRL1 | 4 | 0.39508636 | 0.20938481 | 0.18570155 | 0.03275895 | 0.116044411 |
| ITGAM | 4 | 0.56923182 | 0.3915038 | 0.17772802 | 0.03275895 | 0.116044411 |
| SLITRK6 | 4 | 0.73832727 | 0.61076709 | 0.12756018 | 0.03275895 | 0.116044411 |
| VEGFA | 4 | 2.28454545 | 1.96745696 | 0.31708849 | 0.03275895 | 0.116044411 |
| CD93 | 4 | 0.40362273 | 0.2376557 | 0.16596703 | 0.03343586 | 0.117309756 |
| IL17F | 4 | 0.62298636 | 0.43000506 | 0.1929813 | 0.03343637 | 0.117309756 |
| NUDC | 4 | 0.79190455 | 0.59594684 | 0.19595771 | 0.03343689 | 0.117309756 |
| ANGPT2 | 4 | 0.56383182 | 0.38726835 | 0.17656346 | 0.0341268 | 0.119158717 |
| TFRC | 4 | 0.19129545 | 0.01218481 | 0.17911064 | 0.0348283 | 0.119611699 |
| CHEK2 | 4 | 0.14956364 | 0.02334557 | 0.12621807 | 0.03482883 | 0.119611699 |
| ENO1 | 4 | 1.10811818 | 0.76165696 | 0.34646122 | 0.03482883 | 0.119611699 |
| NADK | 4 | 2.92432727 | 2.4846557 | 0.43967158 | 0.03482883 | 0.119611699 |
| OXT | 4 | 0.32635 | −0.6293823 | 0.95573228 | 0.03482883 | 0.119611699 |
| PLAT | 4 | 0.02184091 | −0.3506013 | 0.37244217 | 0.03482883 | 0.119611699 |
| RBP2 | 4 | 0.51600455 | 0.06183797 | 0.45416657 | 0.03554263 | 0.120369569 |
| CALB2 | 4 | 0.49671364 | 0.30195949 | 0.19475414 | 0.03554317 | 0.120369569 |
| CANT1 | 4 | 0.69984091 | 0.60506962 | 0.09477129 | 0.03554317 | 0.120369569 |
| CCL13 | 4 | 1.94825909 | 1.57794304 | 0.37031605 | 0.03554317 | 0.120369569 |
| RNASE3 | 4 | 7.49938636 | 6.7113443 | 0.78804206 | 0.03554317 | 0.120369569 |
| TNC | 4 | 0.65319545 | 0.46150253 | 0.19169292 | 0.03554317 | 0.120369569 |
| CNTNAP2 | 4 | 0.88614545 | 0.6273 | 0.25884545 | 0.03626996 | 0.121983806 |
| FOLR3 | 4 | −2.4046545 | −2.7972975 | 0.39264292 | 0.03626996 | 0.121983806 |
| HAO1 | 4 | 0.4607 | −0.4089468 | 0.86964684 | 0.03626996 | 0.121983806 |
| IGSF3 | 4 | 0.48864545 | 0.33501772 | 0.15362773 | 0.03700939 | 0.124185169 |
| PRTN3 | 4 | 1.90238182 | 1.55071266 | 0.35166916 | 0.03776161 | 0.126419302 |
| ADAM22 | 4 | 0.71540909 | 0.48239873 | 0.23301036 | 0.0385268 | 0.128101608 |
| CD302 | 4 | 0.74050909 | 0.5074962 | 0.23301289 | 0.0385268 | 0.128101608 |
| PXN | 4 | 2.75227727 | 2.2833481 | 0.46892917 | 0.0385268 | 0.128101608 |
| AGR2 | 4 | 1.27536818 | 0.60622785 | 0.66914033 | 0.03930513 | 0.129512166 |
| DLK1 | 4 | 0.18452273 | −0.1252468 | 0.30976956 | 0.03930513 | 0.129512166 |
| PI3 | 4 | 0.57128182 | 0.22147089 | 0.34981093 | 0.0409013 | 0.132388188 |
| FOPNL | 4 | 0.10035909 | −0.0819443 | 0.18230339 | 0.04090189 | 0.132388188 |
| LEPR | 4 | 0.69251364 | 0.49707848 | 0.19543516 | 0.04090189 | 0.132388188 |
| MZB1 | 4 | −0.0177318 | −0.2778937 | 0.26016185 | 0.04090189 | 0.132388188 |
| SKAP1 | 4 | 0.95727273 | 0.58342785 | 0.37384488 | 0.04090189 | 0.132388188 |
| TXNDC15 | 4 | 0.09648182 | −0.0191165 | 0.11559827 | 0.04090189 | 0.132388188 |
| NUCB2 | 4 | 1.15606364 | 1.02641392 | 0.12964971 | 0.04172067 | 0.134147989 |
| TXLNA | 4 | 1.70544091 | 1.42424304 | 0.28119787 | 0.04172067 | 0.134147989 |
| CASP8 | 4 | 2.04481818 | 1.76038734 | 0.28443084 | 0.04255328 | 0.136226361 |
| CTSD | 4 | 0.13590909 | 0.03606456 | 0.09984453 | 0.04255328 | 0.136226361 |
| IPCEF1 | 4 | 3.31197727 | 2.83375823 | 0.47821904 | 0.0433999 | 0.138030552 |
| KITLG | 4 | 0.51557727 | 0.34768608 | 0.1678912 | 0.0433999 | 0.138030552 |
| KYAT1 | 4 | 0.74825 | 0.48643038 | 0.26181962 | 0.0433999 | 0.138030552 |
| CASP10 | 4 | 2.00977273 | 1.65319873 | 0.35657399 | 0.04426071 | 0.140462942 |
| TXNDC5 | 4 | 0.42990455 | 0.25863671 | 0.17126784 | 0.04469587 | 0.141231215 |
| CCL23 | 4 | 1.11199545 | 0.95630506 | 0.15569039 | 0.04784939 | 0.150222453 |
| CTRB1 | 4 | 1.26322273 | 0.92652405 | 0.33669868 | 0.04831406 | 0.151356475 |
| ACAN | 4 | 0.22593636 | 0.01389367 | 0.21204269 | 0.04878383 | 0.151852654 |
| FABP5 | 4 | 1.22043636 | 0.77461899 | 0.44581738 | 0.04878383 | 0.151852654 |
| NOTCH3 | 4 | 0.52263636 | 0.34421266 | 0.17842371 | 0.04878383 | 0.151852654 |
| C1QA | 4 | 0.31376818 | 0.16588354 | 0.14788464 | 0.04973354 | 0.153827011 |
| NMNAT1 | 4 | 3.25897727 | 2.76887975 | 0.49009753 | 0.04973354 | 0.153827011 |
| VAT1 | 4 | 0.14750909 | 0.05090253 | 0.09660656 | 0.04973354 | 0.153827011 |
| ANXA11 | 5 | 2.99842 | 1.91312099 | 1.08529901 | 4.37E−08 | 6.39E−05 |
| HGF | 5 | 1.66484 | 1.04015926 | 0.62468074 | 1.24E−07 | 9.06E−05 |
| ANXA3 | 5 | 1.911405 | 0.74818025 | 1.16322475 | 2.96E−07 | 0.000118729 |
| SAMD9L | 5 | 2.61727 | 1.72258272 | 0.89468728 | 3.39E−07 | 0.000118729 |
| MSRA | 5 | 1.85529 | 1.10635432 | 0.74893568 | 4.06E−07 | 0.000118729 |
| AXIN1 | 5 | 2.23942 | 1.5349716 | 0.7044484 | 6.91E−07 | 0.000168409 |
| SNAP23 | 5 | 2.84602 | 1.61326049 | 1.23275951 | 1.24E−06 | 0.000206147 |
| ERBIN | 5 | 2.17826 | 1.36452593 | 0.81373407 | 1.27E−06 | 0.000206147 |
| LBR | 5 | 3.38229 | 2.26276049 | 1.11952951 | 1.27E−06 | 0.000206147 |
| SCAMP3 | 5 | 2.780105 | 1.69277407 | 1.08733093 | 1.57E−06 | 0.000206147 |
| FKBP5 | 5 | 3.744875 | 2.46755556 | 1.27731944 | 1.86E−06 | 0.000206147 |
| DNAJA2 | 5 | 3.9997925 | 2.89650802 | 1.10328448 | 1.94E−06 | 0.000206147 |
| MMP8 | 5 | 4.3178025 | 2.64932037 | 1.66848213 | 1.94E−06 | 0.000206147 |
| MAP3K5 | 5 | 3.13254 | 2.04215802 | 1.09038198 | 2.02E−06 | 0.000206147 |
| SERPINB1 | 5 | 2.2578675 | 1.29129444 | 0.96657306 | 2.20E−06 | 0.000206147 |
| STK4 | 5 | 0.96891 | 0.49702963 | 0.47188037 | 2.30E−06 | 0.000206147 |
| MESD | 5 | 0.871955 | 0.50237531 | 0.36957969 | 2.40E−06 | 0.000206147 |
| PTPN1 | 5 | 2.92758 | 2.03537284 | 0.89220716 | 2.72E−06 | 0.000217938 |
| AIF1 | 5 | 0.9775 | 0.25843457 | 0.71906543 | 2.83E−06 | 0.000217938 |
| DBNL | 5 | 3.742 | 2.60779012 | 1.13420988 | 3.34E−06 | 0.000242563 |
| ICA1 | 5 | 1.514525 | 0.9296037 | 0.5849213 | 3.48E−06 | 0.000242563 |
| DNMBP | 5 | 2.27268 | 1.49657654 | 0.77610346 | 3.94E−06 | 0.000261026 |
| STX8 | 5 | 1.15065 | 0.56319259 | 0.58745741 | 4.10E−06 | 0.000261026 |
| MNDA | 5 | 6.200785 | 4.75180988 | 1.44897512 | 4.83E−06 | 0.000273819 |
| DAB2 | 5 | 1.41897 | 0.91048889 | 0.50848111 | 5.03E−06 | 0.000273819 |
| PAG1 | 5 | 1.820565 | 1.19331975 | 0.62724525 | 5.03E−06 | 0.000273819 |
| PPP1R12A | 5 | 3.2377 | 2.33851728 | 0.89918272 | 5.14E−06 | 0.000273819 |
| SNAP29 | 5 | 3.80606 | 2.82425432 | 0.98180568 | 5.24E−06 | 0.000273819 |
| CRACR2A | 5 | 4.10304 | 2.93417407 | 1.16886593 | 5.46E−06 | 0.000275288 |
| CDKN2D | 5 | 2.427125 | 1.1829037 | 1.2442213 | 5.92E−06 | 0.000279026 |
| FADD | 5 | 2.41332 | 1.80517901 | 0.60814099 | 5.92E−06 | 0.000279026 |
| FYB1 | 5 | 2.5427525 | 1.74702901 | 0.79572349 | 6.16E−06 | 0.000279026 |
| MPO | 5 | 2.721715 | 1.78830741 | 0.93340759 | 6.41E−06 | 0.000279026 |
| NFATC1 | 5 | 2.3729625 | 1.66004259 | 0.71291991 | 6.68E−06 | 0.000279026 |
| YES1 | 5 | 2.75843 | 1.9972716 | 0.7611584 | 6.68E−06 | 0.000279026 |
| FGR | 5 | 2.57896 | 1.65525185 | 0.92370815 | 6.95E−06 | 0.0002795 |
| APEX1 | 5 | 3.5447225 | 2.42384259 | 1.12087991 | 7.09E−06 | 0.0002795 |
| PDLIM7 | 5 | 3.34302 | 2.0766037 | 1.2664163 | 7.53E−06 | 0.0002795 |
| CEACAM8 | 5 | 2.55302 | 1.70033827 | 0.85268173 | 7.83E−06 | 0.0002795 |
| CORO1A | 5 | 3.6889675 | 2.64439444 | 1.04457306 | 7.83E−06 | 0.0002795 |
| TACC3 | 5 | 2.695905 | 1.83946914 | 0.85643586 | 7.83E−06 | 0.0002795 |
| CIAPIN1 | 5 | 2.428065 | 1.58374815 | 0.84431685 | 8.48E−06 | 0.000295435 |
| OSM | 5 | 3.0695125 | 2.04225 | 1.0272625 | 9.55E−06 | 0.000324962 |
| EGF | 5 | 5.60062 | 4.78366543 | 0.81695457 | 1.07E−05 | 0.00035741 |
| PRDX5 | 5 | 3.64506 | 2.80980741 | 0.83525259 | 1.16E−05 | 0.000377979 |
| ATOX1 | 5 | 2.0907775 | 1.30229444 | 0.78848306 | 1.36E−05 | 0.000423 |
| GP6 | 5 | 3.2602 | 2.43830741 | 0.82189259 | 1.36E−05 | 0.000423 |
| IRAK1 | 5 | 1.486435 | 0.94207901 | 0.54435599 | 1.47E−05 | 0.00043847 |
| NFKBIE | 5 | 1.5295 | 1.06132469 | 0.46817531 | 1.47E−05 | 0.00043847 |
| IPCEF1 | 5 | 3.7898 | 2.72758519 | 1.06221481 | 1.59E−05 | 0.000455137 |
| TBCB | 5 | 2.91495 | 1.98605926 | 0.92889074 | 1.59E−05 | 0.000455137 |
| TBC1D23 | 5 | 2.75645 | 1.90554321 | 0.85090679 | 1.65E−05 | 0.000463929 |
| NAMPT | 5 | 2.768435 | 1.85051852 | 0.91791648 | 1.71E−05 | 0.000473034 |
| MYO9B | 5 | 1.305945 | 0.79530617 | 0.51063883 | 1.85E−05 | 0.000483409 |
| RWDD1 | 5 | 1.609785 | 1.1658037 | 0.4439813 | 1.85E−05 | 0.000483409 |
| S100A11 | 5 | 1.8932 | 1.2348358 | 0.6583642 | 1.85E−05 | 0.000483409 |
| DBI | 5 | 1.5944275 | 0.92764012 | 0.66678738 | 2.00E−05 | 0.000512675 |
| DNAJB8 | 5 | 1.32621 | 0.64497284 | 0.68123716 | 2.08E−05 | 0.000519961 |
| PTPN6 | 5 | 2.326865 | 1.48976914 | 0.83709586 | 2.16E−05 | 0.000519961 |
| CD40LG | 5 | 1.93042 | 1.56850123 | 0.36191877 | 2.24E−05 | 0.000519961 |
| MANF | 5 | 3.94357 | 3.06188272 | 0.88168728 | 2.24E−05 | 0.000519961 |
| OLR1 | 5 | 3.575595 | 2.52784938 | 1.04774562 | 2.24E−05 | 0.000519961 |
| S100A12 | 5 | 3.315755 | 2.25764321 | 1.05811179 | 2.24E−05 | 0.000519961 |
| TBC1D5 | 5 | 2.2242875 | 1.46893519 | 0.75535231 | 2.33E−05 | 0.000529275 |
| SORT1 | 5 | 1.699555 | 1.34453704 | 0.35501796 | 2.42E−05 | 0.000529275 |
| ANXA4 | 5 | 1.17308 | 0.74143951 | 0.43164049 | 2.51E−05 | 0.000529275 |
| IL1RN | 5 | 1.04703 | 0.23507778 | 0.81195222 | 2.51E−05 | 0.000529275 |
| LAT2 | 5 | 1.87618 | 1.32428642 | 0.55189358 | 2.51E−05 | 0.000529275 |
| BAX | 5 | 2.838415 | 1.81293704 | 1.02547796 | 2.60E−05 | 0.000529275 |
| CLEC1B | 5 | 3.5711725 | 3.0040463 | 0.5671262 | 2.60E−05 | 0.000529275 |
| LCN2 | 5 | 1.0127525 | 0.46911173 | 0.54364077 | 2.60E−05 | 0.000529275 |
| MAP2K6 | 5 | 3.195255 | 1.96678395 | 1.22847105 | 2.60E−05 | 0.000529275 |
| NBN | 5 | 1.539975 | 0.60277778 | 0.93719722 | 2.70E−05 | 0.000542053 |
| ARHGEF12 | 5 | 2.883985 | 1.92189259 | 0.96209241 | 2.81E−05 | 0.000555204 |
| MAVS | 5 | 1.5304 | 1.06692346 | 0.46347654 | 2.92E−05 | 0.000561256 |
| BID | 5 | 1.4454225 | 0.58366605 | 0.86175645 | 2.92E−05 | 0.000561256 |
| IRAK4 | 5 | 2.38975 | 1.72863333 | 0.66111667 | 3.39E−05 | 0.000642513 |
| COMT | 5 | 1.366645 | 0.71810864 | 0.64853636 | 3.45E−05 | 0.000642513 |
| SIAE | 5 | 2.630425 | 2.10432469 | 0.52610031 | 3.51E−05 | 0.000642513 |
| TGFA | 5 | 2.163785 | 1.39512346 | 0.76866154 | 3.51E−05 | 0.000642513 |
| PMVK | 5 | 4.31971 | 3.01211728 | 1.30759272 | 3.58E−05 | 0.000646431 |
| HCLS1 | 5 | 2.852025 | 2.05788889 | 0.79413611 | 3.78E−05 | 0.000652719 |
| MIF | 5 | 1.7928125 | 1.12688704 | 0.66592546 | 3.78E−05 | 0.000652719 |
| NCF2 | 5 | 2.6378375 | 1.42868827 | 1.20914923 | 3.78E−05 | 0.000652719 |
| BACH1 | 5 | 2.735145 | 1.89720123 | 0.83794377 | 3.93E−05 | 0.000652719 |
| CRKL | 5 | 3.08063 | 2.05511728 | 1.02551272 | 3.93E−05 | 0.000652719 |
| HSPA1A | 5 | 0.685605 | 0.06611728 | 0.61948772 | 3.93E−05 | 0.000652719 |
| SERPINB8 | 5 | 1.5904275 | 0.77737963 | 0.81304787 | 3.93E−05 | 0.000652719 |
| NADK | 5 | 3.39199 | 2.38003951 | 1.01195049 | 4.07E−05 | 0.000669634 |
| TNFSF14 | 5 | 3.34725 | 2.56678025 | 0.78046975 | 4.23E−05 | 0.000687026 |
| CC2D1A | 5 | 2.333955 | 1.47123333 | 0.86272167 | 4.38E−05 | 0.000697247 |
| PRTN3 | 5 | 2.4654025 | 1.4203784 | 1.0450241 | 4.38E−05 | 0.000697247 |
| EGLN1 | 5 | 3.22705 | 2.1306037 | 1.0964463 | 4.55E−05 | 0.000700453 |
| FOXO1 | 5 | 1.854765 | 1.30579012 | 0.54897488 | 4.55E−05 | 0.000700453 |
| MGMT | 5 | 1.5470675 | 0.76352284 | 0.78354466 | 4.55E−05 | 0.000700453 |
| ARG1 | 5 | 1.5355225 | 0.76313889 | 0.77238361 | 4.72E−05 | 0.000719001 |
| FABP5 | 5 | 1.267885 | 0.77391111 | 0.49397389 | 5.46E−05 | 0.000782875 |
| IKBKG | 5 | 3.6443325 | 2.82888333 | 0.81544917 | 5.46E−05 | 0.000782875 |
| ILKAP | 5 | 1.22336 | 0.62338148 | 0.59997852 | 5.46E−05 | 0.000782875 |
| MMP9 | 5 | 3.2345 | 2.6088 | 0.6257 | 5.46E−05 | 0.000782875 |
| PIK3AP1 | 5 | 4.2232925 | 3.31164877 | 0.91164373 | 5.46E−05 | 0.000782875 |
| USP8 | 5 | 0.12211 | −0.4242333 | 0.54634333 | 5.46E−05 | 0.000782875 |
| EREG | 5 | 3.89002 | 2.89490494 | 0.99511506 | 6.08E−05 | 0.000864188 |
| DAG1 | 5 | 1.35375 | 0.90615185 | 0.44759815 | 6.31E−05 | 0.000878847 |
| SCARF1 | 5 | 1.85246 | 1.53298889 | 0.31947111 | 6.31E−05 | 0.000878847 |
| GOPC | 5 | 1.21344 | 0.72033333 | 0.49310667 | 6.54E−05 | 0.00088574 |
| RARRES2 | 5 | 2.045125 | 1.55643457 | 0.48869043 | 6.54E−05 | 0.00088574 |
| TNFSF11 | 5 | 0.3282175 | −0.4539994 | 0.78221688 | 6.54E−05 | 0.00088574 |
| NMNAT1 | 5 | 3.77071 | 2.65462716 | 1.11608284 | 6.78E−05 | 0.000909706 |
| FEN1 | 5 | 4.596035 | 3.47241358 | 1.12362142 | 7.03E−05 | 0.000925917 |
| RETN | 5 | 1.078415 | 0.46129383 | 0.61712117 | 7.03E−05 | 0.000925917 |
| BCR | 5 | 1.5559425 | 1.09995617 | 0.45598633 | 7.55E−05 | 0.000968355 |
| CASP2 | 5 | 2.2235025 | 1.25281173 | 0.97069077 | 7.55E−05 | 0.000968355 |
| SRC | 5 | 3.0159075 | 2.02364136 | 0.99226614 | 7.55E−05 | 0.000968355 |
| S100P | 5 | 3.023145 | 2.25567778 | 0.76746722 | 7.68E−05 | 0.00096947 |
| PPP1R9B | 5 | 2.76295 | 2.2061 | 0.55685 | 7.82E−05 | 0.00096947 |
| C2CD2L | 5 | 1.52672 | 0.91579012 | 0.61092988 | 7.82E−05 | 0.00096947 |
| METAP2 | 5 | 1.4653425 | 0.98166111 | 0.48368139 | 7.82E−05 | 0.00096947 |
| ABL1 | 5 | 1.544315 | 1.09661728 | 0.44769772 | 8.10E−05 | 0.00098783 |
| EIF4G1 | 5 | 2.5438075 | 1.93863889 | 0.60516861 | 8.10E−05 | 0.00098783 |
| AZU1 | 5 | 4.491655 | 3.4060963 | 1.0855587 | 8.40E−05 | 0.001006748 |
| VPS53 | 5 | 1.25901 | 0.85104938 | 0.40796062 | 8.40E−05 | 0.001006748 |
| DPY30 | 5 | 0.10316 | −0.4457765 | 0.54893654 | 8.70E−05 | 0.001034576 |
| MED18 | 5 | 1.393715 | 0.98963333 | 0.40408167 | 9.01E−05 | 0.001054665 |
| PLPBP | 5 | 1.694965 | 1.20447531 | 0.49048969 | 9.01E−05 | 0.001054665 |
| APBB1IP | 5 | 2.475545 | 1.69037778 | 0.78516722 | 9.33E−05 | 0.00108388 |
| CPPED1 | 5 | 1.2987475 | 0.7028537 | 0.5958938 | 9.67E−05 | 0.001096627 |
| DOK2 | 5 | 2.39998 | 1.45504568 | 0.94493432 | 9.67E−05 | 0.001096627 |
| LSP1 | 5 | 2.975395 | 2.28476543 | 0.69062957 | 9.67E−05 | 0.001096627 |
| ARSB | 5 | 3.63352 | 3.05018148 | 0.58333852 | 0.00010015 | 0.001118521 |
| RNF41 | 5 | 1.365575 | 0.88842222 | 0.47715278 | 0.00010015 | 0.001118521 |
| CSF3 | 5 | 0.469865 | 0.04100741 | 0.42885759 | 0.00010373 | 0.00114104 |
| WWP2 | 5 | 1.306605 | 0.81613951 | 0.49046549 | 0.00010373 | 0.00114104 |
| GYS1 | 5 | 2.57578 | 1.73911605 | 0.83666395 | 0.00011125 | 0.001214596 |
| NSFL1C | 5 | 2.0709 | 1.46358889 | 0.60731111 | 0.0001152 | 0.001248388 |
| DCTN1 | 5 | 3.1460275 | 2.60484383 | 0.54118367 | 0.00012349 | 0.001309207 |
| DECR1 | 5 | 1.3385075 | 0.6975821 | 0.6409254 | 0.00012349 | 0.001309207 |
| NT5C3A | 5 | 2.737235 | 2.01705802 | 0.72017698 | 0.00012349 | 0.001309207 |
| ARHGAP25 | 5 | 3.932625 | 3.1003963 | 0.8322287 | 0.00012785 | 0.001336031 |
| CEBPB | 5 | 2.521575 | 1.77502963 | 0.74654537 | 0.00012785 | 0.001336031 |
| TREML2 | 5 | 1.2400725 | 0.8940821 | 0.3459904 | 0.00014181 | 0.001471353 |
| HEXIM1 | 5 | 1.29155 | 0.82255432 | 0.46899568 | 0.00016265 | 0.001664061 |
| MPIG6B | 5 | 3.41546 | 2.78555309 | 0.62990691 | 0.00016265 | 0.001664061 |
| HDGF | 5 | 3.6603075 | 2.35187469 | 1.30843281 | 0.0001683 | 0.001698066 |
| PFKFB2 | 5 | 3.42623 | 2.48692469 | 0.93930531 | 0.0001683 | 0.001698066 |
| CASP3 | 5 | 2.649465 | 2.16722593 | 0.48223907 | 0.00017413 | 0.001732977 |
| CRADD | 5 | 2.651245 | 2.00125802 | 0.64998698 | 0.00017413 | 0.001732977 |
| SRP14 | 5 | 3.67493 | 2.71774815 | 0.95718185 | 0.00018015 | 0.001780763 |
| CASP8 | 5 | 2.36722 | 1.68780494 | 0.67941506 | 0.00018636 | 0.001829824 |
| STAMBP | 5 | 1.291835 | 0.8650284 | 0.4268066 | 0.00019277 | 0.001880192 |
| HPCAL1 | 5 | 1.55768 | 1.20751481 | 0.35016519 | 0.0001994 | 0.001919189 |
| LPCAT2 | 5 | 1.89835 | 1.23129259 | 0.66705741 | 0.0001994 | 0.001919189 |
| TJAP1 | 5 | 1.618795 | 1.2419716 | 0.3768234 | 0.00020277 | 0.0019389 |
| ADAM8 | 5 | 0.833155 | 0.49591975 | 0.33723525 | 0.00020623 | 0.001959199 |
| MAD1L1 | 5 | 1.375675 | 0.76111235 | 0.61456265 | 0.00021329 | 0.002013148 |
| BANK1 | 5 | 3.0365075 | 2.20565864 | 0.83084886 | 0.00023583 | 0.002169978 |
| GSAP | 5 | −4.00145 | 4.5350765 | 0.53362654 | 0.00023583 | 0.002169978 |
| PLAUR | 5 | 0.85716 | 0.50845926 | 0.34870074 | 0.00023583 | 0.002169978 |
| SKAP2 | 5 | 5.097575 | 4.2663716 | 0.8312034 | 0.00023583 | 0.002169978 |
| WASF1 | 5 | 1.222375 | 0.80290617 | 0.41946883 | 0.00023978 | 0.002192523 |
| NPM1 | 5 | 3.019145 | 2.30934938 | 0.70979562 | 0.00025209 | 0.00227659 |
| PADI4 | 5 | 4.01182 | 2.9855284 | 1.0262916 | 0.00026061 | 0.002339058 |
| ELOA | 5 | 1.82459 | 1.11012099 | 0.71446901 | 0.00026939 | 0.002403167 |
| IL16 | 5 | 1.535085 | 1.00814074 | 0.52694426 | 0.00029744 | 0.002544777 |
| PRKAB1 | 5 | 1.167055 | 0.68056914 | 0.48648586 | 0.00029744 | 0.002544777 |
| PRKRA | 5 | 2.026855 | 1.42272099 | 0.60413401 | 0.00029744 | 0.002544777 |
| RASSF2 | 5 | 2.66439 | 1.72779012 | 0.93659988 | 0.00029744 | 0.002544777 |
| TNFRSF14 | 5 | 1.091535 | 0.83697407 | 0.25456093 | 0.00029744 | 0.002544777 |
| CD63 | 5 | −0.406665 | −0.8918074 | 0.48514241 | 0.00030738 | 0.002599437 |
| PXN | 5 | 3.13843 | 2.19958025 | 0.93884975 | 0.00030738 | 0.002599437 |
| DDX58 | 5 | 1.779265 | 1.23599877 | 0.54326623 | 0.00031764 | 0.002670706 |
| SULT1A1 | 5 | 3.9877075 | 3.00254012 | 0.98516738 | 0.00032821 | 0.002743831 |
| DAPP1 | 5 | 0.5799225 | 0.13404136 | 0.44588114 | 0.0003391 | 0.002802931 |
| TBL1X | 5 | 1.37139 | 0.83843086 | 0.53295914 | 0.00033911 | 0.002802931 |
| DRG2 | 5 | 0.87995 | 0.59698272 | 0.28296728 | 0.00035035 | 0.00287956 |
| CCL7 | 5 | 1.57304 | 0.94564321 | 0.62739679 | 0.00037388 | 0.00303882 |
| SIRT2 | 5 | 1.473125 | 0.93531975 | 0.53780525 | 0.00037388 | 0.00303882 |
| FURIN | 5 | 0.747105 | 0.37125185 | 0.37585315 | 0.00038619 | 0.003121548 |
| CLEC5A | 5 | 0.68555 | 0.37974321 | 0.30580679 | 0.00039888 | 0.003206406 |
| DEFA1_DEFA1B | 5 | 0.8787475 | 0.28521296 | 0.59353454 | 0.00042544 | 0.003364429 |
| CASP10 | 5 | 2.246355 | 1.60358765 | 0.64276735 | 0.00045364 | 0.003511523 |
| MGLL | 5 | 2.325815 | 1.81413827 | 0.51167673 | 0.00045364 | 0.003511523 |
| PGLYRP1 | 5 | 1.318775 | 0.85000494 | 0.46877006 | 0.00045364 | 0.003511523 |
| CAMKK1 | 5 | 0.8981 | 0.47095556 | 0.42714444 | 0.00046837 | 0.003606448 |
| TRIM5 | 5 | 1.8189375 | 1.31105 | 0.5078875 | 0.00049923 | 0.00382395 |
| CASP1 | 5 | 0.811255 | 0.28506667 | 0.52618833 | 0.00053196 | 0.004053416 |
| DFFA | 5 | 1.5615275 | 1.12695 | 0.4345775 | 0.00054906 | 0.004140611 |
| FKBP1B | 5 | 1.8051075 | 1.19781667 | 0.60729083 | 0.00054906 | 0.004140611 |
| PEBP1 | 5 | 1.0564675 | 0.65543395 | 0.40103355 | 0.0006035 | 0.004527775 |
| LACTB2 | 5 | 0.79863 | 0.50175062 | 0.29687938 | 0.00067331 | 0.005025802 |
| SRPK2 | 5 | 1.376145 | 0.85123827 | 0.52490673 | 0.00068391 | 0.005078966 |
| VSIR | 5 | 1.473405 | 1.0827642 | 0.3906408 | 0.00070551 | 0.005212939 |
| SDC4 | 5 | 2.037245 | 1.70055185 | 0.33669315 | 0.00077416 | 0.005663161 |
| APP | 5 | 4.1155525 | 3.76572654 | 0.34982596 | 0.00077418 | 0.005663161 |
| SF3B4 | 5 | 1.08987 | 0.6796037 | 0.4102663 | 0.00079842 | 0.005811397 |
| FMNL1 | 5 | 2.53273 | 1.83514321 | 0.69758679 | 0.00084902 | 0.006149104 |
| ENO1 | 5 | 1.1757325 | 0.75351667 | 0.42221583 | 0.00087542 | 0.006309081 |
| STX6 | 5 | 1.033935 | 0.58198765 | 0.45194735 | 0.00090258 | 0.006472934 |
| FGFR1OP | 5 | 1.880165 | 1.36908519 | 0.51107981 | 0.00093049 | 0.006608514 |
| FXN | 5 | 1.1126075 | 0.80003765 | 0.31256985 | 0.00093052 | 0.006608514 |
| LAMP2 | 5 | 0.4609525 | 0.29404753 | 0.16690497 | 0.00095926 | 0.006779704 |
| SNX9 | 5 | 1.487945 | 1.06732593 | 0.42061907 | 0.00098882 | 0.006955018 |
| GP1BA | 5 | 0.727225 | 0.47930494 | 0.24792006 | 0.00101922 | 0.007100569 |
| TBCC | 5 | 1.47676 | 1.03765926 | 0.43910074 | 0.00105045 | 0.007249331 |
| STX4 | 5 | 2.922505 | 2.42353704 | 0.49896796 | 0.00105048 | 0.007249331 |
| BIRC2 | 5 | 0.65038 | 0.25346914 | 0.39691086 | 0.0010826 | 0.007332833 |
| CD164 | 5 | 1.0834475 | 0.81388086 | 0.26956664 | 0.00108263 | 0.007332833 |
| CD69 | 5 | 1.99495 | 1.52864444 | 0.46630556 | 0.00108263 | 0.007332833 |
| USO1 | 5 | 2.275725 | 1.70623951 | 0.56948549 | 0.00108263 | 0.007332833 |
| GLRX | 5 | 1.265035 | 0.82014938 | 0.44488562 | 0.00114964 | 0.007715497 |
| VTA1 | 5 | 0.8208675 | 0.42626852 | 0.39459898 | 0.00118462 | 0.007913696 |
| BIN2 | 5 | 1.8948275 | 1.22010679 | 0.67472071 | 0.00122054 | 0.008043482 |
| CHMP1A | 5 | 2.04216 | 1.65533333 | 0.38682667 | 0.00122054 | 0.008043482 |
| CDHR5 | 5 | 0.356255 | −0.0284346 | 0.38468957 | 0.00125747 | 0.008249663 |
| CERT | 5 | 1.611135 | 1.1712321 | 0.4399029 | 0.00127627 | 0.008335656 |
| APRT | 5 | 2.043345 | 1.64981975 | 0.39352525 | 0.00129542 | 0.008423121 |
| STAT5B | 5 | 3.48447 | 2.6748642 | 0.8096058 | 0.00133443 | 0.008600322 |
| SEMA4D | 5 | 1.1460325 | 0.97237346 | 0.17365904 | 0.00139494 | 0.008950842 |
| CDKN1A | 5 | 1.414845 | 1.06554691 | 0.34929809 | 0.00141568 | 0.00904425 |
| TXLNA | 5 | 1.79377 | 1.40937654 | 0.38439346 | 0.00145806 | 0.009274504 |
| PTX3 | 5 | 1.34015 | 0.87321235 | 0.46693765 | 0.00150155 | 0.009468856 |
| PLA2G2A | 5 | 1.1754675 | 0.71339074 | 0.46207676 | 0.0015462 | 0.009708527 |
| SERPINB9 | 5 | 1.56593 | 1.34443704 | 0.22149296 | 0.00159211 | 0.009912044 |
| HBEGF | 5 | 2.80524 | 2.37535062 | 0.42988938 | 0.00159216 | 0.009912044 |
| CLEC6A | 5 | 0.953085 | 0.51984691 | 0.43323809 | 0.00163933 | 0.010034871 |
| EIF4B | 5 | 1.02923 | 0.59872222 | 0.43050778 | 0.00163933 | 0.010034871 |
| F9 | 5 | 0.140485 | −0.0562852 | 0.19677019 | 0.00163933 | 0.010034871 |
| NUB1 | 5 | 1.448305 | 1.06308148 | 0.38522352 | 0.00168777 | 0.010288394 |
| VIM | 5 | 3.037965 | 2.36260617 | 0.67535883 | 0.00173748 | 0.010504204 |
| CAPG | 5 | 0.68897 | 0.00965926 | 0.67931074 | 0.00173754 | 0.010504204 |
| LRMP | 5 | 1.23766 | 0.8442716 | 0.3933884 | 0.00178865 | 0.01076868 |
| PLXNB3 | 5 | 1.18539 | 0.86543086 | 0.31995914 | 0.00189503 | 0.011362428 |
| ARID4B | 5 | 0.97223 | 0.61434074 | 0.35788926 | 0.00212537 | 0.012538361 |
| SUGT1 | 5 | 1.62652 | 1.12980988 | 0.49671012 | 0.00212544 | 0.012538361 |
| FOPNL | 5 | 0.22701 | −0.1087148 | 0.33572481 | 0.00218692 | 0.012746857 |
| PPP1R2 | 5 | 1.260955 | 0.91716173 | 0.34379327 | 0.0023148 | 0.013438691 |
| GRAP2 | 5 | 1.113325 | 0.73792099 | 0.37540401 | 0.00244949 | 0.014164466 |
| CDC37 | 5 | 0.4667275 | 0.21942901 | 0.24729849 | 0.0025195 | 0.014511911 |
| CALCOCO1 | 5 | 3.997125 | 3.41138148 | 0.58574352 | 0.00274063 | 0.015540874 |
| DARS | 5 | 2.07932 | 1.67263704 | 0.40668296 | 0.00274063 | 0.015540874 |
| SPARC | 5 | 3.8063275 | 3.51281049 | 0.29351701 | 0.00289776 | 0.016242989 |
| MICB_MICA | 5 | 0.67041 | −0.110021 | 0.78043099 | 0.00297937 | 0.016636688 |
| EDAR | 5 | 2.7701675 | 1.97640432 | 0.79376318 | 0.0031489 | 0.017384327 |
| INHBC | 5 | 0.4609225 | 0.04674753 | 0.41417497 | 0.0031489 | 0.017384327 |
| PRKAR1A | 5 | 1.02657 | 0.55758025 | 0.46898975 | 0.0031489 | 0.017384327 |
| TARBP2 | 5 | 1.49597 | 1.13155556 | 0.36441444 | 0.00323693 | 0.017736437 |
| VNN2 | 5 | 1.68487 | 1.32809136 | 0.35677864 | 0.00332719 | 0.018162996 |
| LEP | 5 | 2.29571 | 1.18299012 | 1.11271988 | 0.00341974 | 0.018598819 |
| NINJ1 | 5 | 0.877465 | 0.56171111 | 0.31575389 | 0.00361181 | 0.019498959 |
| CA13 | 5 | 2.769775 | 1.95532099 | 0.81445401 | 0.00361191 | 0.019498959 |
| THPO | 5 | 1.771085 | 1.55280864 | 0.21827636 | 0.00391861 | 0.021076925 |
| SLC27A4 | 5 | 0.91911 | 0.47518642 | 0.44392358 | 0.00402598 | 0.021575138 |
| CTSS | 5 | 0.27408 | 0.13081728 | 0.14326272 | 0.0041358 | 0.021923912 |
| PLIN3 | 5 | 1.404625 | 1.17561605 | 0.22900895 | 0.00413602 | 0.021923912 |
| POLR2F | 5 | 0.96444 | 0.6120321 | 0.3524079 | 0.00413602 | 0.021923912 |
| STX16 | 5 | 0.673715 | 0.45664815 | 0.21706685 | 0.00424878 | 0.022279459 |
| FHIT | 5 | 1.217295 | 0.6634642 | 0.5538308 | 0.00436432 | 0.022803597 |
| MAX | 5 | 1.34857 | 0.89681358 | 0.45175642 | 0.00448271 | 0.023256035 |
| ATP5IF1 | 5 | 1.75523 | 1.34245432 | 0.41277568 | 0.00472812 | 0.024357111 |
| FUS | 5 | 0.452375 | 0.07650988 | 0.37586512 | 0.00485552 | 0.024837853 |
| S100A4 | 5 | 0.72805 | 0.48020247 | 0.24784753 | 0.00485552 | 0.024837853 |
| LAT | 5 | 2.8400875 | 2.58677222 | 0.25331528 | 0.00491994 | 0.025079685 |
| TRIM21 | 5 | 1.95907 | 1.49700864 | 0.46206136 | 0.00498589 | 0.025327623 |
| MITD1 | 5 | 2.61332 | 2.25424568 | 0.35907432 | 0.00511929 | 0.025826566 |
| FCAR | 5 | 1.2544525 | 0.86383765 | 0.39061485 | 0.00511942 | 0.025826566 |
| TDRKH | 5 | 0.95129 | 0.61914074 | 0.33214926 | 0.00525603 | 0.026334826 |
| PPIB | 5 | 3.182315 | 2.7625284 | 0.4197866 | 0.00525616 | 0.026334826 |
| CLEC4D | 5 | 1.921585 | 1.31972346 | 0.60186154 | 0.00539594 | 0.026761523 |
| HSPB1 | 5 | 1.58633 | 1.1153284 | 0.4710016 | 0.00539621 | 0.026761523 |
| TRAF2 | 5 | 2.030465 | 1.57956296 | 0.45090204 | 0.00539621 | 0.026761523 |
| METAP1D | 5 | 1.33853 | 1.00893951 | 0.32959049 | 0.0056863 | 0.028010284 |
| PLXNA4 | 5 | 1.7360175 | 1.40241667 | 0.33360083 | 0.00583677 | 0.028655022 |
| FABP4 | 5 | 1.0048675 | 0.47836975 | 0.52649775 | 0.00599068 | 0.029214538 |
| PARK7 | 5 | 0.5630575 | 0.26947963 | 0.29357787 | 0.00599068 | 0.029214538 |
| C2 | 5 | 0.5999425 | 0.41047593 | 0.18946657 | 0.00614823 | 0.029883262 |
| TPP1 | 5 | 1.393395 | 1.19387284 | 0.19952216 | 0.00647442 | 0.03105675 |
| RAB6A | 5 | 1.7850625 | 1.41329815 | 0.37176435 | 0.00647458 | 0.03105675 |
| FLI1 | 5 | 1.163935 | 0.81425432 | 0.34968068 | 0.00664353 | 0.031659542 |
| TMSB10 | 5 | 1.7442675 | 1.21819691 | 0.52607059 | 0.00664353 | 0.031659542 |
| CEP85 | 5 | 1.435795 | 1.06738025 | 0.36841475 | 0.00699336 | 0.033110964 |
| CKAP4 | 5 | 0.835985 | 0.61637901 | 0.21960599 | 0.00717424 | 0.033749713 |
| FKBP4 | 5 | 0.793845 | 0.48594444 | 0.30790056 | 0.00717441 | 0.033749713 |
| INPPL1 | 5 | 1.238535 | 0.88450617 | 0.35402883 | 0.007549 | 0.035061731 |
| CLPP | 5 | 1.657025 | 1.30776173 | 0.34926327 | 0.00754918 | 0.035061731 |
| CD84 | 5 | 1.16167 | 0.98078395 | 0.18088605 | 0.00774306 | 0.035735341 |
| CD99L2 | 5 | 0.59759 | 0.4549037 | 0.1426863 | 0.0079414 | 0.036420914 |
| EBAG9 | 5 | 1.482225 | 1.19747037 | 0.28475463 | 0.0079414 | 0.036420914 |
| LBP | 5 | 1.9213675 | 1.45300432 | 0.46836318 | 0.00814428 | 0.037003351 |
| TNFRSF10C | 5 | 1.02043 | 0.61321728 | 0.40721272 | 0.00814428 | 0.037003351 |
| KDR | 5 | 0.38671 | 0.19226914 | 0.19444086 | 0.00922988 | 0.041168639 |
| ABHD14B | 5 | 1.126665 | 0.81058148 | 0.31608352 | 0.01044259 | 0.045878425 |
| HGS | 5 | 0.483635 | 0.27259383 | 0.21104117 | 0.01070154 | 0.046736384 |
| TBC1D17 | 5 | 1.07342 | 0.75971481 | 0.31370519 | 0.01096618 | 0.047187835 |
| RCOR1 | 5 | 0.732925 | 0.61476914 | 0.11815586 | 0.01096641 | 0.047187835 |
| SPINT2 | 5 | 1.570555 | 1.34085432 | 0.22970068 | 0.01096641 | 0.047187835 |
| CXCL6 | 5 | 3.65544 | 3.36614815 | 0.28929185 | 0.01123686 | 0.048209747 |
| RNASE3 | 5 | 7.575695 | 6.71196049 | 0.86373451 | 0.01179557 | 0.050458847 |
| FETUB | 5 | 0.55885 | 0.31022716 | 0.24862284 | 0.01237879 | 0.052493255 |
| ARSA | 5 | 1.69286 | 1.34076173 | 0.35209827 | 0.01298741 | 0.054599364 |
| LGALS9 | 5 | 0.297665 | 0.03171111 | 0.26595389 | 0.01394998 | 0.057979598 |
| GLOD4 | 5 | 0.58012 | 0.41634815 | 0.16377185 | 0.01411611 | 0.05850387 |
| DIABLO | 5 | 0.918315 | 0.60249012 | 0.31582488 | 0.01462589 | 0.060107014 |
| SERPINE1 | 5 | 3.2264875 | 3.0352216 | 0.1912659 | 0.01462618 | 0.060107014 |
| STXBP3 | 5 | 0.399535 | 0.22524815 | 0.17428685 | 0.01497499 | 0.06119667 |
| MZT1 | 5 | 0.943955 | 0.77847778 | 0.16547722 | 0.01533081 | 0.061959694 |
| CD177 | 5 | −0.04238 | −0.6138247 | 0.57144469 | 0.01533111 | 0.061959694 |
| IL1B | 5 | 0.10552 | −0.1426099 | 0.24812988 | 0.01533111 | 0.061959694 |
| AKT1S1 | 5 | 2.0499 | 1.75955309 | 0.29034691 | 0.01606577 | 0.064395147 |
| MPHOSPH8 | 5 | 1.085345 | 0.86896667 | 0.21637833 | 0.01606577 | 0.064395147 |
| PDCD5 | 5 | 1.095935 | 0.84238642 | 0.25354858 | 0.01644458 | 0.065554289 |
| GUSB | 5 | 0.9777725 | 0.61432037 | 0.36345213 | 0.01683121 | 0.066731884 |
| PLA2G4A | 5 | 0.75764 | 0.5967321 | 0.1609079 | 0.01762848 | 0.068774576 |
| PSIP1 | 5 | 1.9581625 | 1.53091667 | 0.42724583 | 0.01762848 | 0.068774576 |
| CXCL1 | 5 | 3.75333 | 3.42281605 | 0.33051395 | 0.01803939 | 0.0700043 |
| F11R | 5 | 1.13519 | 0.90451975 | 0.23067025 | 0.01888643 | 0.072332062 |
| LGMN | 5 | 0.983165 | 0.79091358 | 0.19225142 | 0.01954363 | 0.074265813 |
| PRDX3 | 5 | 0.994835 | 0.72310988 | 0.27172512 | 0.0197677 | 0.074730403 |
| IMPA1 | 5 | 0.707635 | 0.51961728 | 0.18801772 | 0.01976806 | 0.074730403 |
| PSMD9 | 5 | 1.253035 | 1.00949877 | 0.24353623 | 0.02022219 | 0.076054161 |
| GPKOW | 5 | 0.692355 | 0.4753642 | 0.2169908 | 0.02163927 | 0.080352321 |
| VEGFA | 5 | 2.3658925 | 1.95520062 | 0.41069188 | 0.02163965 | 0.080352321 |
| BTC | 5 | 1.73613 | 1.4793037 | 0.2568263 | 0.02213099 | 0.081968694 |
| IL17RA | 5 | 0.588795 | 0.3469642 | 0.2418308 | 0.02237988 | 0.082681225 |
| LYN | 5 | 1.91642 | 1.66517037 | 0.25124963 | 0.0226316 | 0.08319248 |
| ARHGAP1 | 5 | 0.61346 | 0.30669383 | 0.30676617 | 0.02263199 | 0.08319248 |
| PARP1 | 5 | 0.812565 | 0.55228765 | 0.26027735 | 0.02314283 | 0.084644895 |
| IL6 | 5 | 1.0998675 | 0.62626543 | 0.47360207 | 0.02366365 | 0.08633395 |
| FOXO3 | 5 | 1.15705 | 0.51118395 | 0.64586605 | 0.02473586 | 0.089575639 |
| PQBP1 | 5 | 0.963525 | 0.72888272 | 0.23464228 | 0.02528757 | 0.091347436 |
| FES | 5 | 1.127815 | 0.88609877 | 0.24171623 | 0.02642301 | 0.094979998 |
| CALB2 | 5 | 0.56889 | 0.28894691 | 0.27994309 | 0.02700706 | 0.096841501 |
| ERBB3 | 5 | 0.28914 | 0.20258642 | 0.08655358 | 0.02945607 | 0.103841536 |
| ITGAM | 5 | 0.5792675 | 0.3934142 | 0.1858533 | 0.02945607 | 0.103841536 |
| DNAJB1 | 5 | 1.509185 | 1.1736284 | 0.3355566 | 0.03009737 | 0.105593424 |
| WARS | 5 | 0.0330875 | −0.1766574 | 0.20974491 | 0.03009737 | 0.105593424 |
| IGFBP3 | 5 | 0.2784625 | 0.09132037 | 0.18714213 | 0.03075064 | 0.10660706 |
| IQGAP2 | 5 | 1.69976 | 1.35129012 | 0.34846988 | 0.03075064 | 0.10660706 |
| ANGPT1 | 5 | 4.80942 | 4.64364074 | 0.16577926 | 0.03108132 | 0.107498737 |
| PDGFC | 5 | 0.05095 | −0.1207123 | 0.17166235 | 0.03141554 | 0.107638544 |
| TGFB1 | 5 | 1.772755 | 1.59624691 | 0.17650809 | 0.03141554 | 0.107638544 |
| TANK | 5 | 1.23696 | 0.89090123 | 0.34605877 | 0.03141603 | 0.107638544 |
| ESAM | 5 | 1.360135 | 1.2445679 | 0.1155671 | 0.03209325 | 0.109193378 |
| CLIP2 | 5 | 2.771475 | 2.37380494 | 0.39767006 | 0.03209375 | 0.109193378 |
| CHAC2 | 5 | 1.03315 | 0.84250494 | 0.19064506 | 0.0334858 | 0.112623515 |
| RHOC | 5 | 1.91921 | 1.66223457 | 0.25697543 | 0.03348683 | 0.112623515 |
| SELP | 5 | 1.4117625 | 1.19346481 | 0.21829769 | 0.03420255 | 0.114504205 |
| SMAD1 | 5 | 0.858115 | 0.68742222 | 0.17069278 | 0.03420255 | 0.114504205 |
| IL18R1 | 5 | 0.4141775 | 0.17007099 | 0.24410651 | 0.03567329 | 0.118883889 |
| PLAT | 5 | −0.0181475 | −0.3315315 | 0.31338398 | 0.03719764 | 0.123401693 |
| NID2 | 5 | 2.8210575 | 2.49760926 | 0.32344824 | 0.03758672 | 0.124029687 |
| CLEC4G | 5 | 0.431325 | 0.23372222 | 0.19760278 | 0.03797982 | 0.124029687 |
| CXCL3 | 5 | 3.84587 | 3.4960358 | 0.3498342 | 0.03798038 | 0.124029687 |
| OXT | 5 | 0.24666 | −0.5861074 | 0.83276741 | 0.03798038 | 0.124029687 |
| SH2B3 | 5 | 1.07474 | 0.8901642 | 0.1845758 | 0.03798038 | 0.124029687 |
| ACP6 | 5 | 0.47242 | 0.24842099 | 0.22399901 | 0.03877709 | 0.125233737 |
| LGALS8 | 5 | 0.6315975 | 0.4663821 | 0.1652154 | 0.03877709 | 0.125233737 |
| NCK2 | 5 | 0.535485 | 0.37674691 | 0.15873809 | 0.03958738 | 0.127290493 |
| F7 | 5 | 0.35819 | 0.21556296 | 0.14262704 | 0.03958795 | 0.127290493 |
| VEGFC | 5 | 3.53064 | 3.4022642 | 0.1283758 | 0.040412 | 0.129375175 |
| FXYD5 | 5 | 0.43906 | 0.21448765 | 0.22457235 | 0.04253961 | 0.13521518 |
| IL7 | 5 | 3.40209 | 3.19636296 | 0.20572704 | 0.04297619 | 0.13521518 |
| ATXN10 | 5 | −1.33959 | −1.5720741 | 0.23248407 | 0.0429768 | 0.13521518 |
| LYAR | 5 | 0.806785 | 0.57235185 | 0.23443315 | 0.0429768 | 0.13521518 |
| WAS | 5 | 0.95405 | 0.66865432 | 0.28539568 | 0.0429768 | 0.13521518 |
| YTHDF3 | 5 | 1.535515 | 1.1839321 | 0.3515829 | 0.0429768 | 0.13521518 |
| SHMT1 | 5 | −0.022775 | −0.2772531 | 0.25447809 | 0.04386134 | 0.137407139 |
| SSB | 5 | 0.92634 | 0.71857654 | 0.20776346 | 0.04476119 | 0.139628201 |
| CD22 | 5 | 0.6269875 | 0.42132284 | 0.20566466 | 0.04567657 | 0.140980649 |
| FCER2 | 5 | 0.5620075 | 0.33131667 | 0.23069083 | 0.04567657 | 0.140980649 |
| MAP4K5 | 5 | 0.69259 | 0.52052593 | 0.17206407 | 0.04851785 | 0.148497111 |
| CFC1 | 5 | 0.851945 | 0.66241111 | 0.18953389 | 0.04949733 | 0.150237757 |
| MVK | 5 | 0.82529 | 0.63661605 | 0.18867395 | 0.04949733 | 0.150237757 |

TABLE 4

| | Median expression | Median expression | | | | |
|---|---|---|---|---|---|---|
| | in INCOV in | across INCOV is | | | Adjusted | |
| Protein | cluster E | clusters B, C, D | Delta | P-value | P-value | Group |
| APEX1_MET | 1.554366982 | 0.108296408 | 1.446070574 | 3.36E−08 | 1.46E−06 | Higher in INCOV E |
| IKBKG_CVD2 | 1.7123015 | 0.803001126 | 0.909300374 | 3.36E−08 | 1.46E−06 | Higher in INCOV E |
| NBN_ODA | 1.540206161 | 0.284422762 | 1.255783399 | 1.97E−08 | 1.46E−06 | Higher in INCOV E |
| SIRT2_INF | 1.395307866 | 0.608634675 | 0.786673191 | 2.94E−08 | 1.46E−06 | Higher in INCOV E |
| FGR_ODA | 1.78632949 | 0.883833289 | 0.902496202 | 4.67E−08 | 1.62E−06 | Higher in INCOV E |
| THPO_CVD2 | 1.252305845 | 0.157942302 | 1.094363543 | 9.53E−08 | 2.76E−06 | Higher in INCOV E |
| RASSF2_ODA | 1.683928481 | 0.516691577 | 1.167236904 | 1.23E−07 | 3.06E−06 | Higher in INCOV E |
| AXIN1_INF | 1.598321057 | 0.862815529 | 0.735505527 | 1.80E−07 | 3.13E−06 | Higher in INCOV E |
| STAMBP_INF | 1.468267839 | 0.599744835 | 0.868523004 | 1.59E−07 | 3.13E−06 | Higher in INCOV E |
| YES1_ODA | 1.496095999 | 0.786554987 | 0.709541012 | 1.69E−07 | 3.13E−06 | Higher in INCOV E |
| LAT2_ODA | 1.638260828 | 0.550905314 | 1.087355514 | 2.17E−07 | 3.35E−06 | Higher in INCOV E |
| STX8_ODA | 1.593385956 | 0.836687998 | 0.756697958 | 2.31E−07 | 3.35E−06 | Higher in INCOV E |
| DFFA_IRE | 1.484531975 | 0.489161566 | 0.99537041 | 2.96E−07 | 3.65E−06 | Higher in INCOV E |
| EGLN1_IRE | 1.566089051 | 0.259302502 | 1.30678655 | 3.15E−07 | 3.65E−06 | Higher in INCOV E |
| PRKRA_ODA | 1.929100059 | 0.884259406 | 1.044840653 | 2.96E−07 | 3.65E−06 | Higher in INCOV E |
| CD40LG_CVD2 | 1.548882369 | 0.671620141 | 0.877262229 | 4.02E−07 | 4.38E−06 | Higher in INCOV E |
| ICA1_IRE | 1.642923643 | 0.817415319 | 0.825508324 | 4.83E−07 | 4.94E−06 | Higher in INCOV E |
| INPPL1_ODA | 1.466964369 | 0.692566298 | 0.77439807 | 5.45E−07 | 4.99E−06 | Higher in INCOV E |
| SRPK2_IRE | 1.623334749 | 0.873431467 | 0.749903282 | 5.45E−07 | 4.99E−06 | Higher in INCOV E |
| FKBP4_MET | 1.716310882 | 0.351663405 | 1.364647476 | 6.94E−07 | 5.25E−06 | Higher in INCOV E |
| HCLS1_IRE | 1.161306237 | 0.610229869 | 0.551076369 | 6.94E−07 | 5.25E−06 | Higher in INCOV E |
| IL16_CVD2 | 1.443778938 | 0.115699416 | 1.328079522 | 6.15E−07 | 5.25E−06 | Higher in INCOV E |
| TRIM5_IRE | 1.807387755 | 1.077263712 | 0.730124043 | 6.94E−07 | 5.25E−06 | Higher in INCOV E |
| TNFSF14_INF | 1.259973156 | 0.26868266 | 0.991290496 | 9.35E−07 | 6.78E−06 | Higher in INCOV E |
| HDGF_MET | 1.135963641 | 0.110672225 | 1.025291415 | 1.26E−06 | 8.74E−06 | Higher in INCOV E |
| FOXO1_ODA | 1.685256767 | 0.570050831 | 1.115205936 | 1.41E−06 | 9.09E−06 | Higher in INCOV E |
| PXN_ODA | 1.140024949 | 0.341447515 | 0.798577434 | 1.41E−06 | 9.09E−06 | Higher in INCOV E |
| IRAK1_IRE | 1.783560097 | 1.107637898 | 0.675922198 | 1.78E−06 | 1.11E−05 | Higher in INCOV E |
| DECR1_CVD2 | 1.206963852 | 0.50845617 | 0.698507682 | 2.00E−06 | 1.20E−05 | Higher in INCOV E |
| EIF4G1_IRE | 1.151858945 | 0.797015674 | 0.354843272 | 2.82E−06 | 1.63E−05 | Higher in INCOV E |
| DAPP1_IRE | 0.72463341 | −0.085992956 | 0.810626366 | 3.53E−06 | 1.92E−05 | Higher in INCOV E |
| SORT1_CVD2 | 1.126847498 | 0.488060938 | 0.63878656 | 3.53E−06 | 1.92E−05 | Higher in INCOV E |
| PLXNA4_IRE | 1.535132048 | 1.074600317 | 0.460531731 | 3.73E−06 | 1.97E−05 | Higher in INCOV E |
| GLRX_MET | 1.665222811 | 0.462414826 | 1.202807985 | 4.42E−06 | 2.20E−05 | Higher in INCOV E |
| HEXIM1_IRE | 1.563663411 | 0.909588959 | 0.654074452 | 4.42E−06 | 2.20E−05 | Higher in INCOV E |
| CD84_CVD2 | 1.545435244 | 0.804421074 | 0.74101417 | 5.22E−06 | 2.45E−05 | Higher in INCOV E |
| CXCL6_INF | 1.452865068 | 0.415028752 | 1.037836315 | 5.22E−06 | 2.45E−05 | Higher in INCOV E |
| NUB1_ODA | 1.527564286 | 0.633235359 | 0.894328927 | 6.51E−06 | 2.98E−05 | Higher in INCOV E |
| HSPB1_CVD2 | −0.148499944 | 0.692262023 | −0.840761967 | 8.54E−06 | 3.81E−05 | Higher in INCOV B, C, D |
| SH2D1A_IRE | 1.447512836 | 0.211034463 | 1.236478373 | 1.12E−05 | 4.87E−05 | Higher in INCOV E |
| BTN3A2_IRE | 1.552744112 | 0.544624739 | 1.008119373 | 1.18E−05 | 5.01E−05 | Higher in INCOV E |
| ANXA4_MET | 1.625962163 | 0.33950172 | 1.286460443 | 1.24E−05 | 5.16E−05 | Higher in INCOV E |
| BACH1_IRE | 1.766302048 | 1.133381185 | 0.632920863 | 1.31E−05 | 5.31E−05 | Higher in INCOV E |
| FGF21_CVD2 | 0.768645932 | −0.30487085 | 1.073516783 | 1.54E−05 | 6.08E−05 | Higher in INCOV E |
| IRAK4_IRE | 1.281030031 | 0.472838866 | 0.808191166 | 1.62E−05 | 6.27E−05 | Higher in INCOV E |
| HBEGF_CVD2 | 1.468268378 | 0.676271227 | 0.791997151 | 1.71E−05 | 6.47E−05 | Higher in INCOV E |
| PIK3AP1_IRE | 1.686023319 | 0.768780143 | 0.917243176 | 2.00E−05 | 7.40E−05 | Higher in INCOV E |
| BANK1_ODA | 1.572721086 | 1.015916272 | 0.556804814 | 2.11E−05 | 7.64E−05 | Higher in INCOV E |
| PPP1R2_MET | 1.34501919 | 0.462289188 | 0.882730002 | 2.34E−05 | 8.30E−05 | Higher in INCOV E |
| LILRB4_IRE | 1.109681957 | 0.000860411 | 1.108821546 | 2.46E−05 | 8.56E−05 | Higher in INCOV E |
| COMT_MET | 1.711242627 | 0.861048881 | 0.850193746 | 2.87E−05 | 9.80E−05 | Higher in INCOV E |
| USP8_MET | 1.777937864 | 0.314655033 | 1.463282831 | 3.02E−05 | 0.000101121 | Higher in INCOV E |
| LAMP3_IRE | 0.809465807 | −0.651160708 | 1.460626515 | 3.18E−05 | 0.000102472 | Higher in INCOV E |
| TNFRSF11A_CVD2 | 0.518120733 | −0.137813365 | 0.655934098 | 3.18E−05 | 0.000102472 | Higher in INCOV E |
| LGALS9_CVD2 | 0.740214815 | −0.058202896 | 0.798417711 | 3.70E−05 | 0.000117157 | Higher in INCOV E |
| MAX_ODA | 1.69664129 | 1.049433285 | 0.647208005 | 4.76E−05 | 0.000147924 | Higher in INCOV E |
| NCF2_ODA | 1.284910424 | 0.634424471 | 0.650485954 | 5.00E−05 | 0.000152758 | Higher in INCOV E |
| NADK_MET | 1.369851138 | 0.509390147 | 0.86046099 | 6.41E−05 | 0.000192248 | Higher in INCOV E |
| PGF_CVD2 | 0.416526115 | −0.402250172 | 0.818776287 | 8.18E−05 | 0.000241239 | Higher in INCOV E |
| GRAP2_MET | 1.325475487 | 0.468278664 | 0.857196823 | 9.01E−05 | 0.000261313 | Higher in INCOV E |
| CA13_MET | 1.446561115 | 0.811687307 | 0.634873808 | 9.46E−05 | 0.000265364 | Higher in INCOV E |
| CD28_IRE | 0.579926083 | −0.235358587 | 0.81528467 | 9.46E−05 | 0.000265364 | Higher in INCOV E |
| FGF21_INF | 0.696762679 | −0.202865761 | 0.89962844 | 0.000138372 | 0.00038217 | Higher in INCOV E |
| CRKL_MET | 1.23025362 | 0.725593284 | 0.504660336 | 0.000145033 | 0.000388242 | Higher in INCOV E |
| RCOR1_ODA | 1.330059785 | 0.537003252 | 0.793056533 | 0.000145033 | 0.000388242 | Higher in INCOV E |
| HGF_INF | 1.283162255 | 0.317630644 | 0.96553161 | 0.000151995 | 0.000394734 | Higher in INCOV E |
| TNF_INF | 0.654118964 | 0.063293628 | 0.590825336 | 0.000151995 | 0.000394734 | Higher in INCOV E |
| SNAP23_MET | 1.045294036 | 0.389713346 | 0.65558069 | 0.000210285 | 0.000538082 | Higher in INCOV E |
| CDCP1_INF | 0.87533421 | −0.055013367 | 0.930347576 | 0.000230455 | 0.000581147 | Higher in INCOV E |
| PPP1R9B_IRE | 0.821391319 | 0.245793811 | 0.575597508 | 0.000289105 | 0.000718633 | Higher in INCOV E |
| SDC4_MET | 1.55735914 | 0.909395167 | 0.647963972 | 0.000330728 | 0.000810515 | Higher in INCOV E |
| BIRC2_IRE | 1.885758246 | 1.148943519 | 0.736814727 | 0.000345804 | 0.000835694 | Higher in INCOV E |
| CD164_MET | 1.606089583 | 0.321010568 | 1.285079016 | 0.000361522 | 0.000861711 | Higher in INCOV E |
| CEACAM8_CVD2 | 0.164306601 | −0.752537054 | 0.916843655 | 0.000394983 | 0.000928743 | Higher in INCOV E |
| MGMT_IRE | 0.937203832 | 0.712361876 | 0.224841955 | 0.000412778 | 0.000957644 | Higher in INCOV E |
| ADA_INF | 1.439445021 | 0.816219565 | 0.623225456 | 0.000610187 | 0.001397007 | Higher in INCOV E |
| CKAP4_IRE | 1.071203211 | 0.345901517 | 0.725301694 | 0.000723546 | 0.001635026 | Higher in INCOV E |
| DAB2_MET | 1.480149681 | 0.685529773 | 0.794619908 | 0.000754794 | 0.001662458 | Higher in INCOV E |
| FAM3C_MET | 0.855331419 | −0.021439966 | 0.876771385 | 0.000754794 | 0.001662458 | Higher in INCOV E |
| DCTN1_IRE | 1.067661882 | 0.490988491 | 0.576673391 | 0.000892742 | 0.001941714 | Higher in INCOV E |
| PAG1_MET | 1.550684151 | 0.436689661 | 1.113994489 | 0.001098009 | 0.002358685 | Higher in INCOV E |
| PRKAB1_ODA | 1.59084451 | 1.119775116 | 0.471069394 | 0.001241287 | 0.00263395 | Higher in INCOV E |
| SRC_CVD2 | −0.665855466 | −0.326407843 | −0.339447623 | 0.001346194 | 0.002822142 | Higher in INCOV B, C, D |
| SERPINB8_MET | 0.780376399 | 0.035225836 | 0.745150563 | 0.001459228 | 0.003022687 | Higher in INCOV E |
| AMBP_CVD2 | 0.565490292 | 0.247924498 | 0.317565794 | 0.001711964 | 0.003463741 | Higher in INCOV E |
| TNFRSF10B_CVD2 | 0.640341399 | −0.150332756 | 0.790674154 | 0.001711964 | 0.003463741 | Higher in INCOV E |
| ADM_CVD2 | 0.499004774 | −0.305680265 | 0.804685039 | 0.001852895 | 0.00370579 | Higher in INCOV E |
| IL12B_INF | 0.718740532 | 0.136371754 | 0.582368778 | 0.001927288 | 0.003810774 | Higher in INCOV E |
| RTN4R_MET | 0.718136131 | −0.507280263 | 1.225416394 | 0.00216723 | 0.004237057 | Higher in INCOV E |
| IL1RN_CVD2 | 0.595407092 | −0.348782099 | 0.94418919 | 0.002342089 | 0.004478281 | Higher in INCOV E |
| PDCD1_ODA | 0.30403003 | −0.330918762 | 0.634948792 | 0.002342089 | 0.004478281 | Higher in INCOV E |
| DDX58_IRE | 0.526297904 | 0.016287954 | 0.51000995 | 0.00243428 | 0.004603964 | Higher in INCOV E |
| OLR1_CVD2 | 0.637336516 | −0.204269368 | 0.841605883 | 0.002628697 | 0.004918207 | Higher in INCOV E |
| CAPG_ODA | 1.219960336 | 0.290393447 | 0.929566889 | 0.00330019 | 0.006108862 | Higher in INCOV E |
| TGFA_INF | 1.093136971 | 0.372161013 | 0.720975958 | 0.003426212 | 0.006275378 | Higher in INCOV E |
| ANXA11_MET | 1.412363095 | 1.183810996 | 0.228552099 | 0.003691488 | 0.006690822 | Higher in INCOV E |
| CDHR5_MET | 0.859755955 | 0.095268702 | 0.764487253 | 0.004278803 | 0.007520321 | Higher in INCOV E |
| CSF1_INF | 0.792896111 | 0.168467509 | 0.624428602 | 0.004278803 | 0.007520321 | Higher in INCOV E |
| TRAF2_IRE | 1.881414155 | 1.542788019 | 0.338626137 | 0.004278803 | 0.007520321 | Higher in INCOV E |
| TNFRSF10A_CVD2 | 0.832336062 | 0.285401386 | 0.546934675 | 0.004773554 | 0.008305984 | Higher in INCOV E |
| HAVCR1_CVD2 | 0.464204059 | −0.323318929 | 0.787522989 | 0.005131566 | 0.008753848 | Higher in INCOV E |
| PILRB_MET | 0.350542016 | −0.33360894 | 0.684150956 | 0.005131566 | 0.008753848 | Higher in INCOV E |
| IL10RB_INF | 0.701796062 | 0.036343636 | 0.665452426 | 0.005921273 | 0.009906746 | Higher in INCOV E |
| LAG3_IRE | 0.649747055 | 0.081540877 | 0.568206178 | 0.005921273 | 0.009906746 | Higher in INCOV E |
| CTSO_MET | 0.740406902 | 0.193969029 | 0.546437872 | 0.007312038 | 0.012117091 | Higher in INCOV E |
| CD83_IRE | 0.444294139 | −0.163587358 | 0.607881498 | 0.007836952 | 0.01286443 | Higher in INCOV E |
| CCL7_INF | 0.71665827 | −0.001795757 | 0.718454026 | 0.008111863 | 0.013191253 | Higher in INCOV E |
| CLMP_MET | 0.617693546 | −0.050150214 | 0.66784376 | 0.008395375 | 0.013525881 | Higher in INCOV E |
| CXCL11_INF | 1.112663528 | 0.651716869 | 0.460946659 | 0.008687717 | 0.013742389 | Higher in INCOV E |
| HAVCR1_ODA | 0.757549787 | −0.165669872 | 0.923219659 | 0.008687717 | 0.013742389 | Higher in INCOV E |
| NPDC1_MET | 0.953331855 | 0.31260785 | 0.640724004 | 0.009299834 | 0.014578119 | Higher in INCOV E |
| TRIM21_IRE | 0.203770426 | −0.35704538 | 0.560815806 | 0.009620092 | 0.0149455 | Higher in INCOV E |
| CD5_INF | 0.863402077 | 0.093662915 | 0.769739162 | 0.009950146 | 0.015187065 | Higher in INCOV E |
| TNFRSF9_INF | 0.345025849 | −0.079381071 | 0.42440692 | 0.009950146 | 0.015187065 | Higher in INCOV E |
| CLEC4D_IRE | 0.877853095 | 0.052114538 | 0.825738557 | 0.010640662 | 0.015690468 | Higher in INCOV E |
| CLEC5A_MET | 0.299304617 | −0.357842405 | 0.657147022 | 0.010640662 | 0.015690468 | Higher in INCOV E |
| CXCL10_INF | 0.47132128 | −0.106509299 | 0.57783058 | 0.010640662 | 0.015690468 | Higher in INCOV E |
| MMP7_CVD2 | 0.972019387 | 0.429450701 | 0.542568686 | 0.010640662 | 0.015690468 | Higher in INCOV E |
| LEP_CVD2 | 0.827755766 | 0.387707933 | 0.440047833 | 0.017879985 | 0.026143844 | Higher in INCOV E |
| TNFSF10_INF | 0.887051456 | 0.354800953 | 0.532250503 | 0.019036242 | 0.027602551 | Higher in INCOV E |
| CST5_INF | 0.675280854 | 0.074067521 | 0.601213333 | 0.019638507 | 0.028240498 | Higher in INCOV E |
| EDAR_IRE | 1.203349019 | 0.675140697 | 0.528208322 | 0.022905957 | 0.032669152 | Higher in INCOV E |
| TNFSF11_INF | 0.33021203 | −0.432269191 | 0.762481221 | 0.023613339 | 0.033404236 | Higher in INCOV E |
| OSCAR_CVD2 | 0.240692661 | −0.012590129 | 0.25328279 | 0.0243396 | 0.034153954 | Higher in INCOV E |
| TINAGL1_MET | 1.028425539 | 0.524133277 | 0.504292263 | 0.025085141 | 0.034918517 | Higher in INCOV E |
| CCL3_CVD2 | 0.472205289 | −0.011651135 | 0.483856424 | 0.027441565 | 0.037895495 | Higher in INCOV E |
| IFNG_INF | −0.142561787 | −1.085951752 | 0.943389965 | 0.02911655 | 0.039891966 | Higher in INCOV E |
| NCR1_IRE | 0.201873737 | −0.561221049 | 0.763094786 | 0.029986541 | 0.040762955 | Higher in INCOV E |
| PRDX5_IRE | 0.595652951 | 0.325334177 | 0.270318775 | 0.030878784 | 0.041650453 | Higher in INCOV E |
| SEMA3F_MET | 0.893288707 | 0.143575856 | 0.749712851 | 0.032731809 | 0.043810268 | Higher in INCOV E |
| IL12RB1_IRE | 0.654831152 | 0.2286436 | 0.426187552 | 0.033693497 | 0.044753195 | Higher in INCOV E |

1. A method of diagnosing or classifying a subject as having a chronic or long-term infection of a virus, bacterium, fungus, and/or parasite, and/or an autoimmune disease, comprising determining the level of one or more biomarkers in a biological sample obtained from a subject.

2. The method of claim 1, wherein the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV).

3. The method of claim 1, wherein the one or more biomarkers comprise proteins associated with an inflammatory response, wherein the proteins associated with an inflammatory response comprise one or more of TNF, IFNLR1, BCAM, S100A16, and IL5.

4. The method of claim 1, wherein the one or more biomarkers comprise cytokines, chemokines, and/or immunomodulatory proteins, wherein the cytokines, chemokines, and/or immunomodulatory proteins comprise one or more of TNF, IL5, IL11, IL13, IL15, IL1B, CXCL1, CXCL8, CCL3, CCL11, IL1RL2, CD28, HLA-DRA, LAG3, and PDCD1.

5. The method of claim 1, wherein the one or more biomarkers comprise hormones and hormone receptors, wherein the hormones and hormone receptors comprise one or more of CRH, CRHR1, and PTH1R.

6. The method of claim 1, wherein the one or more biomarkers comprise transcription factors and motifs thereof, wherein the transcription factors and motifs thereof comprise one or more of AP-1, BACH, BATF, IRF, and STAT.

7. A method of treating or preventing one or more symptoms in a subject diagnosed or classified as having a chronic or long-term infection of a virus, bacterium, fungus, and/or parasite, and/or an autoimmune disease, comprising administering to the subject one or more therapeutic agents.

8. The method of claim 7, wherein the subject is diagnosed or classified as having post-acute sequelae of SARS-CoV-2 infection (PASC).

9. The method of claim 7, wherein the one or more therapeutic agents comprise an anti-inflammatory agent.

10. The method of claim 7, wherein the one or more therapeutic agents are administered to the subject for a period of time of about 3 days to about 5 years.

11. The method of claim 7, further comprising monitoring the subject for the one or more symptoms of the long-term infection of the virus, bacterium, fungus, and/or parasite, and/or the autoimmune disease.

12. A molecular signature for use in determining whether a subject infected with or previously infected with a virus or other pathogen is likely to suffer from a chronic inflammatory syndrome with or without a chronic or long-term infection of the virus or other pathogen, the molecular signature comprising one or more inflammatory proteins or biomarkers that are enriched in the subject relative to an uninfected or recovered control subject.

13. The molecular signature of claim 12, wherein the one or more inflammatory proteins or biomarkers is selected from the group consisting of: TNF, IFNLR1, BCAM, S100A16, and IL5.

14. The molecular signature of claim 12 or 13, wherein the one or more inflammatory proteins or biomarkers is selected from the group consisting of: TNF, IL5, IL11, IL13, IL15, IL1B, CXCL1, CXCL8, CCL3, CCL11, IL1RL2, CD28, HLA-DRA, LAG3, and PDCD1.

15. The molecular signature of any one of claims 12 to 14, wherein the one or more inflammatory proteins or biomarkers is selected from the group consisting of: CRH, CRHR1, and PTH1R.

16. The molecular signature of any one of claims 12 to 15, wherein the one or more inflammatory proteins or biomarkers is selected from the group consisting of: AP-1, BACH, BATF, IRF, and STAT.

17. The molecular signature of any one of claims A to 12 to 16, wherein the one or more inflammatory proteins is selected from the group consisting of: type II interferon (IFN), NF-κB, NF-κB-activating cytokine, IL-12, p40, IFN-γ-driven chemokine, TNF-driven cytokine and chemokine, Type I IFN, cytokine, IFNλ, and IL-12.

18. The molecular signature of claim 17, wherein the enrichment of the type II IFN is associated with the enrichment of one or more of Type II IFN-γ, IL-27, and TID.

19. The molecular signature of claim 18, wherein the enrichment of the Type II IFN-γ is associated with the enrichment of one or more of IL-27, IL-18, and NF-κB.

20. The molecular signature of claim 17, wherein the enrichment of the NF-κB is associated with the enrichment of TNF.

21. The molecular signature of claim 20, wherein the enrichment of the TNF is associated with the enrichment of one or more of IL-1 and IL-18.

22. The molecular signature of claim 17, wherein the enrichment of the NF-κB-activating cytokine is associated with the enrichment of one or more of IL-18, TNF, and IL-1.

23. The molecular signature of claim 17, wherein the enrichment of the TNF-driven cytokine and chemokine is associated with the enrichment of one or more of IL-6, CCL7, and MCP3.

24. The molecular signature of claim 17, wherein the enrichment of the Type I IFN is associated with the enrichment of one or more of SAMD9L, MNDA, DDX58, and LAMP3.

25. The molecular signature of claim 17, wherein the enrichment of the cytokine is associated with the enrichment of one or more of IFN-γ, IFN-β, IFN-λ1/2/3, TNF, IL-6, IL-1β, and PTX3.

26. The molecular signature of any one of claims 12 to 17, wherein the molecular signature is a serum proteome signature.

27. The molecular signature of any one of claims 12 to 26, wherein the enrichment is between 1.5-fold and 10-fold as compared to an uninfected or recovered control subject.

28. The molecular signature of any one of claims 12 to 27, wherein the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV).

29. The molecular signature of 28, wherein the virus is SARS-CoV-2.

30. The molecular signature of claim 12, wherein the chronic inflammatory syndrome is post-acute sequelae of SARS-CoV-2 infection (PASC).

31. The molecular signature of claim 30, wherein the subject is likely to have persistent symptoms lasting a specific period after onset of the infection.

32. The molecular signature of claim 31, wherein the specific period is between 30 days and 2 years.

33. A molecular signature for use in diagnosing a subject as having a chronic inflammatory syndrome with or without a chronic or long-term infection with a virus or other pathogen, the molecular signature comprising one or more inflammatory proteins or biomarkers that are enriched in the subject relative to an uninfected or recovered control subject.

34. The molecular signature of claim 33, wherein the one or more inflammatory proteins or biomarkers is selected from the group consisting of: TNF, IFNLR1, BCAM, S100A16, and IL5.

35. The molecular signature of claim 33 or 34, wherein the one or more inflammatory proteins or biomarkers is selected from the group consisting of: TNF, IL5, IL11, IL13, IL15, IL1B, CXCL1, CXCL8, CCL3, CCL11, IL1RL2, CD28, HLA-DRA, LAG3, and PDCD1.

36. The molecular signature of any one of claims 33 to 35, wherein the one or more inflammatory proteins or biomarkers is selected from the group consisting of: CRH, CRHR1, and PTH1R.

37. The molecular signature of any one of claims 33 to 36, wherein the one or more inflammatory proteins or biomarkers is selected from the group consisting of: AP-1, BACH, BATF, IRF, and STAT.

38. The molecular signature of any one of claims A to 33 to 37, wherein the one or more inflammatory proteins is selected from the group consisting of: type II interferon (IFN), NF-κB, NF-κB-activating cytokine, IL-12, p40, IFN-γ-driven chemokine, TNF-driven cytokine and chemokine, Type I IFN, cytokine, IFNA, and IL-12.

39. The molecular signature of claim 38, wherein the enrichment of the type II IFN is associated with the enrichment of one or more of Type II IFN-γ, IL-27, and TID.

40. The molecular signature of claim 39, wherein the enrichment of the Type II IFN-γ is associated with the enrichment of one or more of IL-27, IL-18, and NF-κB.

41. The molecular signature of claim 38, wherein the enrichment of the NF-κB is associated with the enrichment of TNF.

42. The molecular signature of claim 41, wherein the enrichment of the TNF is associated with the enrichment of one or more of IL-1 and IL-18.

43. The molecular signature of claim 38, wherein the enrichment of the NF-κB-activating cytokine is associated with the enrichment of one or more of IL-18, TNF, and IL-1.

44. The molecular signature of claim 38, wherein the enrichment of the TNF-driven cytokine and chemokine is associated with the enrichment of one or more of IL-6, CCL7, and MCP3.

45. The molecular signature of claim 38, wherein the enrichment of the Type I IFN is associated with the enrichment of one or more of SAMD9L, MNDA, DDX58, and LAMP3.

46. The molecular signature of claim 38, wherein the enrichment of the cytokine is associated with the enrichment of one or more of IFN-γ, IFN-β, IFN-λ1/2/3, TNF, IL-6, IL-1β, and PTX3.

47. The molecular signature of any one of claims 33 to 38, wherein the molecular signature is a serum proteome signature.

48. The molecular signature of any one of claims 33 to 47, wherein the enrichment is between 1.5-fold and 10-fold as compared to an uninfected or recovered control subject.

49. The molecular signature of any one of claims 33 to 48, wherein the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV).

50. The molecular signature of 49, wherein the virus is SARS-CoV-2.

51. The molecular signature of claim 33, wherein the chronic inflammatory syndrome is post-acute sequelae of SARS-CoV-2 infection (PASC).

52. The molecular signature of claim 51, wherein the subject is likely to have persistent symptoms lasting a specific period after onset of the infection.

53. The molecular signature of claim 52, wherein the specific period is between 30 days and 2 years.

54. A method of identifying whether a subject infected with or previously infected with a virus or other pathogen is likely or not likely to suffer from a chronic inflammatory syndrome with or without a chronic or long-term infection of the virus or other pathogen, comprising:
(a) determining an expression level of one or more inflammatory proteins or biomarkers of the molecular signature of any one of claims 12 to 27 or 33 to 48 in a first sample obtained from the subject;
(b) comparing the first expression level to a control expression level obtained from an uninfected or recovered control subject; and
(c) classifying the subject as likely to suffer from a chronic or long-term infection of the virus or other pathogen when the expression level corresponds to the molecular signature of any one of claims 12 to 27 or 33 to 48.

55. The method of claim 54, wherein the virus is SARS-CoV-2, SARS-CoV, MERS-CoV, Epstein Barr virus (EBV), Ross River virus (RRV), human immunodeficiency virus (HIV), Ebolavirus, or chikungunya virus (CHIKV).

56. The method of claim 55, wherein the virus is SARS-CoV-2.

57. The method of claim 54, wherein the chronic inflammatory syndrome is post-acute sequelae of SARS-CoV-2 infection (PASC).

58. The method of any one of claims 54 to 57, wherein the sample is obtained within the first 15 days of post-symptom onset.

59. The method of claim 57 or 58, wherein the subject is placed into a cohort for a clinical trial to test investigational drugs to treat PASC.

60. The method of any one of claims 57 to 59, wherein the subject is administered a drug for treating PASC.
